(19) United States
(12) Patent Application Publication
(10) Pub. No.: US 2009/0197856 A1
(43) Pub. Date: Aug. 6, 2009

(54) ANTIVIRAL COMPOUNDS

(75) Inventors: Salvador G. Alvarez, Fremont, CA (US); Janos Botyanszki, Fremont, CA (US); Joseph de los Angeles, Cambridge, MA (US); Jiping Fu, Cambridge, MA (US); Roger Fujimoto, Cambridge, MA (US); Joshua Michael Gralapp, Sunnyvale, CA (US); Ronald Conrad Griffith, Escondido, CA (US); Peichao Lu, Cambridge, MA (US); Son Minh Pham, San Francisco, CA (US); Christopher Don Roberts, Belmont, CA (US); Franz Ulrich Schmitz, Mill Valley, CA (US); Mohindra Seepersaud, Cambridge, MA (US); Ruben Tommasi, Cambridge, MA (US); Adam Christopher Villa, Palo Alto, CA (US); Sompong Wattanasin, Cambridge, MA (US); Aregahagn Yifru, Cambridge, MA (US); Rui Zheng, Cambridge, MA (US); Xiaoling Zheng, Fremont, CA (US)

Correspondence Address: GlaxoSmithKline;c/o Foley & Lardner LLP, 975 Page Mill Road, Palo Alto, CA, 94304-1013, US

(73) Assignees: Genelabs Technologies, Inc.; Novartis AG

(21) Appl. No.: 12/339,460

(22) Filed: Dec. 19, 2008

Related U.S. Application Data
(60) Provisional application No. 61/016,421, filed on Dec. 21, 2007.

(51) Int. Cl. A61K 31/407; C07D 487/14; A61K 31/454; A61K 31/5377; A61K 31/551; A61K 31/5355; A61K 31/496; A61K 31/541; A61K 31/437; A61P 31/12

(52) U.S. Cl. 514/210.01; 540/471; 540/555; 514/322; 514/233.2; 514/219; 514/410; 514/228.8; 514/254.09; 514/228.2; 514/280

(57) ABSTRACT

Provided are compounds of Formula (I) or a pharmaceutically acceptable salt or solvate thereof. The compounds and compositions are useful for treating viral infections caused by the Flaviviridae family of viruses.

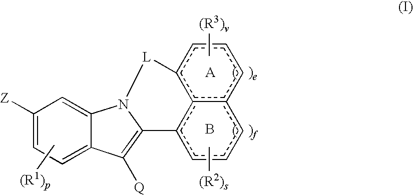

### CROSS-REFERENCE TO RELATED APPLICATION

This application claims the benefit under 35 U.S.C. 119(e) to co-pending provisional application U.S. Ser. No. 61/016,421 filed on Dec. 21, 2007, which is incorporated herein by reference in its entirety.

### BACKGROUND OF THE INVENTION

### Field of the Invention

Compounds and compositions, methods for their preparation, and methods for their use in treating viral infections in patients mediated, at least in part, by a virus in the Flaviviridae family of viruses are disclosed.

### REFERENCES

The following publications are cited in this application as superscript numbers:
1. Szabo, E. et al., Pathol. Oncol. Res. 2003, 9:215-221.
2. Hoofnagle J. H., Hepatology 1997, 26:15 S-20S.
3. Thomson B. J. and Finch R. G., Clin Microbial Infect. 2005, 11:86-94.
4. Moriishi K. and Matsuura Y., Antivir. Chem. Chemother. 2003, 14:285-297.
5. Fried, M. W., et al. N. Engl. J Med 2002, 347:975-982.
6. Ni, Z. J. and Wagman, A. S. Curr. Opin. Drug Discov. Devel. 2004, 7, 446-459.
7. Beaulieu, P. L. and Tsantrizos, Y. S. Curr. Opin. Investig. Drugs 2004, 5, 838-850.
8. Griffith, R. C. et al., Ann. Rep. Med. Chem 39, 223-237, 2004.
9. Watashi, K. et al., Molecular Cell, 19, 111-122, 2005
10. Horsmans, Y. et al., Hepatology, 42, 724-731, 2005

### STATE OF THE ART

Chronic infection with HCV is a major health problem associated with liver cirrhosis, hepatocellular carcinoma, and liver failure. An estimated 170 million chronic carriers worldwide are at risk of developing liver disease.^{1,2} In the United States alone 2.7 million are chronically infected with HCV, and the number of HCV-related deaths in 2000 was estimated between 8,000 and 10,000, a number that is expected to increase significantly over the next years. Infection by HCV is insidious in a high proportion of chronically infected (and infectious) carriers who may not experience clinical symptoms for many years. Liver cirrhosis can ultimately lead to liver failure. Liver failure resulting from chronic HCV infection is now recognized as a leading cause of liver transplantation.

HCV is a member of the Flaviviridae family of RNA viruses that affect animals and humans. The genome is a single ˜9.6-kilobase strand of RNA, and consists of one open reading frame that encodes for a polyprotein of ˜3000 amino acids flanked by untranslated regions at both 5′ and 3′ ends (5′- and 3′-UTR). The polyprotein serves as the precursor to at least 10 separate viral proteins critical for replication and assembly of progeny viral particles. The organization of structural and non-structural proteins in the HCV polyprotein is as follows: C-E1-E2-p7-NS2-NS3-NS4a-NS4b-NS5a-NS5b. Because the replicative cycle of HCV does not involve any DNA intermediate and the virus is not integrated into the host genome, HCV infection can theoretically be cured. While the pathology of HCV infection affects mainly the liver, the virus is found in other cell types in the body including peripheral blood lymphocytes.^{3,4}

At present, the standard treatment for chronic HCV is interferon alpha (IFN-alpha) in combination with ribavirin and this requires at least six (6) months of treatment. IFN-alpha belongs to a family of naturally occurring small proteins with characteristic biological effects such as antiviral, immunoregulatory, and antitumoral activities that are produced and secreted by most animal nucleated cells in response to several diseases, in particular viral infections. IFN-alpha is an important regulator of growth and differentiation affecting cellular communication and immunological control. Treatment of HCV with interferon has frequently been associated with adverse side effects such as fatigue, fever, chills, headache, myalgias, arthralgias, mild alopecia, psychiatric effects and associated disorders, autoimmune phenomena and associated disorders and thyroid dysfunction. Ribavirin, an inhibitor of inosine 5′-monophosphate dehydrogenase (IMPDH), enhances the efficacy of IFN-alpha in the treatment of HCV. Despite the introduction of ribavirin, more than 50% of the patients do not eliminate the virus with the current standard therapy of interferon-alpha (IFN) and ribavirin. By now, standard therapy of chronic hepatitis C has been changed to the combination of pegylated IFN-alpha plus ribavirin. However, a number of patients still have significant side effects, primarily related to ribavirin. Ribavirin causes significant hemolysis in 10-20% of patients treated at currently recommended doses, and the drug is both teratogenic and embryotoxic. Even with recent improvements, a substantial fraction of patients do not respond with a sustained reduction in viral load⁵ and there is a clear need for more effective antiviral therapy of HCV infection.

A number of approaches are being pursued to combat the virus. These include, for example, application of antisense oligonucleotides or ribozymes for inhibiting HCV replication. Furthermore, low-molecular weight compounds that directly inhibit HCV proteins and interfere with viral replication are considered as attractive strategies to control HCV infection. Among the viral targets, the NS3/4a protease/helicase and the NS5b RNA-dependent RNA polymerase are considered the most promising viral targets for new drugs.⁶⁻⁸

Besides targeting viral genes and their transcription and translation products, antiviral activity can also be achieved by targeting host cell proteins that are necessary for viral replication. For example, Watashi et al.⁹ show how antiviral activity can be achieved by inhibiting host cell cyclophilins. Alternatively, a potent TLR7 agonist has been shown to reduce HCV plasma levels in humans.¹⁰

However, none of the compounds described above have progressed beyond clinical trials.^{6,8}

In view of the worldwide epidemic level of HCV and other members of the Flaviviridae family of viruses, and further in view of the limited treatment options, there is a strong need for new effective drugs for treating infections cause by these viruses.

### SUMMARY OF THE INVENTION

In one embodiment, the present invention provides a compound that is Formula (I):

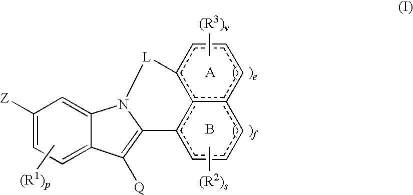

wherein:

ring A and B together contain 1 to 4 ring heteroatoms independently selected from O, N, NR^{b}, S, S(O), and S(O)₂;

[symbol] represents a single or double bond;

e is 0 or 1;

f is 0 or 1;

L is C₂ to C₆ alkylene optionally substituted with (R^{a})ₙ, wherein one —CH₂— group is optionally replaced with —NR^{b}—, >(C═O), —S—, —S(O)—, —S(O)₂—, or —O— and optionally two —CH₂— groups together form a double bond;

R^{a} is selected from the group consisting of halo, amino, substituted amino, acyl, acylamino, aminocarbonyl, alkyl, substituted alkyl, alkenyl, substituted alkenyl, carboxy ester, hydroxyl, alkoxy, substituted alkoxy, oxo, heterocyclyl, and substituted heterocyclyl or two R^{a} attached to a common carbon atom together from a spiro cycloalkyl, substituted cycloalkyl, heterocyclic, or substituted heterocyclic ring;

n is 0, 1, or 2;

R^{b} is independently selected from the group consisting of hydrogen, acyl, aminocarbonyl, alkyl, substituted alkyl, and carboxy ester;

R¹ is selected from the group consisting of alkyl, substituted alkyl, haloalkyl, acyl, acylamino, aminocarbonyl, alkoxy, substituted alkoxy, amino, substituted amino, cyano, halo, and hydroxy;

R² and R³ are independently selected from the group consisting of alkyl, substituted alkyl, alkenyl, substituted alkenyl, alkynyl, substituted alkynyl, amidino, haloalkyl, acyl, acyl-C(O)—, acylamino, aminocarbonyl, alkoxy, substituted alkoxy, amino, substituted amino, aminocarbonylamino, (carboxyl ester)amino, carboxyl, carboxyl ester, cyano, halo, hydroxy, heterocyclyl, substituted heterocyclyl, aryl, substituted aryl, heteroaryl, substituted heteroaryl, and oxo, or two of R² or two of R³ together form a fused or spiro cycloalkyl, substituted cycloalkyl, heterocyclic, or substituted heterocyclic ring or a fused aryl, substituted aryl, heteroaryl, or substituted heteroaryl ring;

p is 0, 1, 2, or 3;

v and s are independently 0, 1, 2, 3, 4, or 5, provided that when ring A is aromatic, at least one of R² or R³ is selected from the group consisting of substituted alkyl, acyl, acyl-C(O)—, aminocarbonyl, acylamino, alkoxy, substituted alkoxy, amino, substituted amino, halo, hydroxy, heterocyclyl, substituted heterocyclyl, aryl, substituted aryl, heteroaryl, substituted heteroaryl, and oxo;

Q is selected from the group consisting of cycloalkyl, substituted cycloalkyl, cycloalkenyl, substituted cycloalkenyl, heterocyclic, and substituted heterocyclic;

Z is selected from the group consisting of
(a) carboxy and carboxy ester;
(b) —C(X⁴)NR¹⁸R¹⁹, wherein X⁴ is ═O, ═NH, or ═N-alkyl, R¹⁸ and R¹⁹ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic or, alternatively, R¹⁸ and R¹⁹ together with the nitrogen atom pendent thereto, form a heterocyclic, a substituted heterocyclic, a heteroaryl or a substituted heteroaryl ring group;
(c) —C(X³)NR²¹S(O)₂R⁴ or —C(X³)NR²¹S(O)R⁴, wherein X³ is selected from ═O, ═NR and ═S, wherein R²⁴ is hydrogen, alkyl, or substituted alkyl; R⁴ is selected from alkyl, substituted alkyl, aryl, substituted aryl, heteroaryl, substituted heteroaryl, heterocyclic, substituted heterocyclic, and NR²²R²³ wherein R²¹, R²², and R²³ are independently hydrogen, alkyl, substituted alkyl, cycloalkyl, or substituted cycloalkyl; or alternatively, R²¹ and R²² or R²² and R²³ together with the atoms bound thereto join together to form an optionally substituted heterocyclic group;
(d) —C(X²)—N(R³¹)CR³²R³³C(═O)R³⁴, wherein X² is selected from ═O, ═S, and ═NR¹¹, where R¹¹ is hydrogen or alkyl, R³⁴ is selected from —OR¹⁷ and —NR¹⁸R¹⁹ where R¹⁷ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R¹⁸ and R¹⁹ are as defined above;
   R³² and R³³ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic;
   or, alternatively, R³² and R³³ as defined are taken together with the carbon atom pendent thereto to form a cycloalkyl, substituted cycloalkyl, heterocyclic or substituted heterocyclic group,
   or, still further alternatively, one of R³² or R³³ is hydrogen, alkyl or substituted alkyl, and the other is joined, together with the carbon atom pendent thereto, with either the R¹⁷ and the oxygen atom pendent thereto or R¹⁸ and the nitrogen atom pendent thereto to form a heterocyclic or substituted heterocyclic group;
   R³¹ is selected from hydrogen and alkyl or, when R³² and R³³ are not taken together to form a ring and when R³² or R³³ and R¹⁷ or R¹⁸ are not joined to form a heterocyclic or substituted heterocyclic group, then R³¹, together with the nitrogen atom pendent thereto, may be taken together with one of R³² and R³³ to form a heterocyclic or substituted heterocyclic ring group;
   (e) —C(X²)—N(R³¹)CR²⁵R²⁶R²⁷, wherein X² and R³¹ are defined above, and R²⁵, R²⁶ and R²⁷ are independently selected from the group consisting of alkyl, substituted alkyl, aryl, substituted aryl, heterocyclic, substituted heterocyclic, heteroaryl and substituted heteroaryl, or R²⁵ and R²⁶ together with the carbon atom pendent thereto form a cycloalkyl, substituted cycloalkyl, heterocyclic or substituted heterocyclic group; and
   (f) a carboxylic acid isostere wherein said isostere is not as defined in (a)-(e).

In one embodiment, the provided is a compound that is Formula (II) or a pharmaceutically acceptable salt thereof:

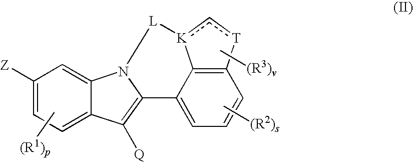

wherein:

Z, Q, L, R^{b}, R¹, R², R³, p, v, s, and [symbol] are previously defined; K is N or C, and

T is selected from the group consisting of N, NR^{b}, CH, CH₂, CHR³, CR³, O, S, S(O), and S(O)₂, wherein at least one of K or T is N or NR^{b}, and when one of [symbol] is a double bond, at least one of R² or R³ is selected from the group consisting of substituted alkyl, acyl, acyl-C(O)—, aminocarbonyl, acylamino, alkoxy, substituted alkoxy, amino, substituted amino, halo, hydroxy, heterocyclyl, substituted heterocyclyl, aryl, substituted aryl, heteroaryl, and substituted heteroaryl or two of R² or two of R³ together form a fused cycloalkyl, substituted cycloalkyl, heterocyclic, substituted heterocyclic, aryl, substituted aryl, heteroaryl, or substituted heteroaryl ring.

In one embodiment, the provided is a compound that is Formula (IIa), or a pharmaceutically acceptable salt thereof:

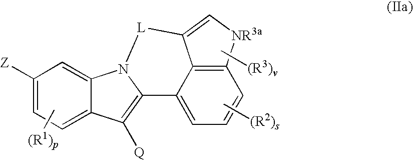

wherein:

Z, Q, L, R¹, R², R³, p, v, and s are previously defined; R^{3a} is H or R³; and at least one of R², R³, or R^{3a} is selected from the group consisting of substituted alkyl, acyl, acyl-C(O)—, aminocarbonyl, acylamino, alkoxy, substituted alkoxy, amino, substituted amino, halo, hydroxy, heterocyclyl, substituted heterocyclyl, aryl, substituted aryl, heteroaryl, substituted heteroaryl, and oxo.

In one embodiment, the provided is a compound that is Formula (IIb) or (IIc) or a pharmaceutically acceptable salt thereof

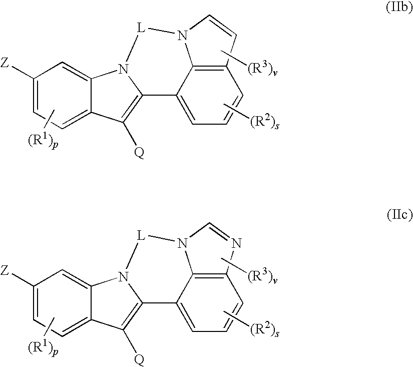

wherein:

Z, Q, L, R¹, R², R³, p, v, and s are previously defined; and at least one of R² or R³, is selected from the group consisting of substituted alkyl, acyl, aminocarbonyl, acylamino, alkoxy, substituted alkoxy, amino, substituted amino, halo, hydroxy, heterocyclyl, substituted heterocyclyl, aryl, substituted aryl, heteroaryl, substituted heteroaryl, and oxo.

In one embodiment, the provided is a compound that is Formula (IId), (IIe), (IIf) or a pharmaceutically acceptable salt thereof

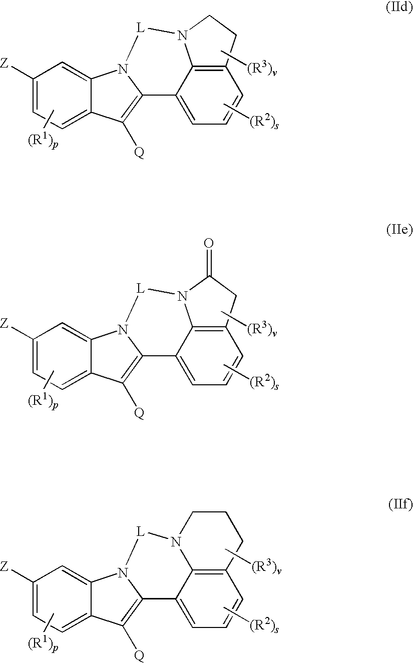

wherein:

Z, Q, L, R¹, R², R³, p, v, and s are previously defined.

In one embodiment, the provided is a compound that is Formula (IIIa)-(IIIc) or a pharmaceutically acceptable salt thereof

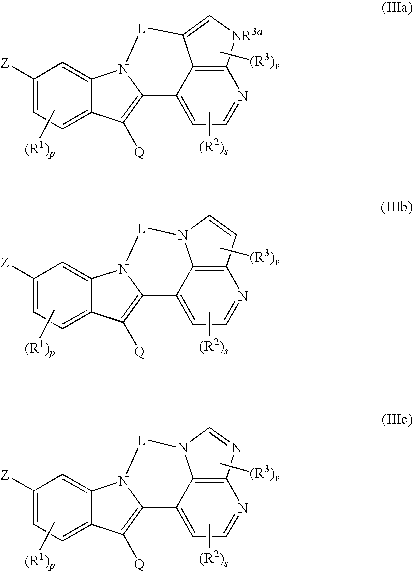

wherein:

Z, Q, L, R¹, R², R³, p, v, and s are previously defined; R^{3a} is H or R³; and at least one of R², R³, or R^{3a} is selected from the group consisting of substituted alkyl, acyl, substituted acyl, alkoxy, substituted alkoxy, amino, substituted amino, halo, hydroxy, heterocyclyl, substituted heterocyclyl, aryl, substituted aryl, heteroaryl, substituted heteroaryl, and oxo.

In one embodiment provided is a pharmaceutical composition comprising a pharmaceutically acceptable carrier and a therapeutically effective amount of a compound of any one of Formulas (I), (II), (IIa)-(IIf) and (IIIa)-(IIIc).

In other embodiments provided are methods for preparing the compounds of any one of Formulas (I), (II), (IIa)-(IIf) and (IIIa)-(IIIc) and compositions thereof and for their therapeutic uses. In one embodiment provided is a method for treating a viral infection in a patient mediated at least in part by a virus in the Flaviviridae family of viruses, comprising administering to said patient a composition comprising a compound or a salt of any one of Formulas (I), (II), (IIa)-(IIf) and (IIIa)-(IIIc). In some aspects, the viral infection is mediated by hepatitis C virus.

These and other embodiments of the invention are further described in the text that follows.

### DETAILED DESCRIPTION OF THE INVENTION

Throughout this application, references are made to various embodiments relating to compounds, compositions, and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

### DEFINITIONS

It is to be understood that the terminology used herein is for the purpose of describing particular embodiments only and is not intended to limit the scope of the present invention. In this specification and in the claims that follow, reference will be made to a number of terms that shall be defined to have the following meanings:

“Alkyl” refers to monovalent linear or branched saturated aliphatic hydrocarbyl groups having from 1 to 10 carbon atoms and, in some embodiments, from 1 to 6 carbon atoms. “C₁₋₆alkyl” refers to alkyl groups having from 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Substituted alkyl” refers to an alkyl group having from 1 to 5 and, in some embodiments, 1 to 3 or 1 to 2 substituents selected from the group consisting of alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, spirocycloalkyl, SO₃H, substituted sulfonyl, sulfonyloxy, thioacyl, thiocyanate, thiol, alkylthio, and substituted alkylthio, wherein said substituents are as defined herein.

“Alkenyl” refers to a linear or branched hydrocarbyl group having from 2 to 10 carbon atoms and in some embodiments from 2 to 6 carbon atoms or 2 to 4 carbon atoms and having at least 1 site of vinyl unsaturation (>C═C<). For example, (Cₓ-C_{y})alkenyl refers to alkenyl groups having from x to y carbon atoms and is meant to include for example, ethenyl, propenyl, 1,3-butadienyl, and the like.

“Substituted alkenyl” refers to alkenyl groups having from 1 to 3 substituents and, in some embodiments, 1 to 2 substituents selected from the group consisting of alkoxy, substituted alkoxy, acyl, acylamino, acyloxy, alkyl, substituted alkyl, alkynyl, substituted alkynyl, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to a vinyl (unsaturated) carbon atom.

“Alkynyl” refers to a linear or branched hydrocarbyl group having from 2 to 10 carbon atoms and in some embodiments from 2 to 6 carbon atoms or 2 to 4 carbon atoms and having at least one triple bond. The term “alkynyl” is also meant to include those hydrocarbyl groups having one triple bond and one double bond. For example, (C₂-C₆)alkynyl is meant to include ethynyl, propynyl, and the like.

“Substituted alkynyl” refers to alkynyl groups having from 1 to 3 substituents and, in some embodiments, from 1 to 2 substituents selected from the group consisting of alkoxy, substituted alkoxy, acyl, acylamino, acyloxy, alkyl, substituted alkyl, alkenyl, substituted alkenyl, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are as defined herein and with the proviso that any hydroxy or thiol substitution is not attached to an acetylenic carbon atom.

“C₂-C₄ alkylene” refers to divalent straight chain alkyl groups having from 1 to 4 carbons.

“C₁-C₅ heteroalkylene” refers to alkylene groups where one or two —CH₂— groups are replaced with —S—, or —O— to give a heteroalkylene having one to five carbons provided that the heteroalkylene does not contain an —O—O—, —S—O—, or —S—S— group. When a —S— group is present, the term “C₁-C₅ heteroalkylene” includes the corresponding oxide metabolites —S(O)— and —S(O)2-. “Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is as defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein. Acyl includes the “acetyl” group CH₃C(O)—.

“Acylamino” refers to the groups —NR⁴⁰C(O)alkyl, —NR⁴⁰C(O)substituted alkyl, —NR⁴⁰C(O)cycloalkyl, —NR⁴⁰C(O)substituted cycloalkyl, —NR⁴⁰C(O)alkenyl, —NR⁴⁰C(O)substituted alkenyl, —NR⁴⁰C(O)alkynyl, —NR⁴⁰C(O)substituted alkynyl, —NR⁴⁰C(O)aryl, —NR⁴⁰C(O)substituted aryl, —NR⁴⁰C(O)heteroaryl, —NR⁴⁰C(O)substituted heteroaryl, —NR⁴⁰C(O)heterocyclic, and —NR⁴⁰C(O)substituted heterocyclic wherein R⁴⁰ is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amino” refers to the group —NH₂.

“Substituted amino” refers to the group —NR⁴¹R⁴² where R⁴¹ and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, and —SO₂-substituted heterocyclic and wherein R⁴¹ and R⁴² are optionally joined together with the nitrogen bound thereto to form a heterocyclic or substituted heterocyclic group, provided that R⁴¹ and R⁴² are both not hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein. When R⁴¹ is hydrogen and R⁴² is alkyl, the substituted amino group is sometimes referred to herein as alkylamino. When R⁴¹ and R⁴² are alkyl, the substituted amino group is sometimes referred to herein as dialkylamino. When referring to a monosubstituted amino, it is meant that either R⁴¹ or R⁴² is hydrogen but not both. When referring to a disubstituted amino, it is meant that neither R⁴¹ nor R⁴² are hydrogen.

“Hydroxyamino” refers to the group —NHOH.

“Alkoxyamino” refers to the group —NHO-alkyl wherein alkyl is defined herein.

“Aminocarbonyl” refers to the group —C(O)NR⁴³R⁴⁴ where R⁴³ and R⁴⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, hydroxy, alkoxy, substituted alkoxy, amino, substituted amino, and acylamino, and where R⁴³ and R⁴⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminothiocarbonyl” refers to the group —C(S)NR⁴³R⁴⁴ where R⁴³ and R⁴⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R⁴³ and R⁴⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminocarbonylamino” refers to the group —NR⁴⁰C(O)NR⁴³R⁴⁴ where R⁴⁰ is hydrogen or alkyl and R⁴³ and R⁴⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R⁴³ and R⁴⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminothiocarbonylamino” refers to the group —NR⁴⁰C(S)NR⁴³R⁴⁴ where R⁴⁰ is hydrogen or alkyl and R⁴³ and R⁴⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R⁴³ and R⁴⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminocarbonyloxy” refers to the group —O—C(O)NR⁴³R⁴⁴ where R⁴³ and R⁴⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R⁴³ and R⁴⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonyl” refers to the group —SO₂NR⁴³R⁴⁴ where R⁴³ and R⁴⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R⁴³ and R⁴⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonyloxy” refers to the group —O—SO₂NR⁴³R⁴⁴ where R⁴³ and R⁴⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R⁴³ and R⁴⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonylamino” refers to the group —NR⁴⁰—SO₂NR⁴³R⁴⁴ where R⁴⁰ is hydrogen or alkyl and R⁴³ and R⁴⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R⁴³ and R⁴⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amidino” refers to the group —C(═NR⁴⁵)NR⁴³R⁴⁴ where R⁴⁵, R⁴³, and R⁴⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R⁴³ and R⁴⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aryl” or “Ar” refers to an aromatic group of from 6 to 14 carbon atoms and no ring heteroatoms and having a single ring (e.g., phenyl) or multiple condensed (fused) rings (e.g., naphthyl or anthryl). For multiple ring systems, including fused, bridged, and spiro ring systems having aromatic and non-aromatic rings that have no ring heteroatoms, the term “Aryl” or “Ar” applies when the point of attachment is at an aromatic carbon atom (e.g., 5,6,7,8 tetrahydronaphthalene-2-yl is an aryl group as its point of attachment is at the 2-position of the aromatic phenyl ring).

“Substituted aryl” refers to aryl groups which are substituted with 1 to 8 and, in some embodiments, 1 to 5, 1 to 3, or 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are defined herein.

“Aryloxy” refers to the group —O-aryl, where aryl is as defined herein, that includes, by way of example, phenoxy and naphthyloxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Azido” refers to the group —N₃.

“Hydrazino” refers to the group —NHNH₂.

“Substituted hydrazino” refers to the group —NR⁴⁶NR⁴⁷R⁴⁸ where R⁴, R⁴⁷, and R⁴⁸ are independently selected from the group consisting of hydrogen, alkyl, substituted alkyl, alkenyl, substituted alkenyl, alkynyl, substituted alkynyl, aryl, substituted aryl, carboxyl ester, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, and —SO₂-substituted heterocyclic and wherein R⁴⁷ and R⁴⁸ are optionally joined, together with the nitrogen bound thereto to form a heterocyclic or substituted heterocyclic group, provided that R⁴⁷ and R⁴⁸ are both not hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cyano” or “carbonitrile” refers to the group —CN.

“Carbonyl” refers to the divalent group —C(O)— which is equivalent to —C(═O)—.

“Carboxyl” or “carboxy” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)amino” refers to the group —NR⁴⁰—C(O)O-alkyl, —NR⁴⁰—C(O)O-substituted alkyl, —NR⁴⁰—C(O)O-alkenyl, —NR⁴⁰—C(O)O-substituted alkenyl, —NR⁴⁰—C(O)O-alkynyl, —NR⁴⁰—C(O)O-substituted alkynyl, —NR⁴⁰—C(O)O-aryl, —NR⁴⁰—C(O)O-substituted aryl, —NR⁴⁰—C(O)O-cycloalkyl, —NR⁴⁰—C(O)O-substituted cycloalkyl, —NR⁴⁰—C(O)O-heteroaryl, —NR⁴⁰—C(O)O-substituted heteroaryl, —NR⁴⁰—C(O)O-heterocyclic, and —NR⁴⁰—C(O)O-substituted heterocyclic wherein R⁴⁰ is alkyl or hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cycloalkyl” refers to a saturated or partially saturated cyclic group of from 3 to 14 carbon atoms and no ring heteroatoms and having a single ring or multiple rings including fused, bridged, and spiro ring systems. For multiple ring systems having aromatic and non-aromatic rings that have no ring heteroatoms, the term “cycloalkyl” applies when the point of attachment is at a non-aromatic carbon atom (e.g. 5,6,7,8,-tetrahydronaphthalene-5-yl). The term “Cycloalkyl” includes cycloalkenyl groups but does not include aromatic rings. Examples of cycloalkyl groups include, for instance, adamantyl, cyclopropyl, cyclobutyl, cyclopentyl, cyclooctyl, and cyclohexenyl. “Cᵤ₋ᵥcycloalkyl” refers to cycloalkyl groups having u to v carbon atoms.

“Cycloalkenyl” refers to a partially saturated cycloalkyl ring having at least one site of >C═C< ring unsaturation. Cycloalkenyl does not include aromatic rings.

“Substituted cycloalkyl” refers to a cycloalkyl group, as defined herein, having from 1 to 8, or 1 to 5, or in some embodiments 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are as defined herein. The term “substituted cycloalkyl” includes substituted cycloalkenyl groups.

“Cycloalkyloxy” refers to —O-cycloalkyl wherein cycloalkyl is as defined herein.

“Substituted cycloalkyloxy” refers to —O-(substituted cycloalkyl) wherein substituted cycloalkyl is as defined herein.

“Cycloalkylthio” refers to —S-cycloalkyl wherein cycloalkyl is as defined herein.

“Substituted cycloalkylthio” refers to —S-(substituted cycloalkyl).

“Guanidino” refers to the group —NHC(═NH)NH₂.

“Substituted guanidino” refers to —NR⁴⁹C(═NR⁴⁹)N(R⁴⁹)₂ where each R⁴⁹ is independently selected from the group consisting of hydrogen, alkyl, substituted alkyl, aryl, substituted aryl, heteroaryl, substituted heteroaryl, heterocyclyl, and substituted heterocyclyl and two R⁴⁹ groups attached to a common guanidino nitrogen atom are optionally joined together with the nitrogen bound thereto to form a heterocyclic or substituted heterocyclic group, provided that at least one R⁴⁹ is not hydrogen, and wherein said substituents are as defined herein.

“Halo” or “halogen” refers to fluoro, chloro, bromo, and iodo.

“Haloalkyl” refers to substitution of alkyl groups with 1 to 5 or in some embodiments 1 to 3 halo groups. Haloalkyl groups include —CF₃.

“Hydroxy” or “hydroxyl” refers to the group —OH.

“Heteroaryl” refers to an aromatic group of from 1 to 14 carbon atoms and 1 to 6 heteroatoms selected from the group consisting of oxygen, nitrogen, and sulfur and includes single ring (e.g. imidazolyl) and multiple ring systems (e.g. benzimidazol-2-yl and benzimidazol-6-yl). For multiple ring systems, including fused, bridged, and spiro ring systems having aromatic and non-aromatic rings, the term “heteroaryl” applies if there is at least one ring heteroatom and the point of attachment is at an atom of an aromatic ring (e.g. 1,2,3,4-tetrahydroquinolin-6-yl and 5,6,7,8-tetrahydroquinolin-3-yl). In one embodiment, the nitrogen and/or the sulfur ring atom(s) of the heteroaryl group are optionally oxidized to provide for the N-oxide (N→O), sulfinyl, or sulfonyl moieties. More specifically the term heteroaryl includes, but is not limited to, pyridyl, furanyl, thienyl, thiazolyl, isothiazolyl, triazolyl, imidazolyl, isoxazolyl, pyrrolyl, pyrazolyl, pyridazinyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, quinazolinonyl, benzimidazolyl, benzisoxazolyl, or benzothienyl.

“Substituted heteroaryl” refers to heteroaryl groups that are substituted with from 1 to 8 or in some embodiments 1 to 5, or 1 to 3, or 1 to 2 substituents selected from the group consisting of the substituents defined for substituted aryl.

“Heteroaryloxy” refers to —O-heteroaryl wherein heteroaryl is as defined herein.

“Substituted heteroaryloxy refers to the group —O-(substituted heteroaryl) wherein substituted heteroaryl is as defined herein.

“Heteroarylthio” refers to the group —S-heteroaryl wherein heteroaryl is as defined herein.

“Substituted heteroarylthio” refers to the group —S-(substituted heteroaryl) wherein substituted heteroaryl is as defined herein.

“Heterocyclic” or “heterocycle” or “heterocycloalkyl” or “heterocyclyl” refers to a saturated or partially saturated and not aromatic cyclic group having from 1 to 14 carbon atoms and from 1 to 6 heteroatoms selected from the group consisting of nitrogen, sulfur, or oxygen and includes single ring and multiple ring systems including fused, bridged, and spiro ring systems. For multiple ring systems having aromatic and/or non-aromatic rings, the terms “heterocyclic”, “heterocycle”, “heterocycloalkyl”, or “heterocyclyl” apply when there is at least one ring heteroatom and the point of attachment is at an atom of a non-aromatic ring (e.g. 1,2,3,4-tetrahydroquinoline-3-yl, 5,6,7,8-tetrahydroquinoline-6-yl, and decahydroquinolin-6-yl). In one embodiment, the nitrogen and/or sulfur atom(s) of the heterocyclic group are optionally oxidized to provide for the N-oxide, sulfinyl, sulfonyl moieties. More specifically the heterocyclyl includes, but is not limited to, tetrahydropyranyl, piperidinyl, N-methylpiperidin-3-yl, piperazinyl, N-methylpyrrolidin-3-yl, 3-pyrrolidinyl, 2-pyrrolidon-1-yl, morpholinyl, and pyrrolidinyl. A prefix indicating the number of carbon atoms (e.g., C₃-C₁₀) refers to the total number of carbon atoms in the portion of the heterocyclyl group exclusive of the number of heteroatoms.

“Substituted heterocyclic” or “Substituted heterocycle” or “substituted heterocycloalkyl” or “substituted heterocyclyl” refers to heterocyclic groups, as defined herein, that are substituted with from 1 to 5 or in some embodiments 1 to 3 of the substituents as defined for substituted cycloalkyl.

“Heterocyclyloxy” refers to the group —O-heterocycyl wherein heterocyclyl is as defined herein.

“Substituted heterocyclyloxy” refers to the group —O-(substituted heterocycyl) wherein substituted heterocyclyl is as defined herein.

“Heterocyclylthio” refers to the group —S-heterocycyl wherein heterocyclyl is as defined herein.

“Substituted heterocyclylthio” refers to the group —S-(substituted heterocycyl) wherein substituted heterocyclyl is as defined herein.

Examples of heterocycle and heteroaryl groups include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl. Other examples of heterocycles and heteroaryls include oxindole, isoquinoline, tetrahydroquinoline, and tetrahydroisoquinoline.

“Nitro” refers to the group —NO₂.

“Oxo” refers to the atom (═O).

“Oxide” refers to products resulting from the oxidation of one or more heteroatoms. Examples include N-oxides, sulfoxides, and sulfones.

“Spirocycloalkyl” refers to a 3 to 10 member cyclic substituent formed by replacement of two hydrogen atoms at a common carbon atom with an alkylene group having 2 to 9 carbon atoms, as exemplified by the following structure wherein the methylene group shown here attached to bonds marked with wavy lines is substituted with a spirocycloalkyl group:

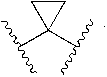

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-alkynyl, —SO₂-substituted alkynyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein. Substituted sulfonyl includes groups such as methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cylcoalkyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thiol” refers to the group —SH.

“Alkylthio” refers to the group —S-alkyl wherein alkyl is as defined herein.

“Substituted alkylthio” refers to the group —S-(substituted alkyl) wherein substituted alkyl is as defined herein.

“Thiocarbonyl” refers to the divalent group —C(S)— which is equivalent to —C(═S)—.

“Thione” refers to the atom (═S).

“Thiocyanate” refers to the group —SCN.

“Compound” and “compounds” as used herein refers to a compound encompassed by the generic formulae disclosed herein, any subgenus of those generic formulae, and any forms of the compounds within the generic and subgeneric formulae, including the isotopes, racemates, stereoisomers, and tautomers of the compound or compounds.

“Isotopes” refer to pharmaceutically acceptable isotopically-labeled compounds wherein (1) one or more atoms are replaced by atoms having the same atomic number, but an atomic mass or mass number different from the atomic mass or mass number usually found in nature, and/or (2) the isotopic ratio of one or more atoms is different from the naturally occurring ratio.

Examples of isotopes suitable for inclusion in the compounds of the invention comprises isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labeled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

In one embodiment, the one or two hydrogen atoms of substituent Q is replaced with deutero atoms.

“Racemates” refers to a mixture of enantiomers.

“Solvate” or “solvates” of a compound refer to those compounds, where compounds is as defined above, that are bound to a stoichiometric or non-stoichiometric amount of a solvent. Solvates of a compound includes solvates of all forms of the compound. Preferred solvents are volatile, non-toxic, and/or acceptable for administration to humans in trace amounts. Suitable solvates include water.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Isosteres” are different compounds that have different molecular formulae but exhibit the same or similar properties. For example, tetrazole is an isostere of carboxylic acid because it mimics the properties of carboxylic acid even though they both have very different molecular formulae. Tetrazole is one of many possible isosteric replacements for carboxylic acid. Other carboxylic acid isosteres contemplated by the present invention include —COOH, —SO₃H, —SO₂HNR^{k′}, —PO₂(R^{k′})₂, —CN, —PO₃(R^{k′})₂, —OR^{k}, —SR^{k′}, —NHCOR^{k′}, —N(R^{k′})₂, —CON(R^{k′})₂, —CONH(O)R^{k′}, —CONHNHSO₂R^{k′}, —COHNSO₂R^{k′}, and —CONR^{k′}CN, where R^{k′} is selected from hydrogen, hydroxy, halo, haloalkyl, thiocarbonyl, alkoxy, alkenoxy, alkylaryloxy, aryloxy, arylalkyloxy, cyano, nitro, imino, alkylamino, aminoalkyl, thiol, thioalkyl, alkylthio, sulfonyl, alkyl, alkenyl or alkynyl, aryl, aralkyl, cycloalkyl, heteroaryl, heterocycle, and CO₂R^{m′} where R^{m′} is hydrogen alkyl or alkenyl. In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of preferred carboxylic acid isosteres contemplated by this invention.

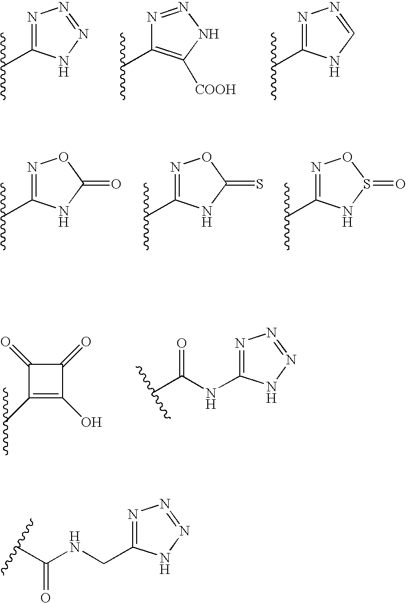

“Carboxylic acid bioisosteres” are compounds that behave as isosteres of carboxylic acids under biological conditions.

Other carboxylic acid isosteres not specifically exemplified or described in this specification are also contemplated by the present invention.

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

“Patient” refers to mammals and includes humans and non-human mammals.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

Unless indicated otherwise, the nomenclature of substituents that are not explicitly defined herein are arrived at by naming the terminal portion of the functionality followed by the adjacent functionality toward the point of attachment. For example, the substituent “arylalkyloxycabonyl” refers to the group (aryl)-(alkyl)-O—C(O)—.

It is understood that in all substituted groups defined above, polymers arrived at by defining substituents with further substituents to themselves are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

Accordingly in one embodiment, provided is a compound that is Formula (I):

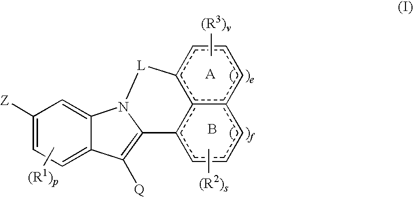

wherein:

ring A and B together contain 1 to 4 ring heteroatoms independently selected from O, N, NR^{b}, S, S(O), and S(O)₂;

[symbol] represents a single or double bond;

e is 0 or 1;

f is 0 or 1;

L is C₂ to C₆ alkylene optionally substituted with (R^{a})ₙ, wherein one —CH₂— group is optionally replaced with —NR^{b}—, >(C═O), —S—, —S(O)—, —S(O)₂—, or —O— and optionally two —CH₂— groups together form a double bond;

R^{a} is selected from the group consisting of halo, amino, substituted amino, acyl, acylamino, aminocarbonyl, alkyl, substituted alkyl, alkenyl, substituted alkenyl, carboxy ester, hydroxyl, alkoxy, substituted alkoxy, oxo, heterocyclyl, and substituted heterocyclyl or two R^{a} attached to a common carbon atom together from a spiro cycloalkyl, substituted cycloalkyl, heterocyclic, or substituted heterocyclic ring;

n is 0, 1, or 2;

R^{b} is independently selected from the group consisting of hydrogen, acyl, aminocarbonyl, alkyl, substituted alkyl, and carboxy ester;

R¹ is selected from the group consisting of alkyl, substituted alkyl, haloalkyl, acyl, acylamino, aminocarbonyl, alkoxy, substituted alkoxy, amino, substituted amino, cyano, halo, and hydroxy;

R² and R³ are independently selected from the group consisting of alkyl, substituted alkyl, alkenyl, substituted alkenyl, alkynyl, substituted alkynyl, amidino, haloalkyl, acyl, acyl-C(O)—, acylamino, aminocarbonyl, alkoxy, substituted alkoxy, amino, substituted amino, aminocarbonylamino, (carboxyl ester)amino, carboxyl, carboxyl ester, cyano, halo, hydroxy, heterocyclyl, substituted heterocyclyl, aryl, substituted aryl, heteroaryl, substituted heteroaryl, and oxo, or two of R² or two of R³ together form a fused or spiro cycloalkyl, substituted cycloalkyl, heterocyclic, or substituted heterocyclic ring or a fused aryl, substituted aryl, heteroaryl, or substituted heteroaryl ring;

p is 0, 1, 2, or 3;

v and s are independently 0, 1, 2, 3, 4, or 5, provided that when ring A is aromatic, at least one of R² or R³ is selected from the group consisting of substituted alkyl, acyl, acyl-C(O)—, aminocarbonyl, acylamino, alkoxy, substituted alkoxy, amino, substituted amino, halo, hydroxy, heterocyclyl, substituted heterocyclyl, aryl, substituted aryl, heteroaryl, and substituted heteroaryl;

Q is selected from the group consisting of cycloalkyl, substituted cycloalkyl, cycloalkenyl, substituted cycloalkenyl, heterocyclic, and substituted heterocyclic;

Z is selected from the group consisting of
(a) carboxy and carboxy ester;
(b) —C(X⁴)NR¹⁸R¹⁹, wherein X⁴ is ═O, ═NH, or ═N-alkyl, R¹⁸ and R¹⁹ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic or, alternatively, R¹⁸ and R¹⁹ together with the nitrogen atom pendent thereto, form a heterocyclic, a substituted heterocyclic, a heteroaryl or a substituted heteroaryl ring group;
(c) —C(X³)NR²¹S(O)₂R⁴ or —C(X³)NR²¹S(O)R⁴, wherein X³ is selected from ═O, ═NR²⁴, and ═S, wherein R²⁴ is hydrogen, alkyl, or substituted alkyl; R⁴ is selected from alkyl, substituted alkyl, aryl, substituted aryl, heteroaryl, substituted heteroaryl, heterocyclic, substituted heterocyclic, and NR²²R²³ wherein R²¹, R²², and R²³ are independently hydrogen, alkyl, substituted alkyl, cycloalkyl, or substituted cycloalkyl; or alternatively, R²¹ and R²² or R²² and R²³ together with the atoms bound thereto join together to form an optionally substituted heterocyclic group;
(d) —C(X²)—N(R³¹)CR³²R³³C(═O)R³⁴, wherein X² is selected from ═O, ═S, and ═NR¹¹, where R¹¹ is hydrogen or alkyl, R³⁴ is selected from —OR¹⁷ and —NR¹⁸R¹⁹ where R¹⁷ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R¹⁸ and R¹⁹ are as defined above;
   R³² and R³³ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic;
   or, alternatively, R³² and R³³ as defined are taken together with the carbon atom pendent thereto to form a cycloalkyl, substituted cycloalkyl, heterocyclic or substituted heterocyclic group,
   or, still further alternatively, one of R³² or R³³ is hydrogen, alkyl or substituted alkyl, and the other is joined, together with the carbon atom pendent thereto, with either the R¹⁷ and the oxygen atom pendent thereto or R¹⁸ and the nitrogen atom pendent thereto to form a heterocyclic or substituted heterocyclic group;
   R³¹ is selected from hydrogen and alkyl or, when R³² and R³³ are not taken together to form a ring and when R³² or R³³ and R¹⁷ or R¹⁸ are not joined to form a heterocyclic or substituted heterocyclic group, then R³¹, together with the nitrogen atom pendent thereto, may be taken together with one of R³² and R³³ to form a heterocyclic or substituted heterocyclic ring group;
(e) —C(X²)—N(R³¹)CR²⁵R²⁶R²⁷, wherein X² and R³¹ are defined above, and R²⁵, R²⁶ and R²⁷ are independently selected from the group consisting of alkyl, substituted alkyl, aryl, substituted aryl, heterocyclic, substituted heterocyclic, heteroaryl and substituted heteroaryl, or R²⁵ and R²⁶ together with the carbon atom pendent thereto form a cycloalkyl, substituted cycloalkyl, heterocyclic or substituted heterocyclic group; and
(f) a carboxylic acid isostere wherein said isostere is not as defined in (a)-(e).

In one embodiment, provided is a compound of Formula (I) wherein L is C₂ to C₄ alkylene optionally substituted with R^{a}, wherein one —CH₂— group is optionally replaced with —NR^{b}—, >(C═O), —S—, or —O— and optionally two —CH₂— groups together form a double bond;

R^{a} is selected from the group consisting of halo, amino, substituted amino, acyl, acylamino, aminocarbonyl, alkyl, substituted alkyl, carboxy ester, hydroxyl, alkoxy, substituted alkoxy, heterocyclyl, and substituted heterocyclyl;

R^{b} is independently selected from the group consisting of hydrogen, acyl, aminocarbonyl, alkyl, substituted alkyl, (carboxyl ester) amino, and carboxy ester;

R¹ is selected from the group consisting of alkyl, substituted alkyl, alkoxy, substituted alkoxy, amino, substituted amino, halo, and hydroxy;

R² and R³ are independently selected from the group consisting of alkyl, substituted alkyl, acyl, acyl-C(O)—, acylamino, aminocarbonyl, alkoxy, substituted alkoxy, amino, substituted amino, halo, hydroxy, heterocyclyl, substituted heterocyclyl, aryl, substituted aryl, heteroaryl, substituted heteroaryl, and oxo;

p, v, and s are independently 0, 1, 2, or 3, provided that at least one of R² or R³ is selected from the group consisting of substituted alkyl, acyl, acyl-C(O)—, aminocarbonyl, acylamino, alkoxy, substituted alkoxy, amino, substituted amino, halo, hydroxy, heterocyclyl, substituted heterocyclyl, aryl, substituted aryl, heteroaryl, substituted heteroaryl, and oxo;

Q is selected from the group consisting of cycloalkyl, substituted cycloalkyl, cycloalkenyl, substituted cycloalkenyl, heterocyclic, and substituted heterocyclic;

Z is selected from the group consisting of
(a) carboxy and carboxy ester;
(b) —C(X⁴)NR¹⁸R¹⁹, wherein X⁴ is ═O, ═NH, or ═N-alkyl, R¹⁸ and R¹⁹ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic or, alternatively, R¹⁸ and R¹⁹ together with the nitrogen atom pendent thereto, form a heterocyclic, a substituted heterocyclic, a heteroaryl or a substituted heteroaryl ring group;
(c) —C(X³)NR²¹S(O)₂R⁴, wherein X³ is selected from ═O, ═NR²⁴, and ═S, wherein R²⁴ is hydrogen, alkyl, or substituted alkyl; R⁴ is selected from alkyl, substituted alkyl, aryl, substituted aryl, heteroaryl, substituted heteroaryl, heterocyclic, substituted heterocyclic, and NR²²R²³ wherein R²¹, R²², and R²³ are independently hydrogen, alkyl, substituted alkyl, cycloalkyl, or substituted cycloalkyl; or alternatively, R²¹ and R²² or R²² and R²³ together with the atoms bound thereto join together to form an optionally substituted heterocyclic group;
(d) —C(X²)—N(R³¹)CR³²R³³C(═O)R³⁴, wherein X² is selected from ═O, ═S, and ═NR¹¹, where R¹¹ is hydrogen or alkyl, R³⁴ is selected from —OR¹⁷ and —NR¹⁸R¹⁹ where R¹⁷ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R¹⁸ and R¹⁹ are as defined above;
   R³² and R³³ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic;
   or, alternatively, R³² and R³³ as defined are taken together with the carbon atom pendent thereto to form a cycloalkyl, substituted cycloalkyl, heterocyclic or substituted heterocyclic group,
   or, still further alternatively, one of R³² or R³³ is hydrogen, alkyl or substituted alkyl, and the other is joined, together with the carbon atom pendent thereto, with either the R¹⁷ and the oxygen atom pendent thereto or R¹⁸ and the nitrogen atom pendent thereto to form a heterocyclic or substituted heterocyclic group;
   R³¹ is selected from hydrogen and alkyl or, when R³² and R³³ are not taken together to form a ring and when R³² or R³³ and R¹⁷ or R¹⁸ are not joined to form a heterocyclic or substituted heterocyclic group, then R³¹, together with the nitrogen atom pendent thereto, may be taken together with one of R³² and R³³ to form a heterocyclic or substituted heterocyclic ring group;
(e) —C(X²)—N(R³¹)CR²⁵R²⁶R²⁷, wherein X² and R³¹ are defined above, and R²⁵, R²⁶ and R²⁷ are independently selected from the group consisting of alkyl, substituted alkyl, aryl, substituted aryl, heterocyclic, substituted heterocyclic, heteroaryl and substituted heteroaryl, or R²⁵ and R²⁶ together with the carbon atom pendent thereto form a cycloalkyl, substituted cycloalkyl, heterocyclic or substituted heterocyclic group; and
(f) a carboxylic acid isostere wherein said isostere is not as defined in (a)-(e).

In one embodiment, the provided is a compound that is Formula (II) or a pharmaceutically acceptable salt thereof:

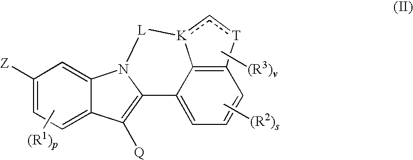

wherein:

Z, Q, L, R^{b}, R¹, R², R³, p, v, s, and [symbol] are previously defined; K is N or C, and

T is selected from the group consisting of N, NR^{b}, CH, CH₂, CHR³, CR³, O, S, S(O), and S(O)₂, wherein at least one of K or T is N or NR^{b}, and when one of [symbol] is a double bond, at least one of R² or R³ is selected from the group consisting of substituted alkyl, acyl, acyl-C(O)—, aminocarbonyl, acylamino, alkoxy, substituted alkoxy, amino, substituted amino, halo, hydroxy, heterocyclyl, substituted heterocyclyl, aryl, substituted aryl, heteroaryl, and substituted heteroaryl or two of R² or two of R³ together form a fused cycloalkyl, substituted cycloalkyl, heterocyclic, substituted heterocyclic, aryl, substituted aryl, heteroaryl, or substituted heteroaryl ring.

In one embodiment, the provided is a compound that is Formula (IIa), or a pharmaceutically acceptable salt thereof:

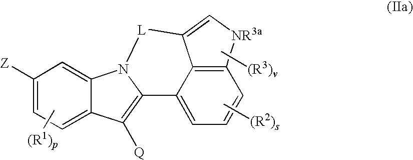

wherein:

Z, Q, L, R¹, R², R³, p, v, and s are previously defined; R^{3a} is H or R³; and at least one of R², R³, or R^{3a} is selected from the group consisting of substituted alkyl, acyl, acyl-C(O)—, aminocarbonyl, acylamino, alkoxy, substituted alkoxy, amino, substituted amino, halo, hydroxy, heterocyclyl, substituted heterocyclyl, aryl, substituted aryl, heteroaryl, and substituted heteroaryl.

In one embodiment, the provided is a compound that is Formula (IIb) or (IIc) or a pharmaceutically acceptable salt thereof

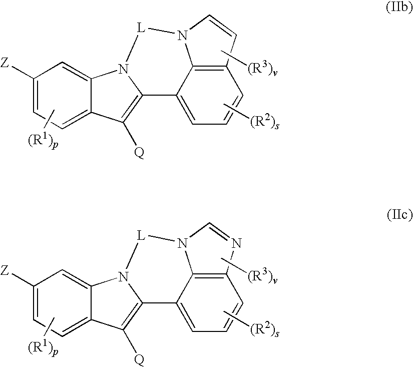

wherein:

Z, Q, L, R¹, R², R³, p, v, and s are previously defined; and at least one of R² or R³, is selected from the group consisting of substituted alkyl, acyl, aminocarbonyl, acylamino, alkoxy, substituted alkoxy, amino, substituted amino, halo, hydroxy, heterocyclyl, substituted heterocyclyl, aryl, substituted aryl, heteroaryl, and substituted heteroaryl.

In one embodiment, the provided is a compound that is Formula (IId), (IIe), or (IIf) or a pharmaceutically acceptable salt thereof

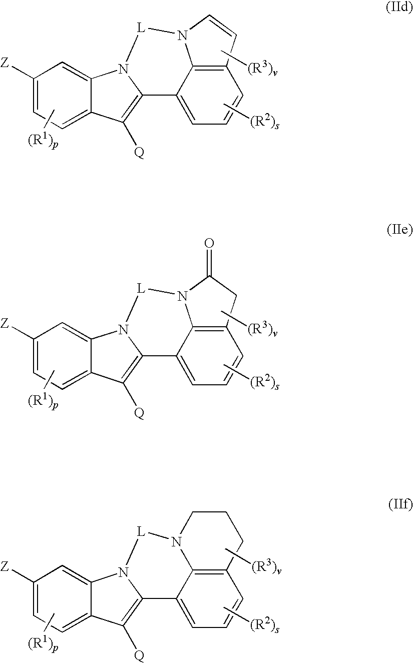

wherein:

Z, Q, L, R¹, R², R³, p, v, and s are previously defined.

In one embodiment, the provided is a compound that is Formula (IIIa)-(IIIc) or a pharmaceutically acceptable salt thereof

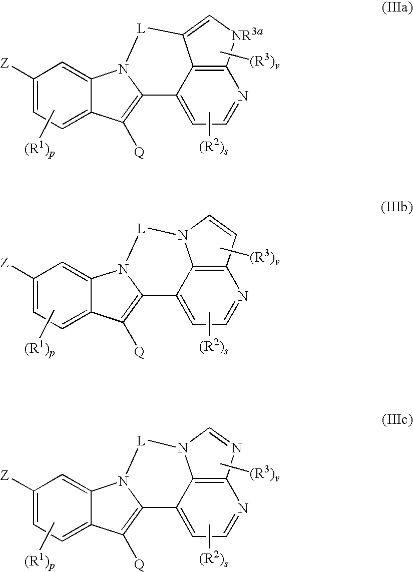

wherein:

Z, Q, L, R¹, R², R², p, v, and s are previously defined; R^{3a} is H or R³; and at least one of R², R³, or R^{3a} is selected from the group consisting of substituted alkyl, acyl, substituted acyl, alkoxy, substituted alkoxy, amino, substituted amino, halo, hydroxy, heterocyclyl, substituted heterocyclyl, aryl, substituted aryl, heteroaryl, and substituted heteroaryl.

In one embodiment, provided is a compound that is a pharmaceutically acceptable salt of any one of Formula (I), (II), (IIa)-(IIf), or (IIIa)-(IIIc).

In one embodiment, provided is a compound that is a solvate of any one of Formula (I), (II), (IIa)-(IIf), or (IIIa)-(IIIc). In some aspects, the solvate is a solvate of a pharmaceutically acceptable salt of any one of Formula (I), (II), (IIa)-(IIf), or (IIIa)-(IIIc).

Various features relating to the embodiments above are given below. These features when referring to different substituents or variables can be combined with each other or with any other embodiments described in this application. In some aspects, provided are compounds of Formula (I), (II), (IIa)-(IIf), or (IIIa)-(IIIc) having one or more of the following features below.

In some embodiments, v is 0 or 1; 0, 1, or 2; 0, 1, 2, or 3; or 0, 1, 2, 3, or 4.

In some embodiments, s is 0 or 1; 0, 1, or 2; or 0, 1, 2, or 3.

In some embodiments, L is —CH₂(CH₂)ₙCH₂— where n is 0, 1 or 2.

In some embodiments, L is C₂ to C₄ alkylene optionally substituted with R^{a}, wherein one —CH₂— group is —NR^{b}—.

In some embodiments, R^{b} is selected from the group consisting of

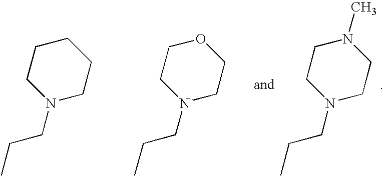

In some embodiments, L is substituted with R^{a}, and R^{a} is selected from the group consisting of substituted alkyl, amino, substituted amino, heterocyclyl, hydroxy, and substituted alkoxy. In some embodiment, R^{a} is aminocarbonyl.

In some embodiments, R^{a} is selected from the group consisting of:

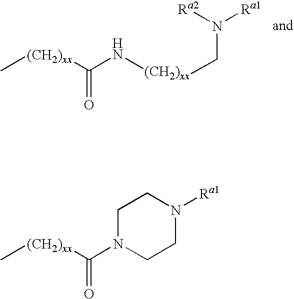

where each xx is independently 0, 1, 2, 3, or 4; and

R^{a1} and R^{a2} are independently selected from the group consisting of hydrogen, alkyl, substituted alkyl, acyl, sulfonyl and substituted sulfonyl.

In some embodiments, R^{a} is selected from the group consisting of:

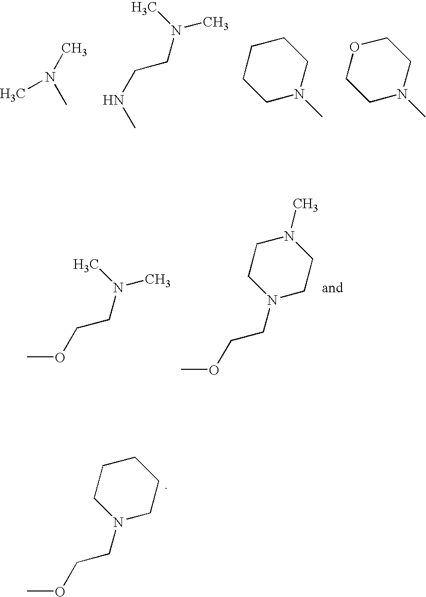

In some embodiments, at least one of R² or R³ is selected from the group consisting of substituted alkyl, acyl, acyl-C(O)—, alkoxy, substituted alkoxy, amino, substituted amino, halo, hydroxy, and oxo.

In some embodiments, R³ is selected from the group consisting of substituted alkyl, amino, substituted amino, acyl, acyl-C(O)—, heterocyclyl, hydroxy, and substituted alkoxy.

In some embodiments two R³ attached to a common carbon atom together form a spiro cycloalkyl, substituted cycloalkyl, heterocyclic, or substituted heterocyclic ring.

In some embodiments, R³ is selected from the group consisting of

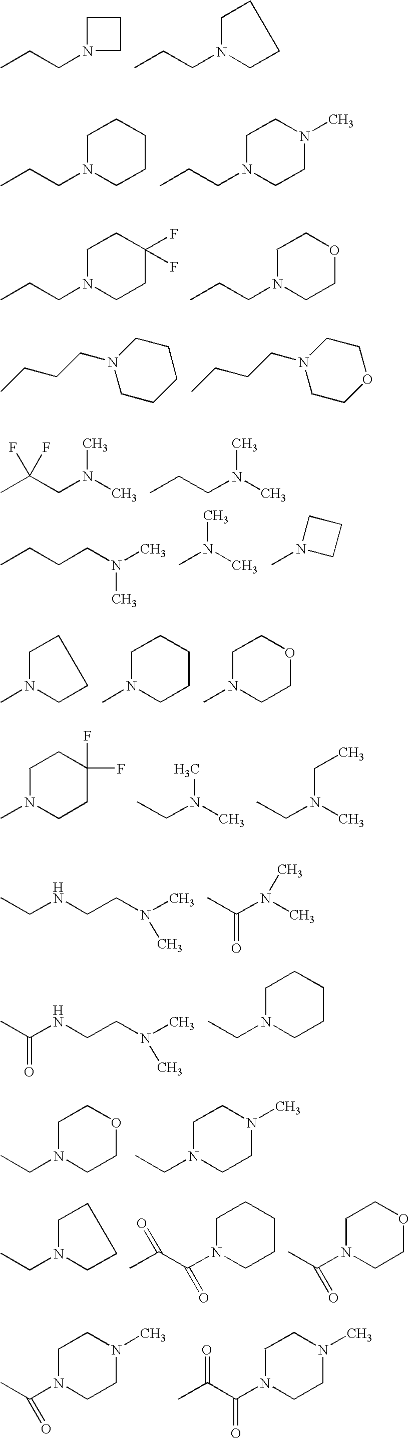

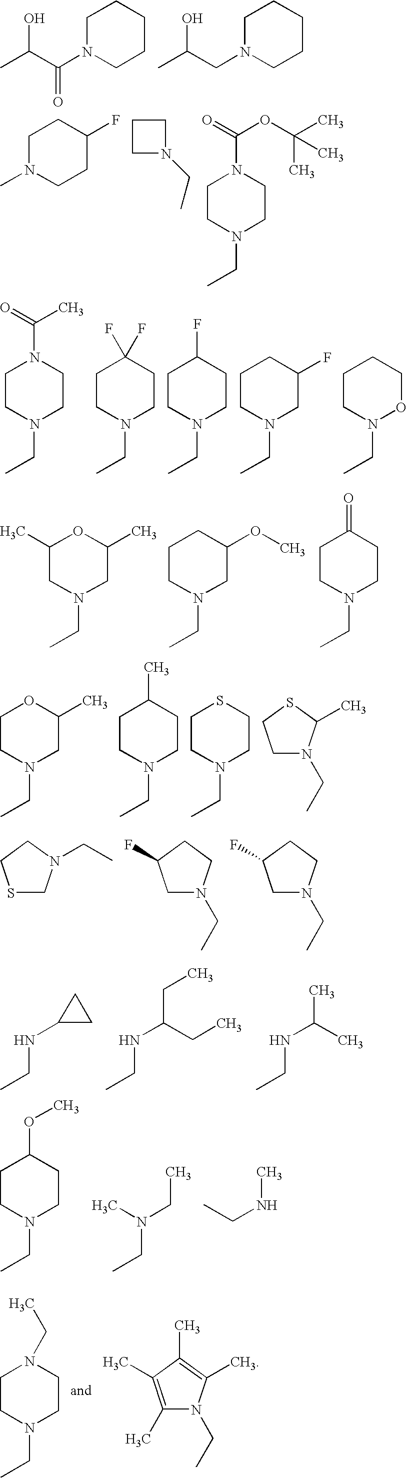

In some embodiments, R² is selected from the group consisting of substituted alkoxy and heteroaryl.

In some embodiments, R² is

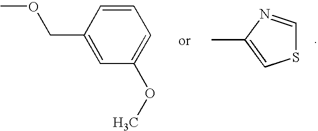

In some embodiments, Z is carboxy, carboxy ester, carboxylic acid isostere, —C(O)NR¹⁸R¹⁹, or —C(O)NHS(O)₂R⁴, wherein R¹⁸ and R¹⁹ are as defined in claim 1 and R⁴ is alkyl or aryl.

In some embodiments, Z is carboxy, methyl carboxylate, ethyl carboxylate, 6-(β-D-glucuronic acid) ester, 1H-tetrazol-5-yl, 5-oxo-4,5-dihydro-1,2,4-oxadiazol-3-yl, N-2-cyanoethylamide, N-2-(1H-tetrazol-5-yl)ethylamide, methylsulfonylaminocarbonyl, trifluoromethylsulfonylaminocarbonyl, cyclopropylsulfonylamino, or phenylsulfonylaminocarbonyl.

In some embodiments, Z is carboxy.

In some embodiments, Q is cycloalkyl or substituted cycloalkyl.

In some embodiments, Q is cyclohexyl or fluoro substituted cyclohexyl.

In some embodiments, p is 0.

In other embodiments, the provided is a compound of one of the following structures:

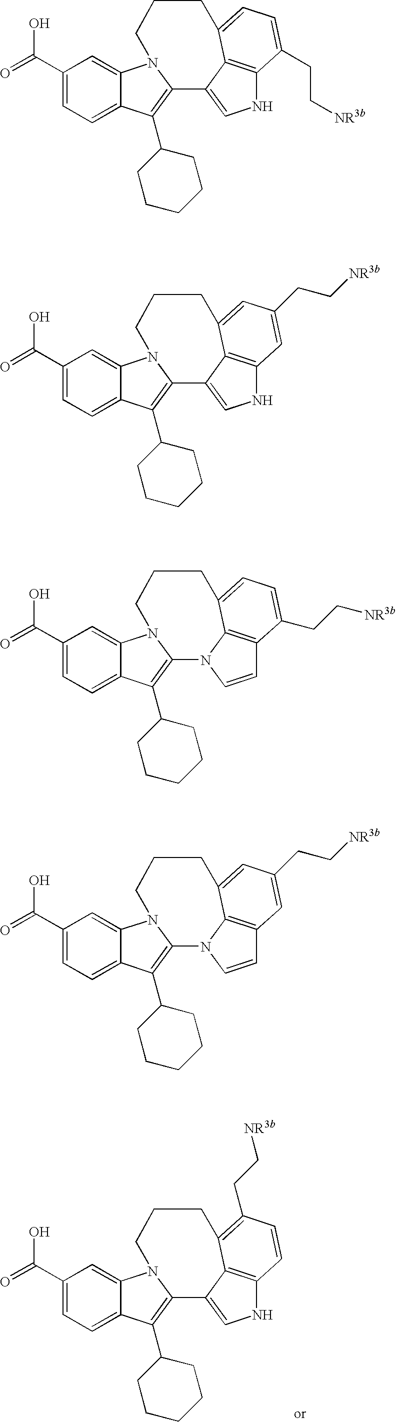

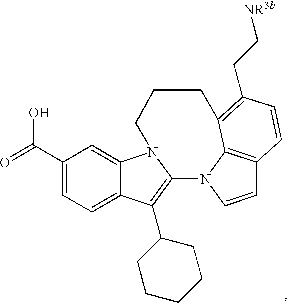

wherein R^{3b} is selected from the group consisting of hydrogen, alkyl, substituted alkyl, acyl, sulfonyl, substituted sulfonyl, and aminocarbonyl.

In other embodiments, the provided is a compound selected from Table 1 or Table 2 or a pharmaceutically acceptable salt or solvate thereof.

TABLE 1

| Compound # | Structure |
|---|---|
| 105 | 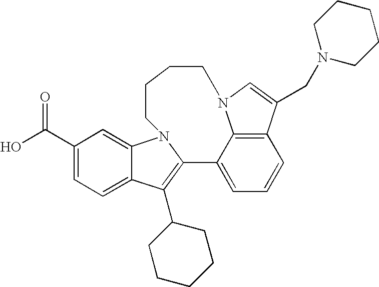 |
| 106 | 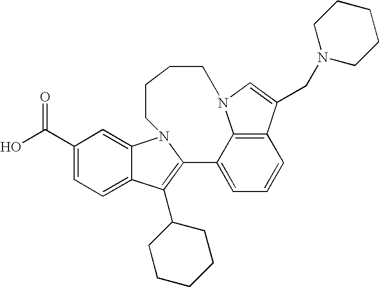 |
| 107 | 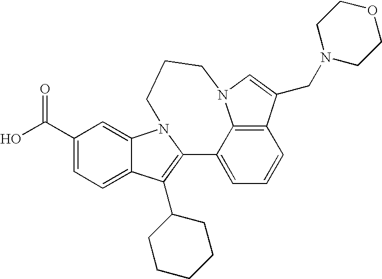 |
| 108 | 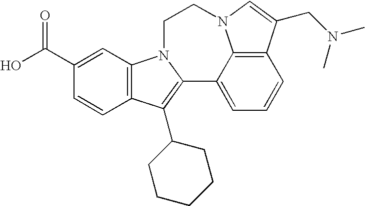 |
| 109 | 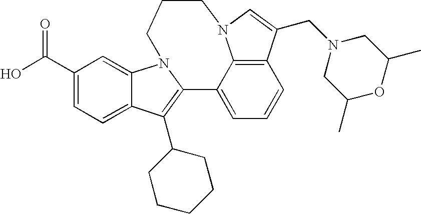 |
| 110 | 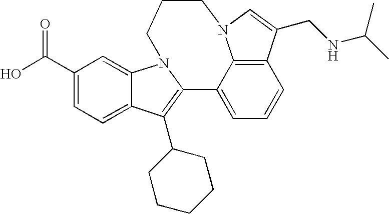 |
| 111 | 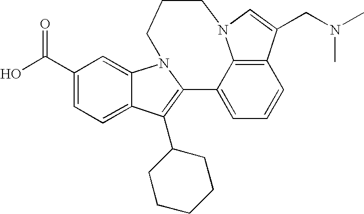 |
| 112 | 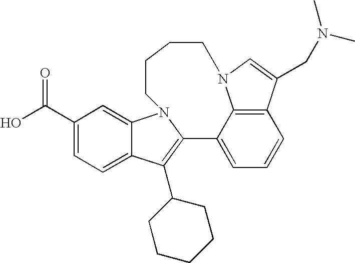 |
| 113 | 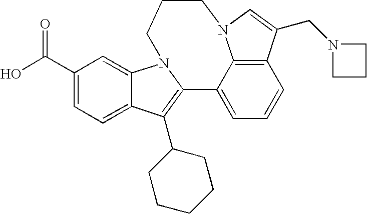 |
| 114 | 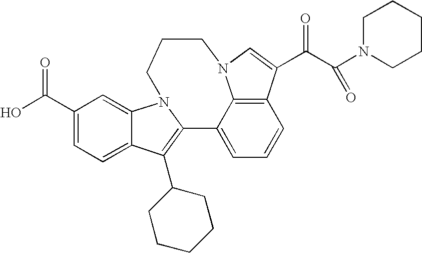 |
| 115 | 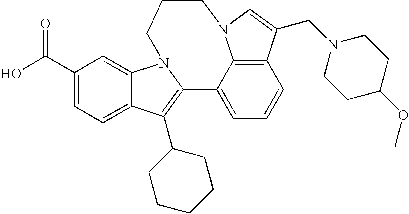 |
| 116 | 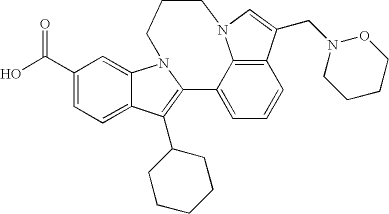 |
| 117 | 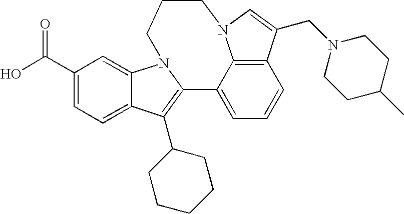 |
| 118 | 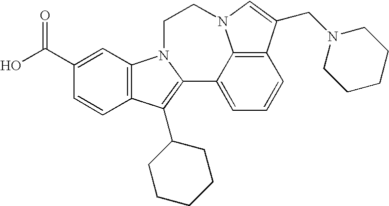 |
| 119 | 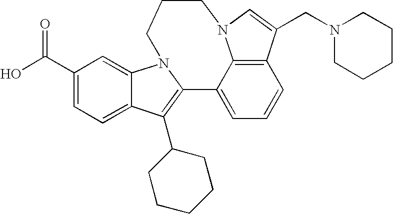 |
| 120 | 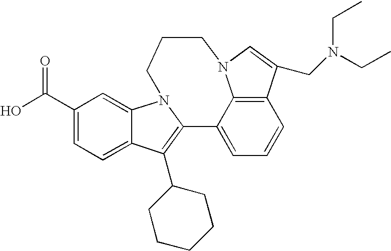 |
| 121 | 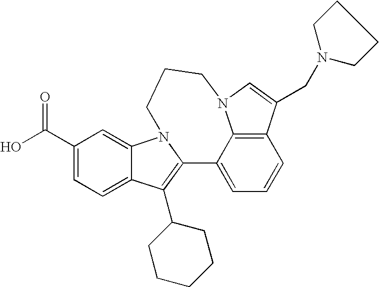 |
| 122 | 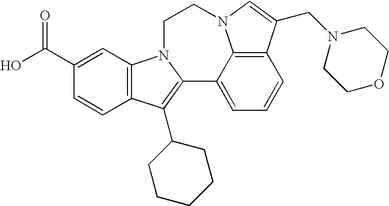 |
| 123 | 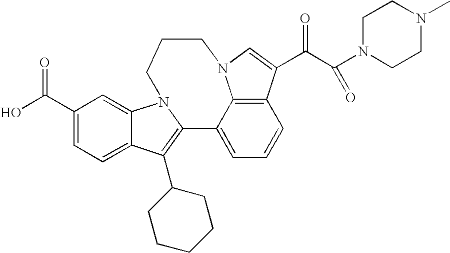 |
| 124 | 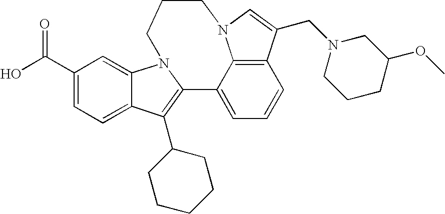 |
| 125 | 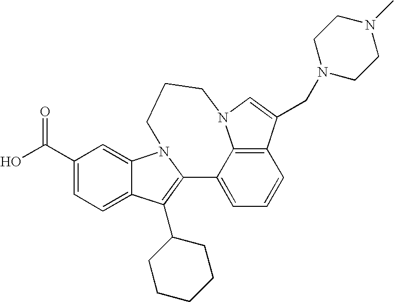 |
| 126 | 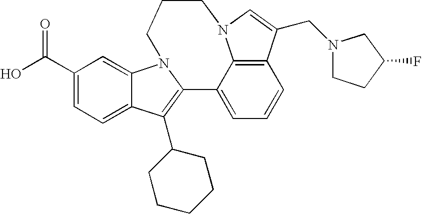 |
| 127 | 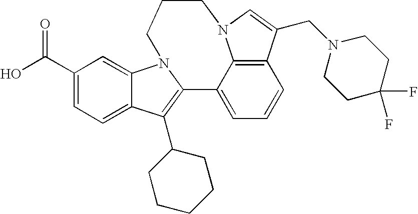 |
| 128 | 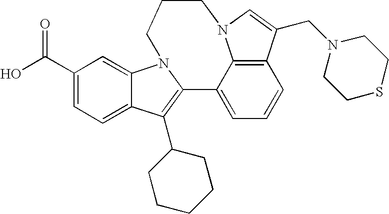 |
| 129 | 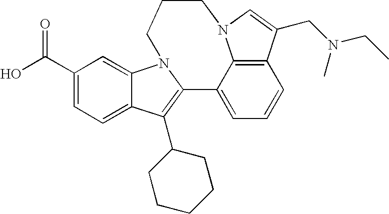 |
| 130 | 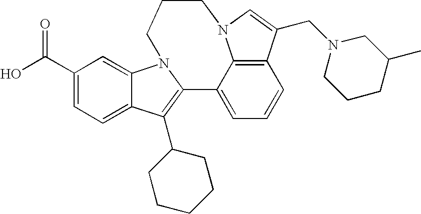 |
| 131 | 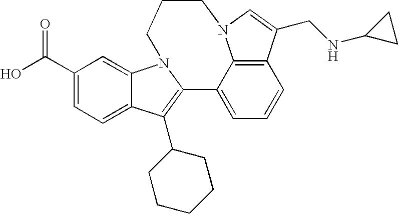 |
| 132 | 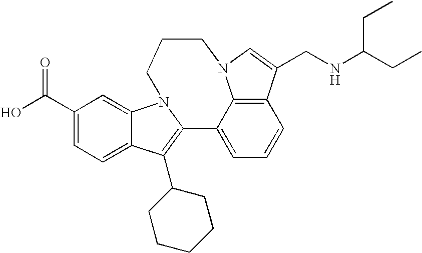 |

TABLE 2

| Compound # | Structure |
|---|---|
| 201 | 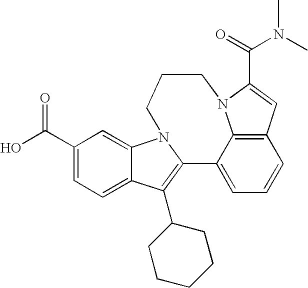 |
| 204 | 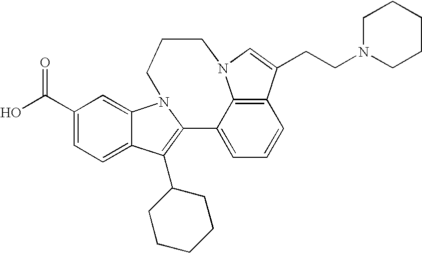 |
| 206 | 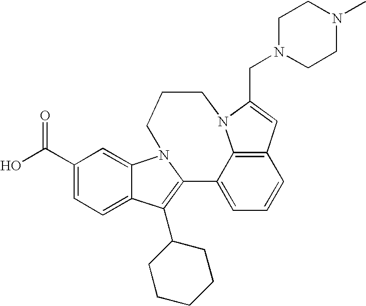 |
| 207 | 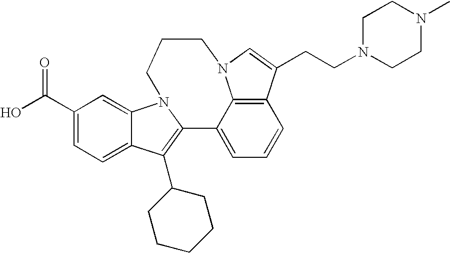 |
| 208 | 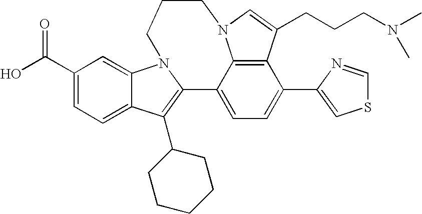 |
| 210 | 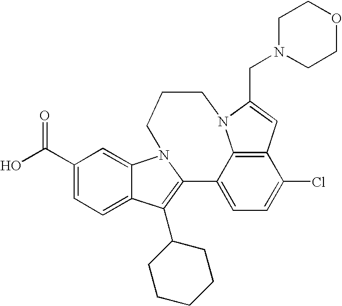 |
| 211 | 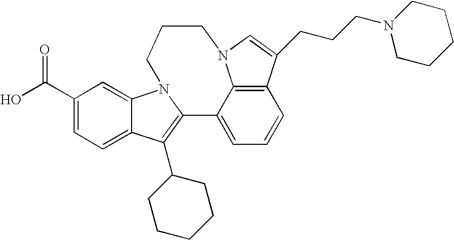 |
| 212 | 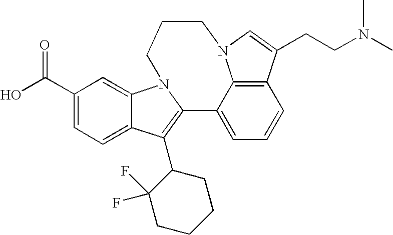 |
| 213 | 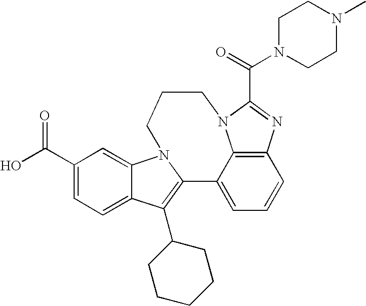 |
| 214 | 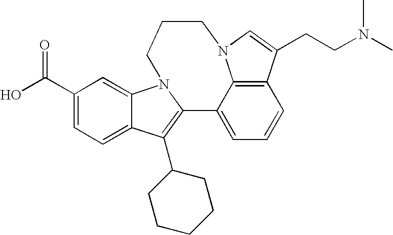 |
| 216 | 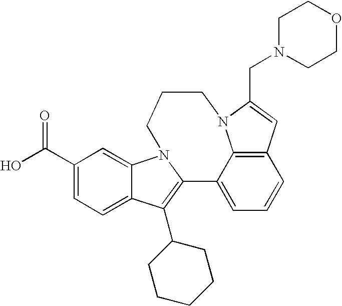 |
| 217 | 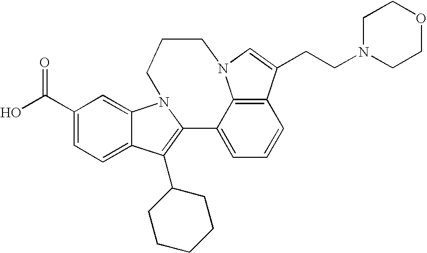 |
| 218 | 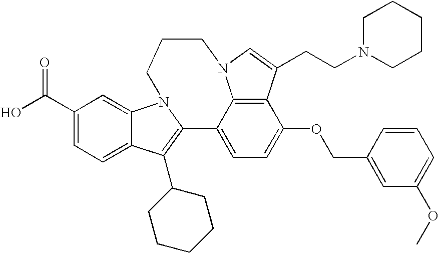 |
| 219 | 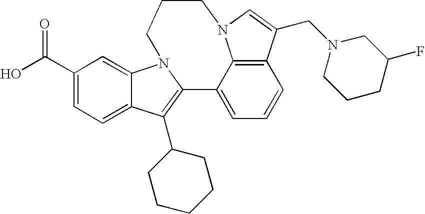 |
| 221 | 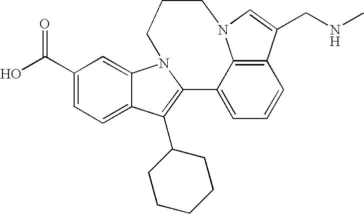 |
| 222 | 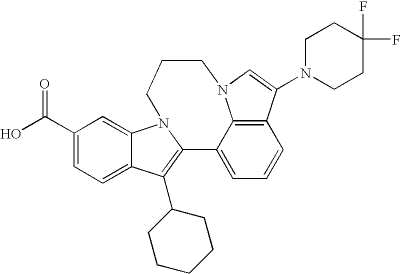 |
| 224 | 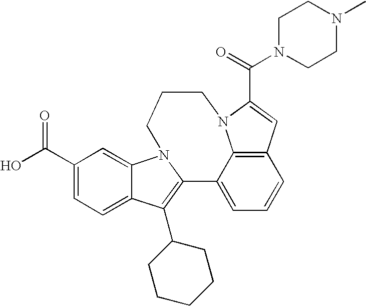 |
| 226 | 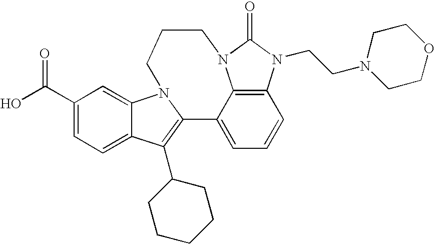 |
| 228 | 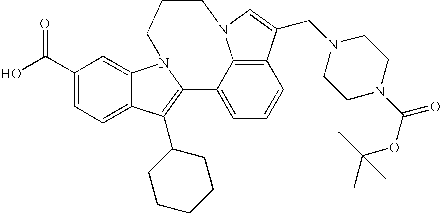 |
| 230 | 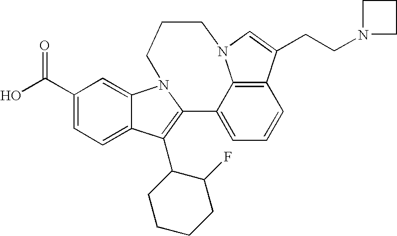 |
| 231 | 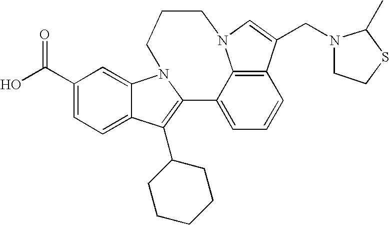 |
| 232 | 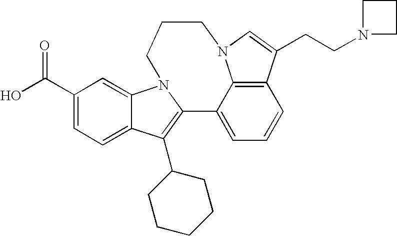 |
| 233 | 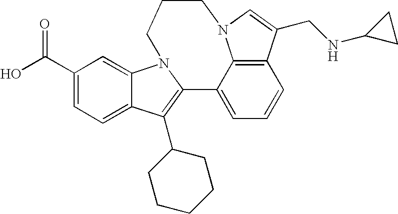 |
| 235 | 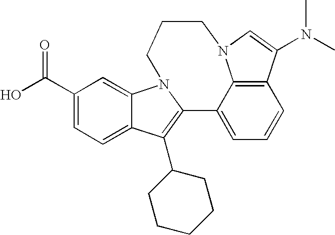 |
| 236 | 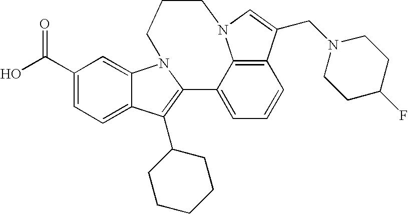 |
| 237 | 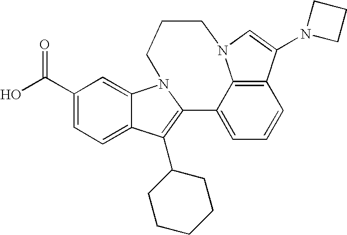 |
| 239 | 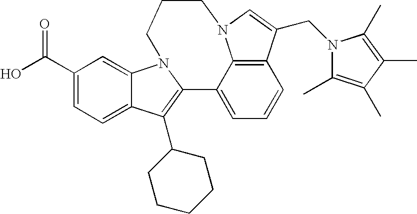 |
| 240 | 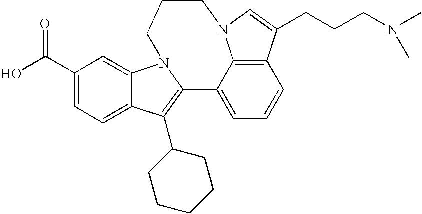 |
| 241 | 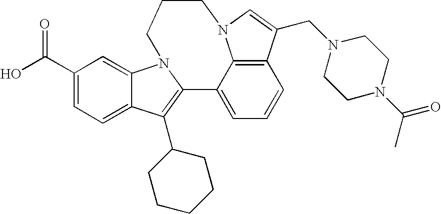 |
| 242 | 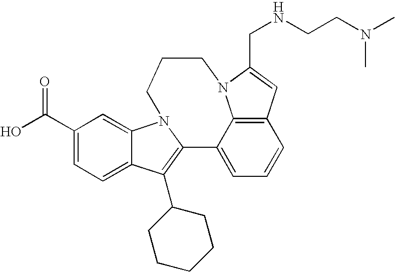 |
| 244 | 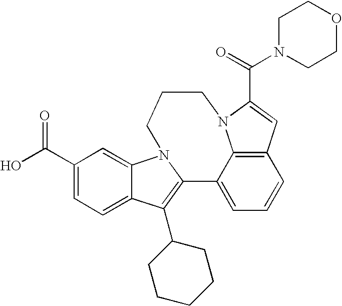 |
| 245 | 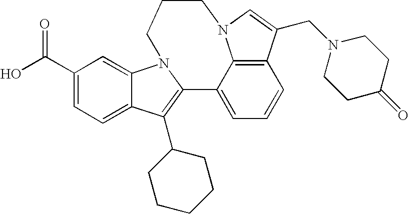 |
| 247 | 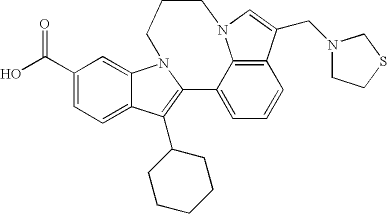 |
| 248 | 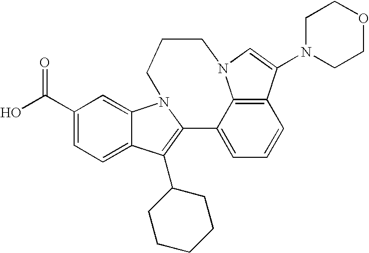 |
| 249 | 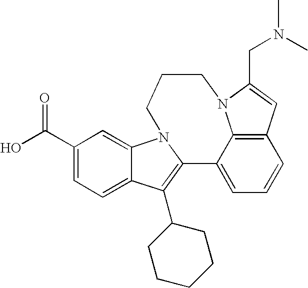 |
| 250 | 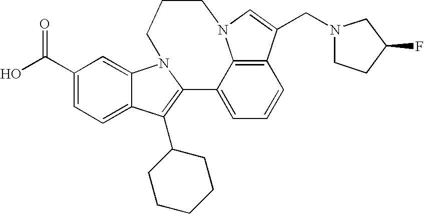 |
| 251 | 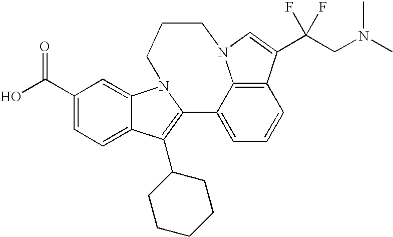 |
| 253 | 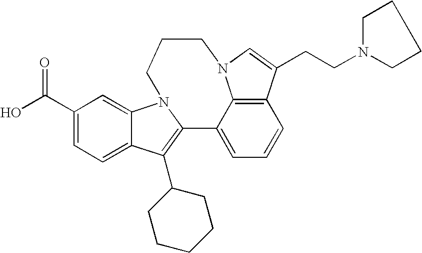 |
| 254 | 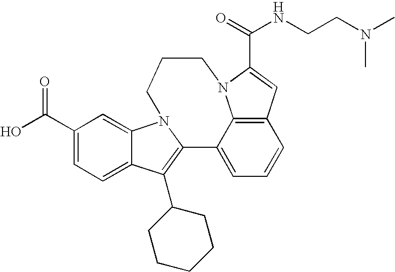 |
| 256 | 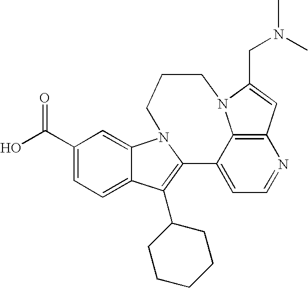 |
| 258 | 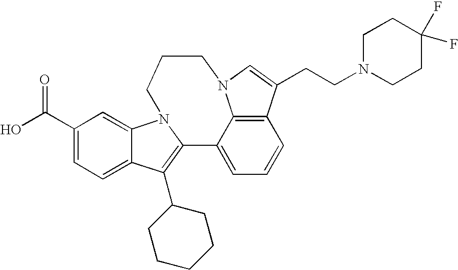 |
| 259 | 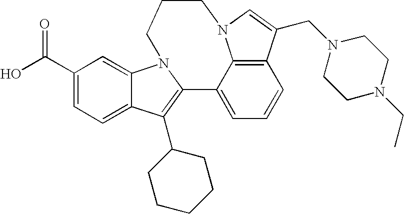 |
| 260 | 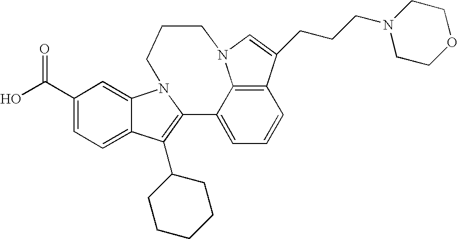 |
| 261 | 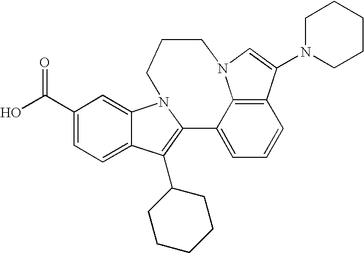 |
| 263 | 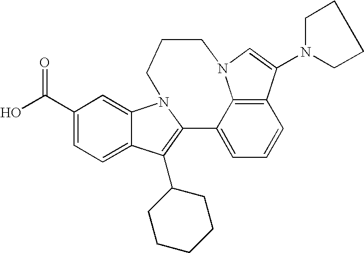 |
| 264 | 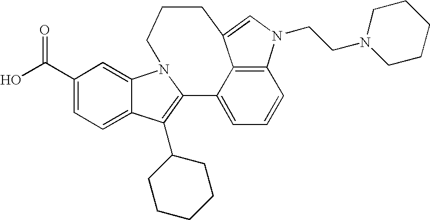 |
| 265 | 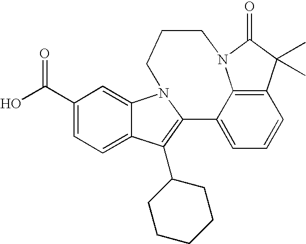 |
| 266 | 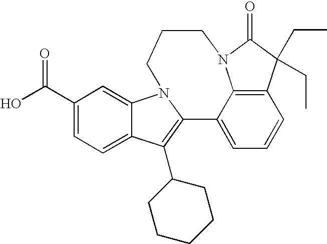 |
| 267 | 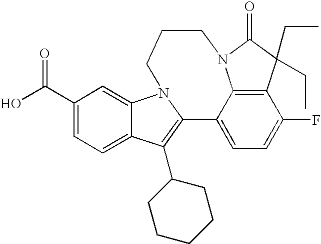 |
| 268 | 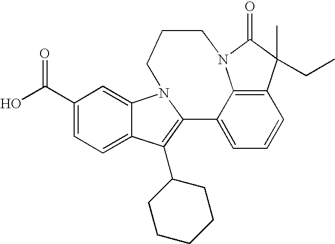 |
| 269 | 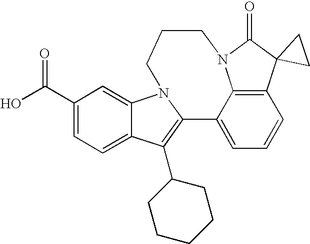 |
| 270 | 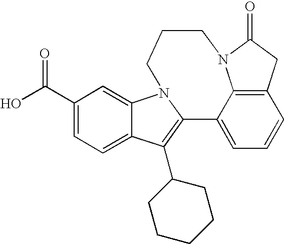 |
| 271 | 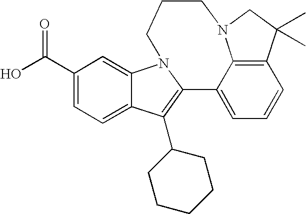 |
| 272 | 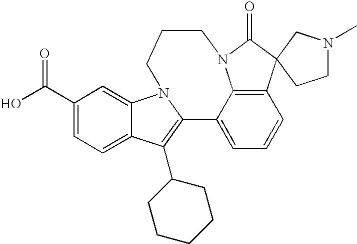 |
| 273 | 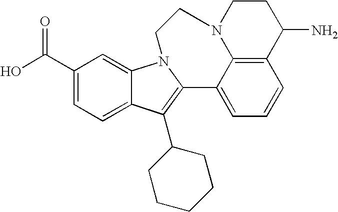 |
| 274 | 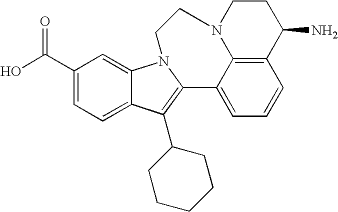 |
| 275 | 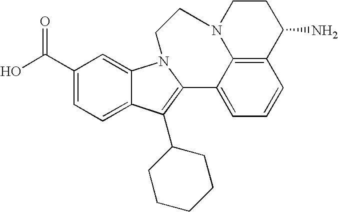 |
| 276 | 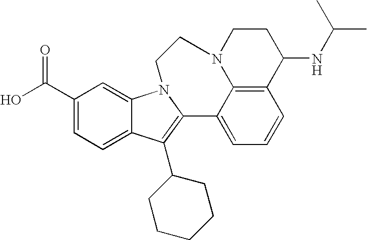 |
| 277 | 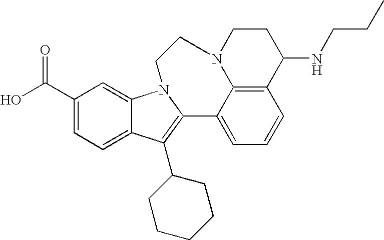 |
| 278 | 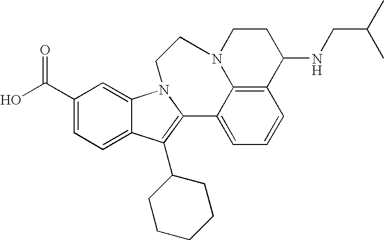 |
| 279 | 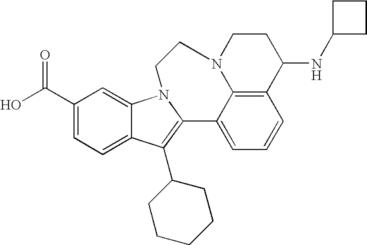 |
| 280 | 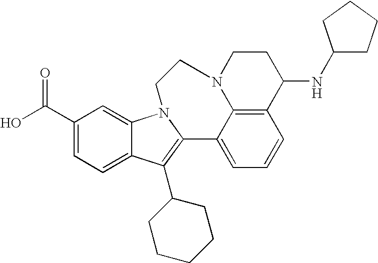 |
| 281 | 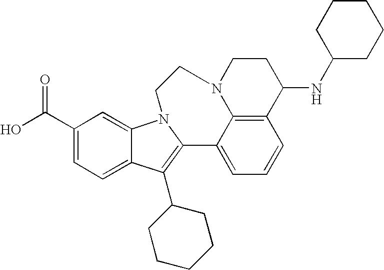 |
| 282 | 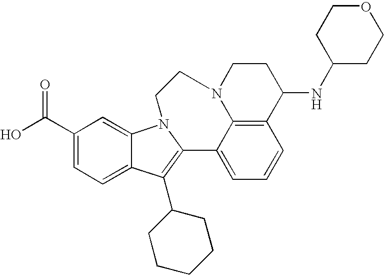 |
| 283 | 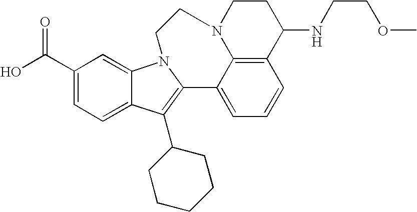 |
| 284 | 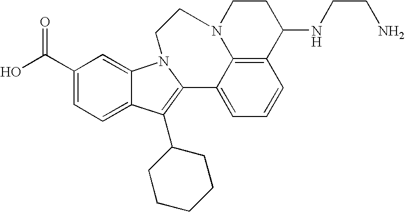 |
| 285 | 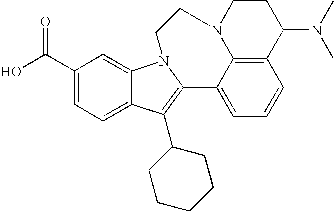 |
| 286 | 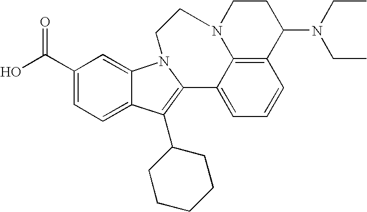 |
| 287 | 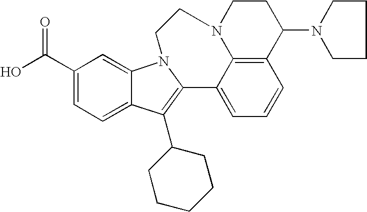 |
| 288 | 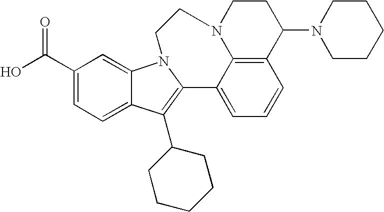 |
| 289 | 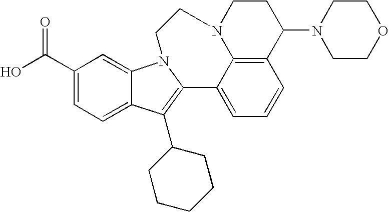 |
| 290 | 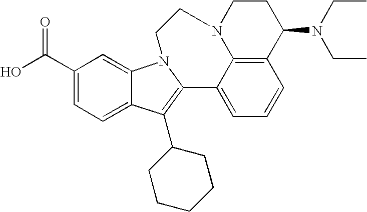 |
| 291 | 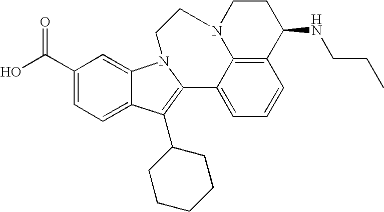 |
| 292 | 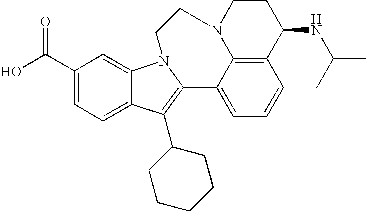 |
| 293 | 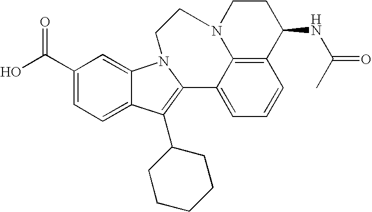 |
| 294 | 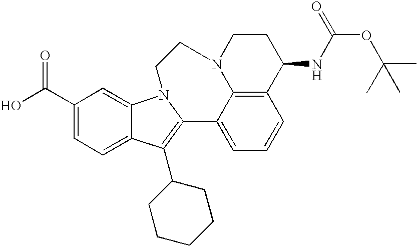 |
| 295 | 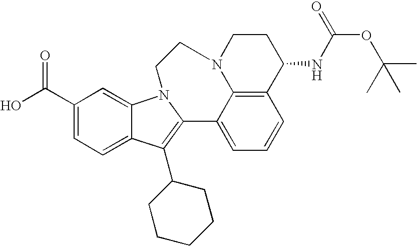 |
| 296 | 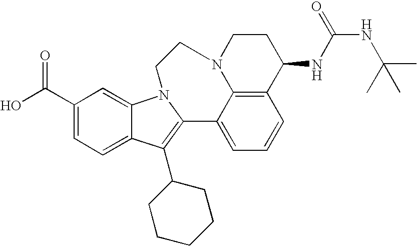 |
| 297 | 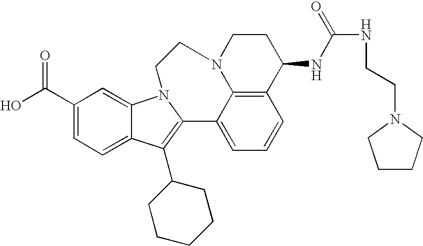 |
| 298 | 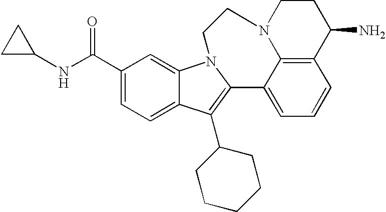 |
| 299 | 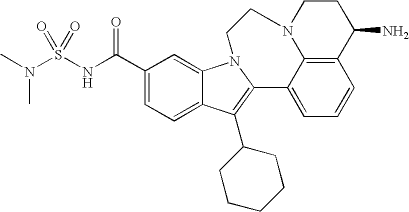 |
| 300 | 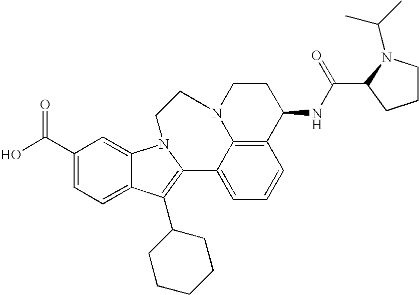 |
| 301 | 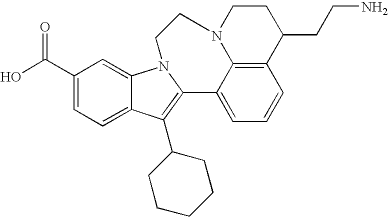 |
| 302 | 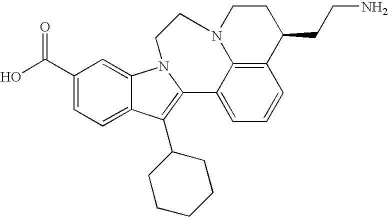 |
| 303 | 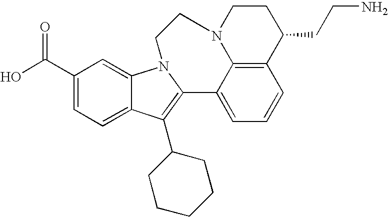 |
| 304 | 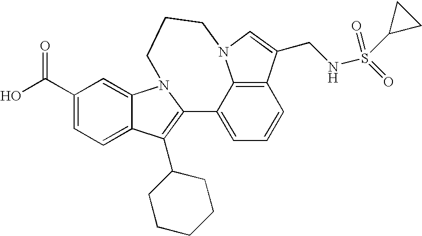 |
| 305 | 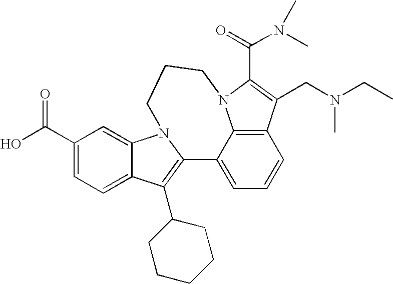 |
| 306 | 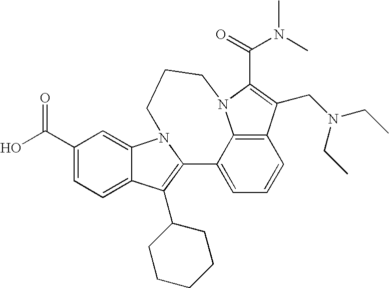 |
| 307 | 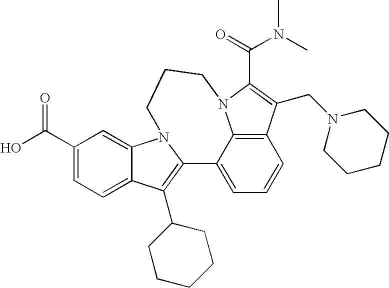 |
| 308 | 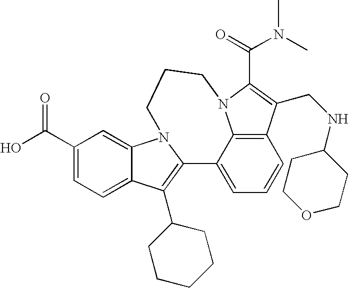 |
| 309 | 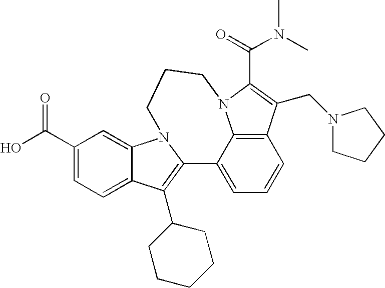 |
| 310 | 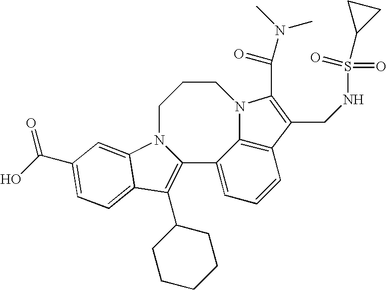 |
| 311 | 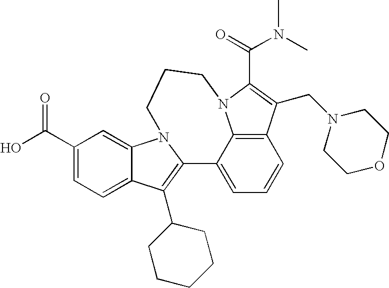 |
| 312 | 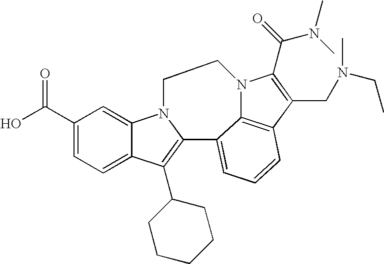 |
| 313 | 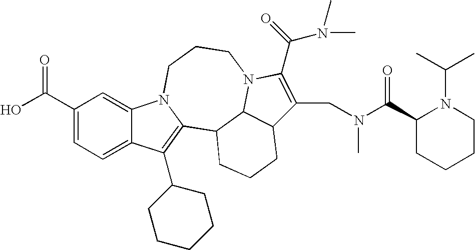 |
| 314 | 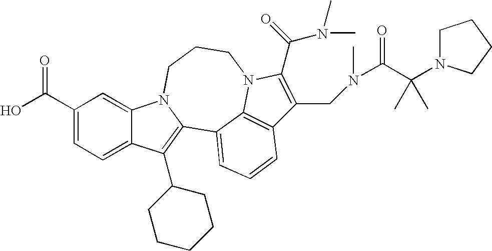 |
| 315 | 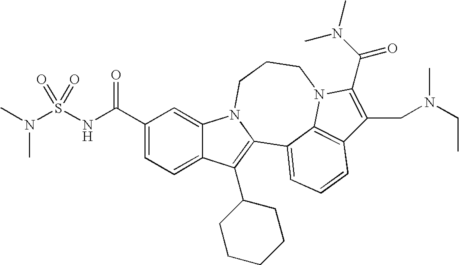 |
| 316 | 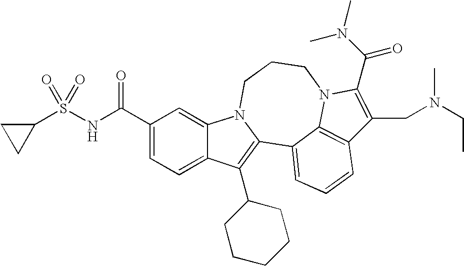 |
| 317 | 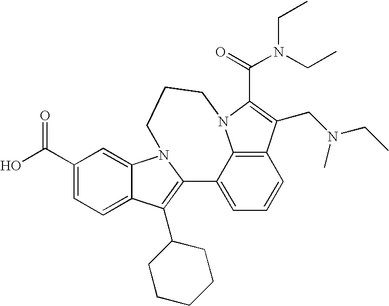 |
| 318 | 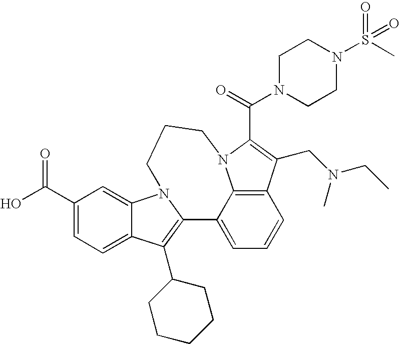 |
| 319 | 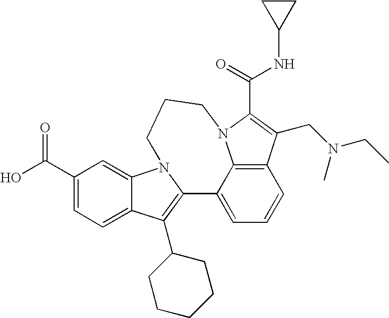 |
| 320 | 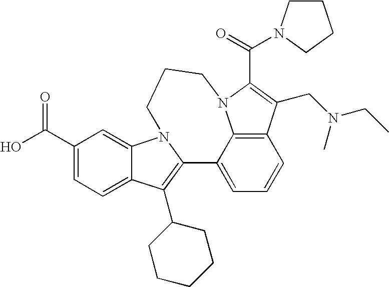 |
| 321 | 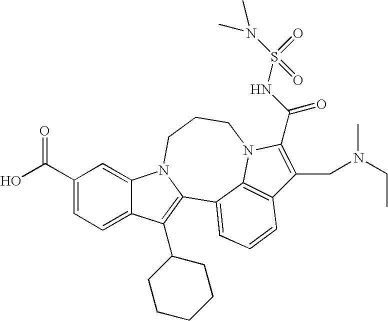 |
| 322 | 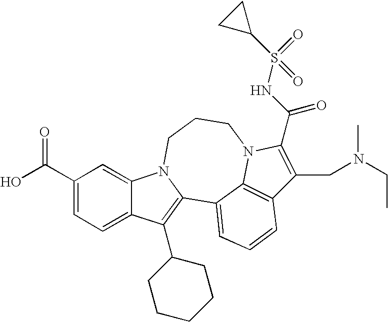 |
| 323 | 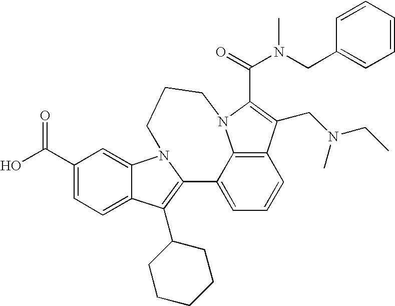 |
| 324 | 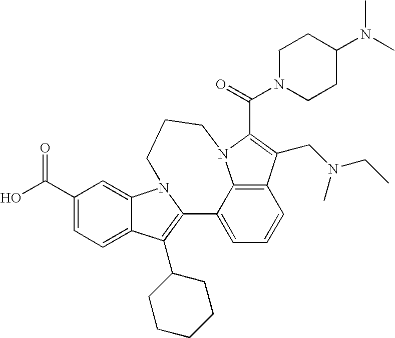 |
| 325 | 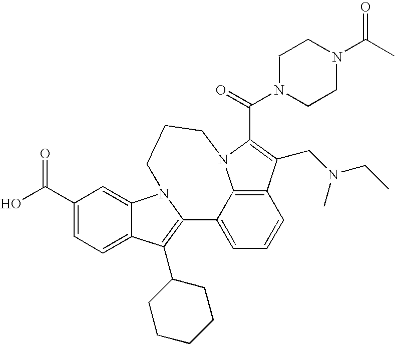 |
| 326 | 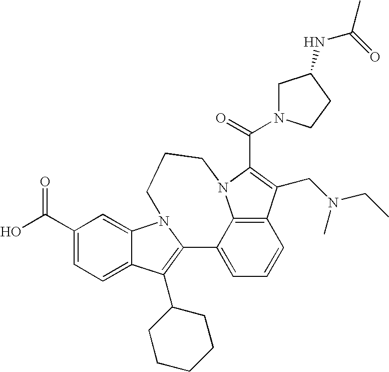 |
| 329 | 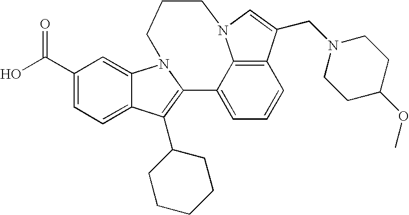 |
| 330 | 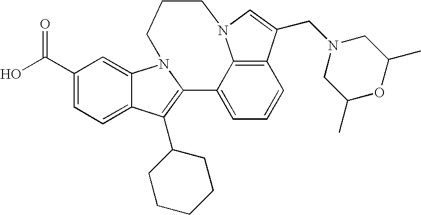 |
| 331 | 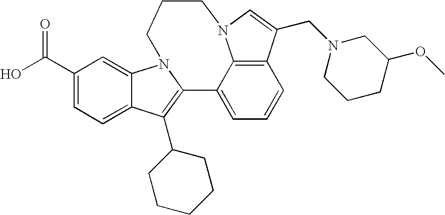 |
| 332 | 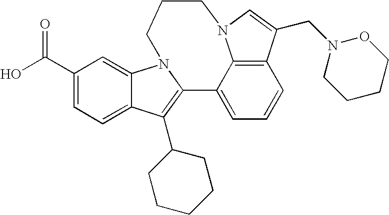 |
| 333 | 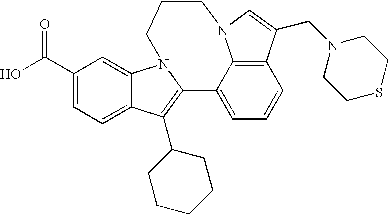 |
| 334 | 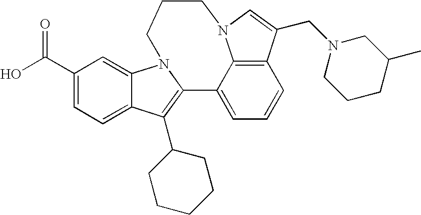 |
| 335 | 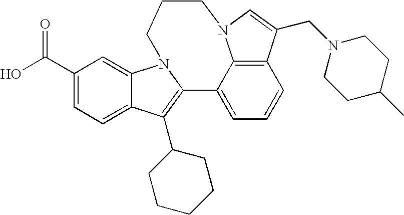 |
| 336 | 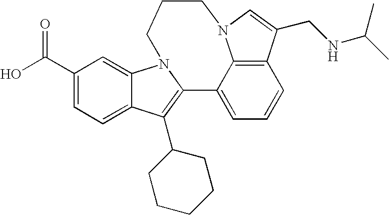 |
| 337 | 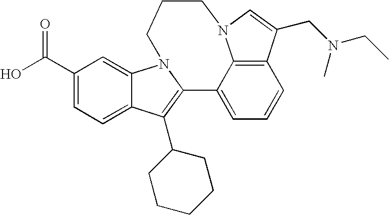 |
| 338 | 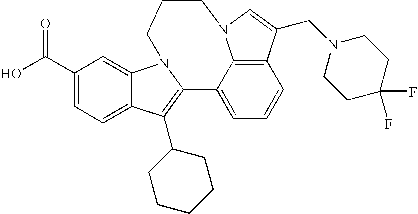 |
| 341 | 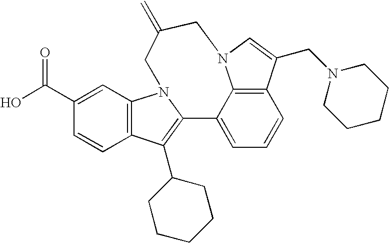 |
| 343 | 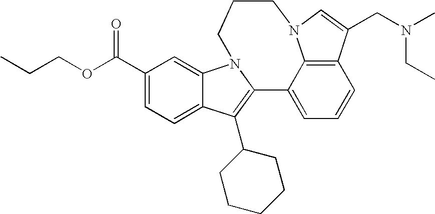 |
| 344 | 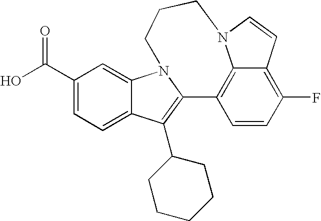 |
| 345 | 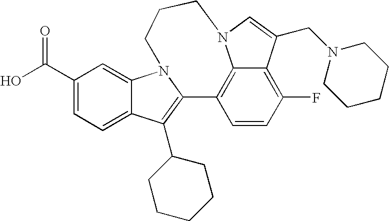 |
| 346 | 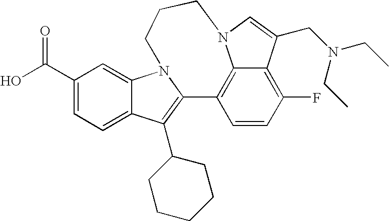 |
| 347 | 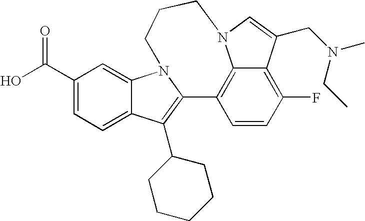 |
| 348 | 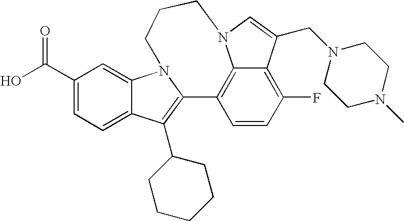 |
| 349 | 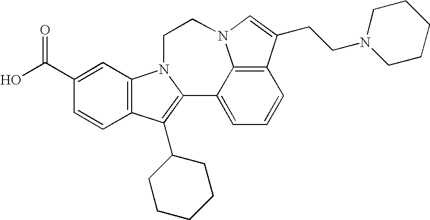 |
| 350 | 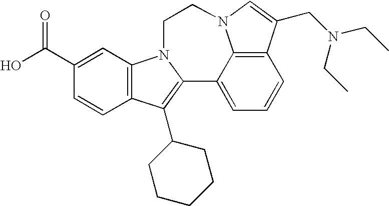 |
| 351 | 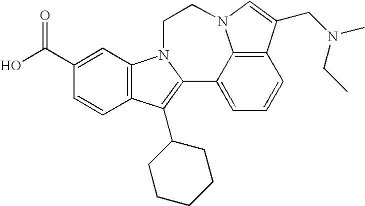 |
| 352 | 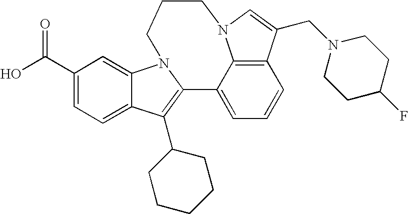 |
| 353 | 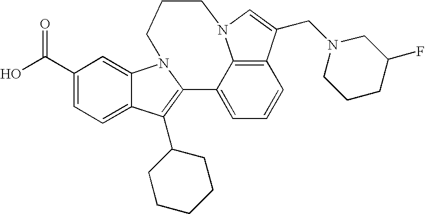 |
| 354 | 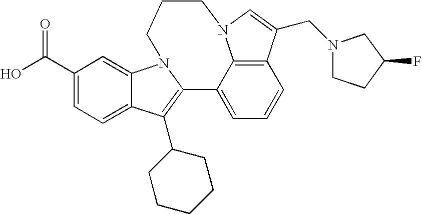 |
| 356 | 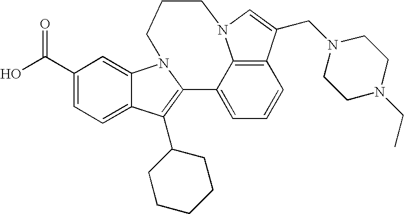 |
| 357 | 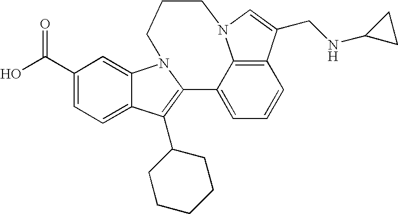 |
| 358 | 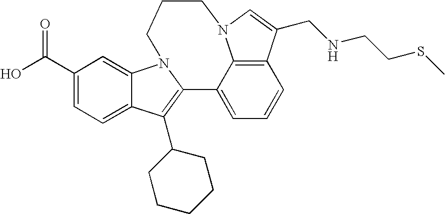 |
| 359 | 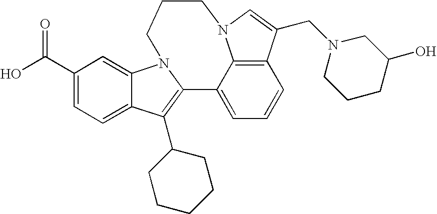 |
| 360 | 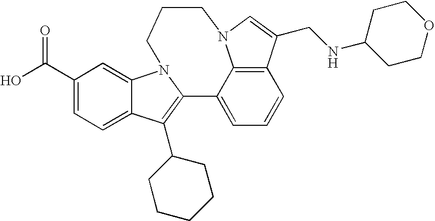 |
| 361 | 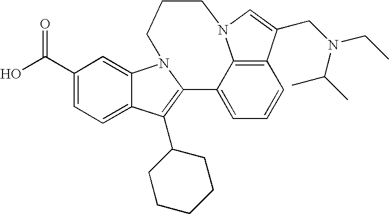 |
| 362 | 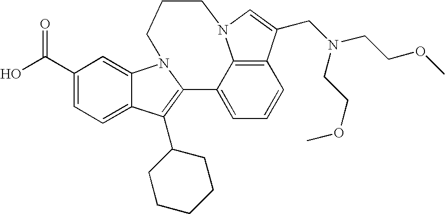 |
| 363 | 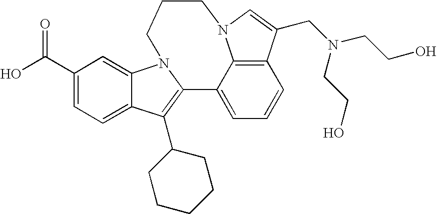 |
| 364 | 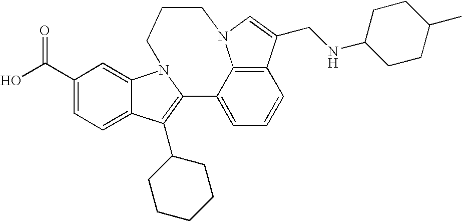 |
| 365 | 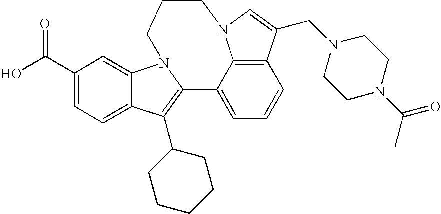 |
| 366 | 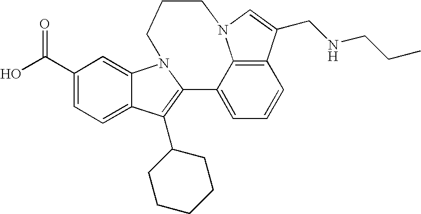 |
| 367 | 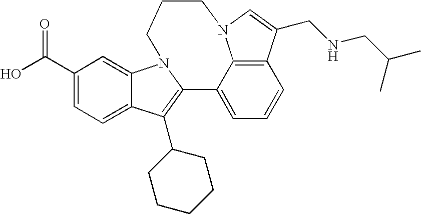 |
| 368 | 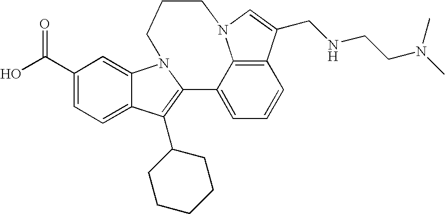 |
| 369 | 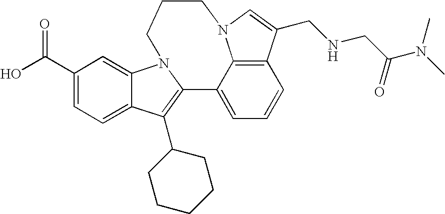 |
| 370 | 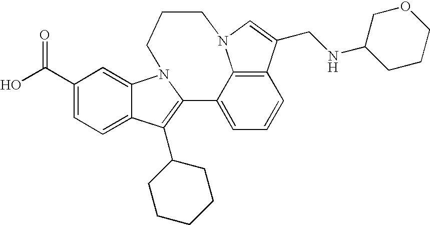 |
| 371 | 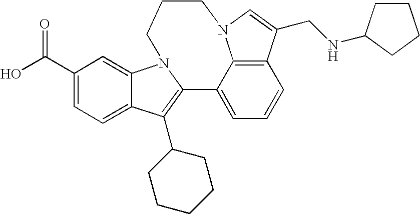 |
| 372 | 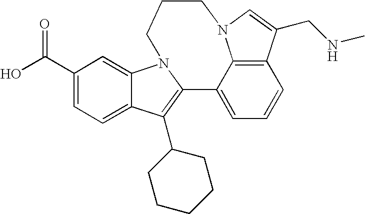 |
| 373 | 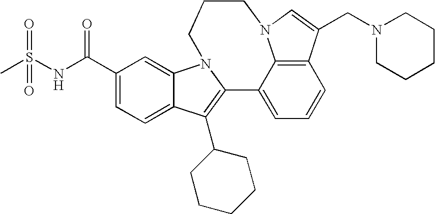 |
| 374 | 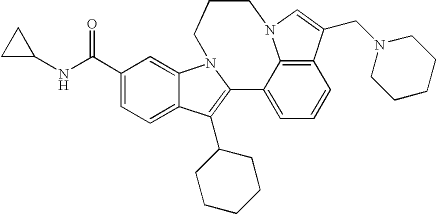 |
| 375 | 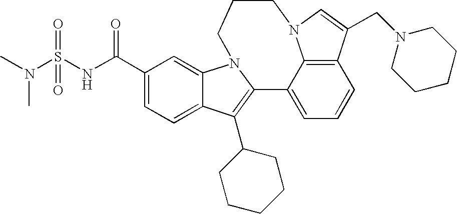 |
| 376 | 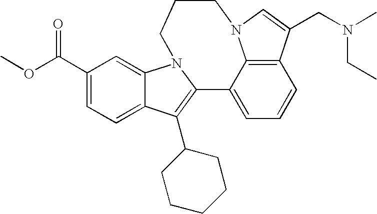 |
| 377 | 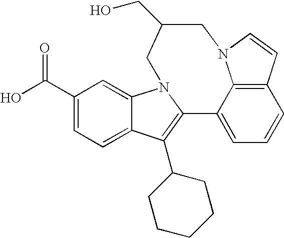 |
| 378 | 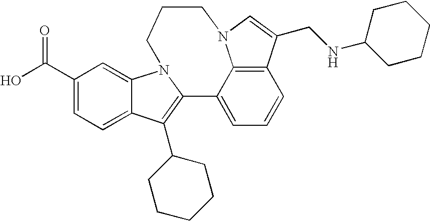 |
| 379 | 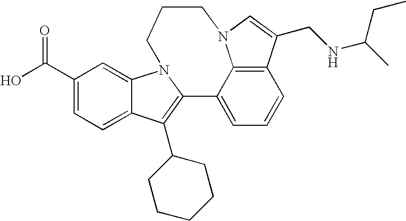 |
| 380 | 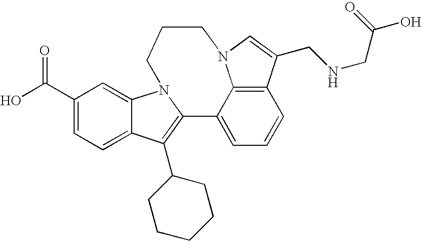 |
| 381 | 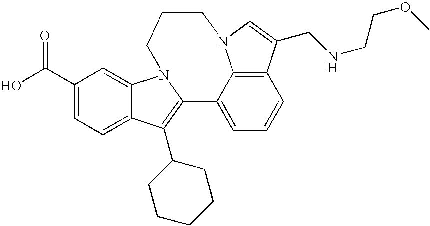 |
| 382 | 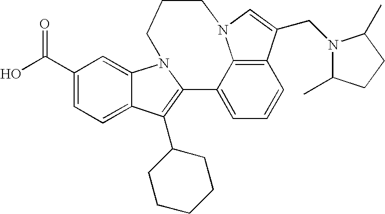 |
| 383 | 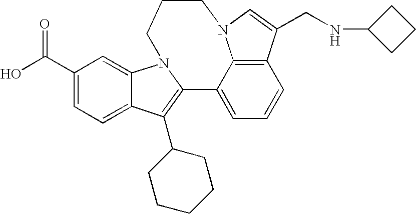 |
| 384 | 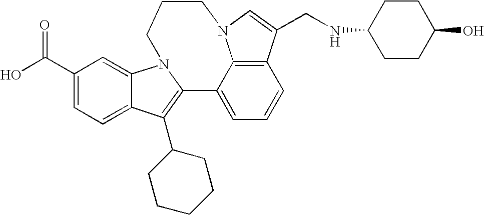 |
| 385 | 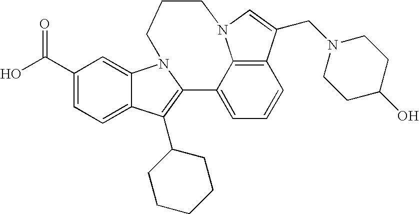 |
| 386 | 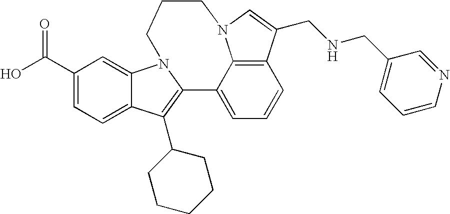 |
| 387 | 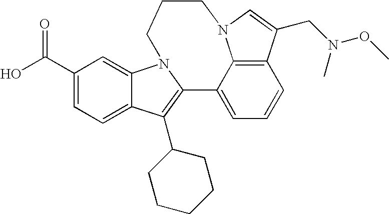 |
| 388 | 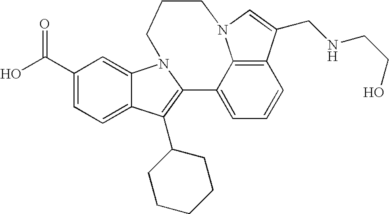 |
| 389 | 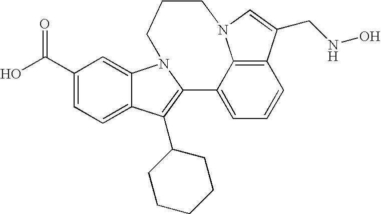 |
| 390 | 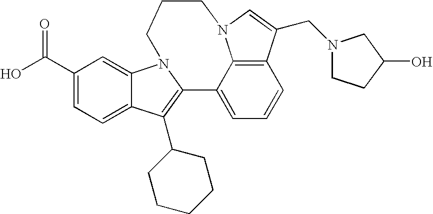 |
| 391 | 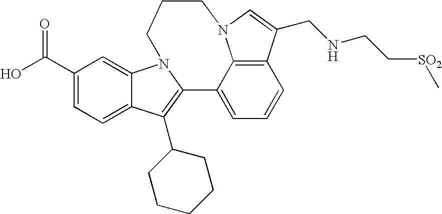 |
| 392 | 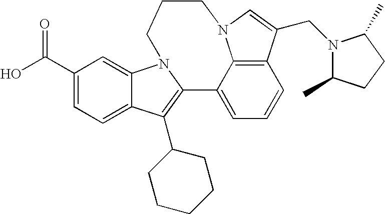 |
| 393 | 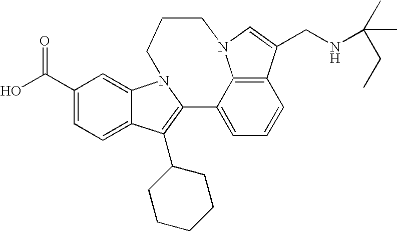 |
| 394 | 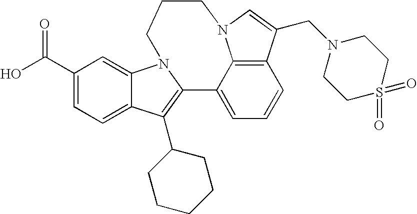 |
| 395 | 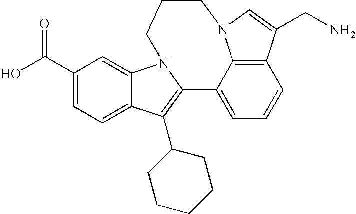 |
| 396 | 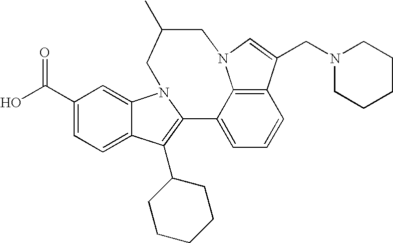 |
| 397 | 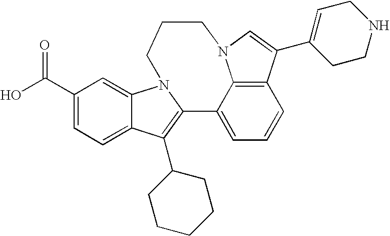 |
| 398 | 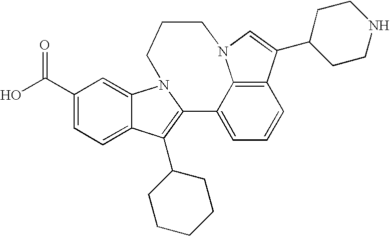 |
| 399 | 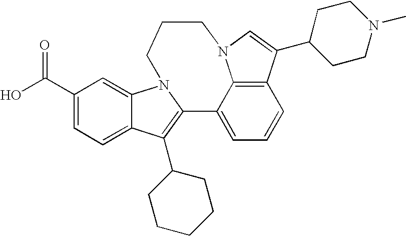 |
| 400 | 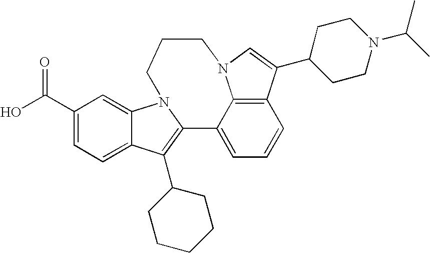 |
| 401 | 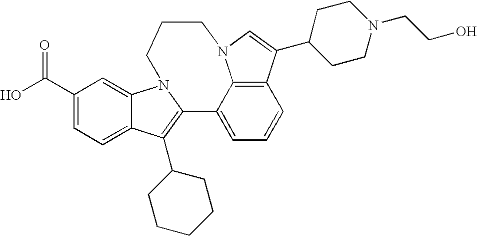 |
| 402 | 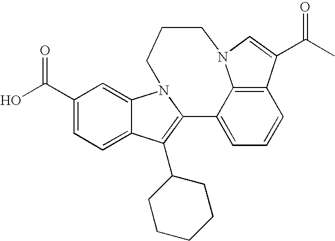 |
| 403 | 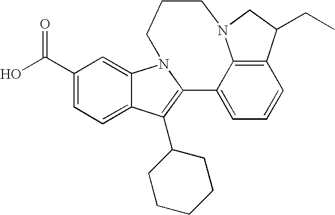 |
| 404 | 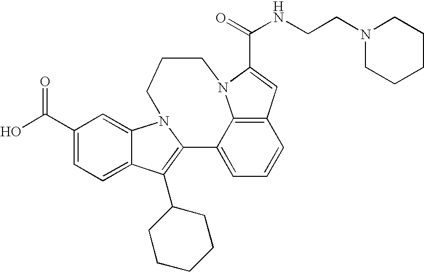 |
| 405 | 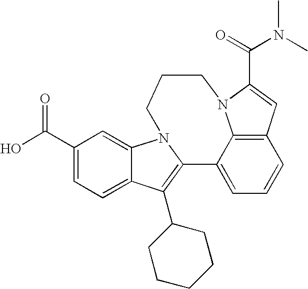 |
| 406 | 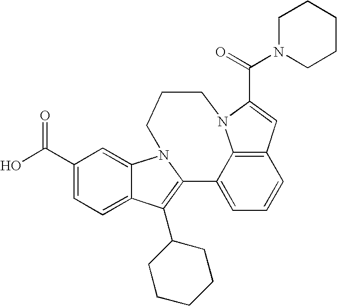 |
| 407 | 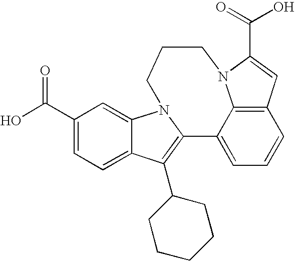 |
| 408 | 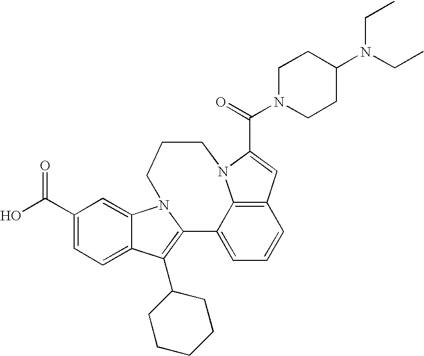 |
| 409 | 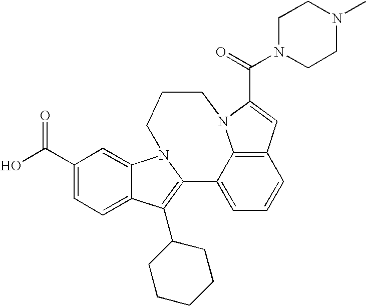 |
| 410 | 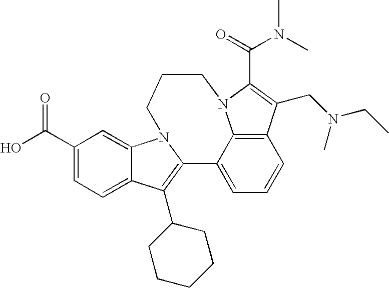 |
| 411 | 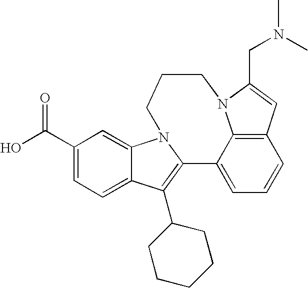 |
| 412 | 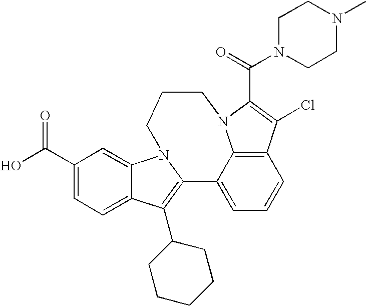 |
| 413 | 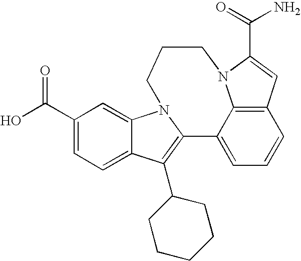 |
| 414 | 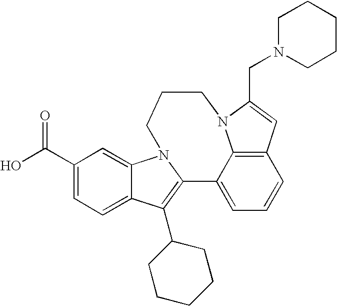 |
| 415 | 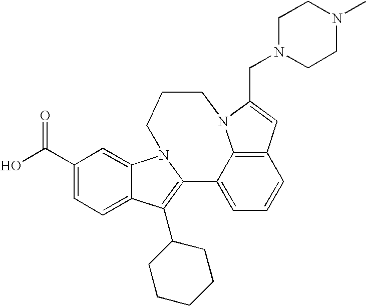 |
| 416 | 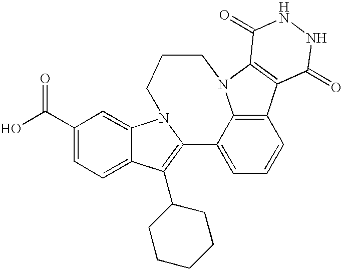 |
| 417 | 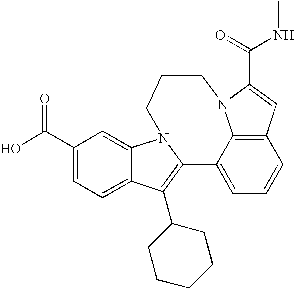 |
| 418 | 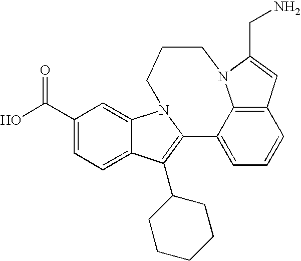 |
| 419 | 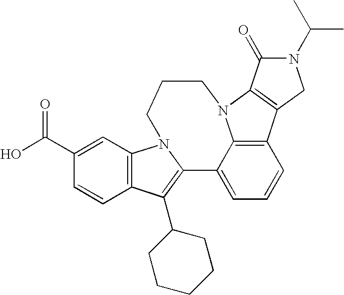 |
| 420 | 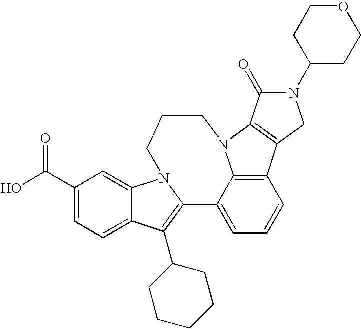 |
| 421 | 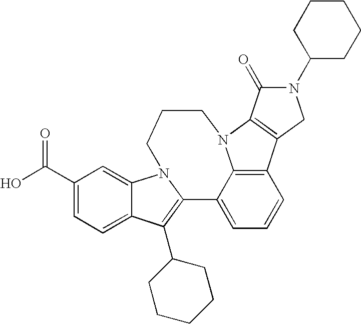 |
| 427 | 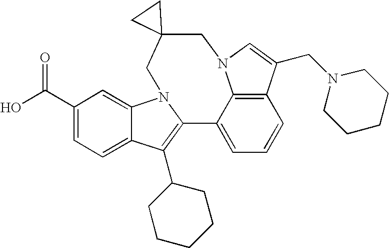 |
| 428 | 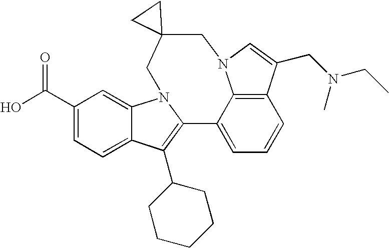 |
| 429 | 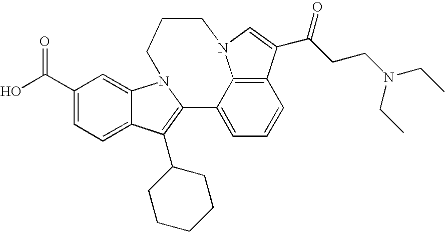 |
| 430 | 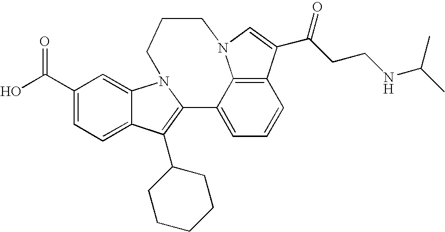 |
| 431 | 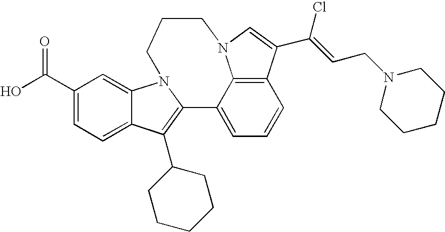 |
| 432 | 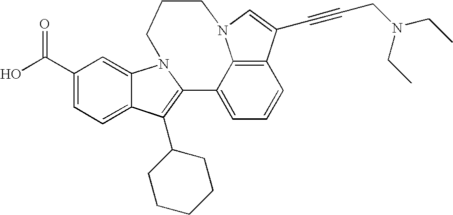 |
| 433 | 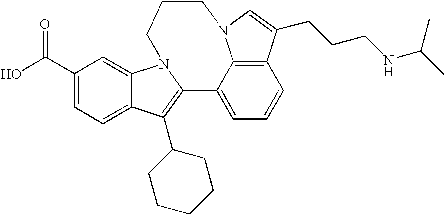 |
| 434 | 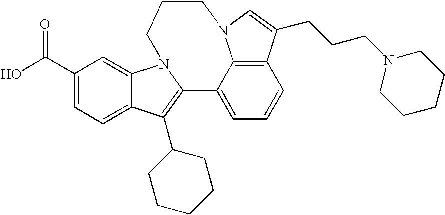 |
| 435 | 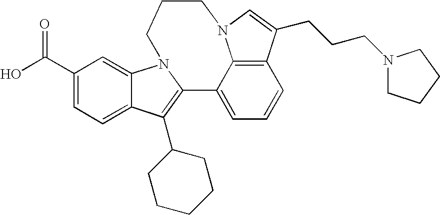 |
| 436 | 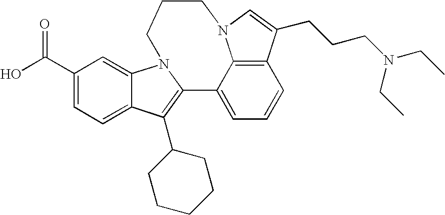 |
| 437 | 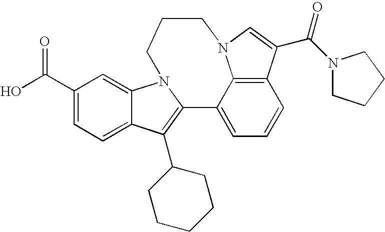 |
| 438 | 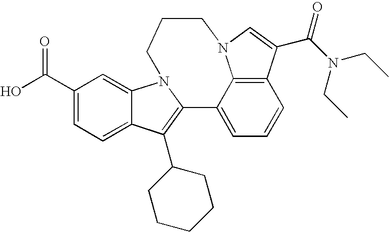 |
| 439 | 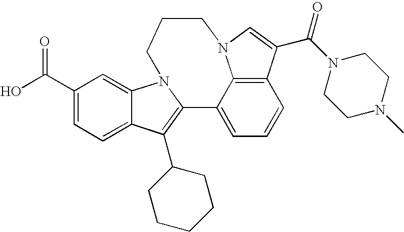 |
| 440 | 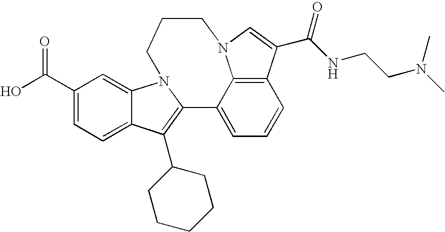 |
| 441 | 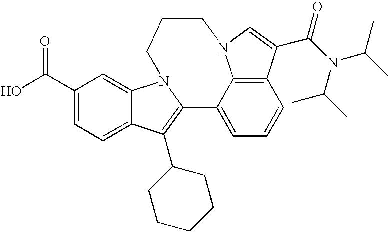 |
| 442 | 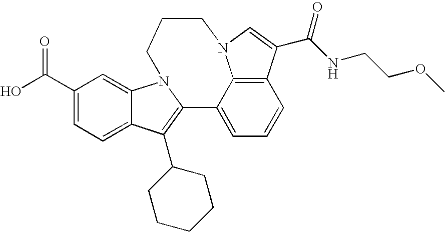 |
| 443 | 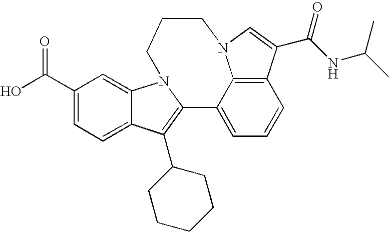 |
| 444 | 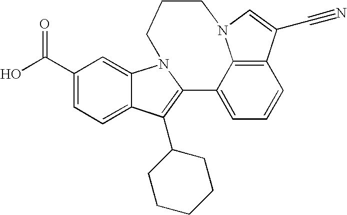 |
| 445 | 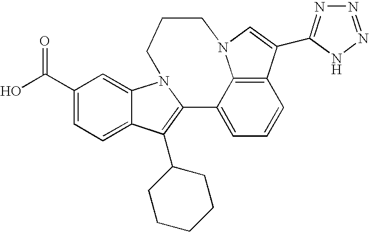 |
| 446 | 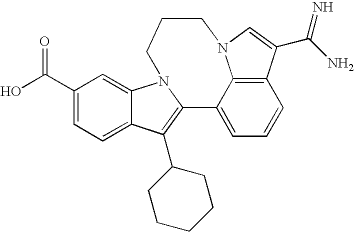 |
| 447 | 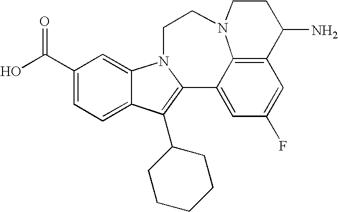 |
| 448 | 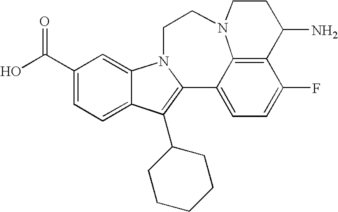 |
| 449 | 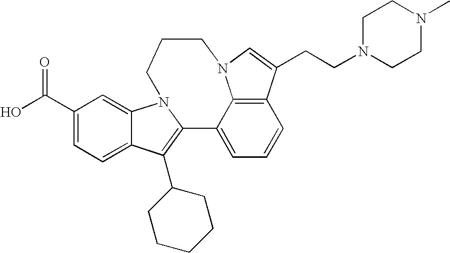 |
| 451 | 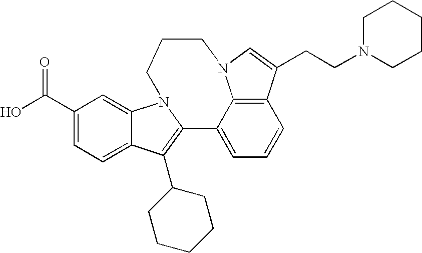 |
| 452 | 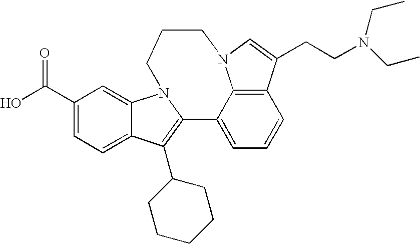 |
| 453 | 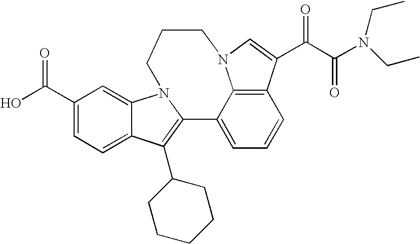 |
| 454 | 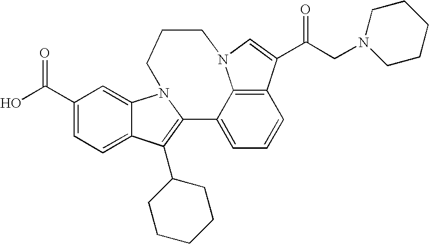 |
| 455 | 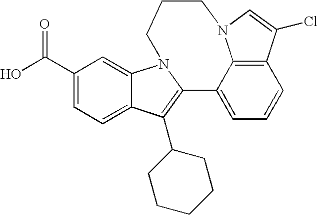 |
| 456 | 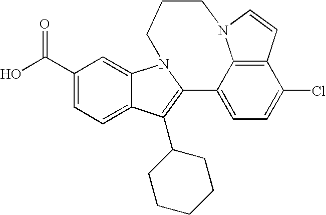 |
| 457 | 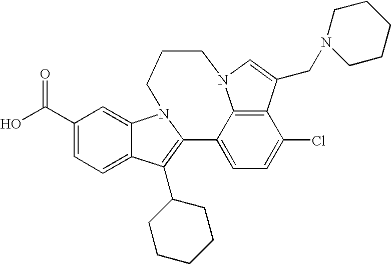 |
| 458 | 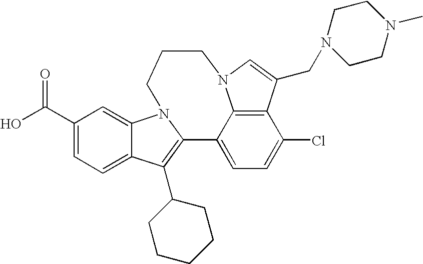 |
| 459 | 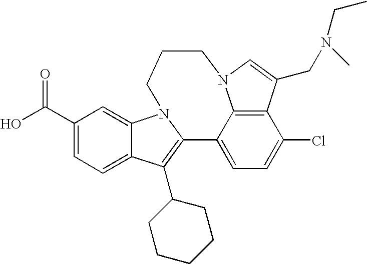 |
| 460 | 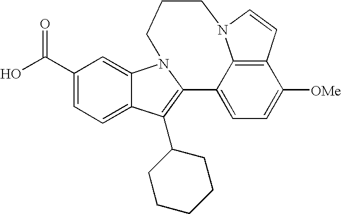 |
| 461 | 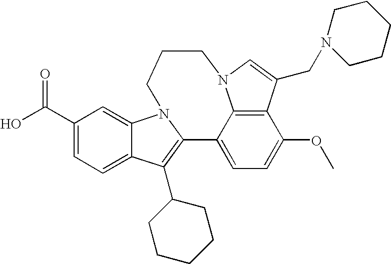 |
| 462 | 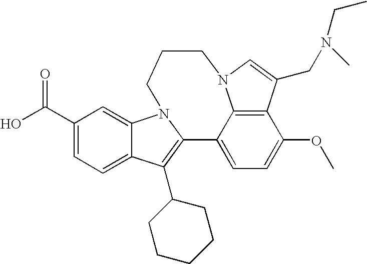 |
| 463 | 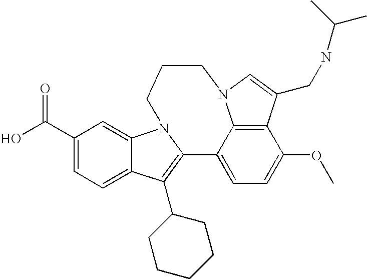 |
| 464 | 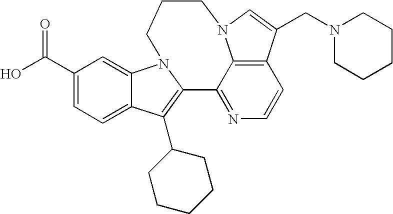 |
| 465 | 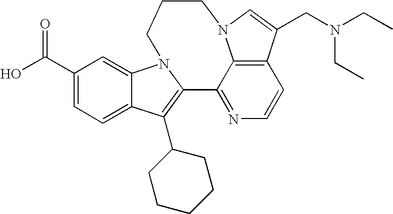 |
| 466 | 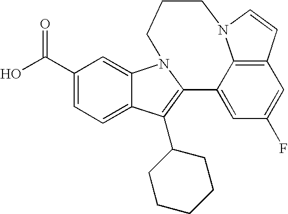 |
| 467 | 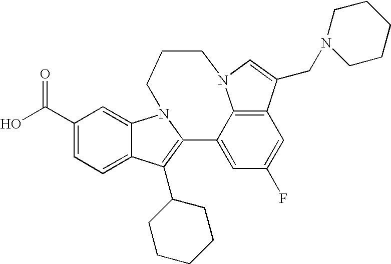 |
| 468 | 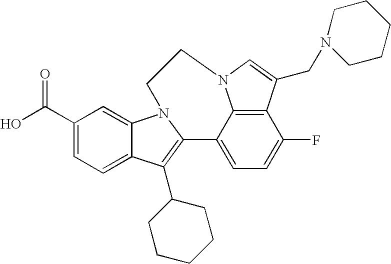 |
| 469 | 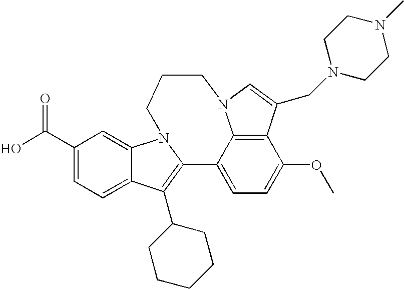 |
| 470 | 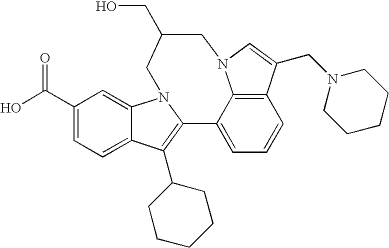 |
| 471 | 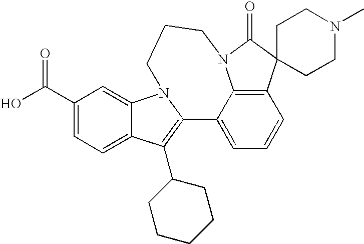 |
| 474 | 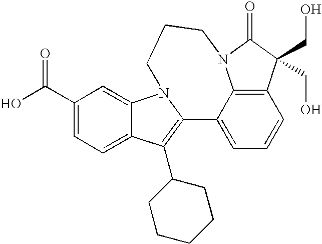 |
| 475 | 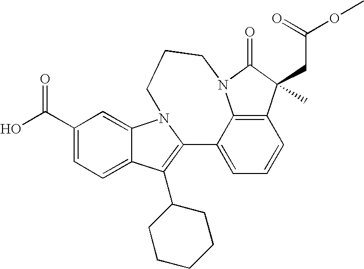 |
| 476 | 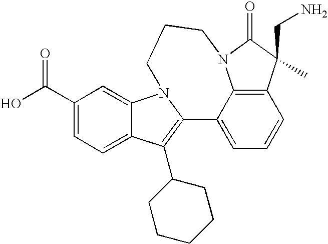 |
| 477 | 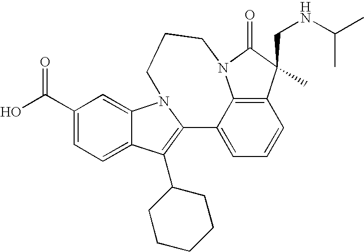 |
| 478 | 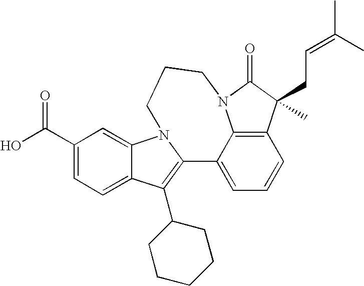 |
| 479 | 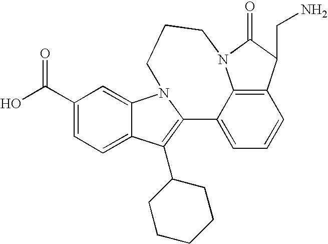 |
| 480 | 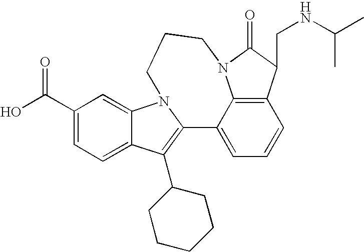 |
| 481 | 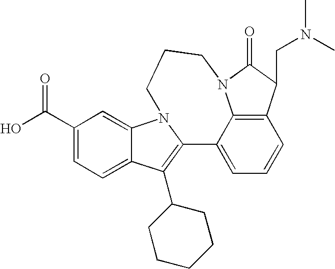 |
| 482 | 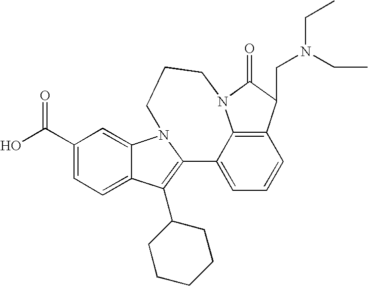 |
| 483 | 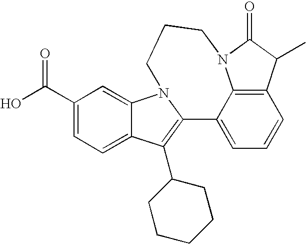 |
| 484 | 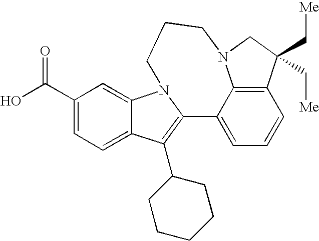 |
| 485 | 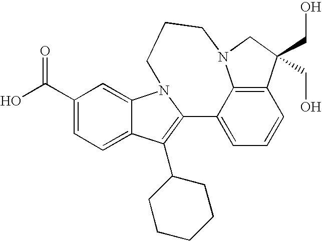 |
| 486 | 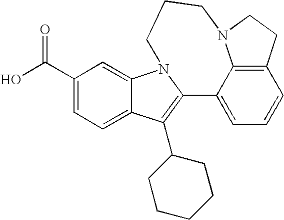 |
| 487 | 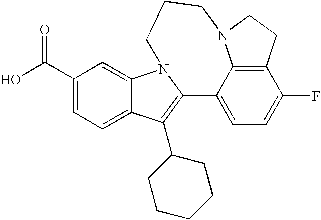 |
| 488 | 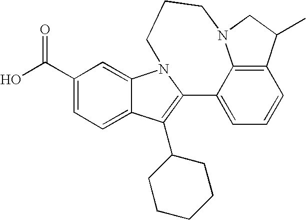 |
| 489 | 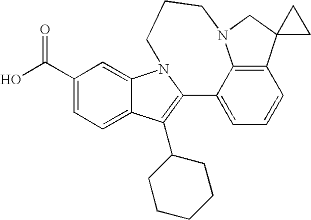 |
| 490 | 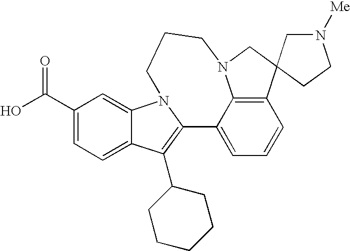 |
| 491 | 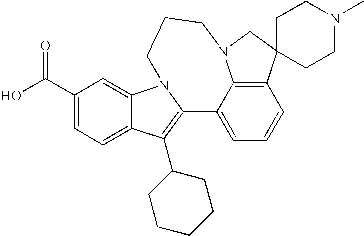 |
| 492 | 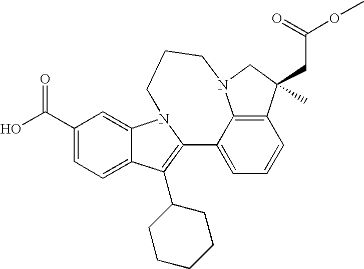 |
| 493 | 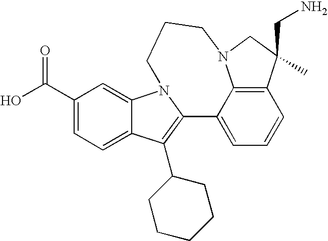 |
| 494 | 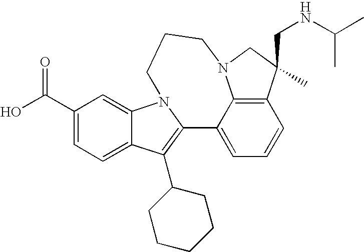 |
| 495 | 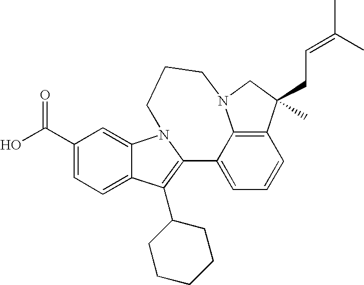 |
| 496 | 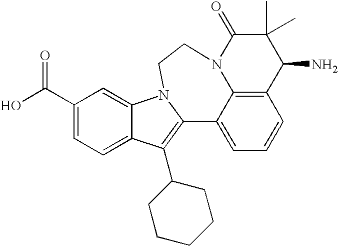 |
| 497 | 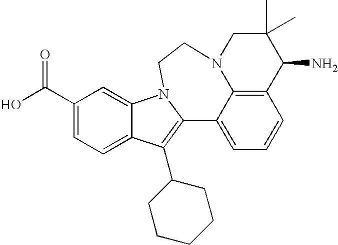 |
| 498 | 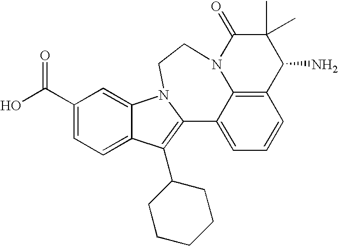 |
| 499 | 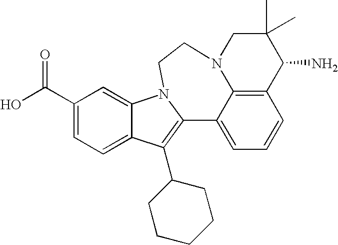 |

In other embodiments, provided are pharmaceutical compositions comprising a pharmaceutically acceptable diluent and a therapeutically effective amount of one of the compounds described herein or mixtures of one or more of such compounds.

In other embodiments, provided are methods for treating in patients a viral infection mediated at least in part by a virus in the Flaviviridae family of viruses, such as HCV, which methods comprise administering to a patient that has been diagnosed with said viral infection or is at risk of developing said viral infection a pharmaceutical composition comprising a pharmaceutically acceptable diluent and a therapeutically effective amount of one of the compounds described herein or mixtures of one or more of such compounds. In another aspect, present provided are use of the compounds of Formula (I) for the preparation of a medicament for treating or preventing said infections. In other aspects the patient is a human.

In yet another embodiment provided are methods of treating or preventing viral infections in patients in combination with the administration of a therapeutically effective amount of one or more agents active against HCV. Active agents against HCV include ribavirin, levovirin, viramidine, thymosin alpha-1, an inhibitor of NS3 serine protease, and inhibitor of inosine monophosphate dehydrogenase, interferon-alpha, pegylated interferon-alpha, alone or in combination with ribavirin or viramidine. In one example, the additional agent active against HCV is interferon-alpha or pegylated interferon-alpha alone or in combination with ribavirin or viramidine. In another example, the active agent is interferon.

### Administration and Pharmaceutical Composition

The present invention provides novel compounds possessing antiviral activity, including Flaviviridae family viruses such as hepatitis C virus. The compounds of this invention inhibit viral replication by inhibiting the enzymes involved in replication, including RNA dependent RNA polymerase. They may also inhibit other enzymes utilized in the activity or proliferation of Flaviviridae viruses.

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors. The drug can be administered more than once a day, preferably once or twice a day.

Therapeutically effective amounts of compounds of the present invention may range from approximately 0.01 to 200 mg per kilogram body weight of the recipient per day; preferably about 0.01-25 mg/kg/day, more preferably from about 0.1 to 50 mg/kg/day. Thus, for administration to a 70 kg person, the dosage range would most preferably be about 7-3500 mg per day.

This invention is not limited to any particular composition or pharmaceutical carrier, as such may vary. In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of this invention is inhalation.

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of the present invention in combination with at least one pharmaceutically acceptable excipient. Acceptable excipients are non-toxic, aid administration, and do not adversely affect the therapeutic benefit of the claimed compounds. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of a compound of the present invention based on the total formulation, with the balance being one or more suitable pharmaceutical excipients. Preferably, the compound is present at a level of about 1-80 wt %. Representative pharmaceutical formulations are described in the Formulation Examples section below.

Additionally, the present invention is directed to a pharmaceutical composition comprising a therapeutically effective amount of a compound of the present invention in combination with a therapeutically effective amount of another active agent against RNA-dependent RNA virus and, in particular, against HCV. Agents active against HCV include, but are not limited to, ribavirin, levovirin, viramidine, thymosin alpha-1, an inhibitor of HCV NS3 serine protease, or an inhibitor of inosine monophosphate dehydrognease, interferon-α, pegylated interferon-α (peginterferon-α), a combination of interferon-α and ribavirin, a combination of peginterferon-α and ribavirin, a combination of interferon-α and levovirin, and a combination of peginterferon-α and levovirin. Interferon-α includes, but is not limited to, recombinant interferon-α2a (such as ROFERON interferon available from Hoffman-LaRoche, Nutley, N.J.), interferon-α2b (such as Intron-A interferon available from Schering Corp., Kenilworth, N.J., USA), a consensus interferon, and a purified interferon-α product. For a discussion of ribavirin and its activity against HCV, see J. O, Saunders and S. A. Raybuck, “Inosine Monophosphate Dehydrogenase: Consideration of Structure, Kinetics and Therapeutic Potential,” Ann. Rep. Med. Chem., 35:201-210 (2000).

The agents active against hepatitis C virus also include agents that inhibit HCV proteases, HCV polymerase, HCV helicase, HCV NS4B protein, HCV entry, HCV assembly, HCV egress, HCV NS5A protein, and inosine 5′-monophosphate dehydrogenase. Other agents include nucleoside analogs for the treatment of an HCV infection. Still other compounds include those disclosed in WO 2004/014313 and WO 2004/014852 and in the references cited therein. The patent applications WO 2004/014313 and WO 2004/014852 are hereby incorporated by references in their entirety.

Specific antiviral agents include Omega IFN (BioMedicines Inc.), BILN-2061 (Boehringer Ingelheim), Summetrel (Endo Pharmaceuticals Holdings Inc.), Roferon A (F. Hoffman-La Roche), Pegasys (F. Hoffman-La Roche), Pegasys/Ribaravin (F. Hoffman-La Roche), CellCept (F. Hoffman-La Roche), Wellferon (GlaxoSmithKline), Albuferon-α (Human Genome Sciences Inc.), Levovirin (ICN Pharmaceuticals), IDN-6556 (Idun Pharmaceuticals), IP-501 (Indevus Pharmaceuticals), Actimmune (InterMune Inc.), Infergen A (InterMune Inc.), ISIS 14803 (ISIS Pharamceuticals Inc.), JTK-003 (Japan Tobacco Inc.), Pegasys/Ceplene (Maxim Pharmaceuticals), Ceplene (Maxim Pharmaceuticals), Civacir (Nabi Biopharmaceuticals Inc.), Intron A/Zadaxin (RegeneRx), Levovirin (Ribapharm Inc.), Viramidine (Ribapharm Inc.), Heptazyme (Ribozyme Pharmaceuticals), Intron A (Schering-Plough), PEG-Intron (Schering-Plough), Rebetron (Schering-Plough), Ribavirin (Schering-Plough), PEG-Intron/Ribavirin (Schering-Plough), Zadazim (SciClone), Rebif (Serono), IFN-β/EMZ701 (Transition Therapeutics), T67 (Tularik Inc.), VX-497 (Vertex Pharmaceuticals Inc.), VX-950/LY-570310 (Vertex Pharmaceuticals Inc.), Omniferon (Viragen Inc.), XTL-002 (XTL Biopharmaceuticals), SCH 503034 (Schering-Plough), isatoribine and its prodrugs ANA971 and ANA975 (Anadys), R1479 (Roche Biosciences), Valopicitabine (Idenix), NIM811 (Novartis), and Actilon (Coley Pharmaceuticals).

In some embodiments, the compositions and methods of the present invention contain a compound of the invention and interferon. In some aspects, the interferon is selected from the group consisting of interferon alpha 2B, pegylated interferon alpha, consensus interferon, interferon alpha 2A, and lymphoblastiod interferon tau.

In other embodiments the compositions and methods of the present invention contain a compound of the invention and a compound having anti-HCV activity is selected from the group consisting of interleukin 2, interleukin 6, interleukin 12, a compound that enhances the development of a type 1 helper T cell response, interfering RNA, anti-sense RNA, Imiquimod, ribavirin, an inosine 5′-monophospate dehydrogenase inhibitor, amantadine, and rimantadine.

In still other embodiments, the compound having anti-HCV activity is Ribavirin, levovirin, viramidine, thymosin alpha-1, an inhibitor of NS3 serine protease, and inhibitor of inosine monophosphate dehydrogenase, interferon-alpha, or pegylated interferon-alpha alone or in combination with Ribavirin or viramidine.

In another embodiments, the compound having anti-HCV activity is said agent active against HCV is interferon-alpha or pegylated interferon-alpha alone or in combination with Ribavirin or viramidine.

In other embodiments, provided are methods for preparing compounds of Formula (I). Details of the such methods can be found in the general syntheses examples I-X and in the synthetic Examples.

### General Synthetic Methods

The compounds disclosed herein can be prepared by following the general procedures and examples set forth below. It will be appreciated that where typical or preferred process conditions

(i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and P. G. M. Wuts, Protecting Groups in Organic Synthesis, Third Edition, Wiley, New York, 1999, and references cited therein.

If the compounds of this invention contain one or more chiral centers, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

Unless otherwise stated, in the following general schemes, Z, Q, L, R¹, R², R³, p, v, and s are as defined for Formula (I).

### Example I

Compounds according to formula IIb, where L is —CH₂CH₂NH—, —CH₂C(O)NR—, or —CH₂CH₂NR— can be synthesized by the following general methods. A substituted 2-bromoindole according to structure I-1 can be alkylated at the indole nitrogen by deprotonation with a base such as sodium hydride followed by the addition of tert-butyl 2-bromoacetate. A second indole fragment can be appended by utilizing standard Suzuki coupling conditions, to yield compounds according to structure I-2. Potassium tert-butoxide with monochloramine would give the corresponding hydrazine, and the addition of an acid such as trifluoroacetic acid (TFA) can liberate the carboxylic acid in structure I-3. The pentacyclic ring structure of 1-4, specifically wherein L is —CH₂C(O)NH—, can be formed by the addition of a peptide coupling agent such as O-(7-azabenzotriazol-1-yl)-N,N,N′,N′-tetramethyluronium hexafluorophosphate (HATU) under standard reaction conditions. Compounds according to structure I-4 can then be subjected to further chemical transformations in order to modify L. For example, reduction of the hydrazide carbonyl with a suitable reducing agent such as borane tetrahydrofuran complex would yield compounds according to structure I-5, wherein L is —CH₂CH₂NH—. Also, alkylation of the hydrazide of compound I-4 with the use of a base such as sodium hydride (NaH) and an appropriate electrophile, such as an alkyl halide for example, would give compounds according to structure I-6, wherein L is —CH₂C(O)NR—. Again, reduction of the hydrazide carbonyl with a suitable reducing agent such as borane tetrahydrofuran complex would yield compounds according to structure I-7, wherein L is —CH₂CH₂NR—.

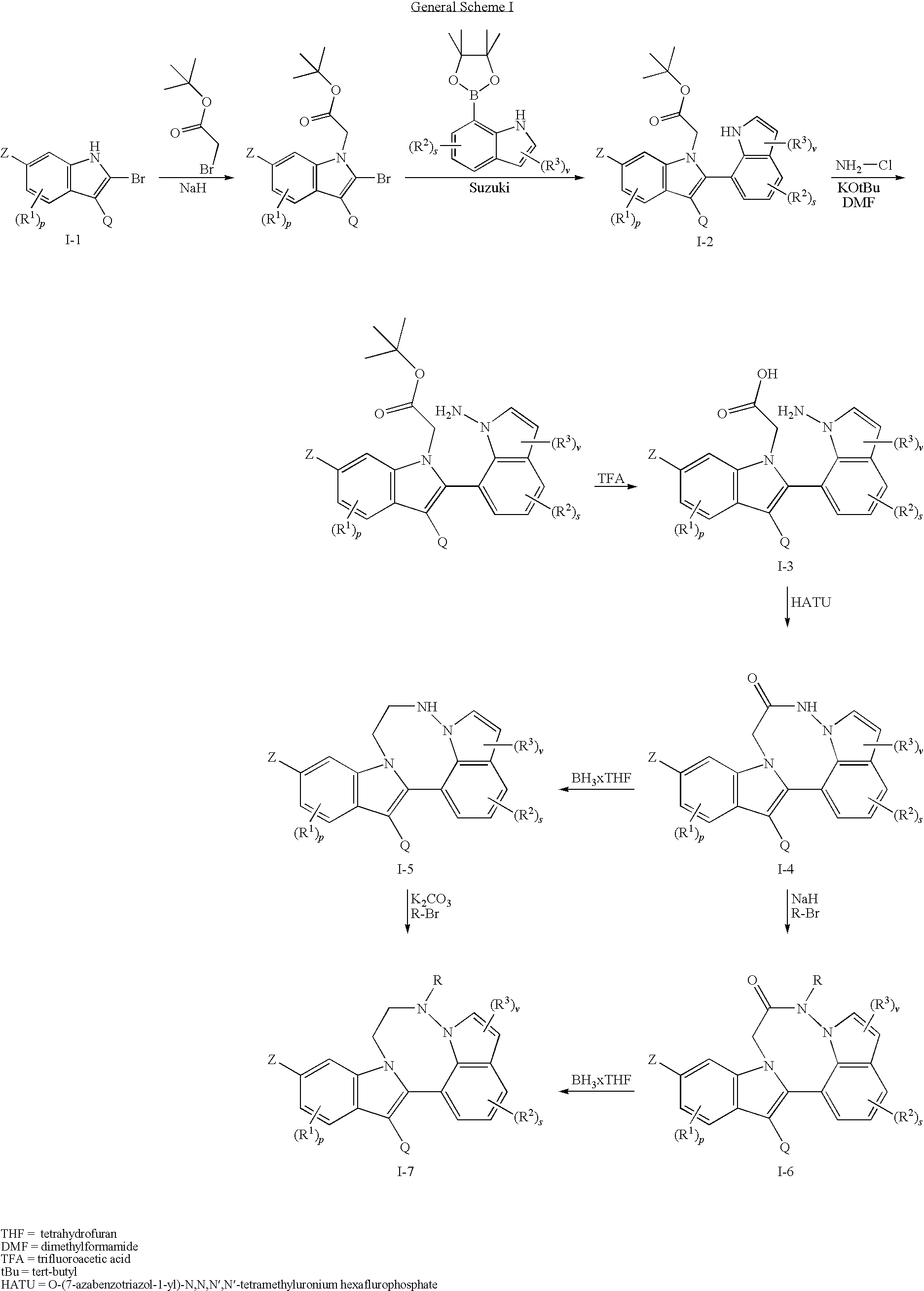

### Example II

Further derivatives of compounds according to formula IIb where L is —CH₂CH(OH)CH₂—, —CH₂CH(OR)CH₂—, —CH₂COCH₂—, or —CH₂CH(NHR)CH₂— can be synthesized by the following general methods. A substituted 2-bromoindole according to structure I-1 can be coupled with a second indole fragment under standard Suzuki coupling conditions to yield compounds according to structure II-2. Ring closure using 2-(bromomethyl)oxirane under basic conditions would yield compounds according to structure II-3 wherein L is —CH₂CH(OH)CH₂—. Such compounds can be used as intermediates for the synthesis of further derivatives, some of which are shown below. For example, alkylation of the newly formed hydroxy moiety of structure II-3 using a base such as sodium hydride (NaH) and an appropriate electrophile, such as a alkyl halide for example, would give compounds according to structure II-4, wherein L is —CH₂CH(OR)CH₂—. Oxidation of the newly formed hydroxy moiety of structure II-3 using an oxidizing agent such as Dess-Martin Periodinane, would give compounds according to structure II-5, wherein L is —CH₂COCH₂—. Furthermore, reductive amination of compounds according to structure II-5 can give compounds according to structure II-6, wherein L is —CH₂CH(NHR)CH₂—.

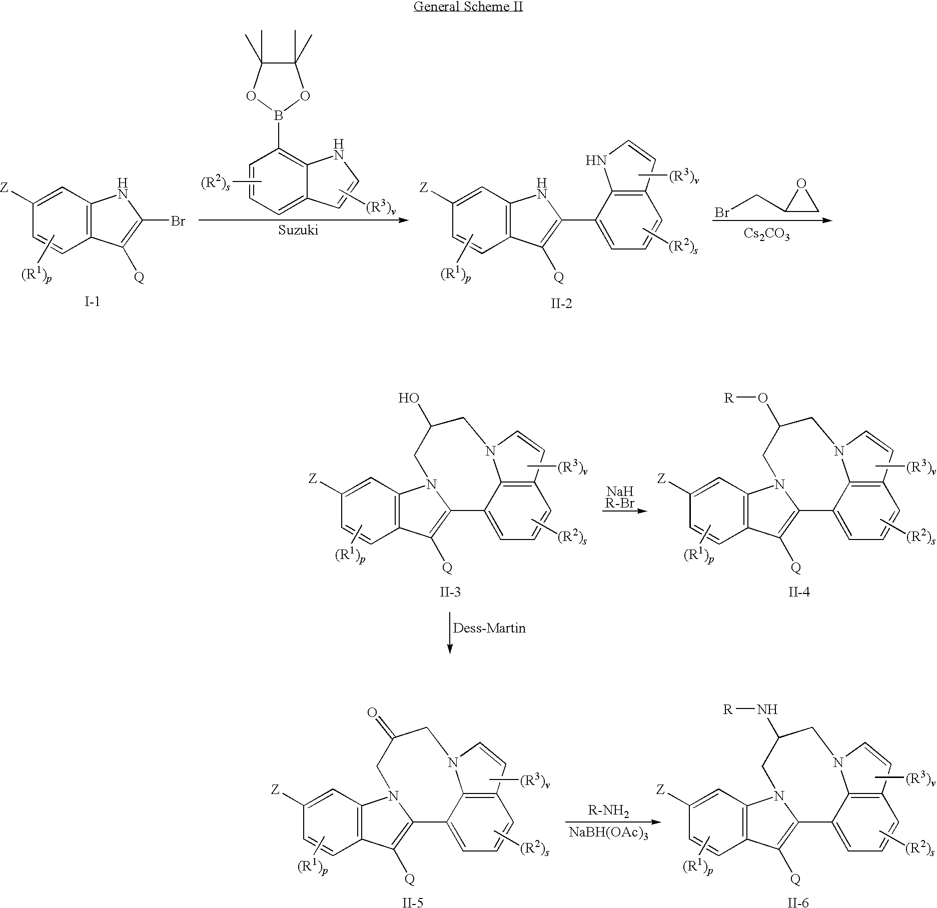

### Example III

Further compounds according to formula IIb, where L is —(CH₂)₃— can be synthesized by the following general methods. The substituted 2-bromoindole according to structure I-1 can be alkylated at the indole nitrogen by deprotonation with a base such as sodium hydride followed by the addition of 1-bromo-2-(methoxymethoxy)ethane. A second indole fragment can be appended by utilizing standard Suzuki coupling conditions, to yield compounds according to structure III-2. The addition of an acid such as trifluoroacetic acid (TFA) can liberate the amino ethanol moiety of structure III-3. The pentacyclic ring structure can be formed by suitable derivatization of the ethanol amine with a reagent such as methanesulfonyl chloride (MsCl) to provide a suitable leaving group. The subsequent nucleophilic substitution reaction can be facilitated by the use of an appropriate base such as sodium hydride to yield compounds according to structure III-5.

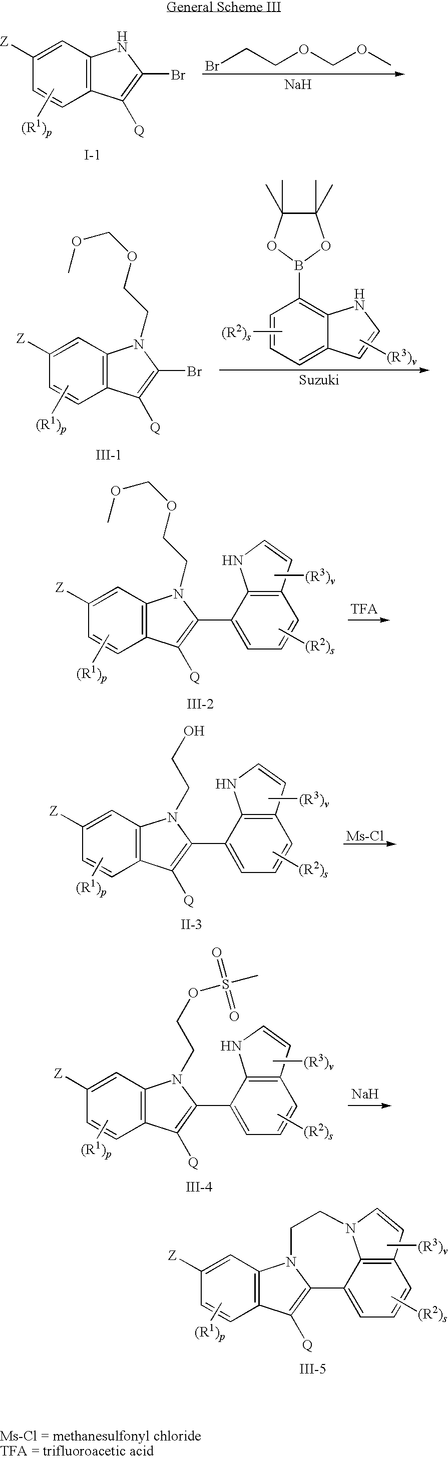

### Example IVa

Compounds according to structure IV-5 can be synthesized by the following general method. Compounds according to structure IV-1 can be synthesized starting with 7-bromo-1H-indole-2-carboxylic acid. Benzylation of 7-bromo-1H-indole-2-carboxylic acid using benzyl bromide (Bn-Br) and subsequent conversion to a suitable borane for a Suzuki coupling reaction using bis(pinacolato)diboron and a palladium source would yield compounds according to structure IV-1. The bromoindoles according to structure IV-2 can be synthesized via alkylation of I-1 using a silyl protected 3-bromopropanol under basic conditions. With both Suzuki reagents prepared, coupling under standard coupling conditions would provide the compounds according to structure IV-3. Deprotection of the silyl protecting group with a fluoride source such as tetrabutylammonium fluoride (TBAF) would liberate the free alcohol, which could then be converted into a mesylate with methanesulfonyl chloride (Ms-Cl) to give compounds according to structure IV-4. Then, ring closure could ensue under basic conditions to yield compounds according to structure IV-5, wherein L is —(CH₂)₃—.

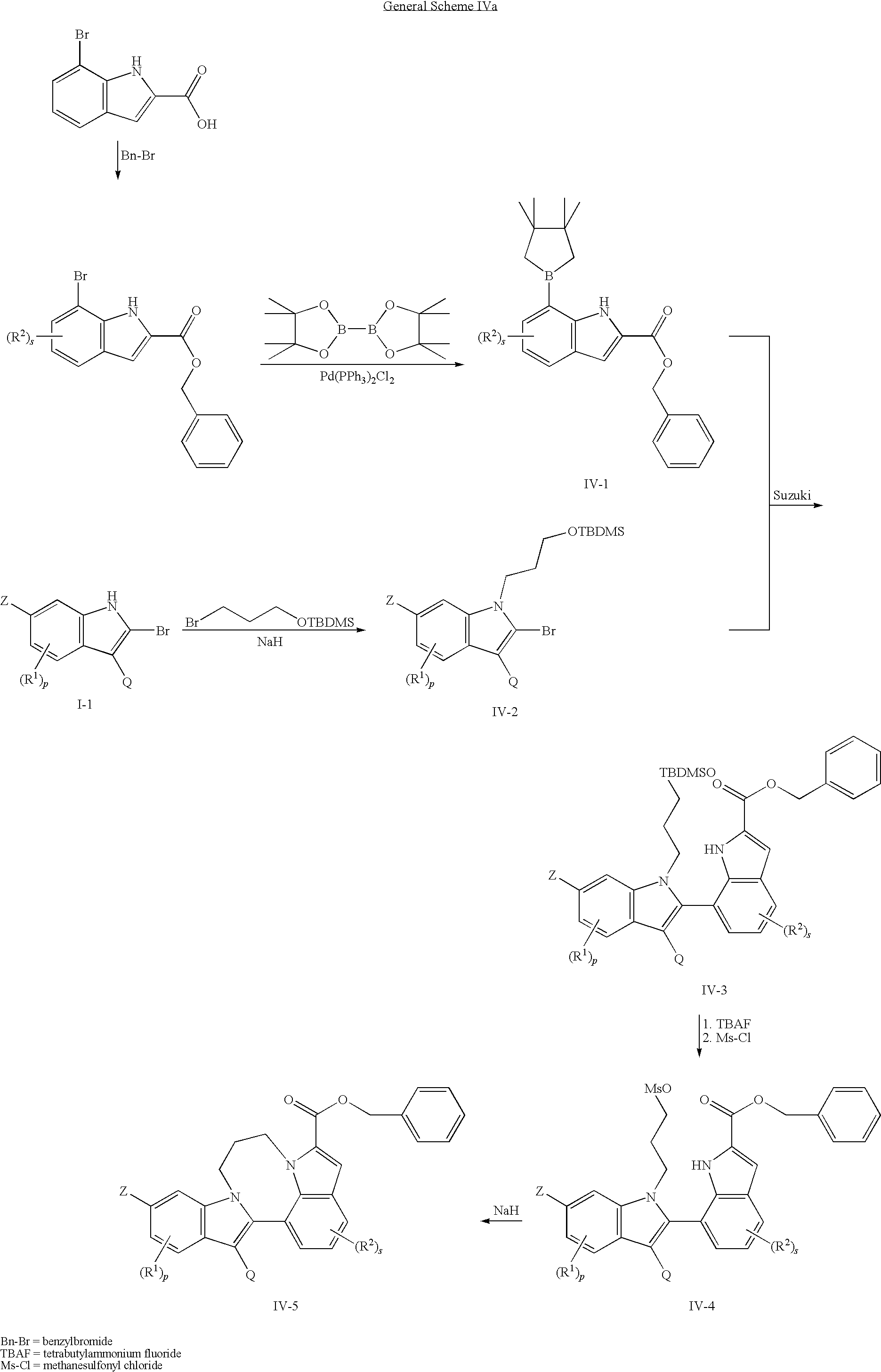

### Example IVb

Compounds of structure IV-5 can be debenzylated under hydrogenolysis conditions to yield the corresponding free acid (IV-6). Conversion of the newly formed carboxylic acid to amide IV-7 can be accomplished using standard peptide coupling reagents such as O-benzotriazole-N,N,N′,N′-tetramethyl-uronium-hexafluoro-phosphate (HBTU) with a desired amine. Compounds of structure IV-7 can also be reduced with a reducing agent such as borane tetrahydrofuran complex to yield the corresponding amine IV-8.

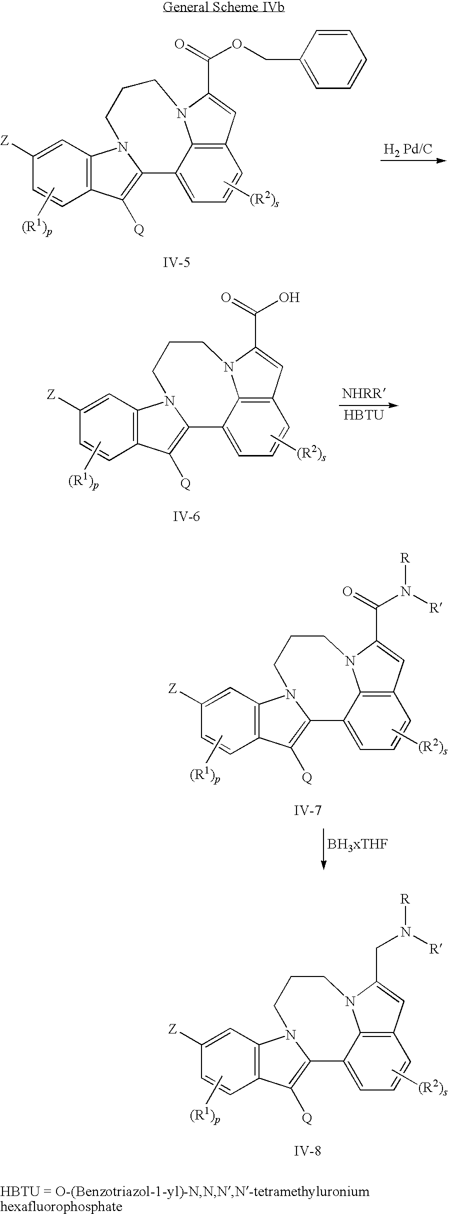

### Example V

The compounds described above in example 1V can be further used as intermediates for the synthesis of many structurally unique compounds. Compounds according to structure V wherein R³ is hydrogen, can be synthesized using the methods shown in Schemes IVa and IVb. Likewise, the addition of an amine/formaldehyde solution would result in the formation of V-3. Subsequent reduction with a reagent such as sodium cyanoborohydride would give V-4. Similarly, reduction of V with a reagent such as sodium cyanoborohydride would give V-5. The selective fluorination of V would give compounds of structure V-6. In addition, compounds of structure V-7 can synthesized from V and nitroethene. Iodination of V using reagents such as N-iodosuccinimide would yield a compound according to structure V-1, which could be reacted with trimethylsilyl cyanide (TMS-CN) under palladium catalyzed reaction conditions to give V-8. Amination of V-1 under conditions such as those reported by Buckwald and coworkers would yield V-2. The reaction of V-1 under standard Suzuki coupling conditions could give compounds according to structure V-9. Likewise, alkynylation of V-1 would produce compounds according to structure V-10, and subsequent reduction under standard alkyne reducing conditions would provide a route to V-11.

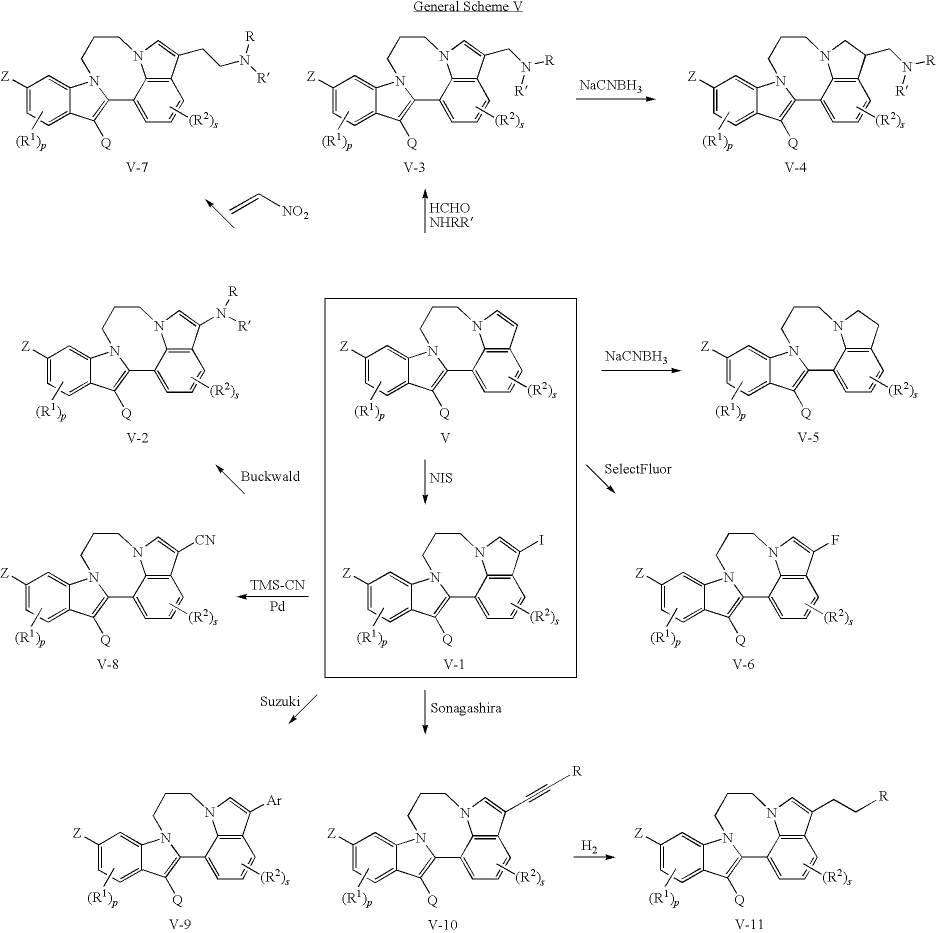

### Example VI

The synthesis of substituted compounds according to formula IIb, where L is —(CH₂)₃— and R² is varied (VI-7 and VI-9), can be synthesized according to the following methods. For example, 4-methoxy-1H-indole (VI-1) can be protected and brominated to yield VI-3. At this stage, the indole can be derivatized and the boron-moiety can be appended via a boron-halogen exchange to give compounds according to structure VI-4. Compounds according to structure VI-5 can then be synthesized by reacting VI-4 and a compound according to structure IV-2 under standard Suzuki coupling conditions, followed by the steps outlined in Scheme IVa to complete the pentacyclic ring system. Deprotection of the methyl ether using boron tribromide would yield phenols according to structure VI-6. The phenol of structure VI-6 can then be used to synthesize various derivatives (VI-7) by the addition of an electrophile. In addition, conversion of the phenol to a suitable leaving group such as trifluoroacetate would allow for a variety of aromatic substitution reactions and a pathway to compounds according to structure VI-9.

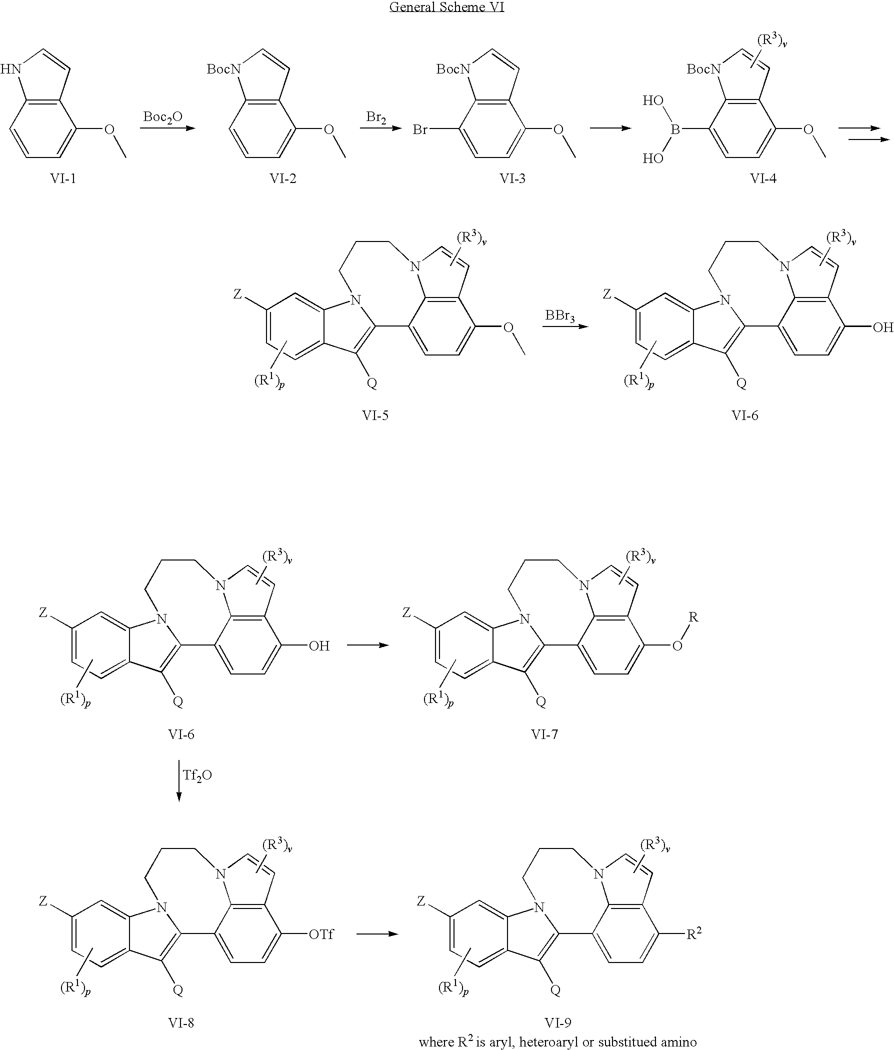

### Example VII

Compounds according to structure VII-10 and VII-12 can be synthesized starting with substituted indoles of structure VII-1 by the following methods. Deprotection of the acetate of VII-1 using methanolic ammonia, for example, would yield the corresponding phenol. Then, either the benzyl ether or methyl ether VII-3 can be formed by the reaction of VII-2 with the appropriate organohalide under basic conditions.

The substituted 2-bromoindole according to structure VII-3 can be alkylated at the indole nitrogen by deprotonation with a base such as sodium hydride followed by the addition of 1-bromo-2-(methoxymethoxy)ethane. A second indole fragment can be appended by utilizing standard Suzuki coupling conditions, to yield compounds according to structure VII-5. The addition of an acid such as trifluoroacetic acid (TFA) can liberate the amino ethanol moiety of structure VII-6. The pentacyclic ring structure can be formed by suitable derivatization of the ethanol amine with a reagent such as methanesulfonyl chloride (MsCl) to provide a suitable leaving group. The subsequent nuclephilic substitution reaction can be facilitated by the use of an appropriate base such as sodium hydride to yield compounds according to structure VII-8, wherein L is —(CH₂)₃—. Liberation of the phenol using appropriate deprotection chemistry would give compounds of structure VII-9. Subsequent modification of the phenol would provide compounds according to structures VII-10 and VII-11. For example, various derivatives of VII-10 can be synthesized by the addition of a suitable electrophile. In addition, conversion of the phenol to a suitable leaving group such as trifluoroacetate would allow for a variety of aromatic substitution reactions and a pathway to compounds according to structure VII-12.

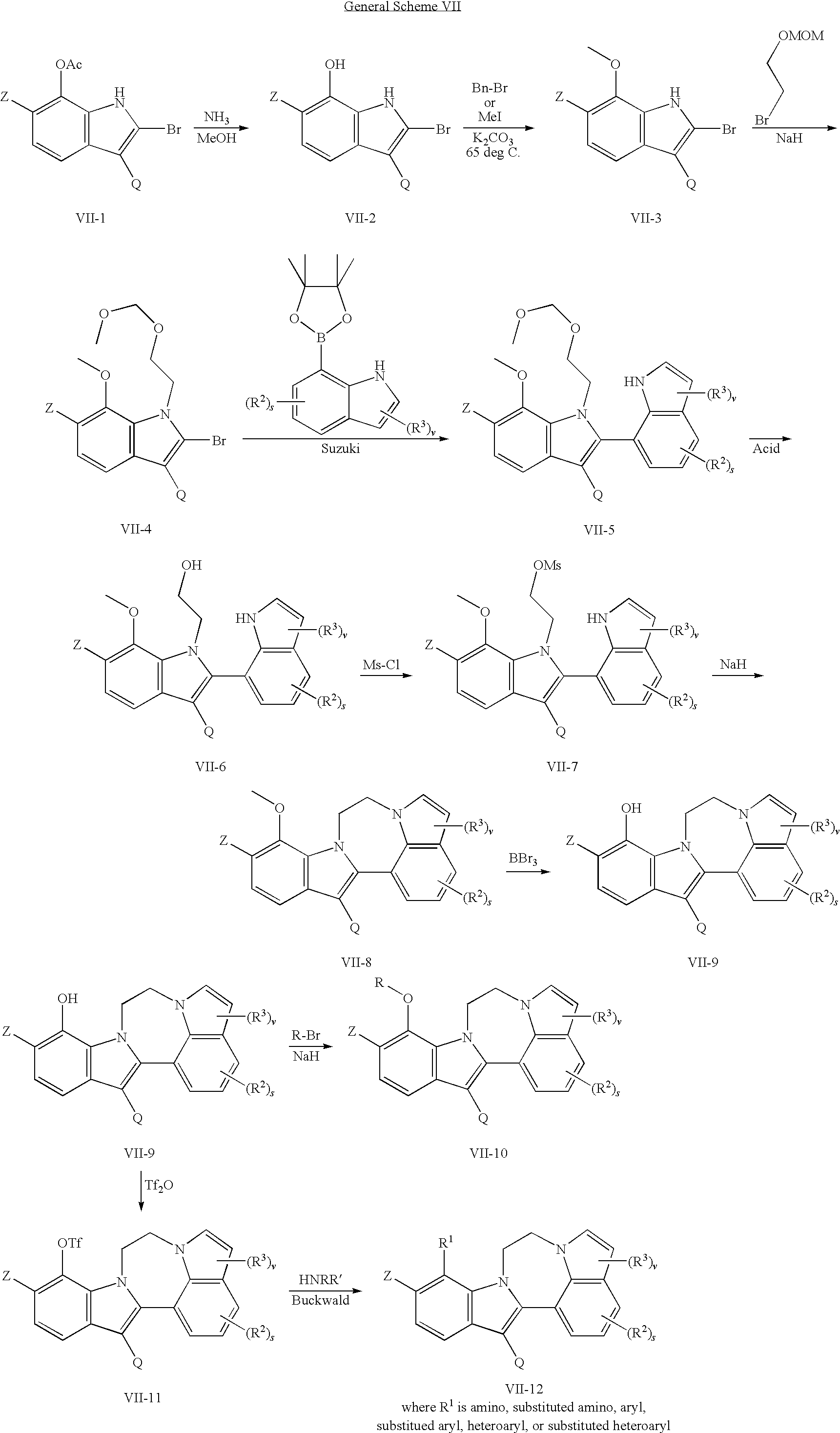

### Example VIII

Compounds according to formula IIc wherein L is —(CH₂)₃— can be synthesized by the following general methods. A substituted 2-bromoindole according to structure VIII-1 can be coupled to a substituted 3-amino-2-nitrophenylboronic acid by utilizing standard Suzuki coupling conditions, to yield compounds according to structure VIII-2. Reduction of the nitro group followed by the addition of acetic acid with heat would yield the benzimidazole VIII-4. Finally, formation of the pentacyclic ring structure can be accomplished with 1,3-dibromopropane under basic conditions, yielding compounds of structure VIII-5.

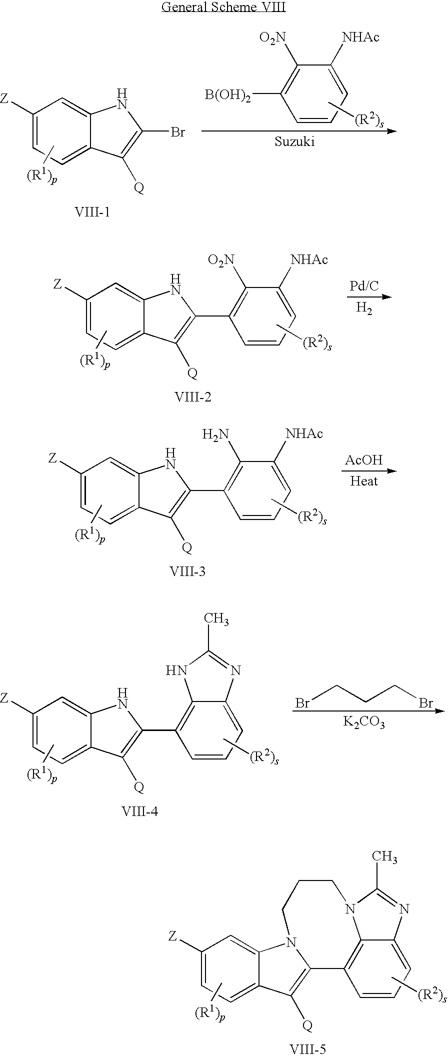

### Example IX

Compounds according to formula IIa wherein L is —(CH₂)₃— can be synthesized by the following general methods. A substituted 2-bromoindole according to structure IX-1 can be alkylated at the indole nitrogen by deprotonation with a base such as sodium hydride followed by the addition of 1-bromo-2-(methoxymethoxy)ethane. Then, IX-2 can be coupled to 1H-indol-4-ylboronic acid utilizing standard Suzuki coupling conditions, to yield compounds according to structure IX-3. The addition of an acid such as trifluoroacetic acid (TFA) can liberate the amino ethanol moiety of structure IX-4. Substitution of the alcohol to a chlorine with phosphorus oxychloride (POCl₃) for example would provide IX-5. Formation of the pentacyclic ring structure can be accomplished via Friedel-Craft alkylation of the indole using a Lewis acid such as diethylaluminum chloride, yielding compounds of structure IX-6. Various derivatives can then be formed from intermediate IX-6. For example, alklation of the indole nitrogen using a base such as sodium hydride in conjunction with an organohalide would yield compounds according to structure IX-7. Alternatively, bromination of the indole followed by amination under palladium-catalyzed reaction conditions would provide compounds according to structure IX-9.

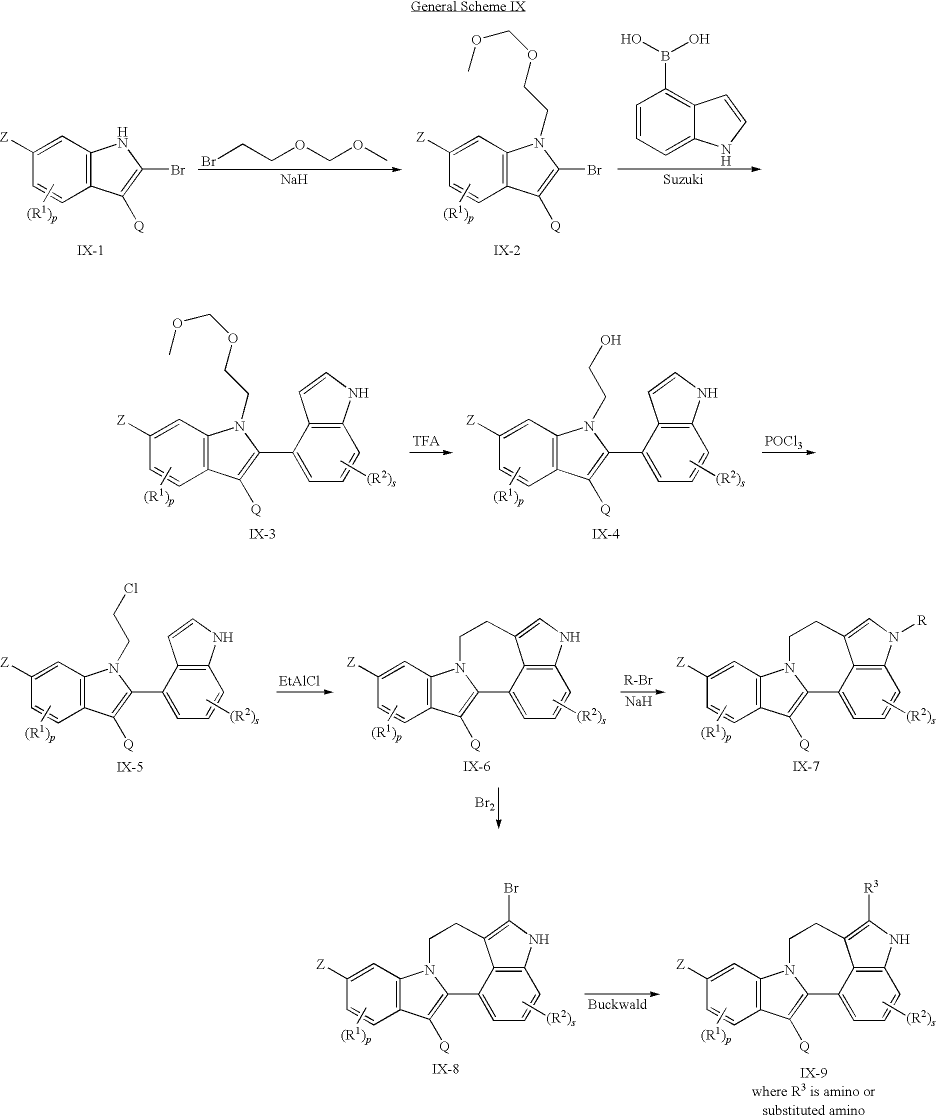

### Example X

Further to each of the above reactions is the ability to further modify the compounds at Z. The compounds according to structures I-4 to I-7, II-3 to II-6, III-5, IV-5 to IV-8, V to V-11, VI-6, VI-7, VI-9, VII-9, VII-10, VII-12, VIII-5, IX-6, IX-7 and IX-9 can be further modified at Z. For example, when Z is a methyl ester, hydrolysis using reagents such as sodium hydroxide, lithium hydroxide or potassium hydroxide would produce the corresponding carboxylic acid.

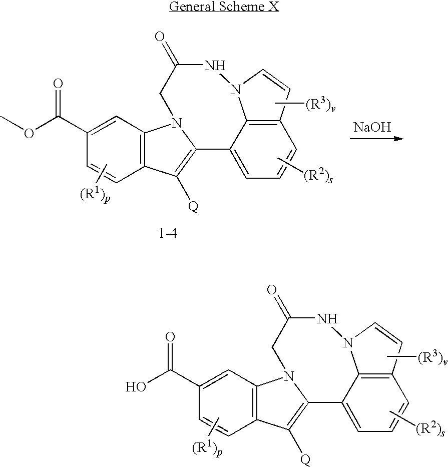

### Example XI

Compounds according to the structures XI-6 and XI-8 can be synthesized by the following general method. Michael addition of aniline XI-1 to acrylic acid followed by cyclization under dehydration conditions gives XI-3. Condensation of Ketone XI-3 with hydroxylamine gives oxime XI-4. Reduction of XI-4 using titanium tetrachloride and sodium borohydride gives amine XI-5, which could then be protected as Boc-amine XI-6. Optically active material XI-8 is prepared from XI-3 by formation of sulfinylimine XI-7 followed by reduction with sodium borohydride.

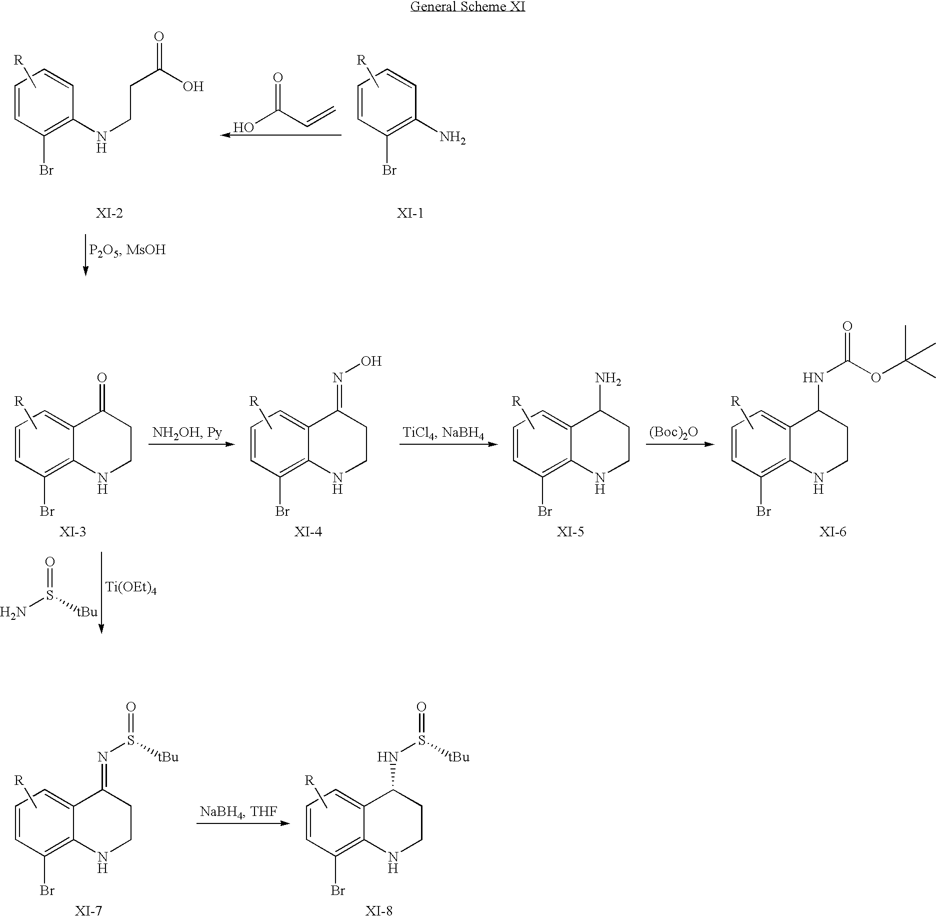

### Example XII

Compounds according to structure XII-3 can be synthesized the following general method. Ketone XI-3 is converted to α-,β-unsaturated nitrile XII-1 via a Horner-Wadsworth-Emmons reaction. Reduction of XII-1 with L-selectride followed by protection of the resulting amine XII-2 provides XII-3.

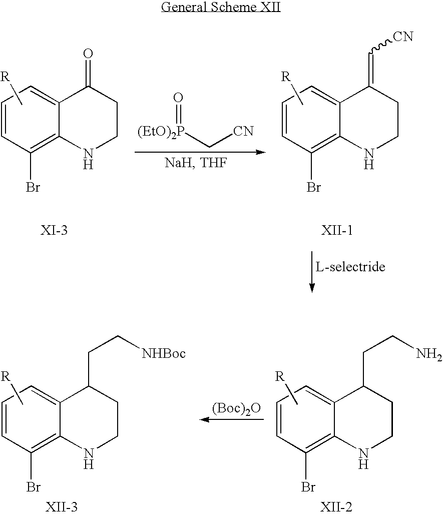

### Example XIII

Compounds according to structure XIII-3 can be synthesized by the following general method. The 8-bromotetrahydroquinoline XI-6 or XII-3 is coupled with XIII-1 under standard Suzuki coupling conditions to yield XIII-2. Acylation of XIII-2 with chloroacetyl chloride followed by intramolecular displacement and borane reduction provides XIII-3.

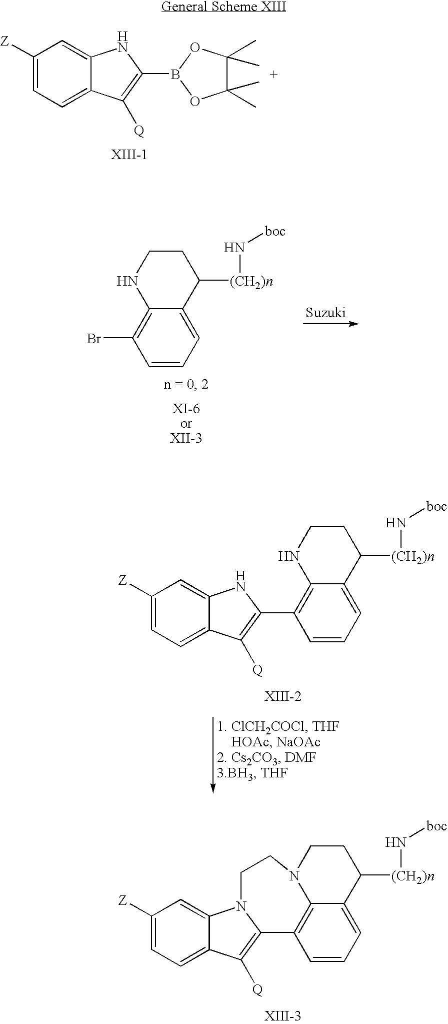

### Example XIV

XIII-3 can be used as intermediates for further synthetic transformation. Deprotection of XIII-3 under acid condition gives amine XIV-1. Reductive amination with aldehyde(s) or ketone(s) provides XIV-2. Amide coupling with carboxylic acid or reacting with acyl chloride yields XIV-3. Reacting with isocyanate gives urea XIV-4.

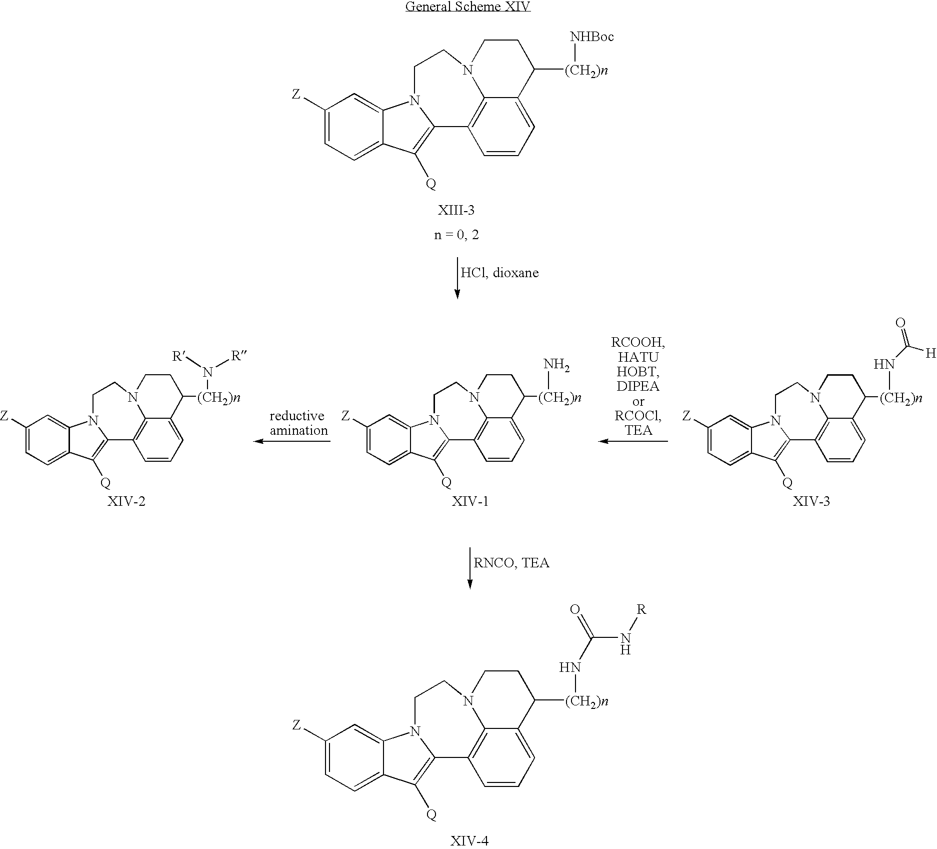

### Example XV

Compounds according to structures XV-5 and XV-6 can be synthesized starting with substituted indoles of structure XV-1 as shown in General Scheme XV. Chlorination of XV-1 using N-chlorosuccinimide (NCS), for example, yields the corresponding chloride XV-2. Hydrolysis of the chloroindole XV-2 under acidic conditions gives oxindole XV-3. Alkylation of the intermediate XV-3 using a base such as potassium carbonate in conjunction with an organohalide followed by hydrolysis with a base such as lithium hydroxide gives compounds according to structure XV-5. Reduction of intermediate XV-4 with an reducing agent such as borane followed by hydrolysis gives compounds according to structure XV-6.

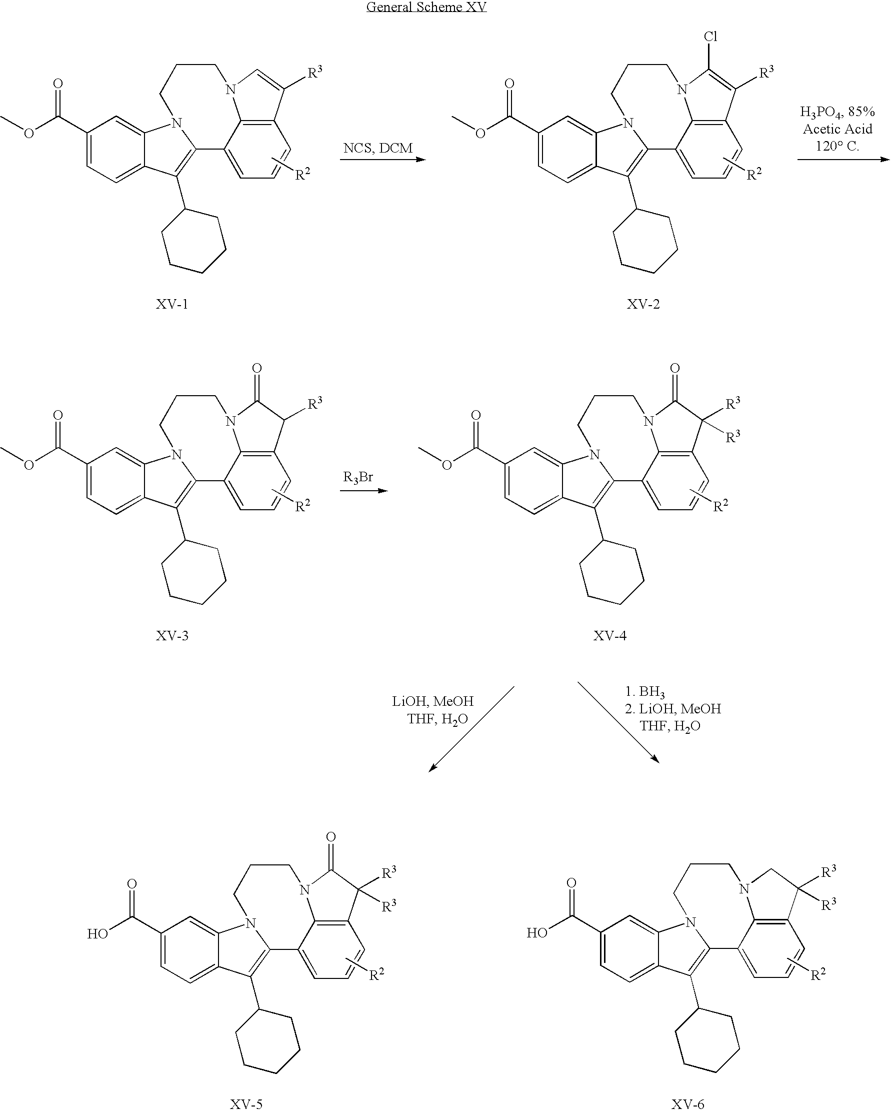

### EXAMPLES

In the examples below the following abbreviations have the indicated meanings. If an abbreviation is not defined, it has its generally accepted meaning.
aq.=aqueous
μL=microliters
μm=micromolar
NMR=nuclear magnetic resonance
br=broad
d=doublet
δ=chemical shift
° C.=degrees celcius
dd=doublet of doublets
DMEM=Dulbeco's Modified Eagle's Medium
DMF=N,N-dimethylformamide
DMSO=dimethylsulfoxide
DTT=dithiothreotol
EDTA=ethylenediaminetetraacetic acid
EtOH=ethanol
g=gram
h or hr=hours
HCV=hepatitus C virus
HPLC=high performance liquid chromatography
Hz=hertz
IU=International Units
IC₅₀=inhibitory concentration at 50% inhibition
J=coupling constant (given in Hz unless otherwise indicated)
m=multiplet
M=molar
M+H⁺=parent mass spectrum peak plus H⁺
MeOH=methanol
mg=milligram
mL=milliliter
mM=millimolar
mmol=millimole
MS=mass spectrum
nm=nanomolar
ng=nanogram
ppm=parts per million
HPLC=high performance liquid chromatography
s=Singlet
t=triplet
wt %=weight percent

### Example 5

### Preparation of Compound 105

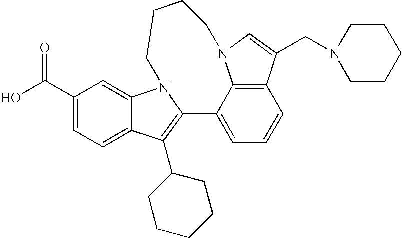

### 13-cyclohexyl-4,5,6,7-tetrahydro-[1,5]diazonino[1,2-a:5,4,3-h′i′]diindole-10-carboxylic Acid (Compound 102)

Following the full procedure and work up for compound 101 (Example 7), 3-Cyclohexyl-1H,1′H-[2,7′]biindolyl-6-carboxylic acid methyl ester (150 mg, 0.4 mmole) was reacted with 1,4-dibromobutane (130 mg, 0.6 mmole, 1.5 eq) to produce compound 102 (45 mg, 27% yield). MS: 413.2 (M+H⁺); H¹-NMR (DMSO d₆): 8.15 (s, 1H), 7.85 (d, 1H, J=8.7 Hz), 7.68 (m, 2H), 7.29 (d, 1H, J=3 Hz), 7.15 (t, 1H, J=7.5 Hz), 7.03 (m, 1H, J=6.3 Hz), 6.57 (d, 1H, J=3 Hz), 4.50 (m, 1H), 3.87 (m, 1H), 3.62 (m, 1H), 3.00 (m, 1H), 1.68 (m, 1H), 1.18 (m, 5H).

Compound 105

This compound was prepared as described for compound 121 in Example 21 in 0.125 mmole scale, using compound 102 and piperidine. ¹H NMR (DMSO-d₆, 300 MHz): δ 9.360 (s, 1H), 8.151 (s, 1H), 7.96 (d, 1H, J=8.1 Hz), 7.855 (d, 1H, J=8.7 Hz), 7.658 (d, 1H, J=8.4 Hz), 7.533 (s, 1H), 7.279 (t, 1H, J=7.8 Hz) 7.121 (d, 1H, J=6.9 Hz), 4.458 (m, 3H), 3.94 (m, 1H), 3.55-3.24 (m, 3H), 3.05-2.84 (m, 3H), 2.424 (m, 1H), 1.94-1.52 (m, 14H), 1.52-0.95 (m, 6H). MS (M+H⁺): 510.3.

### Example 6

### Preparation of Compound 106

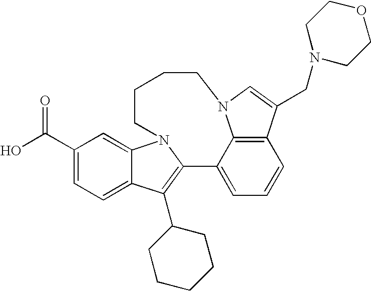

This compound was prepared as described for compound 121 in Example 21 in 0.125 mmole scale, using compound 102 in Example 5 and morpholine. ¹H NMR (DMSO-d₆, 300 MHz): δ 9.929 (s, 1H), 8.093 (s, 1H), 7.932 (d, 1H, J=7.5 Hz), 7.800 (d, 1H, J=8.1 Hz), 7.600 (d, 1H, J=8.4 Hz), 7.487 (s, 1H), 7.231 (t, 1H, J=7.8 Hz), 7.07 (d, 1H, J=6.6 Hz), 4.465 (m, 3H), 3.900 (m, 3H), 3.591 (m, 3H), 3.40-2.90 (m, 6H), 2.358 (m, 1H), 1.85-1.50 (m, 10H), 1.50-0.90 (m, 8H); MS (M+H⁺): 512.2.

### Example 7

### Preparation of Compound 107

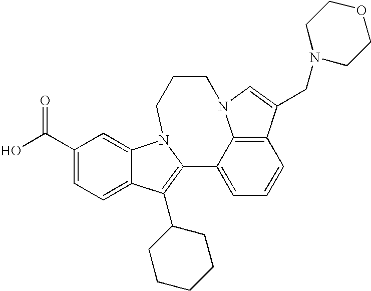

### 3-Cyclohexyl-1H,1′H-[2,7′]biindolyl-6-carboxylic Acid Methyl Ester

2-Bromo-3-cyclohexyl-1H-indole-6-carboxylic acid methyl ester (1 g, 2.98 mmole), 7-(4,4,5,5-Tetramethyl-[1,3,2]dioxaborolan-2-yl)-1H-indole (1.46 g, 5.96 mmole, 2 eq), and tetrakis(triphenylphosphine)palladium(0) (332 mg, 0.298 mmole, 0.1 eq) were dissolved in a 1:1 mixture of methanol and DMF (32 mL) and aqueous saturated sodium bicarbonate (3.2 mL) was added. The reaction was run in 2 batches in 20 mL vials in a microwave synthesis unit at 130° C. for 15 minutes each. The resulting crude was concentrated and purified via silica gel chromatography to yield 3-Cyclohexyl-1H,1′H-[2,7′]biindolyl-6-carboxylic acid methyl ester (1.10 g, 99% yield). MS: 373.1 (M+H⁺); H¹-NMR (DMSO d₆): 11.58 (s, 1H), 10.92 (s, 1H), 7.99 (s, 1H), 7.84 (d, 1H, J=8.4 Hz), 7.62 (m, 2H), 7.29 (m, 1H), 7.13 (m, 2H), 6.53 (m, 1H), 3.85 (s, 1H), 2.71 (m, 1H), 1.79 (m, 7H), 1.25 (m, 3H).

### 12-cyclohexyl-5,6-dihydro-4H-[1,5]diazocino[1,2-a:5,4,3-h′i′]diindole-9-carboxylic Acid (compound 101)

3-Cyclohexyl-1H,1′H-[2,7′]biindolyl-6-carboxylic acid methyl ester (150 mg, 0.4 mmole) was dissolved in DMF (5 mL) in a 40 mL screw cap vial with a stir bar. 60% NaH (64 mg, 1.6 mmole, 4 eq) was added and the flask was placed under vacuum until the vigorous bubbling had stopped. The reaction was then back filled with argon, and 1,3-dibromopropane (61 μL, 0.6 mmole, 1.5 eq) was added. The reaction was stirred under vacuum at ambient temperature for 1 hour, and purified via RP-HPLC to yield compound 101 (20 mg, 13% yield). MS: 399.2 (M+H⁺); H¹-NMR (DMSO d₆): 8.11 (d, 1H, J=0.9 Hz), 7.89 (d, 1H, J=8.4 Hz), 7.66 (m, 2H), 7.38 (d, J=3.3 Hz), 7.16 (t, 1H, J=7.2 Hz), 7.07 (m, 1H), 6.54 (d, 1H, J=3 Hz), 4.59 (m, 1H), 4.12 (m, 1H), 3.57 (m, 1H), 3.21 (m, 1H), 2.85 (m, 1H), 1.94 (m, 6H), 1.68 (m, 2H), 1.54 (m, 1H), 1.29 (m, 3H).

Compound 107

This compound was prepared as described for compound 121 in Example 21 in 0.125 mmole scale, using compound 101 and morpholine. ¹H NMR (DMSO-d₆, 300 MHz): δ 10.2 (s, 1H), 8.07 (d, 1H, J=1.2 Hz), 7.91 (d, 1H, J=7.8 Hz), 7.845 (d, 1H, J=8.4 Hz), 7.595 (d, 1H, J=8.4 Hz), 7.574 (s, 1H), 7.248 (t, 1H, J=7.8 Hz) 7.10 (d, 1H, J=6.9 Hz), 4.565 (m, 1H), 4.463 (s, 2H), 4.13 (m, 1H), 3.905 (m, 2H), 3.67-3.44 (m, 3H), 3.40-3.04 (m, 5H), 2.82-2.70 (m, 1H), 2.05-1.85 (m, 5H), 1.85-1.76 (m, 1H), 1.68-1.58 (m, 2H), 1.52-1.44 (m, 1H), 1.33-1.10 (m, 2H), 1.10-0.90 (m, 1H); MS (M+H⁺): 498.3.

### Example 8

### Preparation of Compound 108

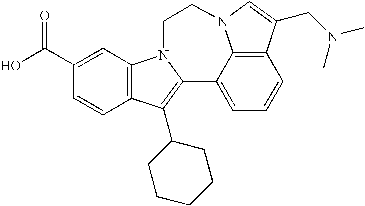

### 2-Bromo-3-cyclohexyl-1-(2-methoxymethoxy-ethyl)-1H-indole-6-carboxylic Acid Methyl Ester

To a solution of 11.0 g (2.974 mmole) 2-bromo-3-cyclohexyl-1H-indole-6-carboxylic acid methyl ester in 7.5 mL DMF, 149 mg (3.720 mmole) 60% suspension of NaH in mineral oil was added at room temperature. The evolving hydrogen was pooled out by keeping under mild vacuum for 15 minutes when 438.1 μL (3.720 mmole) 1-bromo-2-methoxymethoxy-ethane was added. The reaction was complete after overnight agitation. It was evaporated to dryness and the resulting oily product was used without further purification. MS (M+H⁺): 424.1; 426.1

### 3-Cyclohexyl-1-(2-methoxymethoxy-ethyl)-1H,1′H-[2,7′]biindolyl-6-carboxylic Acid Methyl Ester

The whole amount of 2-bromo-3-cyclohexyl-1-(2-methoxymethoxy-ethyl)-1H-indole-6-carboxylic acid methyl ester from the previous step (2.974 mmole) was combined with 794 mg (3.27 mmole) 7-(4,4,5,5-Tetramethyl-[1,3,2]dioxaborolan-2-yl)-1H-indole, 172 mg (0.149 mmole) tetrakis(triphenylphosphine)palladium(0), 12 mL DMF and 3 mL saturated aqueous NaHCO₃ solution. The mixture was heated in a microwave reactor at 130° C. for 15 minutes then it was evaporated to dryness and the residue was purified on a silica gel pad using toluene-ethyl acetate gradient. Yield: 1.034 g (75.5% for two steps). MS (M+H⁺): 461.2; H¹-NMR (DMSO d₆): δ (ppm) 10.84 (s, 1H), 8.15 (d, 1H, J=1.5 Hz), 7.85 (d, 1H, J=8.7 Hz), 7.67 (m, 2H), 7.25 (m, 1H), 7.14 (m, 1H), 7.05 (dd, 1H, J=7.2 Hz and 1.2 Hz), 6.51 (m, 1H), 4.20 (m, 3H), 3.87 (m, 4H), 3.41 (m, 2H), 2.89 (s, 3H), 2.45 (m, 1H), 1.9-1.1 (m, 10H).

### 3-Cyclohexyl-1-(2-hydroxy-ethyl)-1H,1′H-[2,7′]biindolyl-6-carboxylic Acid Methyl Ester

1.034 g (2.245 mmole) 3-cyclohexyl-1-(2-methoxymethoxy-ethyl)-1H,1′H-[2,7′]biindolyl-6-carboxylic acid methyl ester was dissolved in 50 mL MeOH-THF 1:1 mixture. 5 mL cc HCl was added and was heated at 50 C for 1 h when it was evaporated and purified on RP-HPLC to give 390 mg (42%) 3-cyclohexyl-1-(2-hydroxy-ethyl)-1H,1′H-[2,7′]biindolyl-6-carboxylic acid methyl ester. MS (M+H⁺): 417.2; H¹-NMR (DMSO d₆): δ (ppm) 10.83 (s, 1H), 8.16 (d, 1H, J=1.5 Hz), 7.85 (d, 1H, J=8.4 Hz), 7.66 (m, 2H), 7.25 (m, 1H), 7.14 (m, 1H), 7.03 (dd, 1H, J=7.2 Hz and 1.2 Hz), 6.51 (m, 1H), 4.02 (m, 1H), 3.87 (s, 1H), 3.75 (m, 1H), 3.46-3.29 (m, 2H under water signal), 2.42 (m, 1H), 1.9-1.02 (m, 10H).

### 3-Cyclohexyl-1-(2-methanesulfonyloxy-ethyl)-1H,1′H-[2,7′]biindolyl-6-carboxylic Acid Methyl Ester

To a cold solution of 369 mg (0.886 mmole) 3-cyclohexyl-1-(2-hydroxy-ethyl)-1H,1′H-[2,7′]biindolyl-6-carboxylic acid methyl ester and 0.494 mL (3.54 mmole) TEA in 9 mL THF 0.167 mL mesyl chloride was added. The mixture was stirred for 30 minutes while warmed up to room temperature. Ice was added and the product was extracted with 30 mL ethyl acetate. The organic phase was washed with brine (2×), dried with sodium sulfate and was evaporated to dryness. The oily residue crystallized upon standing. Yield: 431 mg (93%). MS (M+H⁺): 495.1; H¹-NMR (DMSO d₆): δ (ppm) 10.90 (s, 1H), 8.20 (d, 1H, J=1.8 Hz), 7.87 (d, 1H, J=8.4 Hz), 7.69 (m, 2H), 7.26 (m, 1H), 7.16 (m, 1H), 7.07 (dd, 1H, J=1.2 Hz and 7.5 Hz), 6.52 (m, 1H), 4.45 (m, 1H), 4.10 (m, 2H), 3.99 (m, 1H), 3.87 (s, 3H), 2.75 (s, 3H), 1.84-1.14 (m, 11H).

### 14-cyclohexyl-7,8-dihydro-[1,4]diazepino[1,7-a:4,5,6-h′i′]diindole-11-carboxylic Acid Methyl Ester (Methyl Ester of Compound 103)

To a cold solution of 406 mg (0.821 mmole) 3-cyclohexyl-1-(2-methanesulfonyloxy-ethyl)-1H,1′H-[2,7′]biindolyl-6-carboxylic acid methyl ester in 4 mL DMF, 42.5 mg 60% sodium hydride in mineral oil was added in one portion. The mixture was stirred at room temperature for 5 h then it was triturated with water and dried to give 250 mg (76%) methyl ester of compound 103. MS (M+H⁺): 399.2; H¹-NMR (DMSO d₆): δ (ppm) 8.23 (d, 1H, J=1.2 Hz), 7.93 (d, 1H, J=8.7 Hz), 7.64 (m, 2H), 7.46 (d, 1H, J=3.3 Hz), 7.30 (d, 1H, J=6.9 Hz), 7.21 (m, 1H), 6.55 (d, 1H, J=3.0 Hz), 3.87 (s, 3H), 3.34 (m, 1H under water signal), 3.14 (m, 1H), 2.13-1.20 (m, 13H).

### 14-cyclohexyl-7,8-dihydro-[1,4]diazepino[1,7-a:4,5,6-h′i′]diindole-11-carboxylic Acid (Compound 103)

200 mg (0.502 mmole) methyl ester of compound 103 was heated at 60 C.° in a solution of 10 mL MeOH, 10 mL THF and 5 mL 1M LiOH for 1 h. It was then evaporated, suspended in 10 mL water, acidified to pH 1, and the precipitate was spun down, washed with water (2×) and dried to give 175 mg (91%) compound 103 as yellow powder. MS (M+H⁺): 385.2; H¹-NMR (DMSO d₆): δ (ppm) 12.59 (s, 1H), 8.21 (d, 1H, J=1.2 Hz), 7.90 (d, 1H, J=8.4 Hz), 7.63 (m, 2H), 7.46 (d, 1H, J=3.0 Hz), 7.29 (d, 1H, J=7.2 Hz), 7.21 (m, 1H), 6.55 (d, 1H, J=3.0 Hz), 3.34 (m, 1H under the water signal), 3.14 (m, 1H), 2.14-1.21 (m, 13H).

Compound 108

This compound was prepared as described for compound 121 in Example 21 in 0.104 mmole scale, using compound 103 and dimethylamine. Yield: 36 mg. MS (M+H⁺): 442.2; H¹-NMR (DMSO d₆): δ (ppm) 12.64 (br, 1H), 9.75 (s, 1H), 8.23 (d, 1H, J=1.2 Hz), 7.93 (m, 2H), 7.71 (s, 1H), 7.64 (dd, 1H, J=1.2 Hz and 8.4 Hz), 7.37 (m, 2H), 4.48 (m, 2H), 3.34 (m, 3H under the water signal), 3.10 (m, 1H), 2.78 (s, 3H), 2.46 (s, 3H), 2.11-1.09 (m, 11H).

### Example 9

### Preparation of Compound 109

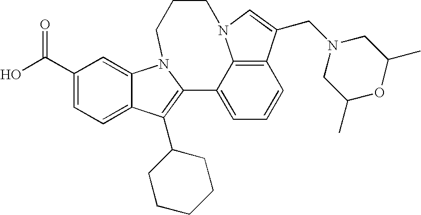

This compound was prepared as described for compound 121 in Example 21 in 0.125 mmole scale, using compound 101 and 2-6-dimethylmorpholine. Yield: 20 mg. MS (M+H⁺): 526.3; H¹-NMR (DMSO d₆): δ (ppm) 10.50 (br s, 1H), 10.23 (br s, 1H), 8.14 (s, 1H), 7.97 (d, 1H, J=7.4 Hz), 7.90 (d, 1H, J=8.3 Hz), 7.67-7.64 (m, 2H), 7.30 (t, 1H, J=7.7 Hz), 7.16 (d, 1H, J=7.2 Hz), 4.64-4.61 (m, 3H), 4.25-4.16 (m, 1H), 3.92-3.83 (m, 1H), 3.65-3.51 (m, 1H), 3.50-3.40 (m, 1H), 3.28-3.20 (m, 2H), 2.88-2.65 (m, 3H), 2.14-1.86 (m, 6H), 1.75-1.64 (m, 2H), 1.58-1.50 (m, 1H), 1.42-1.30 (m, 3H), 1.15 (d, 6H, J=6.3 Hz).

### Example 10

### Preparation of Compound 110

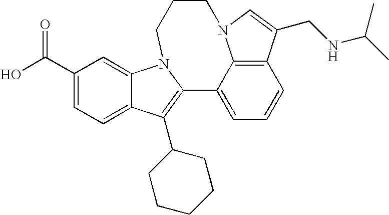

This compound was prepared as described for compound 121 in Example 21 in 0.125 mmole scale, using compound 101 and isopropylamine. Yield: 28 mg. MS (M-C₃H₉N+H⁺): 411.2; H¹-NMR (DMSO d₆): δ (ppm) 8.65 (br s, 2H), 8.13 (s, 1H), 7.90 (d, 2H, J=9.35 Hz), 7.66 (d, 1H, J=8.5 Hz), 7.60 (s, 1H), 7.30 (t, 1H, J=7.7 Hz), 7.17 (d, 1H, J=7.2 Hz), 4.68-4.58 (m, 1H), 4.38-4.30 (m, 2H), 4.24-4.14 (m, 1H), 3.64-3.50 (m, 1H), 3.28-3.16 (m, 1H), 2.88-2.70 (m, 2H), 2.30-1.40 (m, 9H), 1.33 (d, 6H, J=6.3 Hz) 1.20-0.80 (m, 3H).

### Example 11

### Preparation of Compound 111

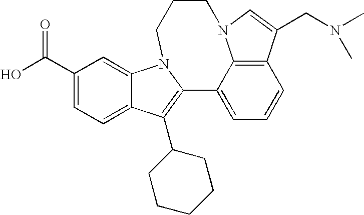

This compound was prepared as described for compound 121 in Example 21 in 0.125 mmole scale, using compound 101 and dimethylamine. ¹H NMR (DMSO-d₆, 300 MHz): δ 9.70 (s, 1H), 8.074 (d, 1H, J=1.2 Hz), 7.89 (d, 1H, J=6.9 Hz), 7.845 (d, 1H, J=8.4 Hz), 7.60 (d, 1H, J=8.4 Hz), 7.564 (s, 1H), 7.242 (t, 1H, J=7.8 Hz) 7.10 (d, 1H, J=6.9 Hz), 4.56 (m, 1H), 4.40 (s, 2H), 4.13 (m, 1H), 3.514 (m, 1H), 3.17 (m, 1H), 2.71 (m, 7H), 2.05-1.80 (m, 5H), 1.70-1.50 (m, 2H), 1.50-1.30 (m, 3H), 1.30-0.80 (m, 3H). MS (M+H⁺): 456.2.

### Example 12

### Preparation of Compound 112

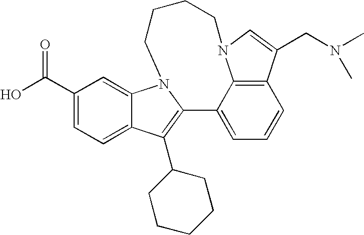

This compound was prepared as described for compound 121 in Example 21 in 0.125 mmole scale, using compound 102 and dimethylamine. ¹H NMR (DMSO-d₆, 300 MHz): δ 9.491 (s, 1H), 8.147 (s, 1H), 7.95 (d, 1H, J=7.8 Hz), 7.830 (d, 1H, J=8.4 Hz), 7.67 (d, 1H, J=8.4 Hz), 7.527 (s, 1H), 7.277 (t, 1H, J=7.8 Hz) 7.138 (d, 1H, J=6.9 Hz), 4.463 (m, 3H), 3.911 (m, 1H), 3.70-2.90 (m, 3H), 2.768 (m, 7H), 2.00-1.80 (m, 7H), 1.70-1.50 (m, 2H), 1.50-1.30 (m, 3H), 1.30-0.80 (m, 3H); MS (M+H⁺): 470.2.

### Example 13

### Preparation of Compound 113

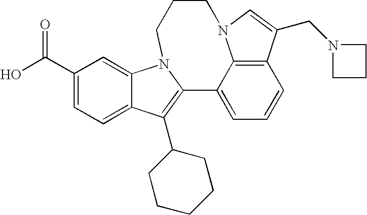

This compound was prepared as described for compound 121 in Example 21 in 0.125 mmole scale, using compound 101 and azetidine. Yield: 21 mg. MS (M+H⁺): 468.2; H¹-NMR (DMSO d₆): δ (ppm) 10.41 (br s, 1H), 8.13 (s, 1H), 7.96 (d, 1H, J=7.9 Hz), 7.90 (d, 1H, J=8.5 Hz), 7.65 (d, 1H, J=8.5 Hz), 7.63 (s, 1H), 7.28 (t, 1H, J=7.6 Hz), 7.15 (d, 1H, J=6.9 Hz), 4.64-4.50 (m, 3H), 4.18-3.98 (m, 5H), 3.58-3.54 (m, 1H), 3.36-3.18 (m, 1H), 2.81 (br s, 1H), 2.36-1.10 (m, 14H).

### Example 14

### Preparation of Compound 114

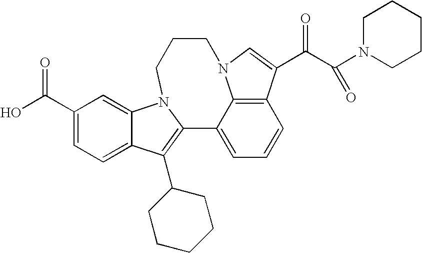

### 12-cyclohexyl-5,6-dihydro-4H-[1,5]diazocino[1,2-a:5,4,3-h′i′]diindole-9-carboxylic Acid Methyl Ester (Methyl Ester of Compound 101)

In a microwave reactor a mixture of 187.5 mg (0.5 mmole) 3-cyclohexyl-1H,1′H-[2,7′]biindolyl-6-carboxylic acid methyl ester, 72 μL (0.75 mmole) 1,3-dichloropropane and 276.4 mg (2 mmole) potassium carbonate in 5 mL DMF was heated at 160 C.° for 10 minutes. Then it was evaporated and purified on a silica gel pad to give 192 mg (92%) of methyl ester of Compound 101. MS (M+H⁺): 413.2; H¹-NMR (DMSO d₆): δ (ppm) 8.14 (d, 1H, J=0.9 Hz), 7.91 (d, 1H, J=8.4 Hz), 7.67 (m, 2H), 7.38 (d, 1H, J=3 Hz), 7.16 (m, 1H), 7.08 (d, 1H, J=8.1 Hz), 6.55 (d, 1H, J=3 Hz), 4.60 (m, 1H), 4.13 (m, 1H), 3.87 (s, 3H), 3.61 (m, 1H), 3.22 (m, 1H), 2.84 (m, 1H), 2.10-1.07 (m, 12H).

Compound 114

To a solution of the product from previous step, methyl ester of compound 101 (50 mg, 0.121 mmole) in ethyl ether (5 mL) was added oxalyl chloride (25.4 μL, 0.29 mmole) and the reaction was stirred at room temperature for 2 hours. Then piperidine (229 μL, 2.32 mmole) was added and the amide formed in 10 minutes at room temperature. The mixture was concentrated to dryness and re-dissolved in 5 mL of mixture of methanol, THF, and water in the ratio of 1:2:1. Saponification by LiOH at 50° C. for 2 hours provided the target molecule. The crude product was concentrated and re-dissolved in DMF (6 mL). Purification by HPLC gave 31 mg (48%) of the title compound. ¹H NMR (DMSO-d₆, 300 MHz): δ 12.58 (s, 1H), 8.230 (m, 2H), 8.084 (d, 1H, J=1.2 Hz), 7.855 (d, 1H, J=8.4 Hz), 7.600 (d, 1H, J=8.7 Hz), 7.388 (t, 1H, J=6.9 Hz) 7.20 (d, 1H, J=7.2 Hz), 4.57 (m, 1H), 4.30 (d, 2H, J=13.5 Hz), 3.65-3.40 (m, 3H), 3.35-3.10 (m, 2H), 2.718 (m, 1H), 2.06-1.90 (m, 5H), 1.80 (m, 1H), 1.70-1.20 (m, 1H), 1.12-0.95 (m, 1H). MS (M+H⁺): 538.2.

### Example 15

### Preparation of Compound 115

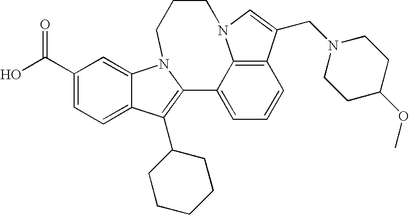

This compound was prepared as described for compound 121 in Example 21 in 0.125 mmole scale, using compound 101 and 4-methoxypiperidine. Yield: 16 mg. MS (M+H⁺): 526.3; H¹-NMR (DMSO d₆): δ (ppm) 9.87 (br s, 1H), 8.13 (s, 1H), 8.0 (d, 1H, J=7.2 Hz), 7.90 (d, 1H, J=8.8 Hz), 7.65 (d, 1H, J=12.4 Hz), 7.64 (s, 1H), 7.30 (t, 1H, J=7.4 Hz), 7.15 (d, 1H, J=7.2 Hz), 4.63 (d, 1H, J=10.2 Hz), 4.48 (s, 3H), 4.18 (d, 1H, J=14.3 Hz), 3.60-3.45 (m, 2H), 3.45-3.30 (m, 2H), 3.23 (d, 2H, J=4.7 Hz), 3.12-3.0 (m, 2H), 2.81 (br s, 2H), 2.20-1.10 (m, 16H).

### Example 16

### Preparation of Compound 116

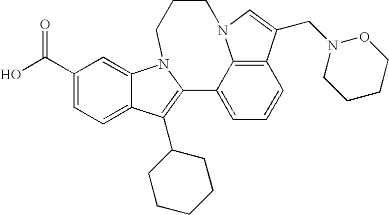

This compound was prepared as described for compound 121 in Example 21 in 0.125 mmole scale, using compound 101 and 1,2-oxazinane. Yield: 32 mg. MS (M-C₄H₉NO+H⁺): 411.2; H¹-NMR (DMSO d₆): δ (ppm) 8.12 (s, 1H), 7.90 (d, 1H, J=8.3 Hz), 7.82 (d, 1H, J=8.0 Hz), 7.67-7.64 (m, 1H), 7.47 (s, 1H), 7.23 (t, 1H, J=7.7 Hz), 7.15-7.10 (m, 1H), 4.64-4.58 (m, 2H), 4.50-4.22 (m, 2H), 4.21-4.16 (m, 3H), 3.30-3.10 (m, 2H), 2.94-2.70 (m, 3H), 2.14-1.06 (m, 16H).

### Example 17

### Preparation of Compound 117

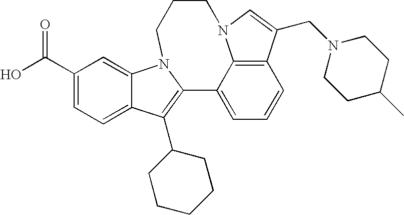

This compound was prepared as described for compound 121 in Example 21 in 0.125 mmole scale, using compound 101 and 4-methylpiperidine. Yield: 37 mg. MS (M+H⁺): 510.3; H¹-NMR (DMSO d₆): δ (ppm) 9.58 (br s, 2H), 8.14 (s, 1H), 7.95 (d, 1H, J=8.0 Hz), 7.91 (d, 1H, J=8.5 Hz), 7.68-7.63 (m, 2H), 7.30 (t, 1H, J=7.7 Hz), 7.16 (d, 1H, J=7.2 Hz), 4.68-4.58 (m, 1H), 4.48-4.42 (m, 2H), 4.24-4.14 (m, 1H), 3.32-3.18 (m, 2H), 3.06-2.76 (m, 3H), 2.14-1.09 (m, 18H), 0.90 (d, 3H, J=6.3 Hz).

### Example 18

### Preparation of Compound 118

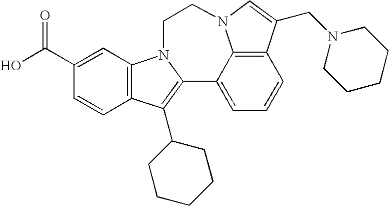

This compound was prepared as described for compound 121 in Example 21 in 0.125 mmole scale, using compound 103 and piperidine. Yield: 36 mg. MS (M+H⁺): 482.3; H¹-NMR (DMSO d₆): δ (ppm) 9.83 (br, 1H), 8.16 (d, 1H, J=0.9 Hz), 7.86 (m, 2H), 7.65 (s, 1H), 7.57 (dd, 1H, J=1.2 Hz and 8.4 Hz), 7.29 (m, 2H), 4.6-3.6 (m, 5H), 3.37 (m, 2H), 3.05 (m, 1H), 2.83 (m, 2H), 2.08-1.1 (m, 13H).

### Example 19

### Preparation of Compound 119

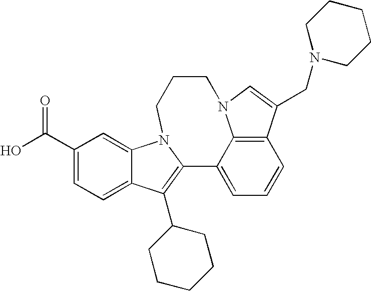

This compound was prepared as described for compound 121 in Example 21 in 0.125 mmole scale, using compound 101 and piperidine. ¹H NMR (DMSO-d₆, 300 MHz): δ 9.31 (s, 1H), 8.14 (d, 1H, J=1.2 Hz), 7.93 (m, 2H), 7.67 (d, 1H, J=8.4 Hz), 7.606 (s, 1H), 7.312 (t, 1H, J=7.8 Hz) 7.17 (d, 1H, J=6.9 Hz), 4.64 (m, 1H), 4.476 (d, 2H, J=3.6 Hz), 4.19 (m, 1H), 3.65-3.44 (m, 3H), 3.32-3.22 (m, 1H), 3.04-2.78 (m, 4H), 2.18-1.95 (m, 5H), 1.94-1.78 (m, 3H), 1.78-1.50 (m, 6H), 1.50-1.25 (m, 3H), 1.2-1.0 (m, 1H); TFA salt. MS (M+H⁺): 496.3.

### Example 20

### Preparation of Compound 120

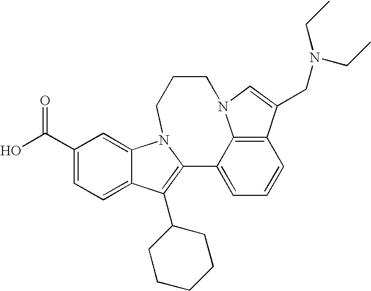

This compound was prepared as described for compound 121 in Example 21 in 0.12 mmole scale, using methyl ester of compound 101 and diethylamine. Subsequent saponification with LiOH gave the target molecule. ¹H NMR (DMSO-d₆, 300 MHz): δ 9.176 (s, 1H), 8.14 (d, 1H, J=1.2 Hz), 7.90 (m, 2H), 7.67 (m, 2H), 7.317 (t, 1H, J=7.8 Hz) 7.17 (d, 1H, J=6.9 Hz), 4.64 (m, 1H), 4.408 (d, 2H, J=3.6 Hz), 4.18 (m, 1H), 3.62-3.50 (m, 1H), 3.32-3.02 (m, 5H), 2.84 (m, 1H), 2.14-1.80 (m, 6H), 1.78-1.50 (m, 3H), 1.42-1.05 (m, 9H); HCl salt. MS (M+H⁺): 483.3.

### Example 21

### Preparation of Compound 121

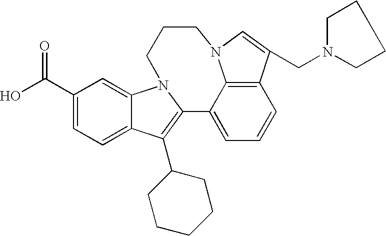

Pyrrolidine (27.3 μL, 0.33 mmole) and formaldehyde (37% aqueous solution) (26.7 μL, 0.33 mmole) were dissolved in a mixture of acetic acid (0.5 ml) and ethanol (1.5 ml) with stirring. In five minutes, compound 101 (44 mg, 0.11 mmole) was added and the reaction was heated at 50° C. for 2 hours. The crude product was concentrated and re-dissolved in DMF (8 mL). Purification by HPLC gave 44 mg (83%) of the title compound. ¹H NMR (DMSO-d₆, 300 MHz): δ 9.742 (s, 1H), 8.074 (s, 1H), 7.882 (m, 2H), 7.584 (m, 2H), 7.245 (t, 1H, J=7.8 Hz) 7.10 (d, 1H, J=7.2 Hz), 4.57 (m, 1H), 4.500 (d, 2H, J=4.5 Hz), 4.121 (m, 1H), 3.58-3.40 (m, 2H), 3.40-3.00 (m, 4H), 2.749 (m, 1H), 2.08-1.75 (m, 10H), 1.75-1.50 (m, 2H), 1.50-0.95 (m, 4H). MS (M+H⁺): 482.2.

### Example 22

### Preparation of Compound 122

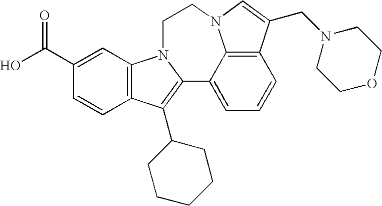

This compound was prepared as described for compound 121 in Example 21 in 0.104 mmole scale, using compound 103 and morpholine. Yield: 34 mg. MS (M+H⁺): 484.2; H¹-NMR (DMSO d₆): δ (ppm) 12.63 (br, 1H), 10.70 (s, 1H), 8.23 (d, 1H), 7.94 (m, 2H), 7.73 (s, 1H), 7.63 (dd, 1H, J=1.2 Hz and 8.7 Hz), 7.35 (m, 2H), 4.52 (m, 2H), 3.95 (m, 2H), 3.71 (m, 2H), 3.43 (m, 4H under water signal), 3.11 (m, 4H), 2.14-1.22 (m, 11H).

### Example 23

### Preparation of Compound 123

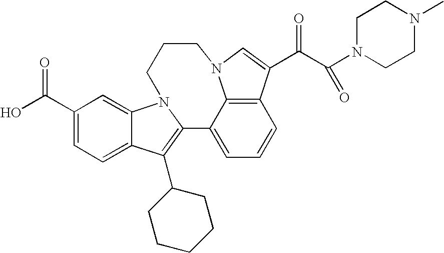

This compound was prepared as described for compound 114 in Example 14 in 0.182 mmole scale, using methyl ester of compound 101 and N-methylpiperazine. ¹H NMR (DMSO-d₆, 300 MHz): δ 10.388 (s, 1H), 8.31 (s, 1H), 7.255 (d, 1H, J=7.8 Hz), 8.097 (s, 1H), 7.860 (d, 1H, J=8.7 Hz), 7.610 (d, 1H, J=8.7 Hz), 7.415 (t, 1H, J=7.8 Hz) 7.230 (d, 1H, J=6.9 Hz), 4.600 (m, 1H), 4.45 (m, 1H), 4.24 (m, 1H), 3.79 (m, 1H), 3.65-3.24 (m, 4H), 3.24-3.04 (m, 3H), 3.04-2.84 (m, 1H), 2.84-2.64 (m, 4H), 2.10-1.95 (m, 5H), 1.85-1.76 (m, 1H), 1.68-1.58 (m, 2H), 1.52-1.44 (m, 1H), 1.40-1.10 (m, 2H), 1.10-0.90 (m, 1H). Yield: 53 mg, (53%). MS (M+H⁺): 553.3.

### Example 24

### Preparation of Compound 124

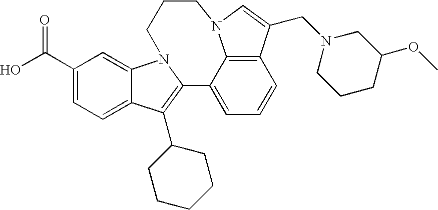

This compound was prepared as described for compound 121 in Example 21 in 0.125 mmole scale, using compound 101 and 3-methoxypiperidine. Yield: 31 mg. MS (M+H⁺): 526.3; H¹-NMR (DMSO d₆): δ (ppm) 9.88 (br s, 1H), 9.32 (br s, 1H), 8.13 (s, 1H), 7.96 (d, 1H, J=7.9 Hz), 7.91 (d, 1H, J=8.3 Hz), 7.68-7.60 (m, 2H), 7.30 (t, 1H, J=6.3 Hz), 7.16 (d, 1H, J=7.2 Hz), 4.68-4.64 (m, 1H), 4.54-4.42 (m, 2H), 4.24-4.18 (m, 1H), 3.72-3.58 (m, 2H), 3.54-3.40 (m, 2H), 3.32-3.26 (m, 2H), 3.12-2.65 (m, 4H), 2.14-1.08 (m, 15H) 0.95-0.8 (m, 1H).

### Example 25

### Preparation of Compound 125

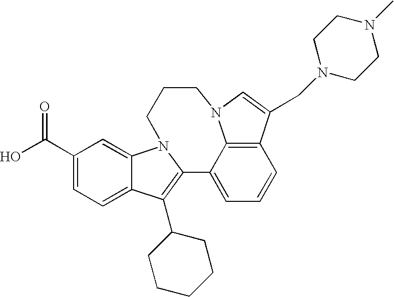

This compound was prepared as described for compound 121 in Example 21 in 0.125 mmole scale, using compound 101 and N-methylpiperazine. ¹H NMR (DMSO-d₆, 300 MHz): δ 10.2 (s, 1H), 8.07 (s, 1H), 7.845 (d, 2H, J=8.4 Hz), 7.595 (d, 1H, J=8.4 Hz), 7.478 (s, 1H), 7.218 (t, 1H, J=7.8 Hz) 7.08 (d, 1H, J=6.9 Hz), 4.565 (m, 1H), 4.290 (s, 2H), 4.10 (m, 1H), 3.515 (m, 3H), 3.40-3.15 (m, 3H), 3.40-3.04 (m, 4H), 2.82-2.60 (m, 4H), 2.05-1.85 (m, 5H), 1.85-1.76 (m, 1H), 1.68-1.58 (m, 2H), 1.52-1.44 (m, 1H), 1.33-1.10 (m, 2H), 1.10-0.90 (m, 1H); MS (M+H⁺): 511.3.

### Example 26

### Preparation of Compound 126

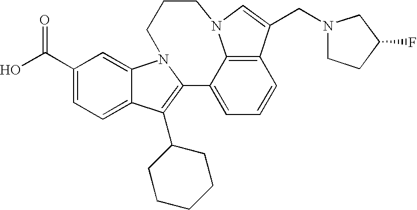

This compound was prepared as described for compound 121 in Example 21 in 0.125 mmole scale, using compound 101 and (R)-(−)-3-fluoropyrrolidine hydrochloride. Yield: 24 mg. MS (M-C₄H₈FN+H⁺): 411.2; H¹-NMR (DMSO d₆): δ (ppm) 10.67 (br s, 1H), 8.13 (s, 1H), 7.97 (d, 1H, J=8.2 Hz), 7.90 (d, 1H, J=8.8 Hz), 7.71-7.64 (m, 2H), 7.30 (t, 1H, J=7.6 Hz), 7.16 (d, 1H, J=7.1 Hz), 5.44 (d, 1H, J=58.3 Hz), 4.68-4.58 (m, 2H), 4.22-4.14 (m, 1H), 3.84-3.68 (m, 2H), 3.68-3.57 (m, 2H), 3.34-3.20 (m, 2H), 2.81 (m, 2H), 2.26-1.10 (m, 14H).

### Example 27

### Preparation of Compound 127

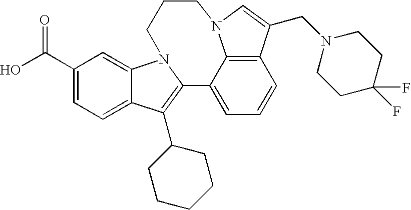

This compound was prepared as described for compound 121 in Example 21 in 0.125 mmole scale, using compound 101 and 4,4-difluoropiperidine. Yield: 30 mg. MS (M-C₅H₉F₂N+H⁺): 411.2; H¹-NMR (DMSO d₆): δ (ppm) 10.50 (br s, 1H), 8.14 (s, 1H), 8.00 (d, 1H, J=7.4 Hz), 7.91 (d, 1H, J=8.5 Hz), 7.68-7.65 (m, 2H), 7.31 (t, 1H, J=7.7 Hz), 7.16 (d, 1H, J=6.9 Hz), 4.68-4.54 (m, 3H), 4.24-4.14 (m, 1H), 3.78-3.48 (m, 3H), 3.32-3.16 (m, 3H), 2.88-2.72 (m, 1H), 2.44-1.04 (m, 16H).

### Example 28

### Preparation of Compound 128

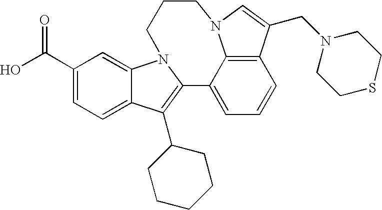

This compound was prepared as described for compound 121 in Example 21 in 0.125 mmole scale, using compound 101 and thiomorpholine. Yield: 29 mg. MS (M-C₄H₉NS+H⁺): 411.2; H¹-NMR (DMSO d₆): δ (ppm) 10.11 (br s, 1H), 8.14 (s, 1H), 7.98 (d, 1H, J=8.0 Hz), 7.91 (d, 1H, J=8.5 Hz), 7.68-7.64 (m, 2H), 7.31 (t, 1H, J=7.5 Hz), 7.18-7.13 (m, 1H), 4.68-4.58 (m, 1H), 4.56-4.50 (m, 2H), 4.24-4.14 (m, 1H), 3.82-3.70 (m, 2H), 3.66-3.52 (m, 1H), 3.28-3.00 (m, 3H), 2.88-2.76 (m, 3H), 2.36-0.80 (m, 14H).

### Example 29

### Preparation of Compound 129

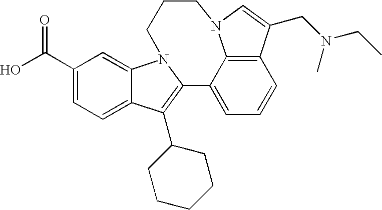

This compound was prepared as described for compound 121 in Example 21 in 0.125 mmole scale, using compound 101 and N-ethylmethylamine. Yield: 29 mg. MS (M+H⁺): 470.3; H¹-NMR (DMSO d₆): δ (ppm) 9.90 (br s, 1H), 8.13 (s, 1H), 7.95 (d, 1H, J=8.3 Hz), 7.90 (d, 1H, J=8.5 Hz), 7.68-7.65 (m, 2H), 7.30 (t, 1H, J=8.0 Hz), 7.16 (d, 1H, J=6.6 Hz), 4.68-4.38 (m, 3H), 4.24-4.14 (m, 1H), 3.66-3.52 (m, 1H), 3.32-3.18 (m, 2H), 3.10-2.98 (m, 1H), 2.90-2.76 (m, 1H), 2.71 (d, 3H, J=4.1 Hz) 2.18-1.34 (m, 9H), 1.29 (t, 3H, J=7.2 Hz), 1.26-0.80 (m, 3H).

### Example 30

### Preparation of Compound 130

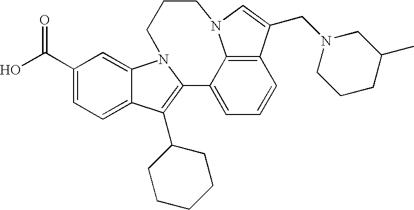

This compound was prepared as described for compound 121 in Example 21 in 0.125 mmole scale, using compound 101 and 3-methylpiperidine. Yield: 29 mg. MS (M+H⁺): 510.3; H¹-NMR (DMSO d₆): δ (ppm) 9.94 (br s, 2H), 8.14 (s, 1H), 7.96 (d, 1H, J=8.0 Hz), 7.91 (d, 1H, J=8.5 Hz), 7.68-7.64 (m, 2H), 7.35-7.27 (m, 1H), 7.18-7.14 (m, 1H), 4.68-4.58 (m, 1H), 4.54-4.36 (m, 2H), 4.24-4.14 (m, 1H), 3.32-3.18 (m, 2H), 2.92-2.70 (m, 2H), 2.68-2.52 (m, 1H), 2.14-0.94 (m, 18H), 0.89 (d, 3H, J=4.7 Hz).

### Example 31

### Preparation of Compound 131

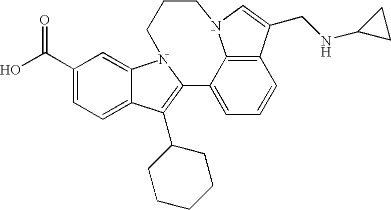

This compound was prepared as described for compound 121 in Example 21 in 0.125 mmole scale, using compound 101 and cyclopropylamine. Yield: 12 mg. MS (M-C₃H₇N+H⁺): 411.2; H¹-NMR (DMSO d₆): δ (ppm) 9.19 (br s, 2H), 8.13 (s, 1H), 7.93 (d, 1H, J=7.9 Hz), 7.90 (d, 1H, J=8.5 Hz), 7.68-7.60 (m, 2H), 7.28 (t, 1H, J=7.7 Hz), 7.15 (d, 1H, J=6.6 Hz), 4.68-4.58 (m, 1H), 4.44-4.36 (m, 2H), 4.22-4.12 (m, 1H), 3.64-3.50 (m, 1H), 3.28-3.14 (m, 1H), 2.88-2.66 (m, 2H), 2.14-0.70 (m, 16H).

### Example 32

### Preparation of Compound 132

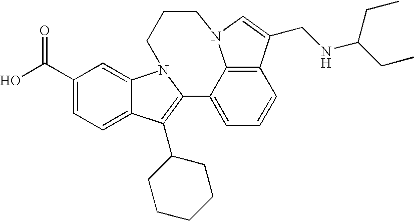

This compound was prepared as described for compound 121 in Example 21 in 0.125 mmole scale, using compound 101 and 1-ethylpropylamine. Yield: 16 mg. MS (M-C₅H₁₃N+H⁺): 411.2; H¹-NMR (DMSO d₆): δ (ppm) 8.66 (br s, 2H), 8.13 (s, 1H), 7.91 (d, 2H, J=8.5 Hz), 7.68-7.63 (m, 2H), 7.29 (t, 1H, J=7.7 Hz), 7.16 (d, 1H, J=6.6 Hz), 4.70-4.58 (m, 1H), 4.42-4.32 (m, 2H), 4.24-4.14 (m, 1H), 3.64-3.50 (m, 1H), 3.28-3.16 (m, 1H), 3.12-3.02 (m, 1H), 2.88-2.70 (m, 1H), 2.16-1.00 (m, 16H), 0.93 (t, 6H, J=7.4 Hz).

### Example 33

### Preparation of Compound 265

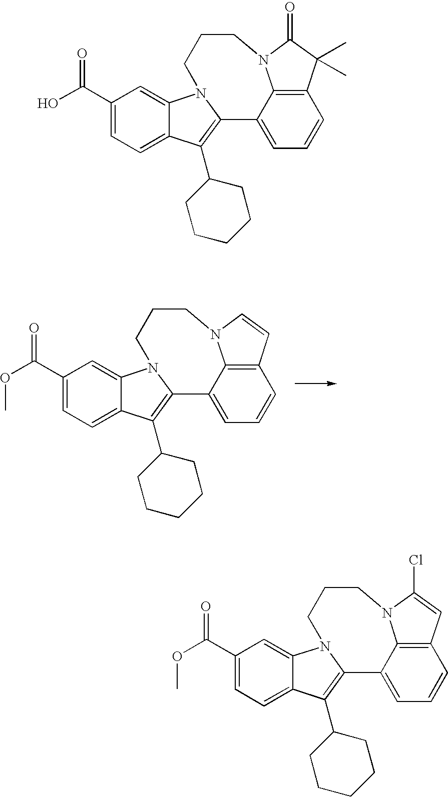

Methyl 2-chloro-12-cyclohexyl-5,6-dihydro-4H-[1,5]diazocinol[1,2-a:5,4,3-h′I′]diindole-9-carboxylate: To a solution of the indole (3.0 g, 7.27 mmol) in DCM (100 mL) was added N-chlorosuccinimide (1.020 g, 7.64 mmol) at room temperature. The reaction mixture was stirred at room temperature for 18 hours after which the solvent was removed in vacuo. The product 3.0 g was used directly in the next step without further purification. MS: 447 [M+H⁺].

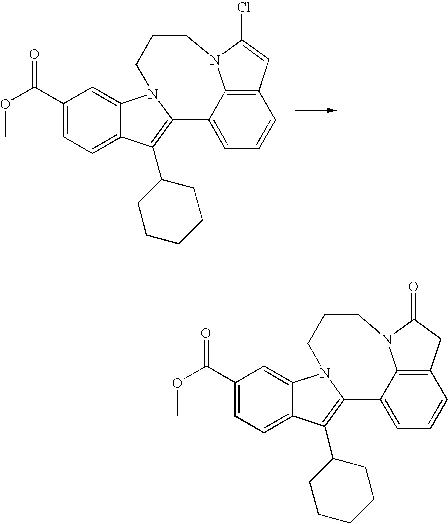

Methyl 12-cyclohexyl-2-oxo-1,2,5,6-tetrahydro-4H-[1,5]diazocino[1,2-a:5,4,3-h′i′]diindole-9-carboxylate: To a solution of the chloroindole (2.6 g, 5.82 mmol) in acetic acid (60 mL) at 120° C. was added 85% H₃PO₄ (2.5 mL). The mixture was heated at reflux for 8 hours. The mixture was poured into ice water (30 mL), adjusted pH to 6.5 and extracted with dichloromethane (125 mL). The combined organic layers were washed with sat. aq. NaHCO₃ solution, brine, and then dried over Na₂SO₄. The solvent was removed and the residue was purified by silica gel column chromatography (EtOAc/heptane, 5% to 40%) to give 1.80 g of product. MS: 429 [M+H⁺].

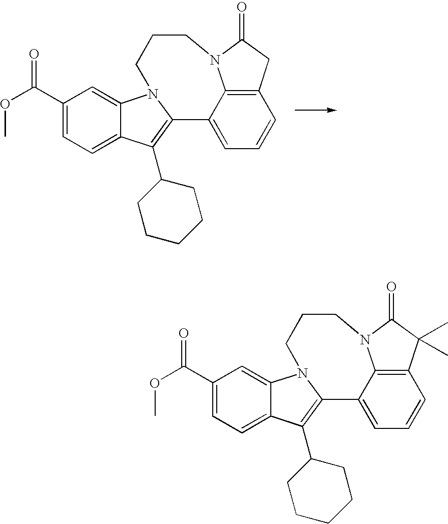

Methyl 12-cyclohexyl-1,1-dimethyl-2-oxo-1,2,5,6-tetrahydro-4H-[1,5]diazocino[1,2-a:5,4,3-h′i′]diindole-9-carboxylate: To a solution of the oxindole (80 mg, 0.187 mmol) in DMF (5 mL) at room temperature was added potassium carbonate (77 mg, 0.560 mmol). The mixture was stirred at room temperature for 20 min after which iodomethane (79 mg, 0.560 mL) was added and the mixture was stirred at room temperature for 18 hours. After DMF was partially removed, EtOAc (60 mL) and water (10 mL) were added and the phases were separated. The organic layers are washed with brine, and then dried over Na₂SO₄ and concentrated. The residue was purified by silica gel column chromatography (EtOAc/heptane, 5% to 25%) to give product 60 mg (70.4%). MS: 457 [M+H⁺].

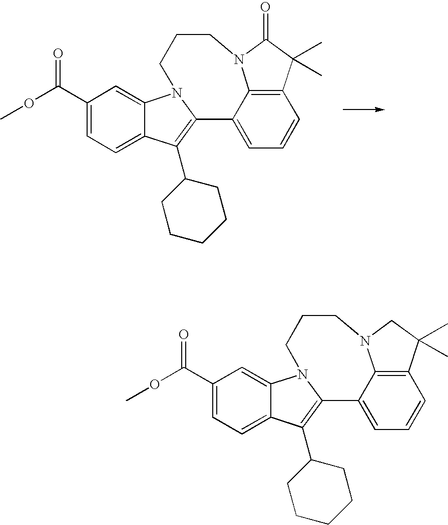

Methyl 12-cyclohexyl-1,1-dimethyl-1,2,5,6-tetrahydro-4H-[1,5]diazocino[1,2-a:5,4,3-h′i′]diindole-9-carboxylate: To a solution of the oxindole (46 mg, 0.101 mmol) in THF (3 mL) at room temperature was added BH₃.THF (0.806 mL, 0.403 mmol). The mixture was heated 60° C. for 2 hours after which it was cooled, quenched with methanol and concentrated. The residue was purified by silica gel column chromatography (EtOAc/heptane) to give product 18 mg. MS: 443

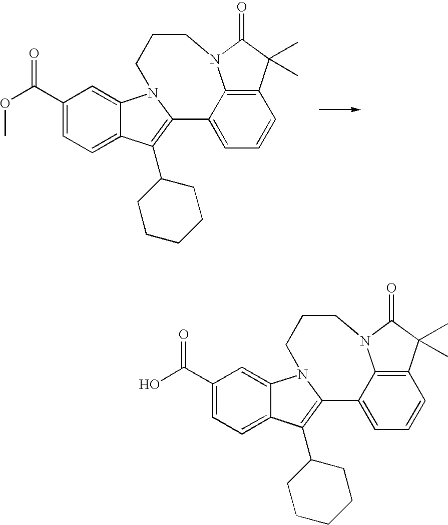

12-cyclohexyl-1,1-dimethyl-2-oxo-1,2,5,6-tetrahydro-4H-[1,5]diazocino[1,2-a:5,4,3-h′i′]diindole-9-carboxylic acid: To a solution of the ester (60 mg, 0.131 mmol) in THF (3.0 mL), MeOH (3.0 mL) and water (3.0 mL) was added 1M LiOH (0.394 mL, 0.394 mmol). The mixture was stirred at 55° C. for 18.0 hours after which the reaction was cooled and quenched by addition of 1.0 N HCl (1.1 mL). All volatiles were concentrated and the solid formed was filtered and dried to afford the product (48 mg, 83%). MS: 443 [M+H⁺]. ¹H NMR (400 MHz, DMSO): NMR data δ 1.10-1.45 (m, 9H), 1.55-2.05 (m, 9H), 2.26-2.40 (m, 1H), 2.65-2.80 (m, 1H), 3.66-3.82 (m, 1H), 3.94-4.02 (m, 1H), 4.62-4.70 (m, 1H), 7.16-7.26 (m, 2H), 7.46-7.52 (d, 1H), 7.66-7.70 (d, 1H), 7.88-7.94 (d, 1H), 8.14 (s, 1H), 12.65 (br, 1H).

### Example 34

### Preparation of Compound 266

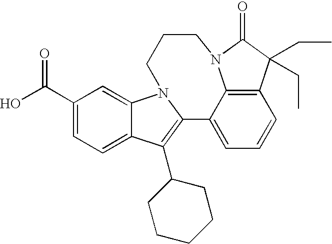

12-cyclohexyl-1,1-diethyl-2-oxo-1,2,5,6-tetrahydro-4H-[1,5]diazocino[1,2-a:5,4,3-h′i′]diindole-9-carboxylic acid: This compound was prepared as described for compound 265 in Example 32. MS: 471 [M+H⁺]. ¹H NMR (400 MHz, MeOD): NMR data δ 0.45-0.62 (t, 6H), 1.10-1.45 (m, 4H), 1.60-2.05 (m, 12H), 2.30-2.45 (m, 1H), 2.70-2.90 (m, 1H), 3.60-3.70 (m, 1H), 4.00-4.10 (m, 1H), 4.60-4.70 (m, 1H), 7.20-7.30 (m, 2H), 7.40-7.48 (d, 1H), 7.66-7.70 (d, 1H), 7.86-7.90 (d, 1H), 8.14 (s, 1H), 12.55 (br, 1H).

### Example 35

### Preparation of Compound 267

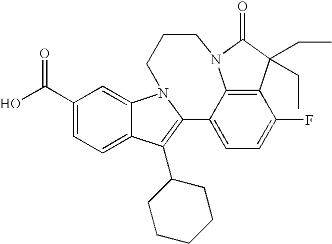

12-cyclohexyl-1,1-diethyl-15-fluoro-2-oxo-1,2,5,6-tetrahydro-4H-[1,5]diazocino[1,2-a:5,4,3-h′i′]diindole-9-carboxylic acid: This compound was prepared as described for compound 265 in Example 32. MS: 489 (M+H⁺). ¹H-NMR (400 MHz, CDCl₃): NMR data δ 0.55-0.65 (t, 6H), 1.10-1.45 (m, 4H), 1.60-2.15 (m, 12H), 2.30.2.42 (m, 1H), 2.70-2.90 (m, 1H), 3.66-3.80 (m, 1H), 4.06-4.16 (m, 1H), 4.40-4.50 (m, 1H), 6.76-6.86 (t, 1H), 7.10-7.16 (m, 1H), 7.74-7.90 (m, 2H), 8.08 (s, 1H), 12.55 (br, 1H).

### Example 36

### Preparation of Compound 268

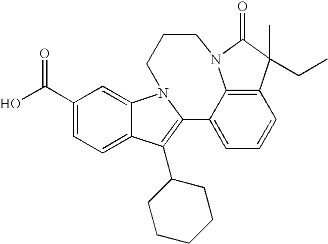

12-cyclohexyl-1-ethyl-1-methyl-2-oxo-1,2,5,6-tetrahydro-4H-[1,5]diazocino[1,2-a:5,4,3-h′i′]diindole-9-carboxylic acid: This compound was prepared as described for compound 265 in Example 32. MS: 457 [M+H⁺]. ¹H NMR (400 MHz, DMSO): NMR data δ 0.50-0.60 (m, 3H), 1.10-1.46 (m, 5H), 1.54-2.10 (m, 12H), 2.22-2.38 (m, 1H), 2.70-2.85 (m, 1H), 3.60-3.80 (m, 1H), 3.95-4.10 (m, 1H), 4.60-4.73 (m, 1H), 7.18-7.26 (m, 2H), 7.40-7.48 (m, 1H), 7.66-7.70 (m, 1H), 7.86-7.90 (m, 1H), 8.14 (s, 1H), 12.55 (br, 1H).

### Example 37

### Preparation of Compound 269

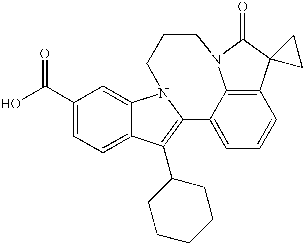

12′-cyclohexyl-2′-oxo-5′,6′-dihydro-4′H-spiro[cyclopropane-1,1′-[1,5]diazocino[1,2-a:5,4,3-h′i′]diindole]-9′-carboxylic acid: This compound was prepared as described for compound 265 in Example 32. MS: 441 (M+H⁺). ¹H-NMR (400 MHz, DMSO): NMR data δ 1.05-1.45 (m, 4H), 1.60-2.15 (m, 12H), 2.30.2.45 (m, 1H), 2.70-2.90 (m, 1H), 3.70-3.80 (m, 1H), 4.00-4.10 (m, 1H), 4.60-4.70 (m, 1H), 7.17 (m, 3H), 7.66-7.70 (d, 1H), 7.86-7.90 (d, 1H), 8.14 (s, 1H), 12.55 (br, 1H).

### Example 38

### Preparation of Compound 270

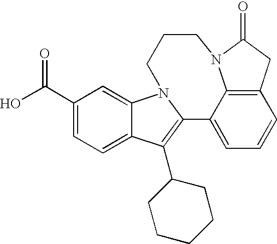

12-cyclohexyl-2-oxo-1,2,5,6-tetrahydro-4H-[1,5]diazocino[1,2-a:5,4,3-h′i′]diindole-9-carboxylic acid: This compound was prepared as described for compound 265 in Example 32. MS: 415 (M+H⁺). ¹H-NMR (400 MHz, DMSO): NMR data δ 1.05-1.45 (m, 5H), 1.65-2.05 (m, 8H), 2.10-2.32 (m, 1H), 2.50-2.80 (m, 2H), 3.18-3.50 (m, 2H), 4.62-4.72 (m, 1H), 7.15-7.20 (m, 2H), 7.36-7.48 (m, 1H), 7.61-7.64 (m, 1H), 7.82-7.88 (m, 1H), 8.16 (m, 1H), 12.55 (br, 1H).

### Example 39

### Preparation of Compound 271

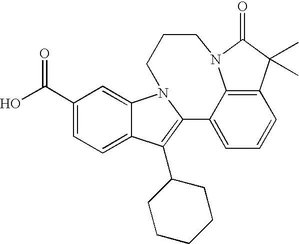

12-cyclohexyl-1,1-dimethyl-1,2,5,6-tetrahydro-4H-[1,5]diazocino[1,2-a:5,4,3-h′i′]diindole-9-carboxylic acid: This compound was prepared as described for compound 265 in Example 32. MS: 429 (M+H⁺). ¹H-NMR (400 MHz, DMSO): NMR data: 429 [M+H⁺]. ¹H-NMR (400 MHz, DMSO HCl Salt): δ 1.19-1.45 (m, 9H), 1.65-2.05 (m, 8H), 2.30-2.48 (m, 2H), 2.70-3.00 (m, 2H), 3.18-3.50 (dd, 2H), 3.60-3.70 (m, 1H), 4.52-4.58 (m, 1H), 6.62-6.66 (t, 1H), 6.84-6.86 (d, 1H), 7.06-7.08 (d, 1H), 7.61-7.64 (d, 1H), 7.82-7.84 (d, 1H), 8.06 (s, 1H), 12.55 (br, 1H).

### Example 40

### Preparation of Compound 272

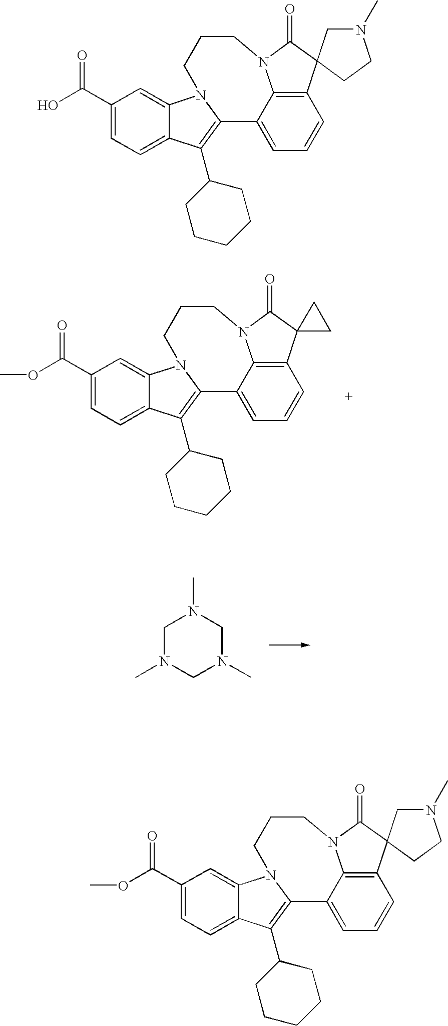

Methyl 12-cyclohexyl-1′-methyl-2-oxo-5,6-dihydro-4H-spiro[1,5-dazocino[1,2-a:5,4,3-h′i′]diindole-1,3′-pyrrolidine]-9-carboxylate: The cyclopropyloxindole (80 mg, 0.176 mmol) and magnesium iodide (24.47 mg, 0.088 mmol) in a seal tube was dried in a drying pistol in the presence of P₂O₅. The tube was flushed several times with nitrogen. THF (0.3 mL) and the triazine (22.74 mg, 0.176 mmol) were added. The tube was sealed and heated at 125° C. for 72 hours. The mixture was cooled after which EtOAc (10 mL) was added and the mixture was filtered through Celite. The filtrate was concentrated and the residue was purified by silica gel column chromatography (EtOAc/heptanes, 5% to 60%) to get product (36 mg, 41%). MS: 498 [M+H⁺].

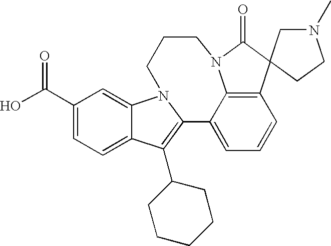

12-cyclohexyl-1′-methyl-2-oxo-5,6-dihydro-4H-spiro[1,5-diazocino[1,2-a:5,4,3-h′i′]diindole-1,3′-pyrrolidine]-9-carboxylic acid: This compound was prepared as described for compound 265 in Example 32. MS: 484 (M+H⁺). ¹H-NMR (400 MHz, DMSO): NMR data: MS: 484 [M+H⁺]. ¹H-NMR (400 MHz, DMSO HCl Salt): δ 1.05-1.45 (m, 3H), 1.51-1.61 (m, 1H), 1.65-2.05 (m, 8H), 2.30-2.48 (m, 2H), 2.50-2.70 (m, 1H), 2.70-2.80 (m, 1H), 3.15-3.25 (br, 3H), 3.35-3.59 (m, 2H), 3.80-4.15 (m, 4H), 4.66-4.70 (m, 1H), 7.22-7.34 (m, 2H), 7.66 (d, 1H), 7.84 (m, 1H), 7.92 (d, 1H), 8.16 (s, 1H), 10.2-11.4 (bs, 1H). 12.65 (br, 1H).

### Example 41

### Preparation of Compound 273

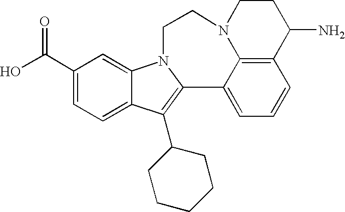

8-bromo-2,3-dihydro-1H-quinolin-4-one oxime: To a solution of 8-bromo-2,3-dihydro-1H-quinoline-4-one (20.0 g, 88 mmol, 1.0 equiv) in EtOH (250 mL) was added hydroxylamine HCl salt (30.5 g, 440 mmol, 5.0 equiv) and pyridine (29.0 mL, 354 mmol, 4.0 equiv). The mixture was heated to reflux for 4 hours. The solvent was then removed under vacuum and to the residue was added EtOAc. The solution was washed with sat. aq. NaHCO₃ solution, brine, dried (over Na₂SO₄) and concentrated. The residue was recrystallized from EtOAc to give 8-bromo-2,3-dihydro-1H-quinolin-4-one oxime 16.0 g. MS: 243 [M+H⁺].

(8-Bromo-1,2,3,4-tetrahydro-quinolin-4-yl)-carbamic acid tert-butyl ester: To a mixture of NaBH₄ (3.0 g, 80 mmol, 4.0 equiv) and DME (60.0 mL) at 0° C. was slowly added TiCl₄ (4.4 mL, 40.0 mmol, 2.0 equiv) and the resultant mixture was stirred at room temperature for 1 hour. The mixture was cooled at 0° C. and a solution of 8-bromo-2,3-dihydro-1H-quinolin-4-one oxime (4.8 g, 20.0 mmol, 1.0 equiv) in DME (10.0 mL) was added. After stirring at room temperature for 24 hours, the solution was cooled at 0° C. and 50% NaOH aq. solution was added until pH=10. To the mixture was then added EtOAc and the phases were separated. The organic layer was washed with brine, dried over Na₂SO₄ and concentrated. The residue was dissolved in CH₂Cl₂ (50.0 mL), cooled to 0° C. and (Boc)₂O (4.4 g, 20.0 mmol, 1.0 equiv) was added. The solution was stirred at room temperature for 2 hours, after which the solvent was removed under vacuum. The residue was purified by silica gel column chromatography (heptane/EtOAc, 5/1) to give product 3.9 g. MS: 329 [M+H⁺].

2-(4-tert-Butoxycarbonylamino-1,2,3,4-tetrahydro-quinolin-8-yl)-3-cyclohexyl-1H-indole-6-carboxylic acid methyl ester: To a solution of (8-bromo-1,2,3,4-tetrahydro-quinolin-4-yl)-carbamic acid tert-butyl ester (6.0 g, 18.3 mmol, 1.05 equiv) in dioxane (36.0 mL) and EtOH (6.0 mL) was added methyl 3-cyclohexyl-2-(4,4,5,5-tetramethyl-1,3,2-dioxaborolan-2-yl)-1H-indole-6-carboxylate (6.7 g, 17.5 mmol, 1.0 equiv), Pd(PPh₃)₄ (1.0 g, 0.87 mmol, 0.05 equiv) and K₂CO₃ (2.0 M solution in water, 26 mL, 52.0 mmol, 3.0 equiv). The mixture was degassed and stirred under N₂ at 95° C. for 3 hours, after which the solvent was removed under vacuum. To the residue was added EtOAc and the solution was washed with water, brine, dried over Na₂SO₄ and concentrated. The crude material was purified by silica gel column chromatography (heptane/EtOAc, 1/1) to give product 8.6 g. MS: 508 [M+H⁺].

Methyl 4-[(tert-butoxycarbonyl)amino]-15-cyclohexyl-5,6,8,9-tetrahydro-4H-indolo[1′,2′:4,5][1,4]diazepino[6,7,1-ij]quinoline-12-carboxylate: To a solution of 2-(4-tert-Butoxycarbonylamino-1,2,3,4-tetrahydro-quinolin-8-yl)-3-cyclohexyl-1H-indole-6-carboxylic acid methyl ester (1.0 g, 2.0 mmol, 1.0 equiv) in THF (25.0 mL) was added acetic acid (0.13 g, 2.2 mmol, 1.1 equiv), sodium acetate (0.18 g, 2.2 mmol, 1.1 equiv) and chloroacetyl chloride (0.36 g, 3.2 mmol, 1.6 equiv). The mixture was stirred at 45° C. for 2 hours, after which the solvent was removed under vacuum. To the residue was added water and the mixture was filtered to obtain product (0.9 g), which was used on the next step without further purification.

The product (0.9 g, 1.5 mmol, 1.0 equiv) from previous step was dissolved in DMF (20 mL) and Cs₂CO₃ (1.6 g, 4.5 mmol, 3.0 equiv) was added. After stirring at 45° C. for 1 hour, the mixture was added to 200 mL of water. The mixture was then filtered to yield 0.7 g of product, which was used in the next step without further purification.

The product (0.7 g, 1.3 mmol, 1.0 equiv) from previous step was dissolved in THF (5.0 mL). To this solution was added BH₃.THF solution (1.0 M, 17 mL, 13.5 equiv) and the resultant solution was stirred at 45° C. for 3 hours. The solution was then placed in an ice-water bath and MeOH (3.0 mL) was slowly added. The solvent was removed under vacuum and to the residue was added water and EtOAc. The mixture was filtered to yield product 600 mg. MS: 530 [M+H⁺].

Methyl 4-amino-15-cyclohexyl-5,6,8,9-tetrahydro-4H-indolo[1′,2′:4,5][1,4]diazepino[6,7,1-ij]quinoline-12-carboxylate: Methyl 4-[(tert-butoxycarbonyl)amino]-15-cyclohexyl-5,6,8,9-tetrahydro-4H-indolo[1′,2′:4,5][1,4]diazepino[6,7,1-ij]quinoline-12-carboxylate (3.5 g, 6.61 mmol) was added to 4.0 N HCl in dioxane (40 mL). After stirring at room temperature for 1 hour, the mixture was concentrated under vacuum. To the residue was added CH₂Cl₂ and heptane. The solvent was again removed under vacuum to give product (3.08 g), which was used in the next step without further purification. MS: 430 [M+H⁺].

4-Amino-15-cyclohexyl-5,6,8,9-tetrahydro-4H-indolo[1′,2′:4,5][1,4]diazepino[6,7,1-ij]quinoline-12-carboxylic acid: To a solution of methyl 4-amino-15-cyclohexyl-5,6,8,9-tetrahydro-4H-indolo[1′,2′:4,5][1,4]diazepino[6,7,1-ij]quinoline-12-carboxylate (850 mg, 1.83 mmol, 1.0 equiv) in THF (9.0 mL) was added MeOH (4.0 mL), water (4.0 mL) and LiOH.H₂O (1.08 g, 25.9 mmol, 14.2 equiv). After stirring at 60° C. for 2 hours, the mixture was concentrated under vacuum and to the residue was added 1.0 N HCl aq. solution until pH=6. To the mixture was added EtOAc and the phases were separated. The organic layer was washed with brine, dried (Na₂SO₄), concentrated to give product 610 mg. MS: 416 [M+H⁺]. ¹H NMR (400 MHz, DMSO-d6): 1.00-1.48 (m, 4H), 1.62-2.12 (m, 8H), 2.65-2.81 (m, 1H), 2.96-3.18 (m, 2H), 3.42-3.65 (m, 3H), 4.45-4.62 (m, 2H), 7.16-7.25 (t, 1H), 7.25-7.34 (d, 1H), 7.56-7.67 (d, 2H), 7.82-7.91 (d, 1H), 8.18 (s, 1H), 8.39 (br, 3H)

### Example 42

### Preparation of Compound 274

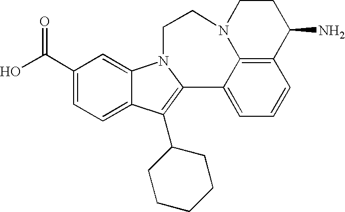

(R)-2-Methyl-propane-2-sulfinic acid ((R)-8-bromo-1,2,3,4-tetrahydro-quinolin-4-yl)-amide: To a solution of (R)-tert-butylsulfinamide (8.85 g, 73.0 mmol, 1.5 equiv) and 8-bromo-2,3-dihydro-1H-quinoline-4-one (11.0 g, 48.7 mmol, 1.0 equiv) in THF (80.0 mL) at room temperature was added Ti(OEt)₄ (30.6 mL, 146 mmol, 3.0 equiv). After stirring at 75° C. for 12 hours, the solution was placed in an ice-water bath and water was added slowly. The solid was filtered and washed with CH₂Cl₂. The phases were separated and the aqueous layer was extracted with EtOAc. The organic layers were combined, washed with brine, dried (Na₂SO₄) and concentrated. The crude material was used in the next step without further purification.

The product from the previous step was dissolved in THF (20 mL). This solution was added to a suspension of NaBH₄ in THF (60 mL) at −48° C. and the resultant solution was warmed to room temperature and stirred at this temperature for 4 hours. The solution was then placed in ice-water bath and to the solution was added MeOH (15 mL) followed by sat. aq. NaHCO₃ solution. The phases were separated. The organic phase was washed with brine, dried over Na₂SO₄ and concentrated. The material was purified by silica gel column chromatography (heptane/EtOAc, 1/1) to give product 8.3 g. MS: 332 [M+H⁺]. ¹H NMR (400 MHz, CDCl₃): 1.16-1.28 (s, 9H), 1.83-1.99 (m, 1H), 2.06-2.20 (m, 1H), 3.08-3.20 (m, 1H), 3.34-3.48 (m, 2H), 4.55-4.72 (br, 2H), 6.49-6.61 (t, 1H), 7.18-7.24 (d, 1H), 7.32-7.41 (d, 1H).

3-Cyclohexyl-2-[(R)-4-((R)2-methyl-propane-2-sulfinylamino)-1,2,3,4-tetrahydro-quinolin-8-yl]-1H-indole-6-carboxylic acid methyl ester: To a solution of S(R)-2-Methyl-propane-2-sulfinic acid ((R)-8-bromo-1,2,3,4-tetrahydro-quinolin-4-yl)-amide (2.0 g, 6.0 mmol, 1.0 equiv) in dioxane (20 mL) and EtOH (4.0 mL) was added 3-cyclohexyl-2-(4,4,5,5-tetramethyl-1,3,2-dioxaborolan-2-yl)-1H-indole-6-carboxylic acid methyl ester (3.01 g, 7.8 mmol, 1.3 equiv), Pd(PPh₃)₄ (0.69 g, 0.60 mmol, 0.1 equiv) and K₂CO₃ (2.0 M solution in water, 18.1 mmol, 3.0 equiv). The mixture was degassed and stirred at 95° C. for 4 hours. The mixture was concentrated under vacuum and the residue was diluted with EtOAc. The solution was washed with water, brine, dried over Na₂SO₄ and concentrated. The residue was purified by silica gel column chromatography (heptane/EtOAc, 1/2) to give product 2.7 g. MS: 508 [M+H⁺].

2-((R)-4-tert-Butoxycarbonylamino-1,2,3,4-tetrahydro-quinolin-8-yl)-3-cyclohexyl-11H-indole-6-carboxylic acid methyl ester: To a solution of sulfinamide (13.2 g, 26 mmol, 1.0 equiv) in MeOH (50 mL) was added 4.0 N HCl in dioxane (150 mL). The solution was stirred at room temperature for 10 minutes, after which the solvent was removed under vacuum. To the crude material was added CH₂Cl₂ and heptane. The solvent was then evaporated under vacuum. To the material was added CH₂Cl₂ (150 mL), sat. aq. NaHCO₃ solution (150 mL) and (Boc)₂O (8.5 g, 39.0 mmol, 1.5 equiv). The mixture was stirred at room temperature for 30 minutes, after which the phases were separated and the aqueous layer was extracted with CH₂Cl₂. The organic layers were combined, washed with brine, dried (Na₂SO₄) and concentrated. The crude material was purified by silica gel column chromatography (heptane/EtOAc, 2/1) to give product 8.1 g. MS: 504 [M+H⁺].

Methyl (4R)-4-[(tert-butoxycarbonyl)amino]-15-cyclohexyl-5,6,8,9-tetrahydro-4H-indolo[1′,2′:4,5][1,4]diazepino[6,7,1-ij]quinoline-12-carboxylate: To a solution of 2-((R)-4-tert-Butoxycarbonylamino-1,2,3,4-tetrahydro-quinolin-8-yl)-3-cyclohexyl-1H-indole-6-carboxylic acid methyl ester (6.0 g, 11.9 mmol, 1.0 equiv) in THF (120 mL) was added acetic acid (0.78 g, 13.1 mmol, 1.1 equiv), sodium acetate (1.07 g, 13.1 mmol, 1.1 equiv) and chloroacetyl chloride (2.0 g, 17.8 mmol, 1.5 equiv). The mixture was stirred at 45° C. for 2 hours, after which the solvent was removed under vacuum. To the resultant solid was added EtOAc. The solution was washed with water, dried (Na₂SO₄) and concentrated. The crude material was used in the next step without further purification.

The product from previous step was dissolved in DMF (60 mL) and to the solution was added Cs₂CO₃ (7.76 g, 23.8 mmol). The mixture was stirred at 45° C. for 1 hour, after which it was added to 600 mL of ice-water. The solid was then collected by filtration and used in the next step without further purification.

The product from previous step was dissolved in THF (55 mL). To this solution was added BH₃.THF solution (1.0 M, 73.9 mL, 73.9 mmol) and the resultant solution was stirred at room temperature for 1 hour. The solution was then placed in an ice-water bath and MeOH (10 mL) was slowly added. After the solvent was evaporated, the solid was dissolved in MeOH and filtered. The filtrate was then concentrated and the residue was dissolved in EtOAc. The resultant solution was washed with sat. aq. NaHCO₃ solution, water, brine, dried (Na₂SO₄) and concentrated. The crude material was recrystallized from heptane/EtOAc to give product 4.7 g. MS: 530 [M+H⁺].

(4R)-4-[(tert-butoxycarbonyl)amino]-15-cyclohexyl-5,6,8,9-tetrahydro-4H-indolo[1′,2′:4,5][1,4]diazepino[6,7,1-ij]quinoline-12-carboxylic acid: To a solution of Methyl (4R)-4-[(tert-butoxycarbonyl)amino]-15-cyclohexyl-5,6,8,9-tetrahydro-4H-indolo[1′,2′:4,5][1,4]diazepino[6,7,1-ij]quinoline-12-carboxylate (800 mg, 1.51 mmol, 1.0 equiv) in THF (5.0 mL) was added MeOH (5.0 mL), water (5.0 mL) and LiOH.H₂O (181 mg, 7.55 mmol, 5.0 equiv). After stirring at 60° C. for 4 hours, the mixture was concentrated under vacuum and to the residue was added 1.0 N HCl aq. solution until pH=4. To the mixture was added EtOAc and the phases were separated. The organic layer was washed with brine, dried (Na₂SO₄), concentrated to give product 769 mg. MS: 516 [M+H]. ¹H NMR (400 MHz, DMSO-d6): 1.00-1.40 (m, 4H), 1.43 (s, 9H), 1.60-2.20 (m, 8H), 2.70-2.85 (m, 1H), 2.90-3.20 (m, 2H), 3.40-3.65 (m, 3H), 4.60-4.90 (m, 2H), 7.05-7.15 (t, 1H), 7.15-7.23 (d, 1H), 7.25-7.36 (d, 1H), 7.36-7.50 (br, 1H), 7.55-7.65 (d, 1H), 7.80-7.93 (d, 1H), 8.20 (s, 1H), 12.60-12.80 (br, 1H).

(4R)-4-amino-15-cyclohexyl-5,6,8,9-tetrahydro-4H-indolo[1′,2′:4,5][1,4]diazepino[6,7,1-ij]quinoline-12-carboxylic acid: To a solution of (4R)-4-[(tert-butoxycarbonyl)amino]-15-cyclohexyl-5,6,8,9-tetrahydro-4H-indolo[1′,2′:4,5][1,4]diazepino[6,7,1-ij]quinoline-12-carboxylic acid (768 mg, 1.48 mmol) in CH₂Cl₂ (25 mL) was added 4.0 N HCl in dioxane (20 mL). After stirring at room temperature for 2 hours, the mixture was concentrated under vacuum. The residue was redissolved in CH₂Cl₂/heptane and the solution was concentrated again. To the residue was added a solution of CH₃CN/water (3.0 mL, 4/1) followed by slow addition of water until all solid dissolved. To the resultant solution was then added CH₃CN (20 mL) with stirring. The solid was collected by filtration to give product 530 mg. The filtrate was concentrated and to the residue was added CH₃CN (10 mL). The solid was collected by filtration to give second fraction product 110 mg. MS: 416 [M+H⁺]. ¹H NMR (DMSO-d₆): 12.6 (s, 1H), 8.45 (br, 2H), 8.19 (s, 1H), 7.87 (d, 1H), 7.65 (d, 1H), 7.62 (d, 1H), 7.29 (d, 1H), 7.21 (t, 1H), 4.54 (br, 1H), 3.52 (br, 2H), 3.06 (br, 2H), 2.71-2.74 (m, 1H), 2.08-2.03 (m, 4H), 1.81-1.68 (m, 6H), 1.42-1.35 (m, 4H).

### Example 43

### Preparation of Compound 301

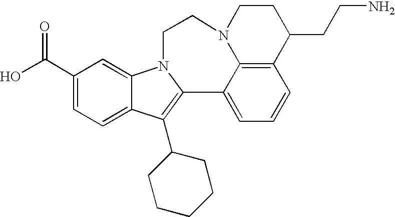

[8-Bromo-2,3-dihydro-1H-quinolin-4-ylidene]-acetonitrile: To a solution of cyanomethyl phosphonic acid diethyl ester (3.64 g, 20.0 mmol, 2.0 equiv) in THF (40.0 mL) at 0° C. was added NaH (0.720 g, 30.0 equiv, 3.0 equiv) and the resultant solution was stirred at room temperature for 10 minutes. The mixture was then placed in an ice-water bath and a solution of 8-bromo-2,3-dihydro-1H-quinoline-4-one (2.26 g, 10.0 mmol, 1.0 equiv) in THF (5.0 mL) was added. After stirring at 0° C. for 1 hour, to the mixture was added sat. aq. NH₄Cl solution and EtOAc. The phases were separated and the organic phase was washed with brine, dried over Na₂SO₄ and concentrated. The residue was purified by silica gel column chromatography (heptane/EtOAc, 4/1) to give product 1.8 g (72%).

[2-(8-Bromo-1,2,3,4-tetrahydro-quinolin-4-yl)-ethyl]-carbamic acid tert-butyl ester: To a solution of L-selectride (18.0 mL, 1.0 M in THF, 6.0 equiv) at −78° C. was added a solution of [8-Bromo-2,3-dihydro-1H-quinolin-4-ylidene]-acetonitrile (750 mg, 3.0 mmol, 1.0 equiv) in THF (2.0 mL). The solution was warmed to room temperature over 3 hours and then stirred at this temperature for 72 hours. The reaction was quenched by addition of sat. sq. NaHCO₃ solution. After EtOAc was added to the solution, the phases were separated and the aqueous layer was extracted with EtOAc. The organic layers were combined, dried over Na₂SO₄ and concentrated. The residue was dissolved in CH₂Cl₂ (2.0 M) and added (Boc)₂O. After stirring at room temperature for 2 hours, the solution was concentrated under vacuum. The residue was purified by silica gel column chromatography (heptane/EtOAc) to give product 420 mg. ¹H NMR (400 MHz, CDCl₃): 1.41-1.54 (s, 9H), 1.65-2.00 (m, 4H), 2.79-2.90 (m, 1H), 3.14-3.36 (m, 2H), 3.38-3.48 (m, 2H), 4.49-4.59 (br, 2H), 6.43-6.54 (t, 1H), 6.90-6.98 (d, 1H), 7.22-7.27 (d, 1H).

2-[4-(2-tert-Butoxycarbonylamino-ethyl)-1,2,3,4-tetrahydro-quinolin-8-yl]-3-cyclohexyl-1H-indole-6-carboxylic acid methyl ester: To a solution of [2-(8-bromo-1,2,3,4-tetrahydro-quinolin-4-yl)-ethyl]-carbamic acid tert-butyl ester (300 mg, 0.84 mmol, 1.0 equiv) in dioxane (2.5 mL) and EtOH (0.3 mL) and water (1.2 mL) was added 3-cyclohexyl-2-(4,4,5,5-tetramethyl-1,3,2-dioxaborolan-2-yl)-1H-indole-6-carboxylic acid methyl ester (388 mg, 1.0 mmol, 1.2 equiv), Pd(PPh₃)₄ (58.5 mg, 0.05 mmol, 0.06 equiv) and K₂CO₃ (350 mg, 2.5 mmol, 3.0 equiv). The mixture was degassed and stirred at 95° C. for 3 hours. The mixture was concentrated and diluted with EtOAc. The solution was washed with water, brine, dried over Na₂SO₄ and concentrated. The crude material was purified by silica gel column chromatography (heptane/EtOAc, 1/1) to give product 350 mg. MS: 532 [M+H⁺].

Methyl 4-{2-[(tert-butoxycarbonyl)amino]ethyl}-15-cyclohexyl-5,6,8,9-tetrahydro-4H-indolo[1′,2′:4,5][1,4]diazepino[6,7,1-ij]quinoline-12-carboxylate: To a solution of 2-[4-(2-tert-Butoxycarbonylamino-ethyl)-1,2,3,4-tetrahydro-quinolin-8-yl]-3-cyclohexyl-1H-indole-6-carboxylic acid methyl ester (300 mg, 0.56 mmol, 1.0 equiv) in THF (2.0 mL) was added acetic acid (37 mg, 0.62 mmol, 1.1 equiv), sodium acetate (51 mg, 0.62 mmol, 1.1 equiv) and chloroacetyl chloride (96 mg, 0.85 mmol, 1.5 equiv). The mixture was stirred at 50° C. for 6 hours, after which it was diluted with EtOAc. The solution was washed with sat. aq. NaHCO₃ solution, dried (Na₂SO₄) and concentrated. The crude material was used in the next step without further purification.

The product from previous step was dissolved in DMF (60 mL) and Cs₂CO₃ (346 mg, 1.0 mmol) was added. After stirring at room temperature for 2 hours, the mixture was purified by silica gel column chromatography (heptane/EtOAc, 4/1) to give product 250 mg.

The product from previous step was dissolved in THF (1.0 mL). To this solution was added BH₃.THF solution (1.0 M, 1.7 mL) and the resulting solution was stirred at room temperature for 1 hour. To the solution was added MeOH (2.0 mL) and then it was heated to reflux for 1 hour. The solution was then concentrated under vacuum and the residue was purified by silica gel column chromatography (heptane/EtOAc, 1/1) to give product 230 mg. MS: 558 [M+H⁺].

Methyl 4-(2-aminoethyl)-15-cyclohexyl-5,6,8,9-tetrahydro-4H-indolo[1′,2′:4,5][1,4]diazepino[6,7,1-ij]quinoline-12-carboxylate: To a solution of Boc-amine (230 mg, 0.41 mmol) in dioxane (1.0 mL) was added 4.0 N HCl solution in dioxane (1.0 mL) and the mixture was stirred at room temperature for 4 hours. The solvent was then removed under vacuum and the residue was added heptane. The solvent was again removed under vacuum to give product 200 mg, which was used in the following step without purification. MS: 558 [M+H⁺].

4-(2-aminoethyl)-15-cyclohexyl-5,6,8,9-tetrahydro-4H-indolo[1′,2′:4,5][1,4]diazepino[6,7,1-ij]quinoline-12-carboxylic acid: To a solution of methyl ester (50 mg, 0.11 mmol, 1.0 equiv) in THF (0.3 mL), MeOH (0.3 mL) and water (0.3 mL) was added LiOH.H₂O (13 mg, 0.55 mmol, 5.0 equiv). After stirring at 50° C. for 4 hours, the mixture was cooled at room temperature and neutralized by addition of 1.0 N HCl aq. solution until pH=6. The solid was then collected by filtration and washed with water. The product was dissolved in 1.0 mL of water and 0.1 mL of 1.0 N aq. HCl solution. The solvent was removed by freeze dry method to give the product as HCl salt (25 mg). MS: 444 [M+H⁺]. ¹H NMR (400 MHz, DMSO-d6): 1.14-1.47 (m, 4H), 1.61-1.87 (m, 8H), 1.96-2.13 (m, 4H), 2.71-2.85 (m, 1H), 2.87-3.02 (br, 5H), 3.40-3.52 (br, 2H), 7.08-7.16 (m, 2H), 7.28-7.35 (d, 1H), 7.58-7.63 (d, 1H), 7.82-7.87 (d, 1H), 7.88-7.95 (br, 3H), 8.17 (s, 1H)

### Example 44

### Preparation of Compound 302

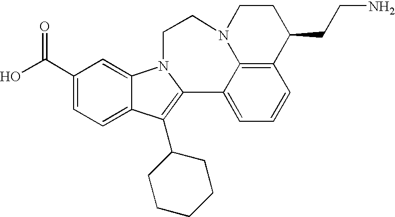

This compound was prepared as described for compound 301 in Example 43. (Prepared of Compound 302 is enantiomerically pure. The absolute configuration was not determined).

Methyl 4-{2-[(tert-butoxycarbonyl)amino]ethyl}-15-cyclohexyl-5,6,8,9-tetrahydro-4H-indolo[1′,2′:4,5][1,4]diazepino[6,7,1-ij]quinoline-12-carboxylate: Methyl 4-(2-aminoethyl)-15-cyclohexyl-5,6,8,9-tetrahydro-4H-indolo[1′,2′:4,5][1,4]diazepino[6,7,1-ij]quinoline-12-carboxylate was separated by chiral SFC to two enantiomers. To a solution of one enantiomer (32 mg, 0.07 mmol, 1.0 equiv) in CH₂Cl₂ (1.0 mL) was added DIPEA (36 mg, 0.28 mmol, 4.0 equiv) and (Boc)₂O (30.5 mg, 0.14 mmol, 2.0 equiv). The solution was stirred at room temperature for 1 hour after which the mixture was separated by silica gel column chromatography (heptane/EtOAc, 1/1) to give product (35 mg). MS: 558 [M+H⁺].

4-{2-[(tert-butoxycarbonyl)amino]ethyl}-15-cyclohexyl-5,6,8,9-tetrahydro-4H-indolo[1′,2′:4,5][1,4]diazepino[6,7,1-ij]quinoline-12-carboxylic acid: To a solution of methyl ester (30 mg, 0.054 mmol, 1.0 equiv) in THF (0.5 mL), MeOH (0.5 mL) and water (0.5 mL) was added LiOH.H₂O (6.4 mg, 0.27 mmol, 5.0 equiv). After stirring at 60° C. for 6 hours, the mixture was cooled at room temperature and acidified to pH=3 by addition of 1.0 N HCl aq. solution. The solution was diluted with EtOAc and the phases were separated. The organic layer was washed with brine, dried over Na₂SO₄ and concentrated to give product 27 mg. MS: 544 [M+H⁺].

4-(2-Aminoethyl)-15-cyclohexyl-5,6,8,9-tetrahydro-4H-indolo[1′,2′:4,5][1,4]diazepino[6,7,1-ij]quinoline-12-carboxylic acid: To a flask containing Boc-amine (27 mg) was added 4.0 N HCl solution in dioxane (2.4 mL) and the resultant solution was stirred at room temperature for 1 hour. The solution was then concentrated under vacuum and the residue was dissolved with water. The solvent was removed by freeze dry method to give product 18 mg. MS: 444 [M+H]. ¹H NMR (400 MHz, CD₃OD): 1.12-1.58 (m, 5H), 1.69-2.46 (m, 11H), 2.84-3.03 (m, 1H), 3.03-3.25 (m, 4H), 3.37-3.52 (m, 2H), 3.54-3.77 (m, 1H), 3.83-4.03 (br, 2H), 7.34-7.46 (d, 1H), 7.46-7.56 (t, 1H), 7.56-7.62 (d, 1H), 7.74-7.80 (d, 1H), 7.89-7.99 (d, 1H), 8.24 (s, 1H).

### Example 45

### Preparation of Compound 303

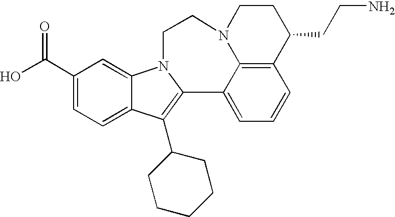

This compound was prepared as described for compound 302 in Example 44. MS: 444 [M+H⁺]. ¹H NMR (400 MHz, DMSO-d6): 1.01-1.47 (m, 5H), 1.48-2.15 (m, 11H), 2.69-2.84 (m, 1H), 2.85-3.04 (m, 4H), 3.36-3.55 (m, 3H), 7.02-7.19 (m, 2H), 7.25-7.39 (d, 1H), 7.54-7.68 (d, 1H), 7.79-7.88 (d, 1H), 7.88-8.00 (br, 3H), 8.16 (s, 1H).

### Example 46

### Preparation of Compound 293

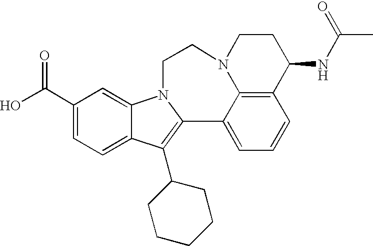

Methyl (4R)-4-amino-15-cyclohexyl-5,6,8,9-tetrahydro-4H-indolo[1′,2′:4,5][1,4]diazepino[6,7,1-ij]quinoline-12-carboxylate: To a solution of methyl (4R)-4-[(tert-butoxycarbonyl)amino]-15-cyclohexyl-5,6,8,9-tetrahydro-4H-indolo[1′,2′:4,5][1,4]diazepino[6,7,1-ij]quinoline-12-carboxylate (550 mg) in CH₂Cl₂ (5.0 mL) was added 4.0 N HCl solution in dioxane (9.1 mL). After stirring at room temperature for 1 hour, the solution was concentrated under vacuum to give 435 mg of methyl (4R)-4-amino-15-cyclohexyl-5,6,8,9-tetrahydro-4H-indolo[1′,2′:4,5][1,4]diazepino[6,7,1-ij]quinoline-12-carboxylate. MS: 430 [M+H⁺].

Methyl (4R)-4-acetamido-15-cyclohexyl-5,6,8,9-tetrahydro-4H-indolo[1′,2′:4,5][1,4]diazepino[6,7,1-ij]quinoline-12-carboxylate: To a suspension of methyl (4R)-4-amino-15-cyclohexyl-5,6,8,9-tetrahydro-4H-indolo[1′,2′:4,5][1,4]diazepino[6,7,1-ij]quinoline-12-carboxylate HCl salt (100 mg) in CH₂Cl₂ (2.0 mL) was added Et₃N (0.049 mL, 0.35 mmol, 1.5 equiv), followed by AcCl (20.1 mg, 0.26 mmol, 1.1 equiv). The solution was stirred at room temperature for 1 hour, after which the solvent was removed under vacuum. The residue was purified by silica gel column chromatography (heptane/acetone, 1/1) to give product 60 mg. MS: 472 [M+H⁺]

(4R)-4-Acetamido-15-cyclohexyl-5,6,8,9-tetrahydro-4H-indolo[1′,2′:4,5][1,4]diazepino[6,7,1-ij]quinoline-12-carboxylic acid: To a solution of methyl ester (60 mg, 0.13 mmol, 1.0 equiv) in THF (1.0 mL) MeOH (0.5 mL) and water (0.5 mL) was added LiOH.H₂O (53 mg, 1.3 mmol, 10.0 equiv). The mixture was stirred at 57° C. for 2 hours. The solution was neutralized to pH=6 by addition of 1.0 N HCl aq. solution. The solid was collected by filtration and washed with water. The solid was dissolved in CH₃CN and water. The solvent was then removed by freeze dry method to give product 42 mg. MS: 458 [M+H⁺]. ¹H NMR (CDCl₃): 1.13-1.38 (m, 3H), 1.65-1.91 (m, 6H), 1.92-2.17 (m, 6H), 2.73-2.85 (m, 1H), 2.90-3.06 (m, 2H), 3.40-3.63 (m, 2H), 3.90-4.55 (m, 2H), 5.10-5.21 (s, 1H), 5.71-5.83 (s, 1H), 7.00-7.09 (t, 1H), 7.18-7.22 (d, 1H), 7.24-7.33 (d, 1H), 7.70-7.77 (d, 1H), 7.80-7.88 (d, 1H), 8.07 (s, 1H).

### Example 47

### Preparation of Compound 300

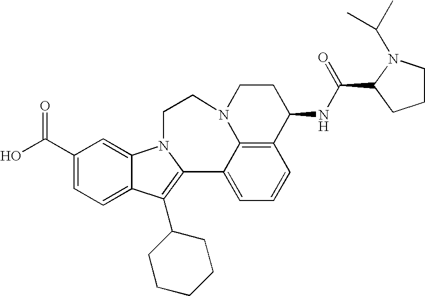

Methyl (4R)-15-cyclohexyl-4-[(1-isopropyl-L-prolyl)amino]-5,6,8,9-tetrahydro-4H-indolo[1′,2′:4,5][1,4]diazepino[6,7,1-ij]quinoline-12-carboxylate: To a solution of methyl (4R)-4-amino-15-cyclohexyl-5,6,8,9-tetrahydro-4H-indolo[1′,2′:4,5][1,4]diazepino[6,7,1-ij]quinoline-12-carboxylate HCl salt (60 mg, 0.14 mmol, 1.0 equiv) in DMF/CH₂Cl₂ (1.0 mL, 1/1) at 0° C. was added HOBT (25.7 mg, 0.168 mmol, 1.2 equiv), HATU (63.7 mg, 0.168 mmol, 1.2 equiv), DIPEA (73 μL, 0.419 mmol, 3.0 equiv). The resultant solution was stirred at 0° C. for 10 minutes, after which 1-isopropyl-L-proline (26.4 mg, 0.168 mmol, 1.2 equiv) was added and the solution was then stirred at room temperature for 1 hour. The solution was then diluted with EtOAc and washed with sat. aq. NaHCO₃ solution, brine, dried over Na₂SO₄ and concentrated. The residue was purified by silica gel column chromatography (heptane/acetone, 1/1) to give product 75 mg. MS: 569 [M+H⁺].

(4R)-15-cyclohexyl-4-[(1-isopropyl-L-prolyl)amino]-5,6,8,9-tetrahydro-4H-indolo[1′,2′:4,5][1,4]diazepino[6,7,1-ij]quinoline-12-carboxylic acid: To a solution of methyl ester (75 mg, 0.13 mmol, 1.0 equiv) in THF (0.5 mL), MeOH (0.5 mL) and water (0.5 mL) was added LiOH.H₂O (15 mg, 0.65 mmol, 5.0 equiv). The mixture was stirred at 40° C. for 8 hours. The solution was neutralized to pH=6 by addition of 1.0 N HCl aq. solution. The solution was then diluted with EtOAc and the phases were separated. The organic phase was washed with brine, dried (Na₂SO₄) and concentrated to give product 52 mg. MS: 556 [M+H⁺]. ¹H NMR (CD₃OD): 12.1 (s, 1H), 8.01 (s, 1H), 7.82 (d, J=8.4 Hz, 1H), 7.69 (dd, J=8.4, 1.2 Hz, 1H), 7.26 (s, 1H), 7.24 (s, 1H), 7.11 (t, J=7.6 Hz, 1H), 5.16 (m, 1H), 3.57-3.55 (m, 3H), 3.34-3.00 (s, 3H), 2.84-2.65 (m, 2H), 2.26-2.21 (m, 1H), 2.14-2.00 (m, 3H), 1.96-1.75 (m, 12H), 1.40-1.31 (m, 4H), 1.16 (t, J=6.6 Hz, 6H).

### Example 48

### Preparation of Compound 296

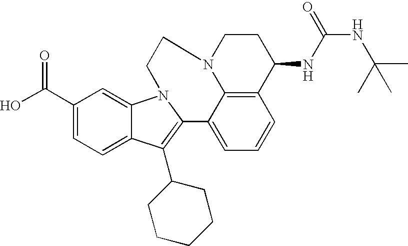

Methyl (4R)-4-[(tert-butylcarbamoyl)amino]-15-cyclohexyl-5,6,8,9-tetrahydro-4H-indolo[1′,2′:4,5][1,4]diazepino[6,7,1-ij]quinoline-12-carboxylate: A mixture of methyl (4R)-4-amino-15-cyclohexyl-5,6,8,9-tetrahydro-4H-indolo[1′,2′:4,5][1,4]diazepino[6,7,1-ij]quinoline-12-carboxylate HCl salt (80 mg, 0.18 mmol, 1.0 equiv), CH₂Cl₂ (0.5 mL) and sat. aq. NaHCO₃ solution (0.5 mL) was stirred at 0° C. for 5 minutes. t-Butylisocyanate (22 mg, 0.22 mmol, 1.2 equiv) was added to the organic layer. The mixture was then stirred at 0° C. for 30 minutes. The phases were separated and the organic phase was dried (Na₂SO₄) and concentrated. The residue was purified by silica gel column chromatography (heptane/EtOAc, 1/1) to give product 80 mg. MS: 529 [M+H⁺].

(4R)-4-[(tert-Butylcarbamoyl)amino]-15-cyclohexyl-5,6,8,9-tetrahydro-4H-indolo[1′,2′:4,5][1,4]diazepino[6,7,1-ij]quinoline-12-carboxylic acid: To a solution of methyl ester (80 mg, 0.15 mmol, 1.0 equiv) in THF (1.0 mL), MeOH (0.5 mL) and water (0.5 mL) was added LiOH.H₂O (63 mg, 1.5 mmol, 10.0 equiv). The mixture was stirred at 58° C. for 2 hours. The solution was neutralized to pH=6 by addition of 1.0 N HCl aq. solution. The solid was collected by filtration and washed with water. The solid was dissolved in CH₃CN and water. The solvent was then removed by freeze drying to give product 64 mg. MS: 515 [M+H⁺]. ¹H NMR (CDCl₃): 1.14-1.36 (m, 3H), 1.37-1.53 (s, 9H), 1.65-1.91 (m, 6H), 1.91-2.15 (m, 4H), 2.73-2.85 (m, 1H), 2.92-3.07 (m, 2H), 3.40-3.59 (m, 2H), 3.90-4.50 (m, 2H), 4.78-4.91 (m, 2H), 6.99-7.06 (t, 1H), 7.14-7.18 (d, 1H), 7.31-7.38 (d, 1H), 7.70-7.77 (d, 1H), 7.80-7.87 (d, 1H), 8.06 (s, 1H).

### Example 49

### Preparation of Compound 298

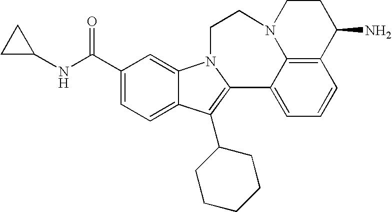

tert-Butyl [(4R)-15-cyclohexyl-12-(cyclopropylcarbamoyl)-5,6,8,9-tetrahydro-4H-indolo[1′,2′:4,5][1,4]diazepino[6,7,1-ij]quinolin-4-yl]carbamate: To a solution of (4R)-4-[(tert-butoxycarbonyl)amino]-15-cyclohexyl-5,6,8,9-tetrahydro-4H-indolo[1′,2′:4,5][1,4]diazepino[6,7,1-ij]quinoline-12-carboxylic acid (100 mg) in CH₂Cl₂ (1.0 mL) at 0° C. was added cyclopropyl amine (80 mg), HATU (90 mg) and DIPEA (0.1 mL). The solution was stirred at room temperature for 1 hour, after which 1.0 N HCl aq. solution and EtOAc were added. The phases were separated. The organic phase was washed with sat. aq. NaHCO₃ solution, brine, dried (Na₂SO₄) and concentrated. The residue was purified by silica gel column chromatography (heptane/EtOAc, 4/1 to 1/1) to give product 50 mg. MS: 555 [M+H⁺].

(4R)-4-amino-15-cyclohexyl-N-cyclopropyl-5,6,8,9-tetrahydro-4H-indolo[1′,2′:4,5][1,4]diazepino[6,7,1-ij]quinoline-12-carboxamide: To a solution of amide (100 mg) in CH₂Cl₂ (2.0 mL) at 0° C. was added 4.0 N HCl in dioxane (2.7 mL). The resultant mixture was stirred at room temperature for 1 hour. The solvent was then removed under vacuum. To the residue was added CH₂Cl₂ and heptane. After removing the solvent under vacuum, the solid was dissolved in CH₃CN and water. The solvent was removed by freeze drying to give product 106 mg. MS: 455 [M+H⁺]. ¹H NMR: 0.56-0.63 (m, 2H), 0.68-0.76 (m, 2H), 1.11-1.48 (m, 4H), 1.64-2.10 (m, 8H), 2.65-2.78 (m, 1H), 2.83-2.92 (m, 1H), 2.98-3.17 (m, 2H), 3.43-3.90 (m, 4H), 4.49-4.58 (m, 1H), 7.17-7.25 (t, 1H), 7.25-7.31 (d, 1H), 7.48-7.54 (d, 1H), 7.59-7.66 (d, 1H), 7.78-7.85 (d, 1H), 8.08 (s, 1H), 8.29-8.35 (d, 1H), 8.35-8.55 (s, 3H).

### Example 50

### Preparation of Compound 299

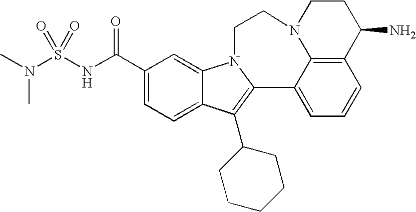

tert-butyl [(4R)-15-cyclohexyl-12-{[(dimethylamino)sulfonyl]carbamoyl}-5,6,8,9-tetrahydro-4H-indolo[1′,2′:4,5][1,4]diazepino[6,7,1-ij]quinolin-4-yl]carbamate: To a solution of (4R)-4-[(tert-butoxycarbonyl)amino]-15-cyclohexyl-5,6,8,9-tetrahydro-4H-indolo[1′,2′:4,5][1,4]diazepino[6,7,1-ij]quinoline-12-carboxylic acid (80 mg, 0.15 mmol) in CH₃CN (2.0 mL) at 0° C. was added N,N-dimethylsulfamide (154 mg, 1.24 mmol, 8.0 equiv), HATU (77 mg, 0.20 mmol, 1.3 equiv) and DMAP (152 mg, 1.24 mmol, 8.0 equiv). The mixture was stirred at room temperature for 1 hour, after which the mixture was separated by HLPC to give 20 mg of product. MS: 622 [M+H⁺].

(4R)-4-amino-15-cyclohexyl-N-[(dimethylamino)sulfonyl]-5,6,8,9-tetrahydro-4H-indolo[1′,2′:4,5][1,4]diazepino[6,7,1-ij]quinoline-12-carboxamide: To a solution of the sulfamide from the previous step (20 mg) in CH₂Cl₂ (1.0 mL) at room temperature was added 4.0 N HCl solution in dioxane (1.6 mL). The solution was stirred at room temperature for 1 hour, after which the solvent was removed under vacuum. To the residue was added CH₂Cl₂/heptane and the solvent was then removed under vacuum. The resultant solid was dissolved in CH₃CN and water. The solvent was removed by freeze drying to give product 17 mg. MS: 522 [M+H⁺]. ¹H NMR (DMSO-d6): 1.17-1.48 (m, 4H), 1.65-2.10 (m, 8H), 2.64-2.79 (m, 1H), 2.87-2.94 (s, 6H), 3.01-3.16 (m, 2H), 3.49-3.61 (m, 4H), 3.64-3.74 (m, 1H), 4.49-4.59 (m, 1H), 7.18-7.26 (t, 1H), 7.27-7.33 (d, 1H), 7.57-7.62 (d, 1H), 7.62-7.68 (d, 1H), 7.86-7.92 (d, 1H), 8.32 (s, 1H), 8.35-8.55 (s, 3H), 11.58 (s, 1H).

### Example 51

### Preparation of Compound 287

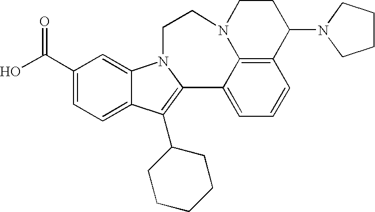

Methyl 15-cyclohexyl-4-pyrrolidin-1-yl-5,6,8,9-tetrahydro-4H-indolo[1′,2′:4,5][1,4]diazepino[6,7,1-ij]quinoline-12-carboxylate: To a solution of methyl 4-amino-15-cyclohexyl-5,6,8,9-tetrahydro-4H-indolo[1′,2′:4,5][1,4]diazepino[6,7,1-ij]quinoline-12-carboxylate HCl salt (80 mg, 0.19 mmol, 1.0 equiv) in DMF (2.0 mL) and Et₃N (188 mg) was added 1,4-dibromobutane (141 mg, 0.65 mmol, 3.5 equiv). The solution was then stirred at 70° C. for 12 hours. The mixture was purified by silica gel column chromatography (heptane/acetone/Et₃N, 1/1/0.02) to give product 37 mg. MS: 484 [M+H⁺].

15-Cyclohexyl-4-pyrrolidin-1-yl-5,6,8,9-tetrahydro-4H-indolo[1′,2′:4,5][1,4]diazepino[6,7,1-ij]quinoline-12-carboxylic acid: To a solution of methyl ester (37 mg, 0.077 mmol, 1.0 equiv) in THF (4.0 mL), MeOH (1.0 mL) and water (1.0 mL) was added LiOH.H₂O (96 mg, 2.3 mmol, 30.0 equiv). After stirring at 58° C. for 2 hours, the solution was neutralized to pH=6 by addition of 1.0 N HCl aq. solution. EtOAc was added and the phases were separated. The aqueous layer was extracted with EtOAc and the combined organic layers were washed with brine, dried (Na₂SO₄) and concentrated. The solid was dissolved in CH₃CN and water. The solvent was then removed by freeze drying to give product 25 mg. MS: 470 [M+H⁺]. ¹H NMR (DMSO-d6): 8.14 (s, 1H), 7.84 (d, J=7.5 Hz, 1H), 7.61 (d, J=7.4 Hz, 1H), 7.35 (d, J=7.4 Hz, 1H), 7.13 (d, J=8.0 Hz, 1H), 6.97 (t, J=7.0 Hz, 1H), 4.46-4.83 (br, 1H), 3.90-4.19 (m, 1H), 3.46-3.67 (m, 2H), 2.37-2.42 (m, 1H), 3.28-3.32 (m, 1H), 2.94-3.12 (m, 2H), 2.71-2.85 (m, 1H), 2.65-2.71 (m, 2H), 1.07-2.19 (m, 17H).

### Example 52

### Preparation of Compound 292

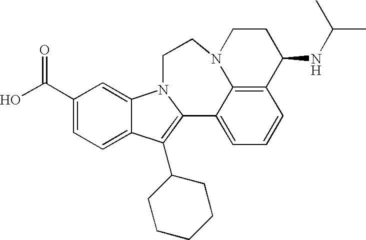

15-cyclohexyl-4-pyrrolidin-1-yl-5,6,8,9-tetrahydro-4H-indolo[1′,2′:4,5][1,4]diazepino[6,7,1-ij]quinoline-12-carboxylic acid: To a solution of methyl (4R)-4-amino-15-cyclohexyl-5,6,8,9-tetrahydro-4H-indolo[1′,2′:4,5][1,4]diazepino[6,7,1-ij]quinoline-12-carboxylate HCl salt (200 mg, 0.46 mmol, 1.0 equiv) in MeOH (4 mL) was added Et₃N (141 mg), acetone (37.9 mg, 0.65 mmol, 1.4 equiv), AcOH (0.1 mL, 0.46 mmol) and 4 Å molecule sieves. The solution was then stirred at room temperature for 20 minutes, after which NaBH(OAc)₃ (296 mg, 1.39 mmol) was added. After stirring at room temperature for 2 hours, the reaction was cooled at 0° C. To the solution was added sat. aq. NaHCO₃ solution and EtOAc. The phases were separated and the aqueous layer was extracted with EtOAc. The organic layers were combined, dried (Na₂SO₄) and concentrated. The residue was purified by silica gel column chromatography (heptane/acetone, 1/4) to give product 120 mg. MS: 472 [M+H⁺].

(4R)-15-cyclohexyl-4-(isopropylamino)-5,6,8,9-tetrahydro-4H-indolo[1′,2′:4,5][1,4]diazepino[6,7,1-ij]quinoline-12-carboxylic acid: To a solution of methyl ester (120 mg, 0.25 mmol, 1.0 equiv) in THF (1.0 mL) MeOH (0.5 mL) and water (0.5 mL) was added LiOH.H₂O (107 mg, 2.5 mmol, 10.0 equiv). The mixture was stirred at 57° C. for 2 hours. The solution was neutralized to pH=6 by addition of 1.0 N HCl aq. solution. EtOAc was added and the phases were separated. The aqueous layer was extracted with EtOAc and the combined organic layers were washed with brine, dried (Na₂SO₄) and concentrated. The solid was dissolved in CH₃CN and water. The solvent was then removed by freeze dry method to give product 84.3 mg. MS: 458 [M+H]. ¹H NMR (DMSO-d6): 8.15 (s, 1H), 7.83 (d, J=7.5 Hz, 1H), 7.60 (d, J=7.4 Hz, 1H), 7.44 (s, 1H), 7.12 (d, J=8.0 Hz, 1H), 7.07 (t, J=7.0 Hz, 1H), 3.81 (s, 1H), 3.46 (s, 2H), 3.10-2.80 (m, 3H), 2.78-2.75 (m, 1H), 2.02-2.00 (m, 1H), 1.90-1.86 (m, 6H), 1.38-1.20 (m, 6H), 1.11 (d, J=5.8 Hz), 1.04 (d, J=6.0 Hz).

### Example 53

### Preparation of Compound 281

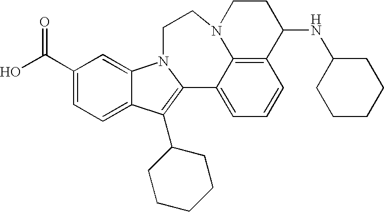

This compound was prepared as described for compound 292 in Example 52 in 0.13 mmol scale, using cyclohexanone. Yield: 15 mg. MS: 498 [M+H⁺]. ¹H NMR (400 MHz, DMSO-d6):1.00-2.15 (m, 23H), 2.57-2.98 (m, 3H), 3.00-3.19 (m, 1H), 3.40-3.56 (m, 2H), 3.76-3.94 (m, 1H), 4.42-4.91 (br, 1H), 6.99-7.10 (t, 1H), 7.10-7.19 (d, 1H), 7.36-7.52 (br, 1H), 7.52-7.64 (d, 1H), 7.78-7.91 (d, 1H), 8.16 (s, 1H).

### Example 54

### Preparation of Compound 281

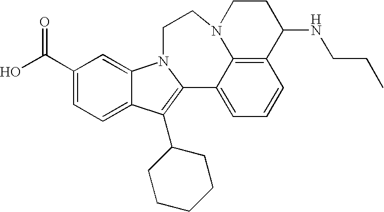

This compound was prepared as described for compound 292 in Example 52 in 0.13 mmol scale, using propionaldehyde. Yield: 21 mg. MS: 458 [M+H⁺]. ¹H NMR (400 MHz, DMSO-d6): 0.86-1.01 (t, 3H), 1.15-2.12 (m, 15H), 2.57-2.71 (m, 2H), 2.71-2.84 (m, 1H), 2.84-3.01 (m, 1H), 3.01-3.19 (m, 1H), 3.42-3.54 (m, 2H), 3.65-3.97 (m, 1H), 4.32-4.96 (br, 1H), 7.01-7.12 (t, 1H), 7.12-7.19 (d, 1H), 7.42-7.52 (d, 1H), 7.54-7.67 (d, 1H), 7.79-7.90 (d, 1H), 8.15 (s, 1H).

### Example 55

### Preparation of Compound 282

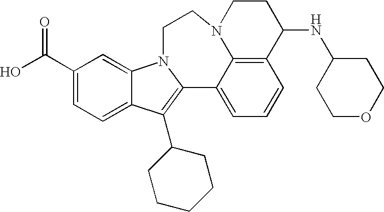

This compound was prepared as described for compound 292 in Example 52 in 0.13 mmol scale, using propionaldehyde. Yield: 20 mg. MS: 500 [M+H⁺]. ¹H NMR (400 MHz, DMSO-d6): 1.12-2.17 (m, 17H), 2.69-2.98 (m, 3H), 3.00-3.16 (m, 1H), 3.36-3.40 (m, 1H), 3.41-3.54 (m, 2H), 3.76-3.95 (m, 3H), 4.40-4.87 (br, 1H), 7.01-7.12 (t, 1H), 7.12-7.17 (d, 1H), 7.38-7.49 (br, 1H), 7.55-7.68 (m, 2H), 7.80-7.90 (d, 1H), 8.14 (s, 1H).

### Example 56

### Preparation of Compound 276

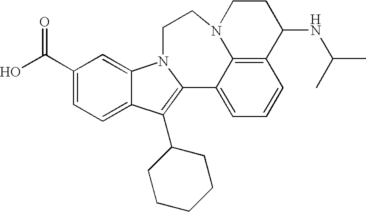

This compound was prepared as described for compound 292 in Example 52 in 0.13 mmol scale, using acetone. Yield: 26 mg. MS: 458 [M+H⁺]. ¹H NMR (400 MHz, DMSO-d6): 0.97-1.07 (d, 3H), 1.07-1.15 (d, 3H), 1.16-1.46 (m, 4H), 1.52-1.95 (m, 7H), 1.95-2.10 (m, 2H), 2.70-2.82 (m, 1H), 2.82-3.15 (m, 3H), 3.40-3.54 (m, 2H), 3.74-3.87 (m, 1H), 4.43-4.87 (br, 1H), 7.01-7.10 (t, 1H), 7.10-7.17 (d, 1H), 7.38-7.51 (br, 1H), 7.56-7.64 (d, 1H), 7.79-7.87 (d, 1H), 8.14 (s, 1H).

### Example 57

### Preparation of Compound 279

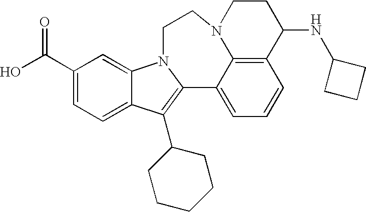

This compound was prepared as described for compound 292 in Example 52 in 0.23 mmol scale, using cyclobutanone (20 equiv.). Yield: 20 mg. MS: 470 [M+H⁺]. ¹H NMR (400 MHz, DMSO-d6): 1.13-1.47 (m, 5H), 1.47-1.90 (m, 11H), 1.90-2.09 (m, 3H), 2.09-2.29 (m, 2H), 2.69-2.83 (m, 1H), 2.83-2.98 (m, 1H), 3.00-3.17 (m, 1H), 3.41-3.57 (m, 2H), 3.66-3.77 (m, 1H), 4.35-4.95 (br, 1H), 6.99-7.09 (t, 1H), 7.09-7.17 (d, 1H), 7.33-7.46 (d, 1H), 7.55-7.66 (d, 1H), 7.77-7.90 (d, 1H), 8.15 (s, 1H).

### Example 58

### Preparation of Compound 280

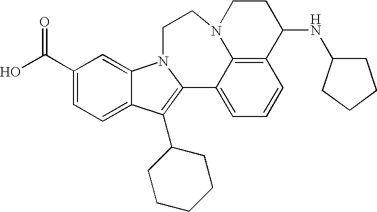

This compound was prepared as described for compound 292 in Example 52 in 0.23 mmol scale, using racemic methyl 4-amino-15-cyclohexyl-5,6,8,9-tetrahydro-4H-indolo[1′,2′:4,5][1,4]diazepino[6,7,1-ij]quinoline-12-carboxylate HCl salt and cycloheptanone (15 equiv.). Yield: 26 mg. MS: 484 [M+H⁺]. ¹H NMR (400 MHz, DMSO-d6): 1.10-1.59 (m, 8H), 1.59-1.94 (m, 10H), 1.94-2.15 (m, 3H), 2.70-2.85 (m, 1H), 2.85-3.00 (m, 1H), 3.01-3.16 (m, 1H), 3.18-3.32 (m, 2H), 3.41-3.56 (m, 2H), 3.66-3.80 (m, 1H), 4.39-4.98 (br, 1H), 6.98-7.10 (t, 1H), 7.10-7.20 (d, 1H), 7.34-7.50 (d, 1H), 7.55-7.66 (d, 1H), 7.76-7.90 (d, 1H), 8.15 (s, 1H).

### Example 59

### Preparation of Compound 278

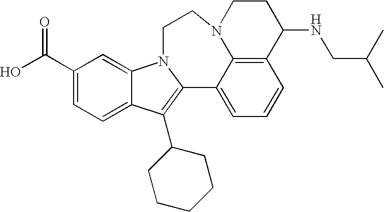

This compound was prepared as described for compound 292 in Example 52 in 0.23 mmol scale, using isobutyraldehyde. Yield: 25 mg. MS: 472 [M+H⁺]. ¹H NMR (400 MHz, DMSO-d6): 0.80-1.00 (d, 6H), 1.10-1.51 (m, 4H), 1.51-1.94 (m, 8H), 1.94-2.14 (m, 2H), 2.40-2.48 (m, 2H), 2.70-2.83 (m, 1H), 2.83-2.98 (m, 1H), 2.99-3.15 (m, 1H), 3.39-3.54 (m, 2H), 3.59-3.80 (m, 1H), 4.27-4.95 (br, 1H), 6.97-7.11 (t, 1H), 7.11-7.18 (d, 1H), 7.42-7.55 (d, 1H), 7.55-7.66 (d, 1H), 7.77-7.92 (d, 1H), 8.15 (s, 1H).

### Example 60

### Preparation of Compound 288

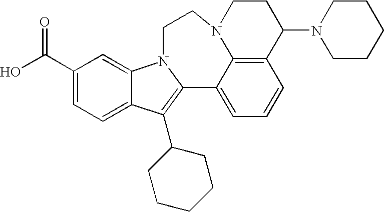

This compound was prepared as described for compound 287 in Example 51 in 0.19 mmol scale, using 1,5-dibromoheptane. Yield: 29 mg. MS: 484 [M+H⁺]. ¹H NMR (400 MHz, DMSO-d6): 1.06-2.12 (m, 19H), 2.50-2.60 (m, 2H), 2.71-2.88 (m, 1H), 2.88-3.10 (m, 2H), 3.35-3.42 (m, 2H), 3.41-3.56 (m, 1H), 3.66-3.94 (m, 2H), 4.37-4.80 (br, 1H), 7.00-7.18 (m, 2H), 7.52-7.61 (d, 1H), 7.62-7.69 (d, 1H), 7.71-7.84 (d, 1H), 8.07 (s, 1H)

### Example 61

### Preparation of Compound 283

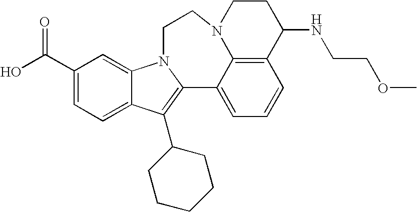

This compound was prepared as described for compound 287 in Example 51 in 0.23 mmol scale, using methoxyethyl bromide (1.1 equiv.). Yield: 13 mg. MS: 474 [M+H⁺]. ¹H NMR (400 MHz, DMSO-d6):1.03-2.30 (m, 13H), 2.69-2.81 (m, 1H), 2.98-3.17 (s, 3H), 3.15-3.30 (m, 5H), 3.44-3.61 (m, 2H), 3.61-3.79 (m, 2H), 4.42-4.66 (br, 1H), 7.03-7.23 (t, 1H), 7.24-7.35 (d, 1H), 7.51-7.71 (m, 2H), 7.79-7.94 (d, 1H), 8.20 (s, 1H), 8.77-9.17 (br, 1H), 12.45-12.70 (br, 1H).

### Example 62

### Preparation of Compound 289

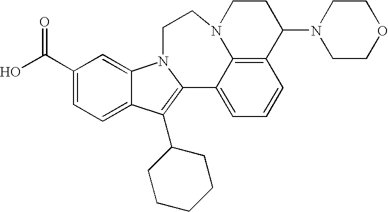

This compound was prepared as described for compound 287 in Example 51 in 0.28 mmol scale, using 1-(bromo-2-(2-bromoethoxy)ethane. Yield: 35 mg. MS: 486 [M+H⁺]. ¹H NMR (400 MHz, DMSO): 1.01-1.45 (m, 4H), 1.45-2.21 (m, 9H), 2.70-2.86 (m, 1H), 2.87-3.11 (m, 2H), 3.37-3.45 (m, 3H), 3.44-3.68 (m, 6H), 3.71-3.97 (m, 2H), 4.42-4.90 (br, 1H), 7.00-7.12 (t, 1H), 7.12-7.22 (d, 1H), 7.49-7.68 (dd, 2H), 7.71-7.87 (d, 1H), 8.12 (s, 1H), 12.29-13.00 (br, 1H).

### Example 63

### Preparation of Compound 291

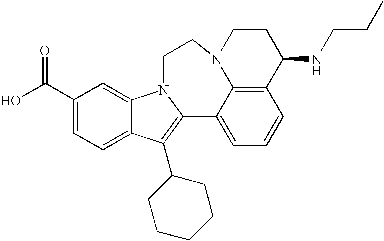

This compound was prepared as described for compound 292 in Example 52 in 0.70 mmol scale, using propionaldehyde. Yield: 30 mg. MS: 458 [M+H⁺]. ¹H NMR (400 MHz, DMSO-d6): 0.89-0.99 (t, 3H), 1.08-2.11 (m, 15H), 2.62-2.83 (m, 3H), 2.83-3.18 (m, 2H), 3.42-3.61 (m, 2H), 3.67-4.20 (br, 1H), 4.37-4.90 (br, 1H), 6.98-7.13 (t, 1H), 7.13-7.27 (d, 1H), 7.41-7.56 (d, 1H), 7.56-7.68 (d, 1H), 7.76-7.90 (d, 1H), 8.15 (s, 1H).

### Example 64

### Preparation of Compound 284

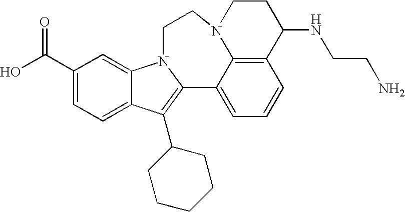

This compound was prepared as described for compound 292 in Example 52 in 0.23 mmol scale, using N—BOC-2-aminoacetaldehyde. Yield: 6 mg. MS: 459 [M+H⁺]. ¹H NMR (400 MHz, DMSO-d6): 1.00-2.22 (m, 13H), 2.69-2.82 (m, 1H), 3.01-3.23 (m, 3H), 3.23-3.36 (m, 4H), 3.60-3.75 (m, 2H), 4.53-4.72 (m, 1H), 7.06-7.19 (br, 1H), 7.23-7.34 (d, 1H), 7.56-7.67 (d, 1H), 7.67-7.79 (br, 1H), 7.80-7.94 (d, 1H), 8.19 (s, 1H), 8.23-8.41 (s, 3H), 9.31-9.64 (br, 1H).

### Example 65

### Preparation of Compound 285

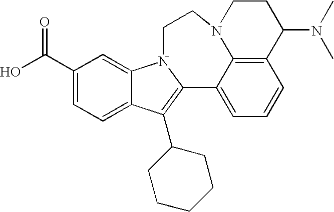

Methyl 15-cyclohexyl-4-(dimethylamino)-5,6,8,9-tetrahydro-4H-indolo[1′,2′:4,5][1,4]diazepino[6,7,1-ij]quinoline-12-carboxylate: To a solution of methyl 4-amino-15-cyclohexyl-5,6,8,9-tetrahydro-4H-indolo[1′,2′:4,5][1,4]diazepino[6,7,1-ij]quinoline-12-carboxylate HCl salt (60 mg, 0.13 mmol, 1.0 equiv) was added MeOH (0.5 mL) and AcOH (0.1 mL) followed by formaldehyde (37%, 24 μL, 0.325 mmol, 2.5 equiv). To the solution was slowly added NaBH₄ (28 mg, 0.78 mmol). After stirring at room temperature for 2 hours, the reaction was cooled at 0° C. and added sat. aq. NaHCO₃ solution and EtOAc. The phases were separated and the aqueous layer was extracted with EtOAc. The organic layers were combined, dried (Na₂SO₄) and concentrated. The residue was purified by silica gel column chromatography (heptane/acetone, 1/4) to give product 50 mg.

15-Cyclohexyl-4-(dimethylamino)-5,6,8,9-tetrahydro-4H-indolo[1′,2′:4,5][1,4]diazepino[6,7,1-ij]quinoline-12-carboxylic acid: To a solution of methyl ester (50 mg, 0.11 mmol, 1.0 equiv) in THF (1.0 mL) MeOH (0.5 mL) water (0.5 mL) was added LiOH.H₂O (46 mg, 1.1 mmol, 10.0 equiv). The mixture was stirred at 57° C. for 2 hours. The solution was neutralized to pH=6 by addition of 1.0 N HCl aq. solution. EtOAc was added and the phases were separated. The aqueous layer was extracted with EtOAc and the combined organic layers were washed with brine, dried (Na₂SO₄) and concentrated. The solid was dissolved in CH₃CN and water. The solvent was then removed by freeze dry method to give product 33 mg. ¹H NMR (400 MHz, DMSO): 1.09-2.11 (m, 12H), 2.23 (s, 6H), 2.73-2.86 (m, 1H), 2.89-3.11 (m, 2H), 3.36-3.45 (m, 1H), 3.36-3.61 (m, 1H), 3.66-4.11 (m, 2H), 4.38-4.99 (br, 1H), 6.98-7.12 (t, 1H), 7.12-7.27 (br, 1H), 7.50-7.69 (t, 2H), 7.76-7.93 (d, 1H), 8.16 (s, 1H), 11.98-12.70 (br, 1H).

### Example 66

### Preparation of Compound 286

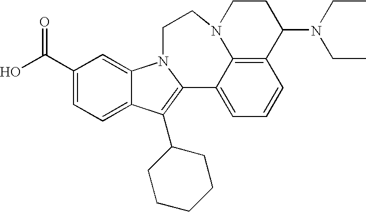

This compound was prepared as described for compound 285 in Example 65, using acetaldehyde. MS: 472 [M+H⁺]. ¹H NMR (400 MHz, DMSO-d6): 0.93-1.12 (t, 6H), 1.12-1.48 (m, 4H), 1.48-1.80 (m, 4H), 1.80-1.95 (m, 3H), 1.95-2.11 (m, 2H), 2.38-2.63 (m, 4H), 2.72-2.89 (m, 1H), 2.89-3.15 (m, 2H), 3.45-3.53 (m, 1H), 3.53-3.69 (m, 2H), 4.00-4.14 (m, 1H), 4.51-4.94 (br, 1H), 7.05-7.19 (m, 2H), 7.51-7.66 (d, 1H), 7.67-7.79 (d, 1H), 7.79-7.89 (d, 1H), 8.15 (s, 1H), 12.37-12.70 (br, 1H).

### Example 67

### Preparation of Compound 290

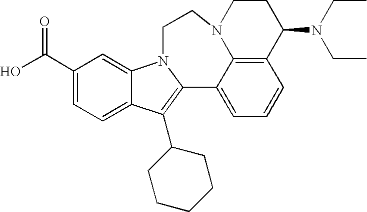

This compound was prepared as described for compound 285 in Example 65 in 0.58 mmol scale, using methyl (4R)-4-amino-15-cyclohexyl-5,6,8,9-tetrahydro-4H-indolo[1′,2′:4,5][1,4]diazepino[6,7,1-ij]quinoline-12-carboxylate HCl salt and acetaldehyde (30 equiv.). Yield: 13 mg. MS: 472 [M+H⁺]. ¹H NMR (400 MHz, DMSO-d6): 0.97-1.07 (t, 6H), 1.09-1.26 (m, 1H), 1.29-1.46 (m, 2H), 1.60-2.10 (m, 7H), 2.41-2.61 (m, 2H), 2.74-2.86 (m, 1H), 2.92-3.10 (m, 2H), 3.30-3.43 (m, 4H), 3.44-3.54 (m, 2H), 3.60-3.90 (m, 1H), 3.99-4.11 (m, 1H), 4.60-4.84 (m, 1H), 7.08-7.16 (m, 2H), 7.57-7.63 (d, 1H), 7.70-7.77 (d, 1H), 7.81-7.88 (d, 1H), 8.13-8.17 (s, 1H).

### Example 68

### Preparation of Compound 297

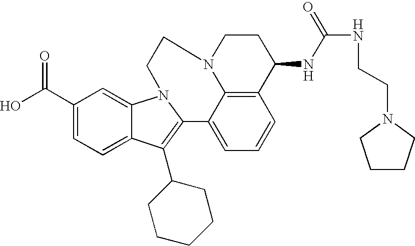

This compound was prepared as described for compound 296 in Example 48 in 0.13 mmol scale. Yield: 42 mg. MS: 556 [M+H⁺]. ¹H NMR (400 MHz, DMSO-d6): 1.00-2.13 (m, 17H), 2.37-2.47 (m, 8H), 2.69-3.07 (m, 3H), 3.07-3.20 (m, 2H), 3.41-3.61 (m, 2H), 4.70-4.94 (m, 1H), 5.57-5.87 (m, 1H), 7.01-7.22 (m, 2H), 7.36 (d, 1H), 7.68 (d, 1H), 7.85 (d, 1H), 8.14 (d, 1H), 12.6 (br, 1H).

### Example 69

### Preparation of Compound 295

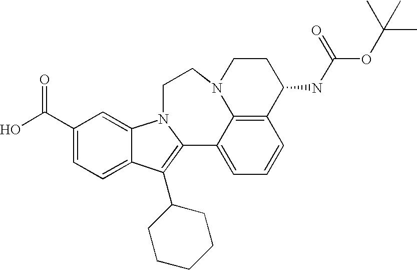

Methyl (4S)-4-[(tert-butoxycarbonyl)amino]-15-cyclohexyl-5,6,8,9-tetrahydro-4H-indolo[1′,2′:4,5][1,4]diazepino[6,7,1-ij]quinoline-12-carboxylate: Methyl 4-[(tert-butoxycarbonyl)amino]-15-cyclohexyl-5,6,8,9-tetrahydro-4H-indolo[1′,2′:4,5][1,4]diazepino[6,7,1-ij]quinoline-12-carboxylate was separated by chiral SFC into two enantiomers. To a solution of one enantiomer (260 mg, 0.56 mmol, 1.0 equiv) in CH₂Cl₂ (3.0 mL) was added DIPEA (144 mg, 1.12 mmol, 2.0 equiv) and (Boc)₂O (146 mg, 0.67 mmol, 1.2 equiv). The solution was then stirred at room temperature for 1 hour, after which the solvent was evaporated under vacuum and the residue was purified by silica gel column chromatography to give product 310 mg. MS: 530 [M+H⁺].

(4S)-4-[(tert-butoxycarbonyl)amino]-15-cyclohexyl-5,6,8,9-tetrahydro-4H-indolo[1′,2′:4,5][1,4]diazepino[6,7,1-ij]quinoline-12-carboxylic acid: To a solution of methyl ester (250 mg, 0.47 mmol, 1.0 equiv) in THF (1.0 mL) MeOH (1.0 mL) water (1.0 mL) was added LiOH.H₂O (56 mg, 2.36 mmol, 5.0 equiv). The mixture was stirred at 55° C. for 4 hours. The solution was neutralized to pH=3 by addition of 1.0 N HCl aq. solution. EtOAc was added and the phases were separated. The aqueous layer was extracted with EtOAc and the combined organic layers were washed with brine, dried (Na₂SO₄) and concentrated to give product 230 mg. MS: 516 [M+H⁺]. ¹H NMR (400 MHz, DMSO-d6): 1.05-1.42 (m, 4H), 1.44 (s, 9H), 1.60-2.20 (m, 8H), 2.70-2.85 (m, 1H), 2.90-3.20 (m, 2H), 3.40-3.65 (m, 3H), 4.60-4.90 (m, 2H), 7.05-7.15 (t, 1H), 7.15-7.23 (d, 1H), 7.25-7.36 (d, 1H), 7.36-7.50 (br, 1H), 7.55-7.65 (d, 1H), 7.80-7.93 (d, 1H), 8.20 (s, 1H), 12.70 (br, 1H).

### Example 70

### Preparation of Compound 275

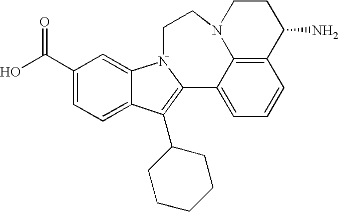

To a solution of one enantionmer of methyl 4-[(tert-butoxycarbonyl)amino]-15-cyclohexyl-5,6,8,9-tetrahydro-4H-indolo[1′,2′:4,5][1,4]diazepino[6,7,1-ij]quinoline-12-carboxylate (80 mg, 0.19 mmol, 1.0 equiv) in THF (4.0 mL), MeOH (1.0 mL) and water (1.0 mL) was added LiOH.H₂O (102 mg, 2.42 mmol, 13.0 equiv). The mixture was stirred at 58° C. for 2 hours, after which the solvent was removed under vacuum. The residue was then neutralized to pH=4 by addition of 1.0 N HCl aq. solution. The precipitate was collected by filtration and the solid was dissolved in CH₃CN and water. The solvent was removed by freeze dry method to give product 29 mg. MS: 416 [M+H⁺]. ¹H NMR (400 MHz, DMSO-d6): 1.00-1.48 (m, 4H), 1.62-2.12 (m, 8H), 2.65-2.81 (m, 1H), 2.96-3.18 (m, 2H), 3.42-3.65 (m, 3H), 4.45-4.62 (m, 2H), 7.16-7.25 (t, 1H), 7.25-7.34 (d, 1H), 7.56-7.67 (d, 2H), 7.82-7.91 (d, 1H), 8.18 (s, 1H), 8.39 (br, 3H).

### Example 71

### Preparation of Compound 304

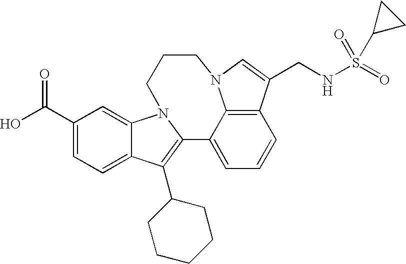

12-Cyclohexyl-1-{[(cyclopropylsulfonyl)amino]methyl}-5,6-dihydro-4H-[1,5]diazocino[1,2-a:5,4,3-h′i′]diindole-9-carboxylic acid was prepared as described for compound 121 in Example 21 in 0.064 mmole scale, using compound 101 and cyclopropanesulfonic acid amide to give 17 mg (Yield 50%). MS: 532.3 [M+H]+; ¹H-NMR (Methyl alcohol-d₄, 400 MHz): δ 8.17 (s, 1H), 7.88 (d, 1H, J=8 Hz), 7.86 (d, 1H, J=4 Hz), 7.85 (d, 1H, J=4 Hz), 7.75 (m, 3H), 4.51 (m, 2H), 4.03 (dd, 1H, J=4, 12 Hz), 3.76 (m, 1H), 2.95 (m, 1H), 2.46 (m, 1H), 2.20-1.85 (m, 6H), 1.79 (m, 1H), 1.60 (m, 1H), 1.41 (m, 3H), 1.20 (m, 2H), 1.06 (m, 2H), 0.92 (m, 2H).

### Example 72

### Preparation of Compound 305

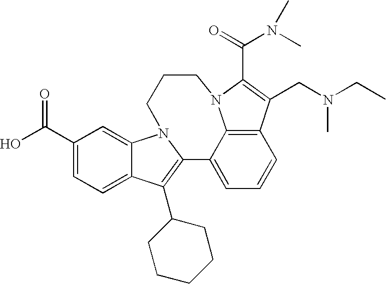

To a solution of 12-cyclohexyl-9-(methoxycarbonyl)-5,6-dihydro-4H-[1,5]diazocino[1,2-a:5,4,3-h′i′]diindole-2-carboxylic acid (100 mg, 0.22 mmole) in DMF (2 mL) was added HATU (83 mg, 0.22 mmole) at 0° C. followed by DIEA (0.17 mL, 0.99 mmole), and stirred for 15 min. Dimethylamine hydrochloride salt (54 mg, 0.96 mmole) was added to the solution. The resultant solution was warmed to room temperature and stirred for another 3 hr. The reaction was diluted with EtOAc (100 mL) and the mixture washed with saturated NaHCO₃ aqueous (20 mL×3) and brine (20 mL×1). The EtOAc extract was dried over MgSO₄, and solvent removed under vacuum. The residue was purified by flash (ISCO, 4 g silica column, with solvent gradient 10-30% EtOAc/heptane) column chromatography to give 90 mg of methyl 12-cyclohexyl-2-(dimethylcarbamoyl)-5,6-dihydro-4H-[1,5]diazocino[1,2-a:5,4,3-h′i′]diindole-9-carboxylate (yield 95%). MS: 484.2 [M+H]+; ¹H-NMR (Chloroform-d, 400 MHz): δ 8.19 (s, 1H), 7.91 (d, 1H, J=8 Hz), 7.80 (d, 1H, J=8 Hz), 7.72 (m, 1H), 7.22 (m, 2H), 6.71 (s, 1H), 4.49 (m, 1H), 4.19 (m, 1H), 3.95 (s, 3H), 3.79 (m, 1H), 3.41 (m, 1H), 3.20 (m, 6H), 2.95-2.84 (m, 2H), 2.36 (m, 1H), 2.15-1.95 (m, 3H), 1.87 (m, 1H), 1.73 (m, 2H), 1.64 (m, 1H), 1.48-1.10 (m, 3H).

To acetic acid (30 ml) was added formaldehyde (0.69 mL, 9.3 mmole) followed by ethylmethylamine (1.6 mL, 18.6 mmole). The mixture was stirred at room temperature for 20 minutes. To this mixture was added methyl 12-cyclohexyl-2-(dimethylcarbamoyl)-5,6-dihydro-4H-[1,5]diazocino[1,2-a:5,4,3-h′i′]diindole-9-carboxylate (1.5 g, 3.10 mmole). The solution was heated at 60° C. overnight. The mixture was evaporated to dryness then re-dissolved using EtOAc (100 mL) and the organic was washed with saturated aqueous NaHCO₃ (25 mL) and then dried over MgSO₄ and concentrated. Chromatography (ISCO, 40 g silica column, with solvent gradient 0-10% MeOH/DCM) gave 1.03 g of methyl 12-cyclohexyl-2-(dimethylcarbamoyl)-1-{[ethyl(methyl)amino]methyl}-5,6-dihydro-4H-[1,5]diazocino[1,2-a:5,4,3-h′i′]diindole-9-carboxylate (yield 60%). MS: 555.3 [M+H]+; ¹H-NMR (Chloroform-d, 400 MHz): δ 8.11 (d, 1H, J=4 Hz), 7.92 (m, 2H), 7.79 (d, 1H, J=8 Hz), 7.22 (m, 2H), 4.32 (m, 1H), 3.96 (m, 0.3H), 3.77 (s, 3H), 3.67 (m, 2H), 3.48 (m, 0.7H), 3.22 (m, 2H), 3.19 (s, 0.85H), 3.16 (s, 2.15H), 3.06 (s, 0.85H), 2.96 (s, 2.15H), 2.49 (m, 2H), 2.21-1.67 (m, 9H), 1.64 (m, 2H), 1.60 (m, 1H) 1.26 (m, 2H), 1.12 (m, 2H), 0.95 (m, 3H).

To the solution of methyl 12-cyclohexyl-2-(dimethylcarbamoyl)-1-{[ethyl(methyl)amino]methyl}-5,6-dihydro-4H-[1,5]diazocino[1,2-a:5,4,3-h′i′]diindole-9-carboxylate (100 mg, 0.18 mmole) in MeOH/H₂O/THF (0.6 mL:0.6 mL:0.6 mL) was added lithium hydroxide (24 mg, 0.54 mmole) and the mixture heated at 58° C. for 3 hr. The solvent was removed by vacuum and neutralized by addition of 3 equivalents of TFA. Chromatography (ISCO, 12 g silica column, with solvent gradient 0-10% MeOH/DCM) gave a crude white solid. Resuspension of the solid in 1 M HCl followed by lypholization gave 70 mg (yield 70%) of compound 266. MS: 541.5 [M+H]+; ¹H-NMR (DMSO-d₆, 600 MHz): δ 9.80 (bm, 1H), 8.14 (m, 1H), 8.07 (m 1H), 7.90 (dd, 1H, J=4.8, 8.4 Hz), 7.67 (d, 1H, J=8.4 Hz), 7.39 (m, 1H), 7.25 (m, 1H), 4.77-4.62 (m, 2H), 4.26-4.00 (m, 2H), 3.85-3.53 (m, 4H), 3.50-3.20 (m, 4H), 3.10-2.40 (m, 8H), 2.13-1.92 (m, 4H), 1.91-1.52 (m, 4H), 1.44-1.25 (m, 3H), 1.21-1.04 (m, 2H).

### Example 73

### Preparation of Compound 306

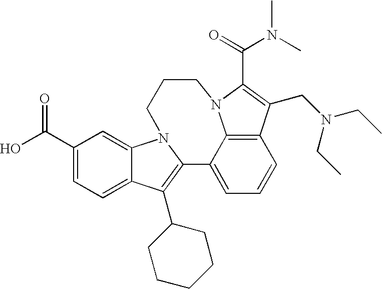

This compound was prepared as described for compound 305 in Example 72 in 0.12 mmole scale, using compound methyl 12-cyclohexyl-2-(dimethylcarbamoyl)-5,6-dihydro-4H-[1,5]diazocino[1,2-a:5,4,3-h′i′]diindole-9-carboxylate and diethylamine. Yield: 13.4 mg. MS: 555.5 [M+H]+; ¹H-NMR (Methyl alcohol-d₄, 400 MHz): δ 8.19 (s, 1H), 7.92 (m, 2H), 7.78 (d, 1H, J=8 Hz), 7.45 (m, 1H), 7.37 (d, 1H, J=8 Hz), 4.76 (d, 1H, J=12 Hz), 4.60 (m, 1H), 4.39-3.65 (m, 3H), 3.50-2.80 (m, 13H), 2.15 (m, 4H), 1.95 (m, 1H), 1.75 (m, 2H), 1.60 (m, 1H), 1.51-1.12 (m, 9H).

### Example 74

### Preparation of Compound 307

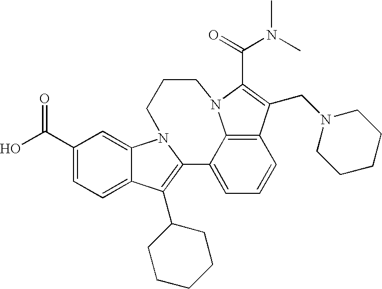

This compound was prepared as described for compound 305 in Example 72 in 0.124 mmole scale, using compound methyl 12-cyclohexyl-2-(dimethylcarbamoyl)-5,6-dihydro-4H-[1,5]diazocino[1,2-a:5,4,3-h′i′]diindole-9-carboxylate and piperidine. Yield: 25.2 mg. MS: 567.5 [M+H]+; ¹H-NMR (Methyl alcohol-d₄, 400 MHz): δ 8.19 (s, 1H), 7.97 (d, 1H, J=8 Hz), 7.93 (d, 1H, J=8 Hz), 7.78 (d, 1H, J=8 Hz), 7.44 (m, 1H), 7.36 (d, 1H, J=8 Hz), 4.72 (m, 1H), 4.61 (m, 1H), 4.32 (m, 1.3H), 3.80 (m, 1.7H), 3.55 (m, 2H), 3.49 (m, 1H), 3.30-2.70 (m, 1H), 2.15 (m, 4H), 1.93 (m, 2H), 1.78 (m, 4H), 1.60-1.10 (m, 6H).

### Example 75

### Preparation of Compound 308

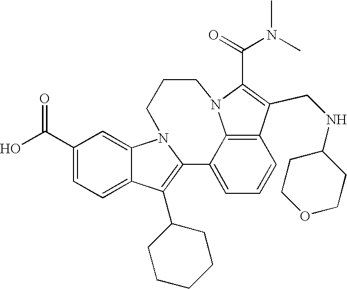

This compound was prepared as described for compound 305 in Example 72 in 0.12 mmole scale, using compound methyl 12-cyclohexyl-2-(dimethylcarbamoyl)-5,6-dihydro-4H-[1,5]diazocino[1,2-a:5,4,3-h′i′]diindole-9-carboxylate and 4-amino morpholine. Yield: 12.5 mg. MS: 583.5 [M+H]+; ¹H-NMR (Methyl alcohol-d₄, 400 MHz): δ 8.19 (s, 1H), 7.93 (m, 2H), 7.78 (d, 1H, J=8 Hz), 7.44 (m, 1H), 7.36 (d, 1H, J=8 Hz), 4.64 (m, 2H), 4.30 (m, 0.86H), 4.15-3.62 (m, 4.14H), 3.48 (m, 4H), 3.20 (s, 0.42H), 3.17 (s, 2.58H), 3.07 (s, 0.42H), 2.88 (s, 2.58H), 2.11 (m, 8H), 1.93 (m, 2H), 1.67 (m, 4H), 1.42 (m, 2H), 1.18 (m, 2H).

### Example 76

### Preparation of Compound 309

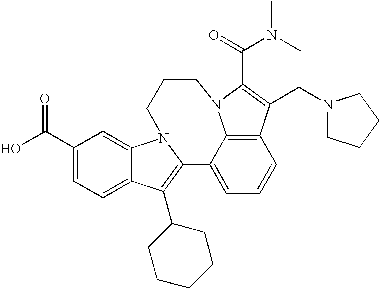

This compound was prepared as described for compound 305 in Example 72 in 0.12 mmole scale, using compound methyl 12-cyclohexyl-2-(dimethylcarbamoyl)-5,6-dihydro-4H-[1,5]diazocino[1,2-a:5,4,3-h′i′]diindole-9-carboxylate and pyrrolidine. Yield: 28.1 mg. MS: 553.5 [M+H]+; ¹H-NMR (Methyl alcohol-d₄, 400 MHz): δ 8.19 (s, 1H), 7.99 (d, 1H, J=8 Hz), 7.93 (d, 1H, J=8 Hz), 7.78 (d, 1H, J=8 Hz), 7.44 (m, 1H), 7.36 (d, 1H, J=8 Hz), 4.60 (m, 1H), 4.30 (m, 1H), 4.11 (m, 0.33H), 3.70 (m, 2.7H), 3.55-2.80 (m, 1H), 2.19 (m, 8H), 1.95 (m, 2H), 1.78 (m, 2H), 1.64 (m, 1H), 1.59-1.11 (m, 4H).

### Example 77

### Preparation of Compound 310

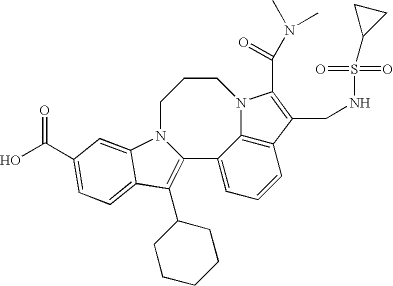

This compound was prepared as described for compound 305 in Example 72 in 0.06 mmole scale, using compound methyl 12-cyclohexyl-2-(dimethylcarbamoyl)-5,6-dihydro-4H-[1,5]diazocino[1,2-a:5,4,3-h′i′]diindole-9-carboxylate and cyclopropanesulfonic acid amide. Yield: 12 mg. MS: 601.1 [M−H]−; ¹H-NMR (Methyl alcohol-d₄, 400 MHz): δ 8.17 (s, 1H), 7.94 (m, 2H), 7.76 (d, 1H, J=8 Hz), 7.29 (m, 2H), 4.57 (m, 3H), 4.05 (m, 0.25H), 3.76 (m, 1.75H), 3.41-2.63 (m, 6H), 2.57 (m, 1H), 2.09 (m, 4H), 2.03 (m, 2H), 1.75 (m, 2H), 1.60 (m, 1H), 1.42 (m, 3H), 1.28-0.92 (m, 6H).

### Example 78

### Preparation of Compound 311

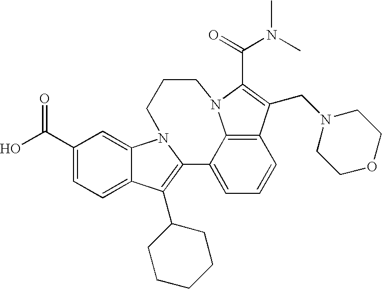

This compound was prepared as described for compound 305 in Example 72 in 0.12 mmole scale, using compound methyl 12-cyclohexyl-2-(dimethylcarbamoyl)-5,6-dihydro-4H-[1,5]diazocino[1,2-a:5,4,3-h′i′]diindole-9-carboxylate and morpholine. Yield: 31.3 mg. MS: 569.5 [M+H]+; ¹H-NMR (Methyl alcohol-d₄, 400 MHz): δ 8.19 (s, 1H), 7.99 (d, 1H, J=8 Hz), 7.93 (d, 1H, J=8 Hz), 7.78 (d, 1H, J=8 Hz), 7.45 (m, 1H), 7.37 (d, 1H, J=8 Hz), 4.82 (m, 1H), 4.60 (m, 1H), 4.32 (m, 0.9H), 4.10 (m, 2.1H), 3.92-3.44 (m, 7H), 3.43-2.80 (m, 10H), 2.17-1.90 (m, 6H), 1.76 (m, 2H), 1.61 (m, 1H), 1.43 (m, 2H).

### Example 79

### Preparation of Compound 312

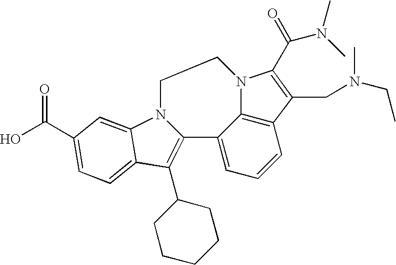

This compound was prepared as described for compound 108 in Example 8 in 0.17 mmole scale. A modification to the procedure involved the use of the C-3′benzyl protected acid. Yield: 17 mg. MS: 527.2 [M+H]+; ¹H-NMR (Methyl alcohol-d₄, 400 MHz): δ 8.21 (s, 1H), 7.94 (m, 2H), 7.77 (d, 1H, J=8 Hz), 7.58 (d, 1H, J=8 Hz), 7.48 (m, 1H), 4.60-4.25 (m, 4H), 3.50-3.15 (m, 7H), 3.13-2.79 (m, 6H), 2.23 (m, 3H), 2.00-1.61 (m, 5H), 1.48-1.27 (m, 6H).

### Example 80

### Preparation of Compound 313

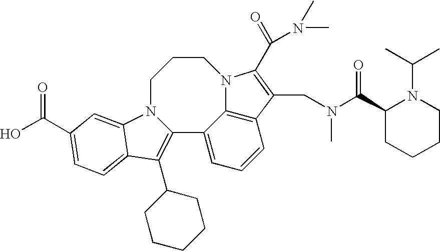

This compound was prepared as described for compound 305 in Example 72 in 0.057 mmole scale, using methyl 12-cyclohexyl-2-(dimethylcarbamoyl)-5,6-dihydro-4H-[1,5]diazocino[1,2-a:5,4,3-h′i′]diindole-9-carboxylate and methylamine. The amine methyl 12-cyclohexyl-2-(dimethylcarbamoyl)-1-[(methylamino)methyl]-5,6-dihydro-4H-[1,5]diazocino[1,2-a:5,4,3-h′i′]diindole-9-carboxylate was coupled with (2S)-1-(1-methylethyl)piperidine-2-carboxylic acid followed by deprotection.

To a solution of (2S)-1-(1-methylethyl)piperidine-2-carboxylic acid (39 mg, 0.23 mmole) in DMF/DCM (0.2/0.2 mL) was added EDCl (43.7 mg, 0.23 mmole) and HOBT (34.9 mg, 0.23 mmole) at followed by NMM (0.05 mL, 0.46 mmole), and stirred for 15 min. The amine methyl 12-cyclohexyl-2-(dimethylcarbamoyl)-1-[(methylamino)methyl]-5,6-dihydro-4H-[1,5]diazocino[1,2-a:5,4,3-h′i′]diindole-9-carboxylate (50 mg, 0.057 mmole) was added to the solution. The resulted solution stirred overnight. The reaction mixture was diluted with EtOAc (50 mL) and the mixture washed with saturated NaHCO₃ aqueous (10 mL×3) and brine (10 mL×1). The EtOAc extract was dried over MgSO₄, and solvent removed under vacuum. The residue was used for next step without further purification.

The residue was dissolved in MeOH/THF/H₂O (0.1/0.1/0.1 mL) and treated with LiOH.H₂O (10 mg, 0.228 mmole) and stirred at 60° C. overnight. The crude reaction mixture was loaded to HPLC (0.1% TFA/water/MeCN) to give 2 mg of compound 313. MS: 666.6 [M+H]+; ¹H-NMR (Methyl alcohol-d₄, 400 MHz): δ 8.18 (s, 1H), 7.80 (d, 1H, J=4 Hz), 7.87 (d, 1H, J=4 Hz), 7.78 (dd, 1H, J=2, 4 Hz), 7.28 (m, 2H), 5.09 (m, 1H), 4.80-4.40 (m, 2H), 4.25-3.60 (m, 3H), 3.51-2.82 (m, 17H), 2.20-1.50 (m, 11H), 1.49-1.00 (m, 10H).

### Example 81

### Preparation of Compound 314

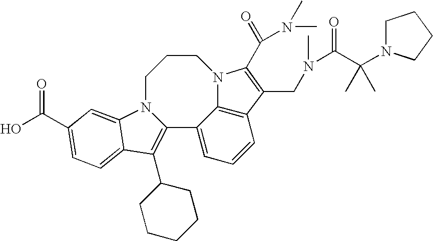

This compound was prepared as described for compound 313 in Example 80. The 2-methyl-2-pyrrolidinylpropanoic acid was employed for coupling to the amine methyl 12-cyclohexyl-2-(dimethylcarbamoyl)-1-[(methylamino)methyl]-5,6-dihydro-4H-[1,5]diazocino[1,2-a:5,4,3-h′i′]diindole-9-carboxylate in a 0.057 mmole scale, Yield: 0.8 mg. MS: 652.5 [M+H]+; ¹H-NMR (Methyl alcohol-d₄, 400 MHz): δ 8.18 (s, 1H), 7.92 (d, 1H, J=4 Hz), 7.85 (dd, 1H, J=4, 8 Hz), 7.77 (d, 1H, J=8 Hz), 7.29 (m, 2H), 5.15-4.80 (m, 1H), 4.65 (m, 1H), 3.80 (m, 1H), 3.64-2.85 (m, 12H), 2.25-1.81 (m, 10H), 1.80-1.10 (m, 17H).

### Example 82

### Preparation of Compound 315

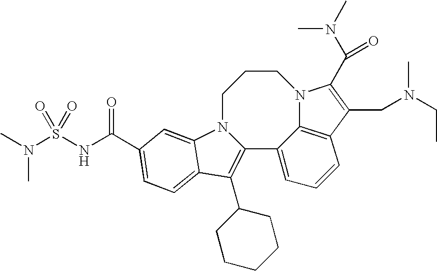

12-Cyclohexyl-2-(dimethylcarbamoyl)-1-{[ethyl(methyl)amino]methyl}-5,6-dihydro-4H-[1,5]diazocino[1,2-a:5,4,3-h′i′]diindole-9-carboxylic acid was coupled under HATU conditions to N-dimethyl sulfamide in 0.108 mmole scale to furnish compound 315, Yield: 27 mg.

To a solution of acid compound 312; 12-cyclohexyl-2-(dimethylcarbamoyl)-1-{[ethyl(methyl)amino]methyl}-5,6-dihydro-4H-[1,5]diazocino[1,2-a:5,4,3-h′i′]diindole-9-carboxylic acid (600 mg, 0.11 mmole) in THF (2 mL) was added HATU (140 mg, 0.37 mmole) and DMAP (180 mg, 1.47 mmole) and stirred for 15 min. N-dimethyl sulfamide (180 mg, 1.47 mmole) was added to the solution. The resultant solution stirred at 70° C. overnight. The reaction mixture was purified by HPLC (0.1% TFA/water/MeCN) to give compound 315. MS: 647.5 [M+H]+; ¹H-NMR (Methyl alcohol-d₄, 400 MHz): δ 8.12 (d, 1H, J=4 Hz), 7.95 (m, 2H), 7.66 (d, 1H, J=8 Hz), 7.44 (m, 1H), 7.36 (d, 1H, J=8 Hz), 4.65 (m, 1.7H), 4.42 (m, 0.3H), 4.22 (m, 1H), 3.85 (m, 2H), 3.44 (m, 2H), 3.28-2.69 (m, 16H), 2.15-1.93 (m, 6H), 1.76 (m, 2H), 1.65 (m, 1H), 1.47-1.13 (m, 7H).

### Example 83

### Preparation of Compound 316

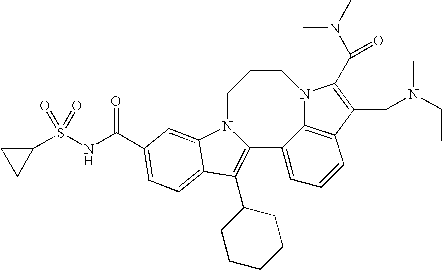

This compound was prepared as described for compound 315 in Example 82 in 0.11 mmole scale, using 12-cyclohexyl-2-(dimethylcarbamoyl)-1-{[ethyl(methyl)amino]methyl}-5,6-dihydro-4H-[1,5]diazocino[1,2-a:5,4,3-h′i′]diindole-9-carboxylic acid and cyclopropanesulfonic acid amide. Yield: 36 mg. MS: 553.5 [M+H]+; ¹H-NMR (Methyl alcohol-d₄, 400 MHz): δ 8.13 (s, 1H), 7.97 (m, 2H), 7.65 (d, 1H, J=8 Hz), 7.45 (m, 1H), 7.36 (d, 1H, J=8 Hz), 4.84 (m, 0.7H), 4.64 (m, 1.3H), 4.45 (m, 0.3H), 4.29-4.10 (m, 1H), 3.90 (m, 1.7H), 3.47 (m, 2H), 3.19 (m, 5H), 3.06 (m, 1H), 2.95-2.72 (m, 6H), 2.13 (m, 5H), 1.93 (m, 1H), 1.76 (m, 2H), 1.64 (m, 1H), 1.46-1.29 (m, 7H), 1.21-1.09 (m, 3H).

### Example 84

### Preparation of Compound 317

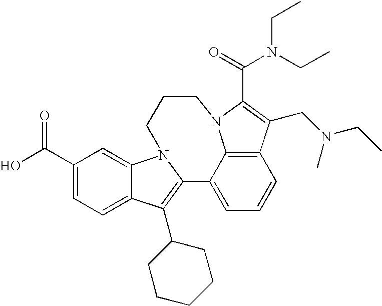

These compounds were prepared by first performing a Mannich reaction using ethylmethylamine which installs the C-3′ amine followed by a one-pot coupling (using HATU) of an amine to access the C-2′ amide and deprotection to afford the final product.

To acetic acid (5 mL) was added formaldehyde (0.10 mL, 1.31 mmole) followed by ethylmethylamine (0.45 mL, 5.26 mmole). The mixture was stirred at room temperature for 20 minutes. To this mixture was added 12-cyclohexyl-9-(methoxycarbonyl)-5,6-dihydro-4H-[1,5]diazocino[1,2-a:5,4,3-h′i′]diindole-2-carboxylic acid (200 mg, 0.438 mmole). The solution was heated at 60° C. for 1 hr. The mixture was evaporated to dryness and the residue was purified by chromatography (ISCO, 12 g silica column, with solvent gradient 0-10% MeOH/DCM) gave 160 mg of 12-cyclohexyl-1-{[ethyl(methyl)amino]methyl}-9-(methoxycarbonyl)-5,6-dihydro-4H-[1,5]diazocino[1,2-a:5,4,3-h′i′]diindole-2-carboxylic acid. MS: 526.3 [M−H]−; ¹H-NMR (Methyl alcohol-d₄, 400 MHz): δ 8.18 (s, 1H), 7.89 (m, 2H), 7.75 (d, 1H, J=8 Hz), 7.31 (m, 2H), 5.15 (m, 1H), 4.62-4.35 (m, 4H), 4.10 (m, 1H), 3.93 (s, 1H), 3.72 (m, 1H), 3.31 (s, 2H), 3.05 (m, 2H), 2.92 (m, 2H), 2.70 (s, 1H), 2.68 (s, 2H), 2.30-1.85 (m, 6H), 1.82-0.90 (m, 8H).

To a solution of 12-cyclohexyl-1-{[ethyl(methyl)amino]methyl}-9-(methoxycarbonyl)-5,6-dihydro-4H-[1,5]diazocino[1,2-a:5,4,3-h′i′]diindole-2-carboxylic acid (25 mg, 0.047 mmole) in DMF (0.2 mL) was added HATU (19 mg, 0.05 mmole) at 0° C. followed by DIEA (0.03 mL, 0.17 mmole), and stirred for 15 min. Diethylamine (4 mg, 0.05 mmole) was added to the solution. The resultant solution was warmed up to room temperature and stirred for another 3 hr. To the crude reaction mixture was added MeOH/H₂O (0.2/0.2 mL) and LiOH*H₂O (6 mg, 0.14 mmole) and the mixture stirred at 50° C. overnight. The reaction mixture was loaded to HPLC (0.1% TFA/water/MeCN) column and purified to give the 13.9 mg of compound 317. MS: 569.6 [M+H]+; ¹H-NMR (Methyl alcohol-d₄, 400 MHz): δ 8.19 (s, 1H), 7.94 (m, 2H), 7.78 (d, 1H, J=8 Hz), 7.45 (m, 1H), 7.37 (d, 1H, J=8 Hz), 5.05-4.60 (m, 2H), 4.60 (m, 1H), 4.30-4.00 (m, 1H), 3.95-2.71 (m, 12H), 2.10 (m, 5H), 1.95 (m, 1H), 1.81 (m, 2H), 1.62 (m, 1H), 1.52-1.25 (m, 6H), 1.23 (t, 3H, J=8 Hz), 1.01 (t, 3H, J=8 Hz).

### Example 85

### Preparation of Compound 318

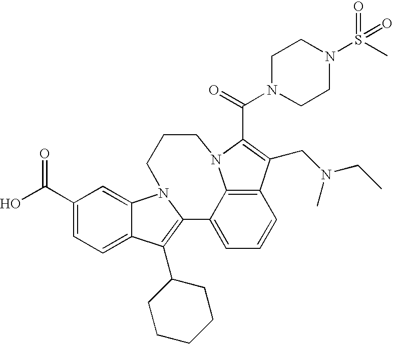

This compound was prepared as described for compound 317 in Example 84 in 0.047 mmole scale, using 12-cyclohexyl-1-{[ethyl(methyl)amino]methyl}-9-(methoxycarbonyl)-5,6-dihydro-4H-[1,5]diazocino[1,2-a:5,4,3-h′i′]diindole-2-carboxylic acid and 4-methlysulfonyl piperazines. Yield: 9.5 mg. MS: 704.6 [M+H]+; ¹H-NMR (Methyl alcohol-d₄, 400 MHz): δ 8.14 (s, 1H), 7.93 (d, 1H, J=8 Hz), 7.88 (d, 1H, J=8 Hz), 7.75 (d, 1H, J=8 Hz), 7.40 (m, 1H), 7.36 (d, 1H, J=8 Hz), 4.72-4.55 (m, 3H), 4.20-3.60 (m, 6H), 3.55-3.05 (m, 8H), 3.10-2.69 (m, 7H), 2.10 (m, 4H), 1.92 (m, 1H), 1.85 (m, 2H), 1.61 (m, 2H), 1.42 (m, 3H), 1.16 (m, 2H).

### Example 86

### Preparation of Compound 319

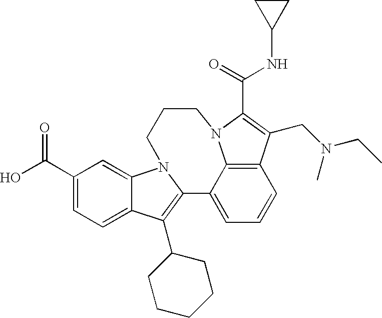

This compound was prepared as described for compound 317 in Example 84 in 0.047 mmole scale, using 12-cyclohexyl-1-{[ethyl(methyl)amino]methyl}-9-(methoxycarbonyl)-5,6-dihydro-4H-[1,5]diazocino[1,2-a:5,4,3-h′i′]diindole-2-carboxylic acid and cyclopropyl amine. Yield: 2.4 mg. MS: 553.5 [M+H]+; ¹H-NMR (Methyl alcohol-d₄, 400 MHz): δ 8.20 (s, 1H), 7.94 (d, 1H, J=4 Hz), 7.93 (d, 1H, J=8 Hz), 7.78 (d, 1H, J=8 Hz), 7.45 (dd, 1H, J=4, 8 Hz), 7.37 (d, 1H, J=8 Hz), 4.72-4.49 (m, 3H), 4.23 (m, 1H), 3.78 (m, 1H), 3.48-3.13 (m, 3H), 2.98-2.89 (m, 2H), 2.85 (s, 3H), 2.10 (m, 5H), 1.93 (m, 1H), 1.76 (m, 2H), 1.60 (m, 1H), 1.44 (m, 5H), 1.17 (m, 1H), 0.86 (m, 2H), 0.64 (m, 2H).

### Example 87

### Preparation of Compound 320

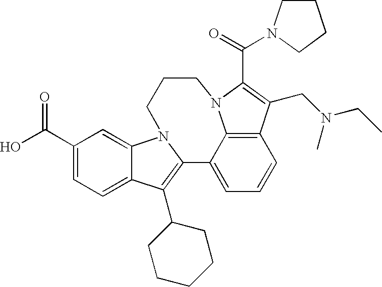

This compound was prepared as described for compound 317 in Example 84 in 0.047 mmole scale, using 12-cyclohexyl-1-{[ethyl(methyl)amino]methyl}-9-(methoxycarbonyl)-5,6-dihydro-4H-[1,5]diazocino[1,2-a:5,4,3-h′i′]diindole-2-carboxylic acid and pyrrolidine. Yield: 5 mg. MS: 567.5 [M+H]+; ¹H-NMR (Methyl alcohol-d₄, 400 MHz): δ 8.19 (s, 1H), 7.95 (m, 2H), 7.77 (d, 1H, J=12 Hz), 7.42 (m, 1H), 7.36 (d, 1H, J=8 Hz), 4.60 (m, 2H), 4.20 (m, 1H), 4.18 (m, 1H), 3.68 (m, 4H), 3.42 (m, 3H), 3.19 (m, 2H), 3.10-2.60 (m, 4H), 2.00 (m, 9H), 1.76 (m, 1H), 1.63 (m, 1H), 1.50-1.05 (m, 7H).

### Example 88

### Preparation of Compound 321

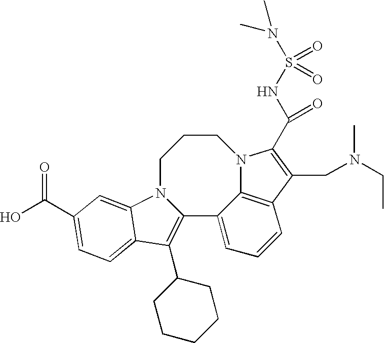

This compound was prepared as described for compound 317 in Example 84 in 0.05 mmole scale, using 12-cyclohexyl-1-{[ethyl(methyl)amino]methyl}-9-(methoxycarbonyl)-5,6-dihydro-4H-[1,5]diazocino[1,2-a:5,4,3-h′i′]diindole-2-carboxylic acid and N-dimethyl sulfamide. Yield: 11 mg. MS: 620.5 [M+H]+; ¹H-NMR (Methyl alcohol-d₄, 400 MHz): δ 8.21 (s, 1H), 7.99 (d, 1H, J=8 Hz), 7.93 (d, 1H, J=8 Hz), 7.79 (d, 1H, J=8 Hz), 7.74 (m, 2H), 4.82-4.41 (m, 4H), 3.77 (m, 1H), 3.41 (m, 2H), 3.31-2.75 (m, 10H), 2.17 (m, 5H), 1.94 (m, 1H), 1.78 (m, 2H), 1.62 (m, 1H), 1.44-1.19 (m, 7H).

### Example 89

### Preparation of Compound 322

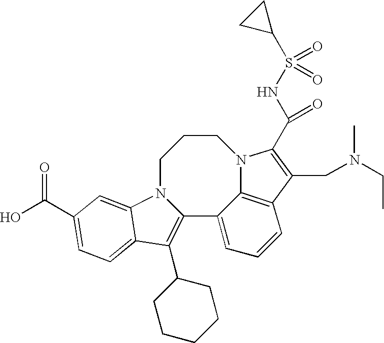

This compound was prepared as described for compound 317 in Example 84 in 0.05 mmole scale, using 12-cyclohexyl-1-{[ethyl(methyl)amino]methyl}-9-(methoxycarbonyl)-5,6-dihydro-4H-[1,5]diazocino[1,2-a:5,4,3-h′i′]diindole-2-carboxylic acid and cyclopropanesulfonic acid amide. Yield: 22 mg. MS: 617.4 [M+H]+; ¹H-NMR (Methyl alcohol-d₄, 400 MHz): δ 8.20 (s, 1H), 7.99 (d, 1H, J=8 Hz), 7.93 (d, 1H, J=8 Hz), 7.77 (d, 1H, J=8 Hz), 7.43 (m, 2H), 4.79 (m, 1H), 4.62 (m, 2H), 3.77 (m, 1H), 3.42-3.14 (m, 1H), 2.98-2.75 (m, 4H), 2.17 (m, 5H), 1.94 (m, 1H), 1.75 (m, 2H), 1.60 (m, 1H), 1.42 (m, 5H), 1.22 (m, 2H), 1.12 (m, 3H).

### Example 90

### Preparation of Compound 323

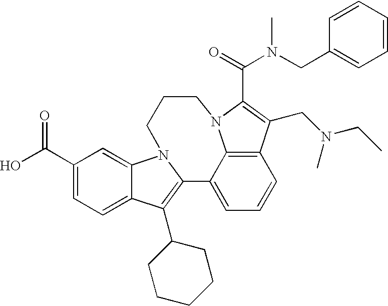

This compound was prepared as described for compound 317 in Example 84 in 0.047 mmole scale, using 12-cyclohexyl-1-{[ethyl(methyl)amino]methyl}-9-(methoxycarbonyl)-5,6-dihydro-4H-[1,5]diazocino[1,2-a:5,4,3-h′i′]diindole-2-carboxylic acid and N-methyl-1-phenylmethanamine. Yield: 10.6 mg. MS: 617.3 [M+H]+; ¹H-NMR (Methyl alcohol-d₄, 400 MHz): δ 8.15 (m, 1H), 7.93 (m, 2H), 7.75 (m, 1H), 7.45 (m, 6H), 7.15 (m, 1H), 5.11-4.77 (m, 2H), 4.72-4.40 (m, 3H), 4.21-3.52 (m, 3H), 3.50-2.50 (m, 10H), 2.23-1.95 (m, 5H), 1.80 (m, 1H), 1.72 (m, 2H), 1.60 (m, 1H), 1.44-0.90 (m, 5H).

### Example 91

### Preparation of Compound 324

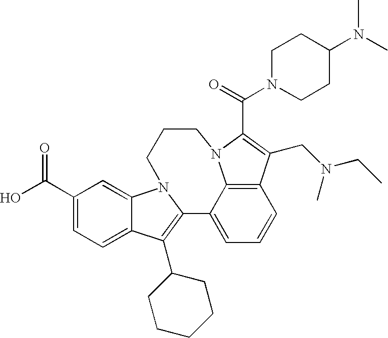

This compound was prepared as described for compound 317 in Example 84 in 0.047 mmole scale, using 12-cyclohexyl-1-{[ethyl(methyl)amino]methyl}-9-(methoxycarbonyl)-5,6-dihydro-4H-[1,5]diazocino[1,2-a:5,4,3-h′i′]diindole-2-carboxylic acid and N,N-dimethylpiperidin-4-amine. Yield: 14.5 mg. MS: 624.3 [M+H]+; ¹H-NMR (Methyl alcohol-d₄, 400 MHz): δ 8.19 (m, 1H), 7.97 (m, 2H), 7.77 (m, 1H), 7.45 (m, 1H), 7.39 (m, 1H), 4.62 (m, 2H), 4.41-4.10 (m, 1H), 4.05-3.61 (m, 2H), 3.50 (m, 5H), 3.20-2.65 (m, 12H), 2.39-1.75 (m, 9H), 1.80 (m, 2H), 1.65 (m, 2H), 1.51-1.05 (m, 7H).

### Example 92

### Preparation of Compound 325

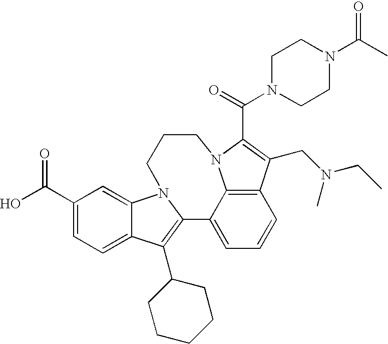

This compound was prepared as described for compound 317 in Example 84 in 0.047 mmole scale, using 12-cyclohexyl-1-{[ethyl(methyl)amino]methyl}-9-(methoxycarbonyl)-5,6-dihydro-4H-[1,5]diazocino[1,2-a:5,4,3-h′i′]diindole-2-carboxylic acid and 1-acetylpiperazine. Yield: 12 mg. MS: 624.2 [M+H]+; ¹H-NMR (Methyl alcohol-d₄, 400 MHz): δ 8.16 (s, 1H), 7.93 (d, 1H, J=8 Hz), 7.90 (d, 1H, J=8 Hz), 7.76 (d, 1H, J=12 Hz), 7.42 (m, 1H), 7.35 (d, 1H, J=8 Hz), 4.66 (m, 2H), 4.15 (m, 1H), 3.92 (m, 2H), 3.84 (m, 3H), 3.62 (m, 1H), 3.50-3.10 (m, 7H), 2.95 (m, 1H), 2.77 (m, 3H), 2.76-1.94 (m, 8H), 1.86 (m, 1H), 1.76 (m, 2H), 1.57 (m, 1H), 1.43 (m, 5H), 1.15 (m, 1H).

### Example 93

### Preparation of Compound 326

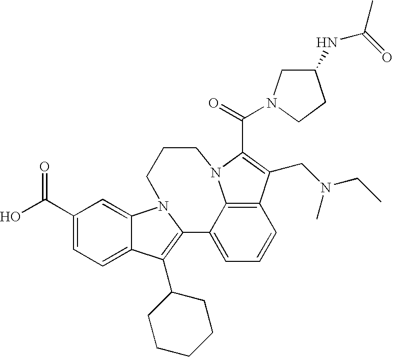

This compound was prepared as described for compound 317 in Example 84 in 0.047 mmole scale, using 12-cyclohexyl-1-{[ethyl(methyl)amino]methyl}-9-(methoxycarbonyl)-5,6-dihydro-4H-[1,5]diazocino[1,2-a:5,4,3-h′i′]diindole-2-carboxylic acid and N[(3R)-pyrrolidin-3-yl]acetamide. Yield: 14 mg. MS: 624.2 [M+H]+; ¹H-NMR (Methyl alcohol-d₄, 400 MHz): δ 8.15 (s, 1H), 7.92 (m, 2H), 7.77 (d, 1H, J=8 Hz), 7.41 (m, 1H), 7.35 (m, 1H), 4.62 (m, 2H), 4.48-4.09 (m, 2H), 3.95 (m, 1H), 3.70 (m, 2H), 3.61-3.15 (m, 6H), 2.95 (m, 1H), 2.72 (m, 3H), 2.60-1.64 (m, 10H), 1.78 (m, 3H), 1.60 (m, 1H), 1.49-0.95 (m, 6H).

### Example 94

### Preparation of Compound 404

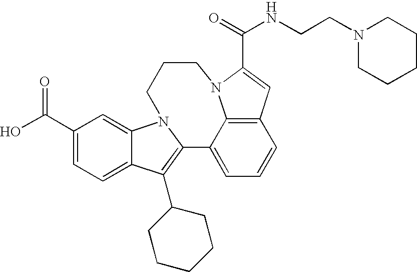

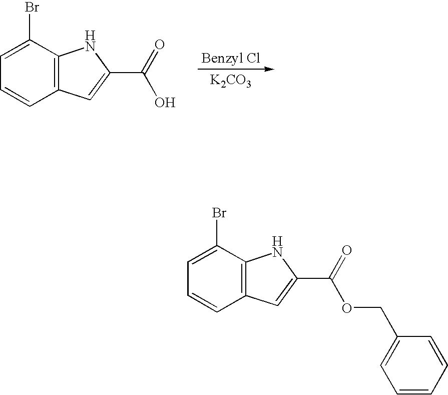

To a solution of 7-Bromo-1H-indole-2-carboxylic acid (1 g, 4.2 mmol) in 40 ml DMF, Benzyl Bromide (0.599 mL, 5.04 mmole) and Potassium Carbonate (580 mg, 4.2 mmole) were added. The reaction was stirred at room temperature over night. The reaction was concentrated and 400 ml of water was added. The aqueous mixture was extracted with 300 ml ethyl acetate, which was then dried with brine, dried over mag sulfate, concentrated, and dried over P₂O₅. Yield: 800 mg (58%). H¹-NMR (DMSO d₆): δ (ppm) 11.96 (s, 1H), 7.68 (d, 1H, J=9 Hz), 7.50 (m, 3H), 7.41 (m, 4H), 7.03 (m, 1H), 5.39 (s, 2H).

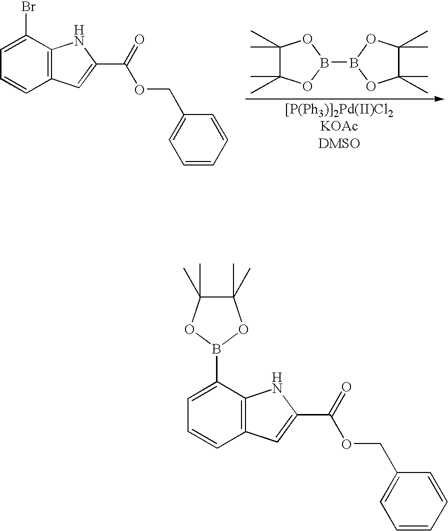

To a solution of 7-Bromo-1H-indole-2-carboxylic acid benzyl ester (390 mg, 1.18 mmole) in 18 ml dioxane, 4,4,5,5,4′,4′,5′,5′-Octamethyl-[2,2′]bi[[1,3,2]dioxaborolanyl] (599 mg, 2.36 mmole), bistriphenylphophene palladium(II) chloride (83 mg, 0.118 mmole), and potassium acetate (347 mg, 3.54 mmole) were added. The reaction mixture was then degassed and refluxed under nitrogen at 130° C. for 25 minutes. The complete reaction was concentrated and purified via silica gel chromatography. Yield: 900 mg (100% product+borane reagent as seen in NMR). H¹-NMR (DMSO d₆): δ (ppm) 9.79 (s, 1H), 7.85 (d, 1H, J=8.1 Hz), 7.62 (dd, 1H, J=6.9 Hz, 1.2 Hz), 7.41 (m, 6H), 7.15 (m, 1H), 5.39 (s, 2H), 1.36 (s, 12H).

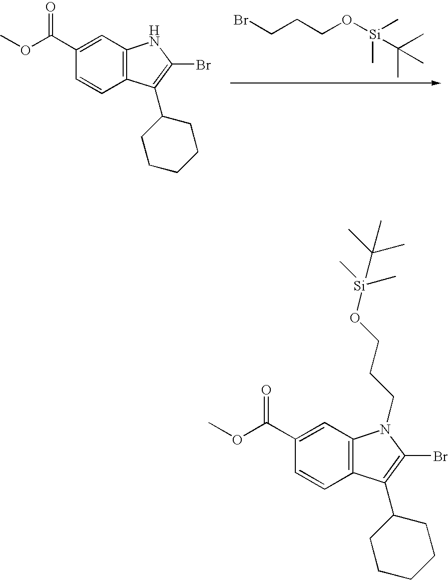

To a solution of the above 2-bromo-1H-indole (1.5 g, 4.46 mmole) in 90 mL DMF, a 60% suspension of NaH in mineral oil (196 mg, 4.91 mmole) was added at room temperature. The evolving hydrogen was pooled out by keeping under mild vacuum for 15 minutes when (3-Bromo-propoxy)-tert-butyl-dimethyl-silane (10.3 mL, 44.6 mmole) was added. The reaction was complete at 1 hour. It was then evaporated to dryness and the resulting oily product was diluted with 500 mL water and extracted with 500 mL ethyl acetate which was then dried with brine, dried over mag sulfate, concentrated, and dried over P205. Yield: 2.12 g (94%). MS (M+Na⁺): 531.2; H¹-NMR (DMSO d₆): δ (ppm) 8.07 (s, 1H), 7.80 (d, 1H, J=8.4 Hz), 7.62 (dd, 1H, J=8.4 Hz, 1.5 Hz), 4.34 (m, 2H), 3.85 (s, 3H), 3.60 (m, 2H), 2.84 (m, 1H), 1.82 (m, 9H), 1.38 (m, 3H), 0.889 (m, 9H), 0.051 (m, 6H).

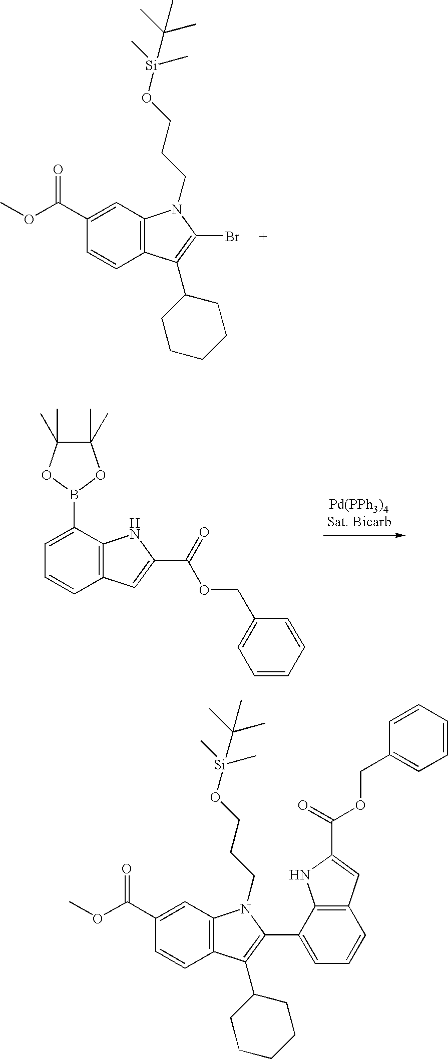

2-Bromo-1-[3-(tert-butyl-dimethyl-silanyloxy)-propyl]-3-cyclohexyl-1H-indole-6-carboxylic acid methyl ester (850 mg, 1.67 mmole), 7-(4,4,5,5-Tetramethyl-[1,3,2]dioxaborolan-2-yl)-1H-indole-2-carboxylic acid benzyl ester (1.26 g, 3.34 mmole), Pd(PPh₃)₄ (193 mg, 0.167 mmole), and aqueous saturated sodium bicarbonate (3 mL) were added to 30 mL DMF. The mixture was degassed and refluxed under argon at 130° C. for 25 minutes. The completed reaction was then concentrated and purified via silica gel chromatography. Yield: 800 mg (71%). MS (M+H⁺): 679.4; H¹-NMR (DMSO d₆): δ (ppm) 11.88 (s, 1H), 8.10 (d, 1H, J=1.2 Hz), 7.81 (m, 2H), 7.66 (m, 1H), 7.39 (m, 7H), 7.21 (m, 2H), 5.33 (m, 3H), 4.07 (m, 1H), 3.87 (s, 1H), 3.80 (m, 1H), 3.37 (m, 2H), 1.37 (m, 15H), 0.70 (s, 9H), −0.16 (s, 6H).

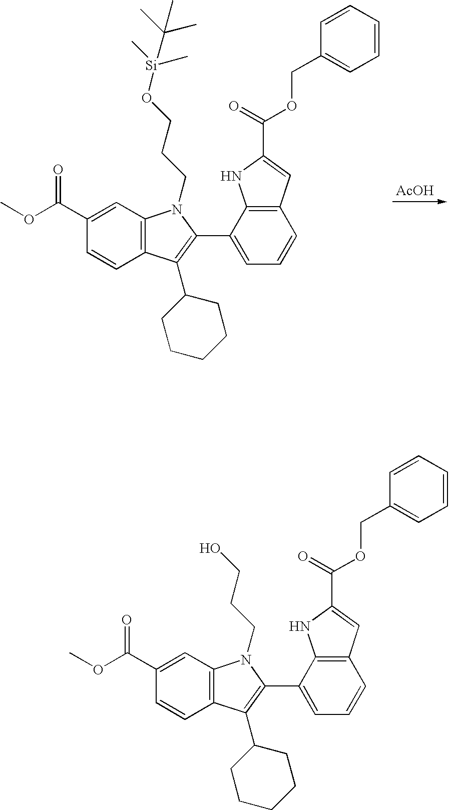

1-[3-(tert-Butyl-dimethyl-silanyloxy)-propyl]-3-cyclohexyl-1H,1′H-[2,7′]biindolyl-6,2′-dicarboxylic acid 2′-benzyl ester 6-methyl ester (800 mg, 1.18 mmole) was dissolved in a solution of 3:1:1 Acetic acid:water:THF (100 mL) and heated to 55° C. for 90 minutes. The completed reaction was then concentrated to an oil, coevaporated 3 times with toluene and foamed with dichloromethane. Yield: 800 mg (100%+). MS (M+H⁺): 565.3.

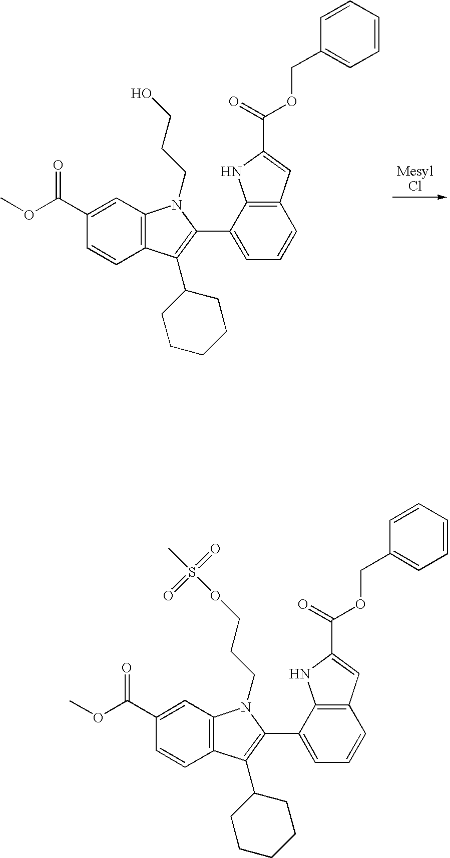

1-[3-(tert-Butyl-dimethyl-silanyloxy)-propyl]-3-cyclohexyl-1H,1′H-[2,7′]biindolyl-6,2′-dicarboxylic acid 2′-benzyl ester 6-methyl ester (750 mg, 1.33 mmole) and triethylamine (0.74 mL, 5.32 mmole) were suspended in anhydrous dichloromethane and the temperature was reduced to 0° C. Methanesulfonyl chloride (0.21 mL, 2.66 mmole) was added drop wise, and the reaction was complete instantaneously. The reaction was then diluted with dichloromethane, washed with water and brine, dried over magnesium sulfate, and concentrated. Yield: 820 mg (96%). MS (M+H⁺): 643.2.

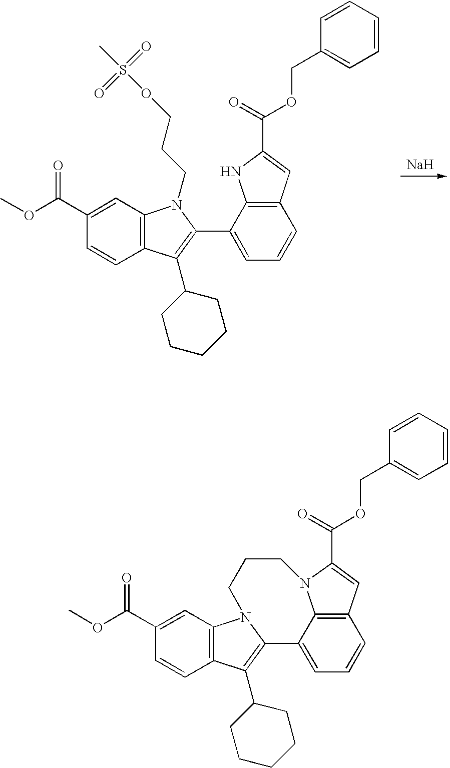

3-Cyclohexyl-1-(3-methanesulfonyloxy-propyl)-1H,1′H-[2,7′]biindolyl-6,2′-dicarboxylic acid 2′-benzyl ester 6-methyl ester (750 mg, 1.17 mmole) was dissolved in 7.5 mL DMF. The temperature was reduced to 0° C. and a 60% suspension of NaH in mineral oil (51 mg, 1.29 mmole) was added. The reaction was complete in 12 minutes, at which point 5 mL cold saturated sodium bicarbonate solution was added to quench. The reaction was diluted with 75 mL water, and extracted with 100 mL ethyl acetate. The organic layer was then washed with water, brine, dried over magnesium sulfate, and concentrated. Yield: 600 mg (94%). MS (M+H⁺): 547.3.

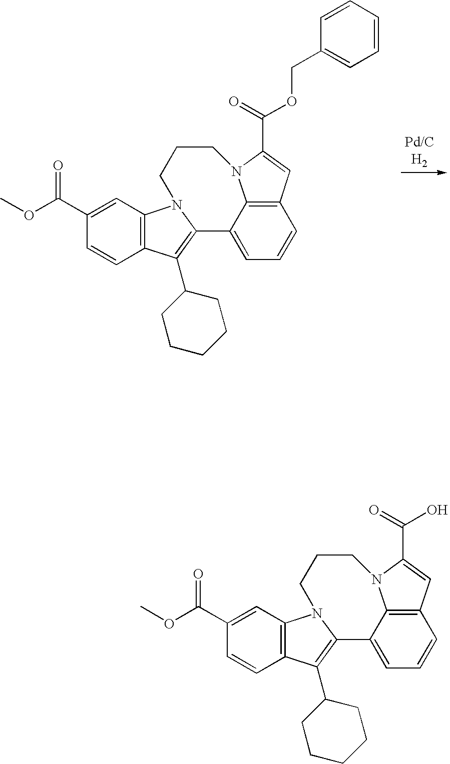

The above diester (560 mg, 1.02 mmole) was dissolved in 35 mL THF, followed by the addition of 250 mg Pd/C₁₀%. The reaction was stirred under a balloon of H₂ gas for 3 hr, after which the reaction was filtered and concentrated. Yield: 500 mg (100%). MS (M+H⁺): 457.2; H¹-NMR (DMSO d₆): δ (ppm) 8.16 (s, 1H), 7.94 (d, 1H, J=8.7 Hz), 7.83 (dd, 1H, J=6.9 Hz, 2.4 Hz), 7.68 (dd, 1H, J=8.4 Hz, 1.2 Hz), 7.37 (s, 1H), 7.27 (m, 2H), 5.02 (m, 1H), 4.67 (m, 1H), 4.13 (m, 1H), 3.88 (s, 3H), 3.85 (m, 1H), 3.60 (m, 1H), 3.18 (m, 1H), 2.85 (m, 1H), 1.89 (m, 7H), 1.15 (m, 3H).

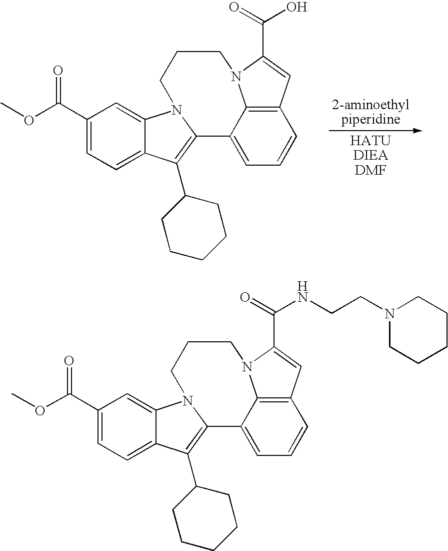

The above acid (100 mg, 0.219 mmole) and HATU (167 mg, 0.438 mmole) were suspended in 2.5 mL DMF and DIEA (0.138 mL, 1.10 mmole) was added. The reaction was stirred at room temperature for 5 minutes before 2-aminoethyl piperidine (0.062 mL, 0.438 mmole) was added. The reaction continued stirring over night, at which point the completed reaction was concentrated, precipitated in water, and dried over phosphorus pentoxide before being taken on to the next step as is. MS (M+H⁺): 567.3.

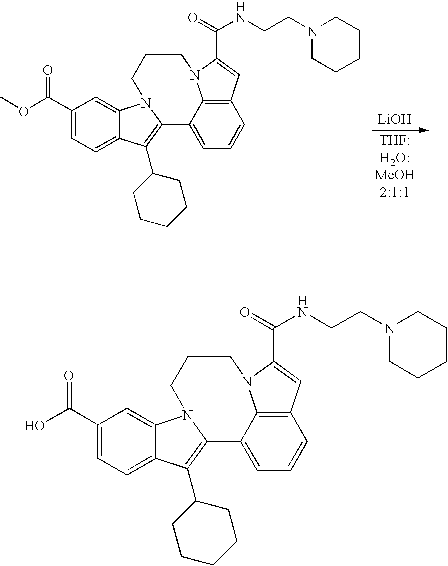

The above ester was saponified with LiOH (46 mg, 1.10 mmole) in 10 mL of a 2:1:1 THF:H₂O:MeOH solution at 50° C. for 3 hours. The completed reaction was then purified via RP HPLC before being converted to the HCl salt. Yield: 35 mg. MS (M+H⁺): 553.3; H¹-NMR (DMSO d₆): δ (ppm) 9.80 (s, 1H), 8.95 (t, 1H, J=5.4 Hz), 8.14 (s, 2H), 7.91 (d, 1H, J=8.1 Hz), 7.81 (m, 1H), 7.66 (m, 1H), 7.26 (m, 3H), 4.83 (m, 1H), 4.62 (m, 1H), 3.65 (m, 3H), 3.37 (m, 2H), 3.22 (m, 3H), 2.93 (m, 2H), 1.91 (m, 14H), 1.38 (m, 6H).

### Example 95

### Preparation of Compound 405

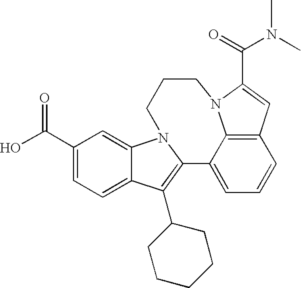

Compound 405 was synthesized as described in Example 94 using dimethylamine in the first step. Yield: 44 mg. MS: 470.3 (M+H⁺); H¹-NMR (DMSO-d₆): δ (ppm) 8.12 (d, 1H, J=0.9 Hz), 7.90 (d, 1H, J=8.4 Hz), 7.74 (dd, 1H, J=7.8 Hz, 1.2 Hz), 7.66 (dd, 1H, J=8.4 Hz, 1.5 Hz), 7.20 (m, 2H), 6.82 (s, 1H), 4.63 (dd, 1H, J=15.3 Hz, 5.4 Hz), 4.10 (m, 1H), 3.60 (m, 1H), 3.10 (m, 7H), 2.86 (m, 1H), 2.00 (m, 6H), 1.69 (m, 2H), 1.56 (m, 1H), 1.36 (m, 2H), 1.12 (m, 1H).

### Example 96

### Preparation of Compound 406

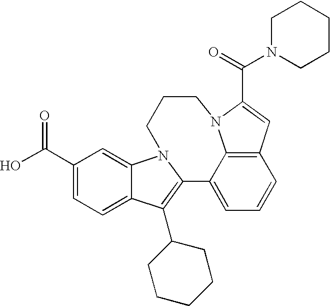

Compound 406 was synthesized as described in Example 94 using piperidine in the first step. Yield: 31 mg. MS: 510.3 (M+H⁺); H¹-NMR (DMSO-d₆): δ (ppm) 8.12 (s, 1H), 7.90 (d, 1H, J=8.4 Hz), 7.74 (dd, 1H, J=7.5 Hz, 1.2 Hz), 7.66 (dd, 1H, J=8.4 Hz, 1.2 Hz), 7.25 (t, 1H, J=7.5 Hz), 7.16 (m, 1H), 6.75 (s, 1H), 4.63 (m, 1H), 4.06 (m, 1H), 3.50 (m, 3H), 3.20 (m, 1H), 2.86 (m, 1H), 1.82 (m, 17H), 1.22 (m, 3H).

### Example 97

### Preparation of Compound 407

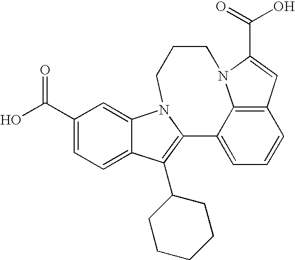

Compound 407 was saponified using the same procedure as Example 94. Yield: 47 mg. MS: 443.2 (M+H⁺); H¹-NMR (DMSO-d₆): δ (ppm) 8.14 (s, 1H), 7.91 (d, 1H, J=8.4 Hz), 7.82 (dd, 1H, J=6.6 Hz, 1.8 Hz), 7.67 (m, 1H), 7.37 (s, 1H), 7.27 (m, 2H), 5.00 (m, 1H), 4.62 (m, 1H), 3.61 (m, 1H), 3.21 (m, 1H), 2.86 (m, 1H), 1.98 (m, 6H), 1.63 (m, 3H) 1.23 (m, 3H).

### Example 98

### Preparation of Compound 408

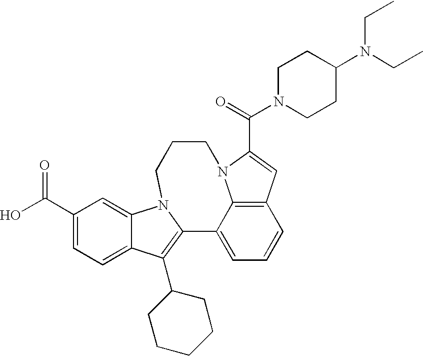

Compound 408 was synthesized as described in Example 94 using 4-diethylamine piperidine in the first step. Yield: 99 mg. MS: 581.4 (M+H⁺); H¹-NMR (DMSO-d₆): δ (ppm) 8.13 (s, 1H), 7.91 (d, 1H, J=8.4 Hz), 7.76 (dd, 1H, J=8.1 Hz, 1.2 Hz), 7.66 (dd, 1H, J=8.4 Hz, 1.2 Hz), 7.27 (t, 1H, J=7.2 Hz), 7.18 (m, 1H), 6.86 (s, 1H), 4.65 (m, 1H), 4.08 (m, 1H), 3.63 (m, 1H), 2.95 (m, 9H), 1.80 (m, 13H), 1.24 (m, 10H).

### Example 99

### Preparation of Compound 409

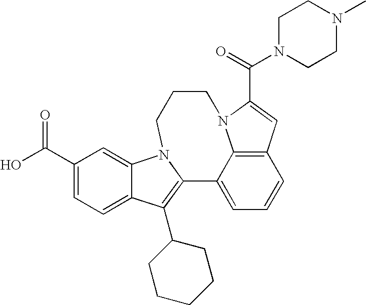

Compound 409 was synthesized as described in Example 94 using 1-methylpiperizine in the first step. Yield: 62 mg. MS: 525.3 (M+H⁺); H¹-NMR (DMSO-d₆): δ (ppm) 8.13 (s, 1H), 7.91 (d, 1H, J=8.4 Hz), 7.77 (dd, 1H, J=7.5 Hz, 0.6 Hz), 7.66 (dd, 1H, J=8.4 Hz, 1.5 Hz), 7.28 (t, 1H, J=7.2 Hz), 7.20 (m, 1H) 6.92, (s, 1H), 4.65 (m, 1H), 4.15 (m, 1H), 3.64 (m, 1H), 3.45 (m, 1H), 3.20 (m, 4H), 2.79 (m, 5H), 1.55 (m, 13H).

### Example 100

### Preparation of Compound 410

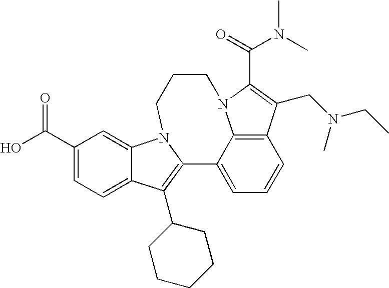

Formaldehyde (0.06 mL, 0.744 mmole) and ethyl methyl amine (0.066 mL, 0.744 mmole) were stirred for 10 minutes in 2 mL glacial acetic acid at room temperature. Compound 405 was then added to the reaction mixture and it was stirred at 60° C. for 2 hours. The completed reaction was concentrated, purified via RP HPLC, and converted to the HCl salt. Yield: 51 mg. MS: 541.3 (M+H⁺); H¹-NMR (DMSO-d₆): δ (ppm) 8.09 (m, 2H), 7.93 (m, 1H), 7.67 (dd, 1H, J=8.Hz, 1.2 Hz), 7.38 (t, 1H, J=7.5 Hz), 7.25 (m, 1H), 4.70 (m, 1H), 4.10 (m, 1H), 3.71 (m, 2H), 3.14 (m, 3H), 2.79 (m, 5H), 2.59 (m, 1H), 1.80 (m, 9H), 1.27 (m, 6H).

### Example 101

### Preparation of Compound 411

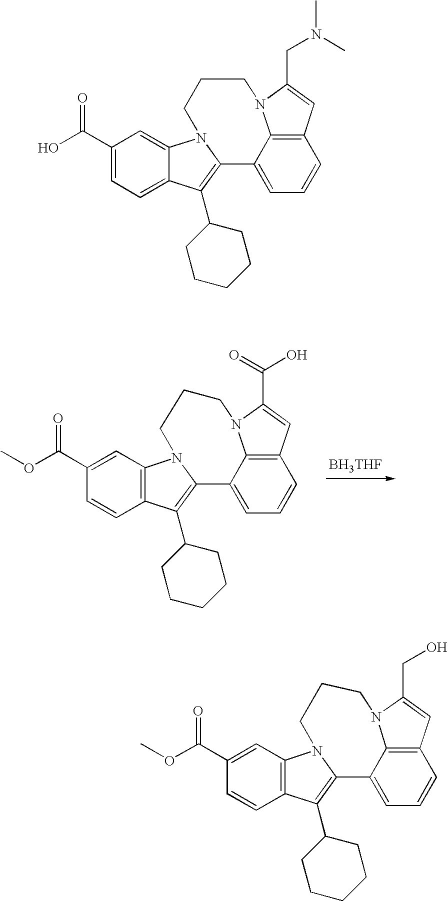

The above acid (300 mg, 0.66 mmole) was dissolved in 6 mL THF. 1M BH₃ THF complex in THF (6.6 mL, 6.6 mmole) was added and the reaction was stirred over night at room temperature before being quenched with 2M HCl (3.3 mL). The completed reaction was then concentrated and purified via RP HPLC. Yield: 170 mg (58%). MS: 443.2 (M+H⁺); H¹-NMR (DMSO-d₆): δ (ppm) 8.16 (s, 1H), 7.93 (d, 1H, J=8.4 Hz), 7.66 (m, 2H), 7.16 (t, 1H, J=7.5 Hz), 7.06 (m, 1H), 6.52 (s, 1H), 4.66 (m, 3H), 4.20 (m, 1H), 3.88 (s, 3H), 3.59 (m, 1H), 2.86 (m, 1H), 2.04 (m, 6H), 1.69 (m, 2H), 1.55 (m, 1H), 1.23 (m, 4H).

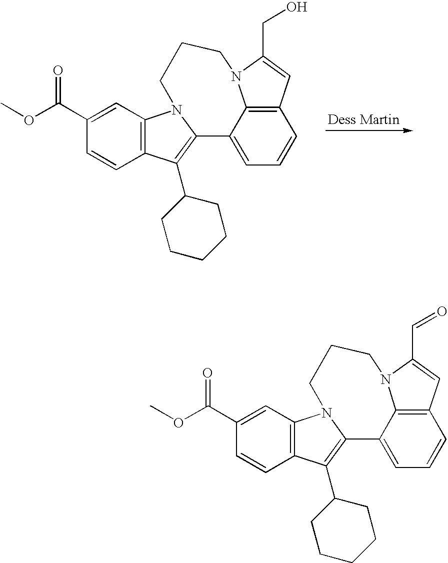

The above alcohol (100 mg, 0.226 mmole) and dess martin periodinane (115 mg, 0.271 mmole) were dissolved in 5 mL of dichloromethane and stirred at room temperature for 15 minutes. The completed reaction was then diluted with 50 mL dichloromethane and washed with water. The organic layer was washed with aqueous sodium bicarbonate and brine, dried over magnesium sulfate, concentrated, and taken on to the next step as is. MS: 441.2 (M+H⁺).

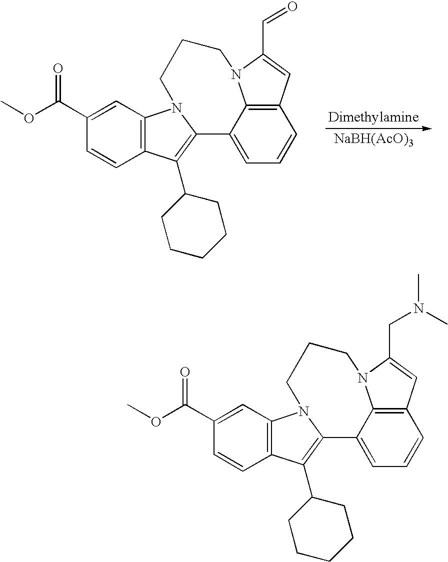

The above aldehyde (99 mg, 0.226 mmole) and IM dimethylamine in THF (0.294 mL, 0.294 mmole) were dissolved in 6 mL THF. Triacetoxyborohydride (73 mg, 0.339 mmole) was then added and the reaction was stirred over night at room temperature. The completed reaction was then diluted with 75 mL ethyl acetate, washed with water and brine, dried, concentrated, and taken on to saponification as is. MS: 470.3 (M+H⁺).

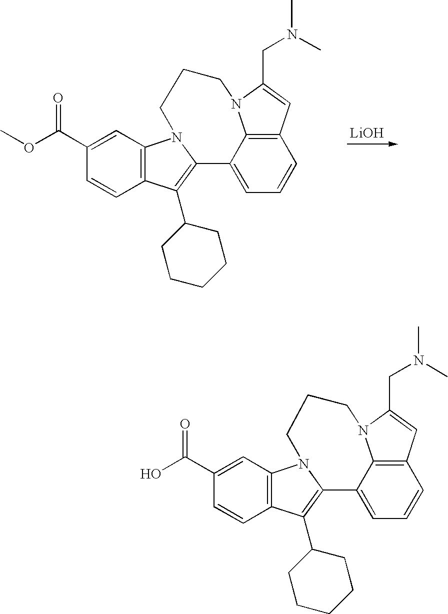

The above ester was saponified and converted to the HCl salt as described in Example 92. Yield: 47 mg. MS: 456.2 (M+H⁺); H¹-NMR (DMSO-d₆): δ (ppm) 8.15 (s, 1H), 7.91 (d, 1H, J=8.4 Hz), 7.75 (dd, 1H, J=7.8 Hz, 1.2 Hz), 7.67 (dd, 1H, J=8.4 Hz, 1.2 Hz), 7.24 (t, J=7.5 Hz), 7.14 (dd, 1H, J=7.2 Hz, 1.2 Hz), 6.97 (s, 1H), 4.63 (m, 2H), 4.51 (m, 1H), 4.30 (m, 1H), 3.54 (m, 1H), 3.22 (m, 1H), 2.84 (m, 6H), 1.97 (m, 6H), 1.68 (m, 2H), 1.55 (m, 1H), 1.37 (m, 2H), 1.11 (m, 2H).

### Example 102

### Preparation of Compound 412

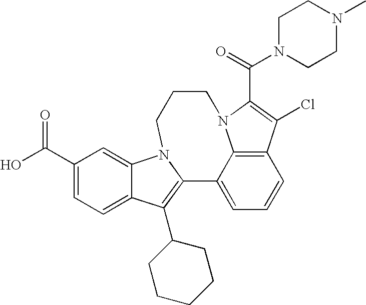

Compound 409 (170 mg, 0.325 mmole) was dissolved in 8 mL of acetonitrile. N-chlorosuccinimde (87 mg, 0.65 mmole) was added and the reaction was stirred at room temperature over night. The completed reaction was concentrated, purified via RP HPLC, and converted to the HCl salt. Yield: 15 mg. MS: 560.2 (M+H⁺); H¹-NMR (DMSO-d₆): δ (ppm) 8.16 (s, 1H), 7.94 (d, 1H, J=8.7 Hz), 7.72 (m, 2H), 7.42 (t, 1H, J=7.2 Hz), 7.31 (m, 1H), 4.66 (m, 2H), 3.97 (m, 1H), 3.58 (m, 5H), 2.85 (m, 5H), 2.07 (m, 6H), 1.56 (m, 6H0, 1.23 (m, 4H).

### Example 103

### Preparation of Compound 413

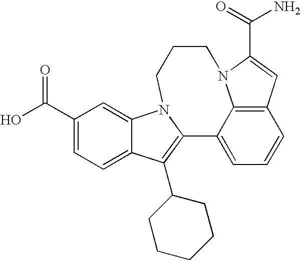

Compound 413w as synthesized as described in Example 94 using ammonium chloride in the first step. Yield: 74 mg. MS: 442.2 (M+H⁺); H¹-NMR (DMSO-d₆): δ (ppm) 8.14 (s, 1H), 8.06 (s, 1H), 2.92 (d, 1H, J=8.4 Hz), 7.78 (dd, 1H, J=7.5 Hz, 1.2 Hz), 7.67 (dd, 1H, J=8.4 Hz, 1.5 Hz), 7.46 (s, 1H), 7.23 (m, 3H), 4.94 (m, 1H), 4.63 (m, 1H), 3.60 (m, 1H), 2.86 (m, 1H), 2.00 (m, 6H), 1.58 (m, 3H), 1.18 (m, 3H).

### Example 104

### Preparation of Compound 414

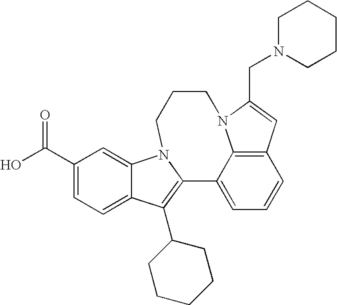

Compound 414 was synthesized as described in Example 101 using piperidine in the first step. Yield: 30 mg. MS: 496.3 (M+H⁺); H¹-NMR (DMSO-d₆): δ (ppm) 9.98 (s, 1H), 8.17 (s, 1H), 7.93 (d, 1H, J=8.4 Hz), 7.76 (d, 1H, J=8.1 Hz), 7.69 (dd, 1H, J=8.7 Hz, 1.5 Hz), 7.26 (t, 1H, J=7.5 Hz), 7.16 (m, 1H), 6.99 (s, 1H), 4.51 (m, 4H), 3.49 (m, 2H), 3.25 (m, 1H), 3.00 (m, 2H), 2.86 (m, 1H), 1.83 (m, 15H), 1.23 (m, 3H).

### Example 105

### Preparation of Compound 415

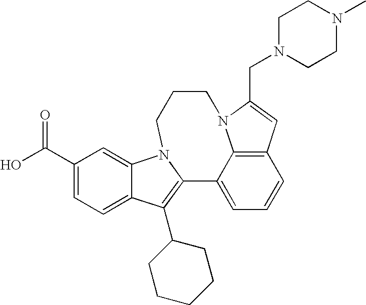

Compound 415 was synthesized as described in Example 101 using 1-methylpiperazine in the first step. Yield: 36 mg. MS: 511.3 (M+H⁺); H¹-NMR (DMSO-d₆): δ (ppm) 8.10 (s, 1H), 7.90 (d, 1H, J=8.4 Hz). 7.66 (dd, 2H, J=8.7 Hz), 7.18 (t, 1H, J=7.5 Hz), 7.09 (m, 1H), 4.55 (m, 1H), 4.24 (m, 1H), 3.38 (m, 4H), 2.98 (m, 7H), 2.75 (m, 4H), 2.03 (m, 7H), 1.69 (m, 2H), 1.52 (m, 1H), 1.34 (m, 2H), 1.12 (m, 2H).

### Example 106

### Preparation of Compound 416

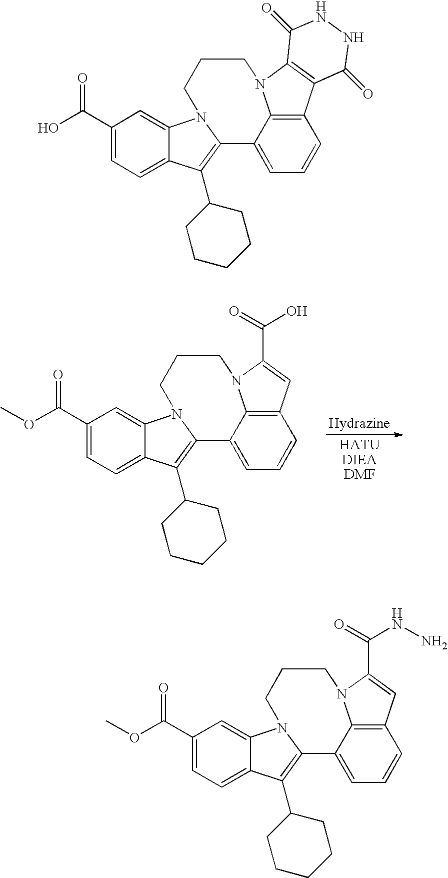

The above hydrazide was synthesized as described in Example 94 on a 0.657 mmole scale with hydrazine. Yield: 315 mg. MS: 471.2 (M+H⁺).

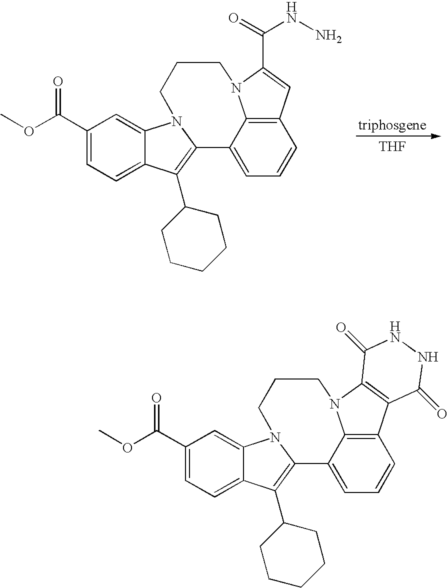

The above hydrazide (150 mg, 0.319 mmole) and triphosgene (189 mg, 0.638 mmole) were dissolved in 5 mL THF and heated at 60° C. for 20 minutes. The completed reaction was then concentrated and the crude oil was taken on to saponification as is. MS: 497.3 (M+H⁺).

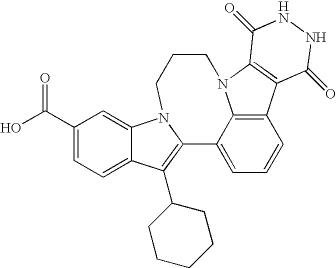

The above ester was saponified as described in Example 94. Yield: 69 mg. MS: 456.2 (M+H⁺); H¹-NMR (DMSO-d₆): δ (ppm) 12.73 (s, 1H), 8.16 (s, 1H), 7.93 (d, 1H, J=8.4 Hz), 7.84 (dd, 1H, J=7.5 Hz), 7.69 (dd, 1H, J=8.7 Hz), 7.30 (m, 3H), 4.72 (m, 2H), 3.64 (m, 1H), 3.32 (m, 1H), 2.89 (m, 1H), 1.96 (m, 6H), 1.64 (m, 3H), 1.22 (m, 3H).

### Example 107

### Preparation of Compound 417

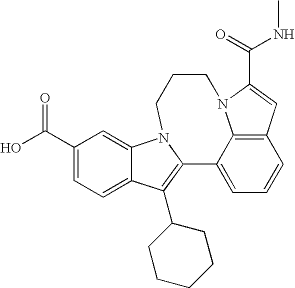

Compound 417 was synthesized as described in Example 94 using 2M methylamine in THF in the first step. Yield: 45 mg. MS: 456.2 (M+H⁺); H¹-NMR (DMSO-d₆): δ (ppm) 12.61 (s, 1H), 8.56 (m, 1H), 8.15 (s, 1H), 7.92 (d, 1H, J=8.4 Hz), 7.79 (dd, 1H, J=7.5 Hz, 1.2 Hz), 7.67 (dd, 1H, J=8.4 Hz, 1.2 Hz), 7.25 (m, 2H), 7.12 (s, 1H), 4.82 (m, 1H), 4.63 (m, 1H), 3.60 (m, 1H), 3.15 (m, 1H), 3.82 (m, 4H), 2.04 (m, 7H), 1.60 (m, 3H), 1.23 (m, 4H).

### Example 108

### Preparation of Compound 418

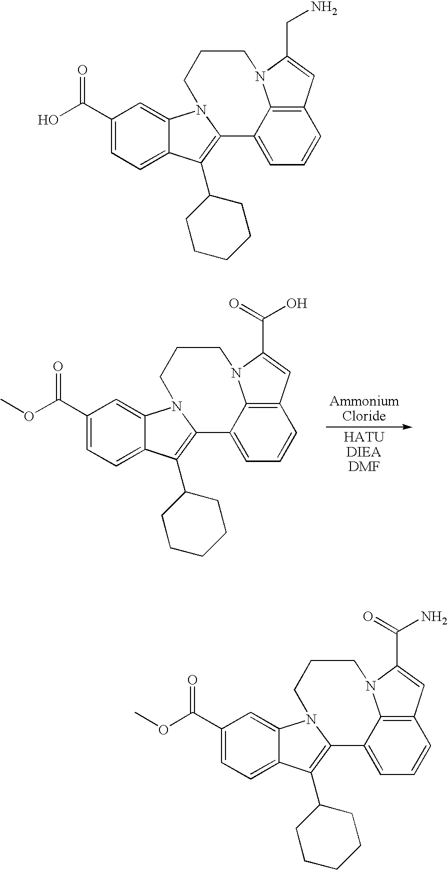

The above amide was synthesized as described in Example 94. MS: 456.2 (M+H⁺)

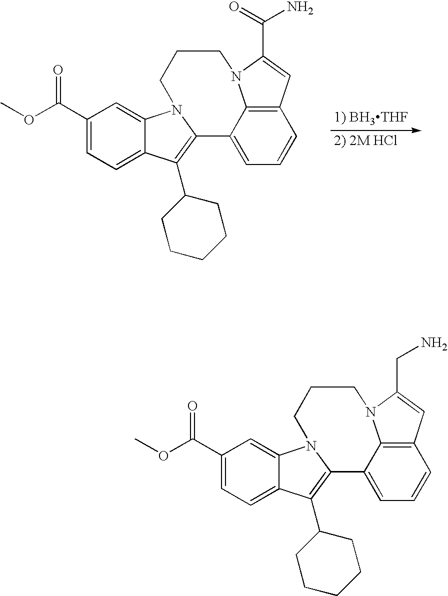

The above amine was synthesized from the corresponding amide as described in Example 101. MS: 442.3 (M+H⁺).

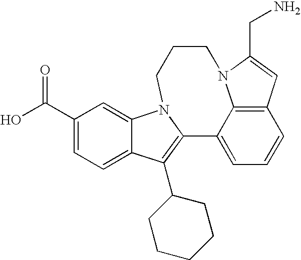

Compound 418 was produced by saponifying the corresponding ester (above) as described in Example 94. Yield: 25 mg. MS: 428.3 (M+H⁺); H¹-NMR (DMSO-d₆): δ (ppm) 8.17 (d, 1H, J=0.9 Hz), 7.92 (d, 1H, J=8.4 Hz), 7.71 (m, 2H), 7.23 (t, 1H, J=7.8 Hz), 7.13 (m, 1H), 6.76 (s, 1H), 4.65 (m, 1H), 4.29 (s, 2H), 4.14 (m, 1H), 3.55 (m, 1H), 2.85 (m, 1H), 2.00 (m, 7H), 1.61 (m, 3H), 1.23 (m, 3H).

### Example 109

### Preparation of Compound 419

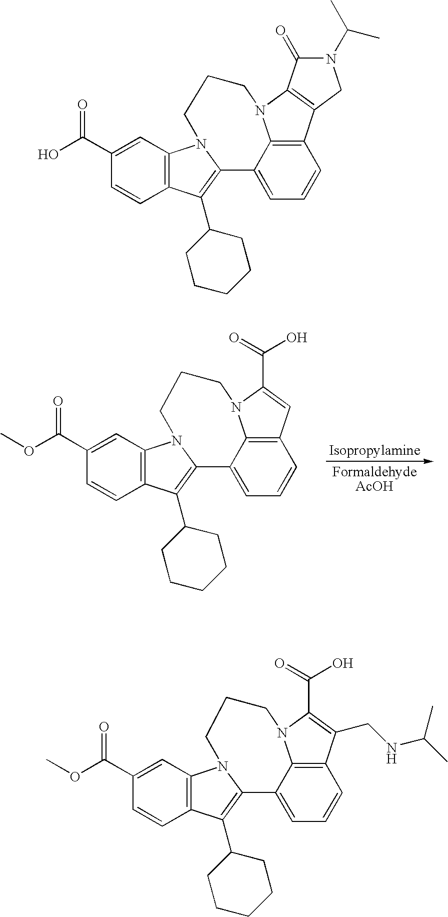

Isopropylamine (0.376 mL, 4.38 mmole) and formaldehyde (0.355 mL, 4.38 mmole) were mixed together in 8 mL of acetic acid for 10 minutes. The above acid (400 mg, 0.876 mmole) was added, and the reaction was heated at 70° C. for 1 hour. Upon completion, the reaction was concentrated, coevaporated with toluene 3 times, precipitated out in water, and dried over phosphorus pentoxide. It was then taken on as is. MS: 528.3 (M+H⁺).

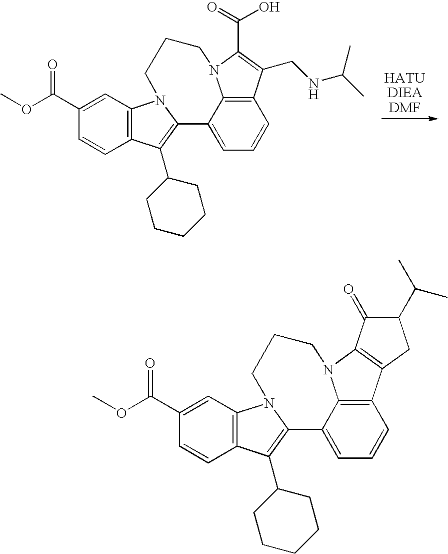

The crude product from the previous step was dissolved in 15 mL of DMF, along with HATU (666 mg, 1.75 mmole) and diisopropylethylamine (0.552 mL, 4.38 mmole and the reaction was heated at 65° C. for 2 hours. The completed reaction was then concentrated, precipitated and washed with water 3 times, spun to a pellet, and dried over phosphorus pentoxide. The resulting crude material was then taken on to the next step as is. MS: 510.2 (M+H⁺).

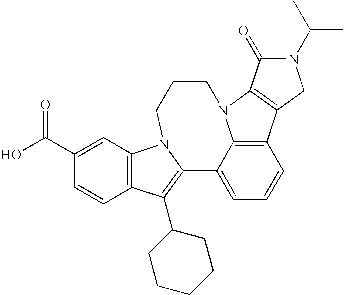

Compound 419 was synthesized from the above ester on a 0.216 mmole scale using the same saponification procedure and work up as Example 92. Yield: 31 mg. MS: 496.2 (M+H⁺); H¹-NMR (DMSO-d₆): δ (ppm) 8.16 (s, 1H), 7.92 (d, 1H, J=8.4 Hz), 7.81 (dd, 1H, J=7.8 Hz, 0.9 Hz), 7.69 (dd, 1H, 8.4 Hz, 1.2 Hz), 7.33 (t, 1H, J=7.2 Hz), 7.25 (m, 1H), 4.72 (m, 2H), 4.42 (m, 3H), 3.66 (m, 1H), 3.10 (m, 1H), 2.81 (m, 1H), 1.97 (m, 6H), 1.60 (m, 3H), 1.59 (m, 9H).

### Example 110

### Preparation of Compound 420

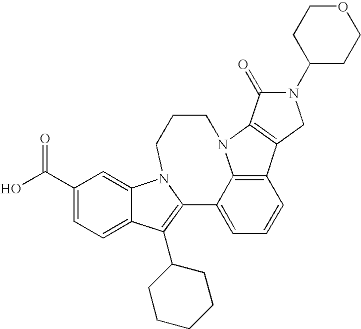

Compound 420 was synthesized according to Example 109 using 4-aminotetrahydropyran in the first step. Yield: 57 mg. MS: 538.3 (M+H⁺); H¹-NMR (DMSO-d₆): δ (ppm) 8.16 (s, 1H), 7.93 (d, 1H, J=8.7 Hz), 7.83 (dd, 1H, J=7.8 Hz, 1.2 Hz), 7.68 (dd, 1H, J=8.7 Hz, 1.2 Hz), 7.34 (t, 1H, J=7.5 Hz), 7.26 (m, 1H), 4.63 (m, 2H), 4.52 (d, 2H, J=2.7 Hz), 4.21 (m, 1H), 3.95 (m, 2H), 3.68 (m, 1H), 3.46 (m, 1H), 3.11 (m, 1H), 2.83 (m, 1H), 1.85 (m, 12H), 1.57 (m, 1H), 1.37 (m, 1H), 1.11 (m, 1H).

### Example 111

### Preparation of Compound 421

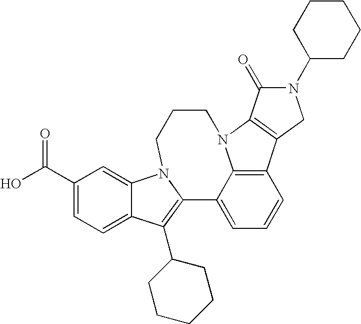

Compound 421 was synthesized according to Example 109 using aminocyclohexane in the first step. Yield: 58 mg. MS: 536.3 (M+H⁺); H¹-NMR (DMSO-d₆): δ (ppm) 8.16 (s, 1H), 7.93 (d, 1H, J=8.4 Hz), 7.82 (dd, 1H, J=8.1 Hz, 1.2 Hz), 7.69 (J=7.8 Hz, 1.2 Hz), 7.34 (t, 1H, J=7.2 Hz), 7.26 (m, 1H), 4.68 (m, 2H), 4.80 (s, 2H), 3.96 (m, 1H), 3.68 (m, 1H), 3.11 (s, 1H), 1.57 (m, 22H).

### Example 112

### Preparation of Compound 427

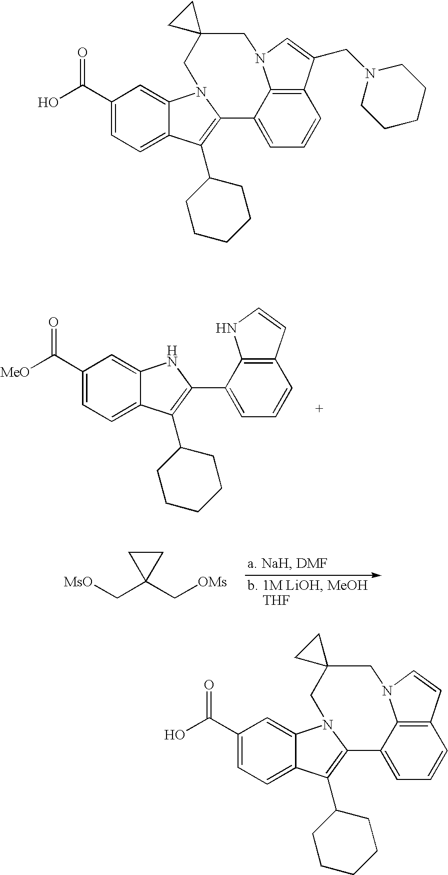

A reaction flask was charged with 190 mg (0.5 mmol) of the above ester and 260 mg (1 mmol, 2 eq) of the dimesylate. To this was added 5 mL DMF and 50 mg (1.25 mmol, 2.5 eq) NaH (60% in mineral oil). The reaction mixture was then heated to 160° C. for 20 min. by microwave. HPLC and LC-MS analyses confirmed complete conversion. The reaction mixture was then quenched with water and concentrated by rotovap. Water was added to the resulting residue to precipitate the desired material. The solids were then collected by centrifuge, washed with additional water. The resulting material was then dissolved with 6 mL THF, 2 mL MeOH and 2 mL LiOH (1M, aqueous). The mixture was heated to 125° C. for 5 min. by microwave. HPLC and LC-MS showed complete conversion to the desired product. The reaction mixture was neutralized with 0.5 mL HCl (2M, aqueous) and concentrated. Water was again added to the resulting residue to precipitate the desired product. The solids were then collected by centrifuge, washed with additional water and dried under vacuum to afford 205 mg (97%) as a rust-colored powder which was used without further purification. MS: 425.2 (M+H⁺).

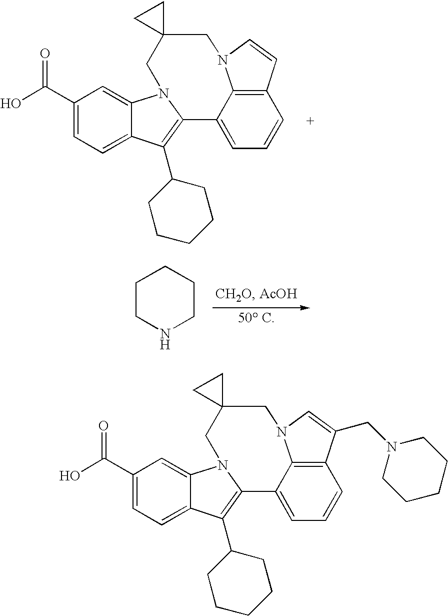

A reaction vessel was charged with 148 μL piperidine (1.5 mmol, 3 eq) and 2 mL AcOH. Formaldehyde (125 μL, 1.5 mmol, 3 eq, 37% aqueous) was then added and the mixture was allowed to stir at 50° C. for 5 min. The above acid (205 mg, 0.48 mmol) was then added and the reaction mixture was allowed to continue stirring at 50° C. for 2 h. The reaction mixture was concentrated and the resulting residue was dissolved with DMF and acidified with TFA. The solution was filtered and then purified by reverse-phase HPLC. The desired fractions were collected and concentrated. The purified residue was dissolved with CH₃CN and acidified with 2M HCl/Et₂O. The acidic solution was concentrated, the resulting residue was suspended with water, frozen and lyophilized to afford 23 mg (9%) Compound 427 as an off-white powder. MS: 522.3 (M+H⁺); ¹H NMR (DMSO-d6): δ 8.21 (s, 1H), 8.02 (d, J=7.5, 1H), 7.95 (d, J=8.6, 1H), 7.71-7.68 (m, 2H), 7.36 (t, J=7.5, 1H), 7.19 (d, J=6.9, 1H), 4.57-4.44 (m, 2H), 4.15-3.88 (m, 3H), 3.56-3.35 (m, 2H), 3.06-2.77 (m, 3H), 2.12-1.11 (m, 16H), 1.01-0.98 (m, 1H), 0.84-0.80 (m, 1H), 0.70-0.59 (m, 2H).

### Example 113

### Preparation of Compound 428

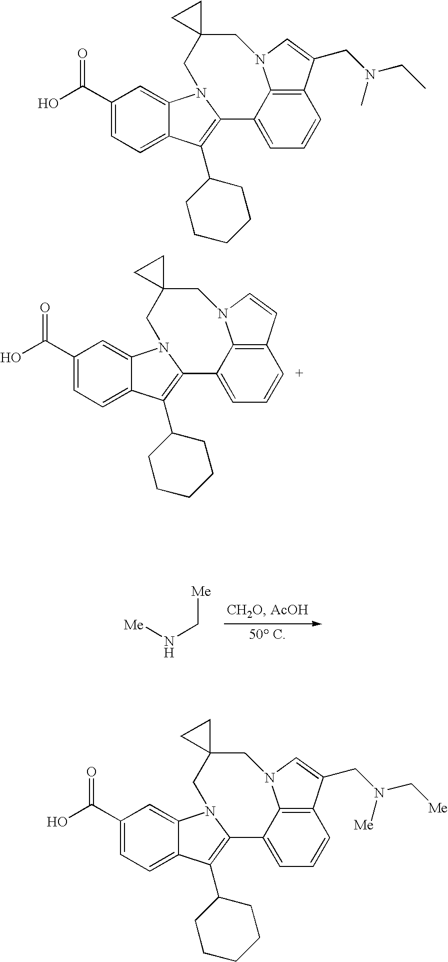

A reaction vessel was charged with 91 μL ethylmethylamine (1.06 mmol, 3 eq) and 2 mL AcOH. Formaldehyde (90 μL, 1.06 mmol, 3 eq, 37% aqueous) was then added and the mixture was allowed to stir at 50° C. for 5 min. The above acid (150 mg, 0.35 mmol) was then added and the reaction mixture was allowed to continue stirring at 50° C. for 2.5 h. The reaction mixture was concentrated and the resulting residue was dissolved with DMF and acidified with TFA. The solution was filtered and then purified by reverse-phase HPLC. The desired fractions were collected and concentrated. The purified residue was dissolved with CH₃CN and acidified with 2M HCl/Et₂O. The acidic solution was concentrated, the resulting residue was suspended with water, frozen and lyophilized to afford 43 mg (25%) Compound 428 as an off-white powder. MS: 437.2 (M⁺-58[NEtMe]); ¹H NMR (DMSO-d6): δ 8.21 (s, 1H), 8.00 (d, J=8.3, 1H), 7.95 (d, J=8.6, 1H), 7.71-7.68 (m, 2H), 7.36 (t, J=7.7, 1H), 7.20 (d, J=7.1, 1H), 4.64-4.43 (m, 2H), 4.15-3.87 (m, 3H), 3.41-3.30 (m, 2H), 3.14-3.08 (m, 1H), 2.86-2.75 (m, 3H), 2.12-1.15 (m, 13H), 1.01-0.98 (m, 1H), 0.82 (br s, 1H), 0.70-0.58 (m, 2H).

### Example 114

### Preparation of Compound 429

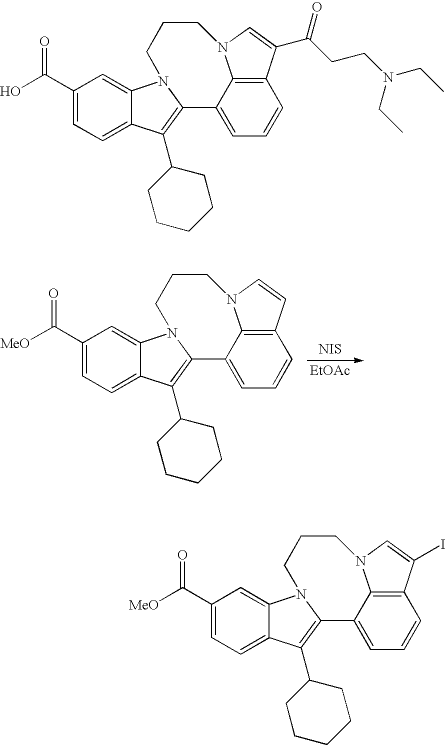

A reaction flask was charged with 3 g (7.3 mmol) of the above ester and dissolved with 365 mL EtOAc. To this was added 1.79 g N-iodosuccinimide (8 mmol, 1.1 eq). The reaction mixture was then allowed to stir at room temperature. The reaction was monitored by HPLC analysis and additional NIS was added in 0.1 eq portions until no starting material remained. The reaction mixture was then concentrated and purified by SiO₂ chromatography (10%→30% EtOAc in hexane) to afford 2.9 g (74%) of the iodo indole. MS: 539.1 (M+H⁺); ¹H NMR (DMSO-d6): δ 8.20 (d, J=1.1, 1H), 7.97 (d, J=8.5, 1H), 7.71 (dd, J=8.3, 1.1, 1H), 7.68 (s, 1H), 7.47 (dd, J=8.0, 1.1, 1H), 7.33 (t, J=7.4, 1H), 7.22 (dd, J=7.1, 1.1, 1H), 4.67 (dd, J=14.2, 3.4, 1H), 4.21 (d, J=14.0, 1H), 3.68-3.57 (m, 1H), 3.29-3.19 (m, 1H), 2.87-2.82 (m, 1H), 2.05-1.11 (m, 12H).

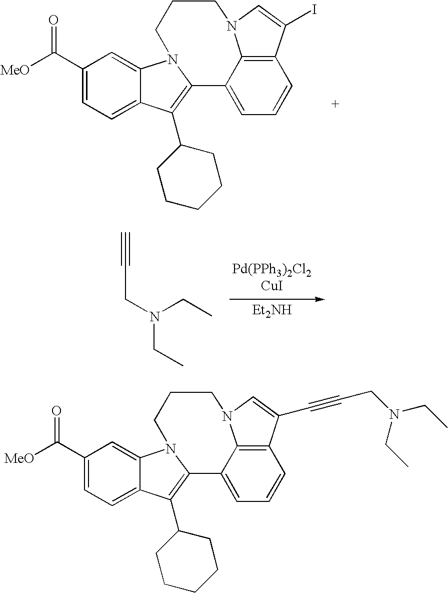

A reaction vessel was charged with 108 mg of the iodo indole (0.2 mmol), 7 mg Pd(PPh₃)₄ (0.01 mmol, 0.05 eq) and 4 mg CuI (0.02 mmol, 0.1 eq). Diethylamine (2 mL) was then added and the reaction vessel was sealed, degassed and back-filled with argon. Diethylpropargylamine (55 μL, 0.4 mmol, 2 eq) was added via syringe and the reaction vessel was allowed to stir at 50° C. until complete by HPLC analysis. Water was then added to the reaction mixture to precipitate the desired material. The solids were collected by centrifuge, washed with additional water, dried under vacuum and used without further purification. MS: 522.3 (M+H⁺).

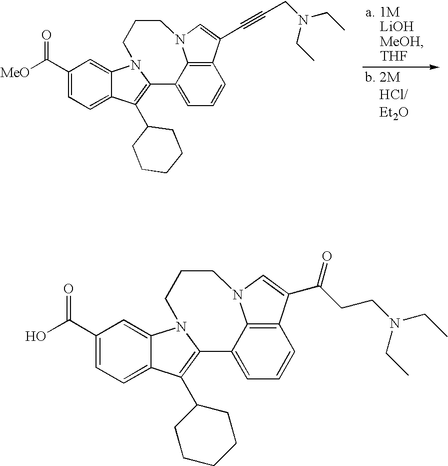

Approximately 104 mg (0.2 mmol) was dissolved with 6 mL THF, 2 mL MeOH and 2 mL LiOH (1M, aqueous). The mixture was then allowed to stir at 50° C. until complete by HPLC analysis. The mixture was neutralized with 1 mL HCl (2M, aqueous) and concentrated. The resulting residue was dissolved with DMF and acidified with TFA. The solution was filtered and then purified by reverse-phase HPLC. The desired fractions were collected and concentrated using a warm water bath. The purified residue was dissolved with CH₃CN and acidified with 2M HCl/Et₂O. The acidic solution was concentrated, the resulting residue was suspended with water, frozen and lyophilized to afford 59 mg (56%) Compound 429 as a pale tan powder. MS: 526.3 (M+H⁺); ¹H NMR (DMSO-d6): δ 8.59 (s, 1H), 8.41 (dd, J=7.2, 0.9, 1H), 8.20 (s, 1H), 7.96 (d, J=8.3, 1H), 7.72 (dd, J=8.3, 1.4, 1H), 7.46 (t, J=7.5, 1H), 7.28 (d, J=6.3, 1H), 4.72 (dd, J=14.7, 5.5, 1H), 4.27 (d, J=12.1, 1H), 3.70-3.61 (m, 1H), 3.48-3.18 (m, 9H), 2.93-2.77 (m, 1H), 2.19-1.11 (m, 18H).

### Example 115

### Preparation of Compound 430

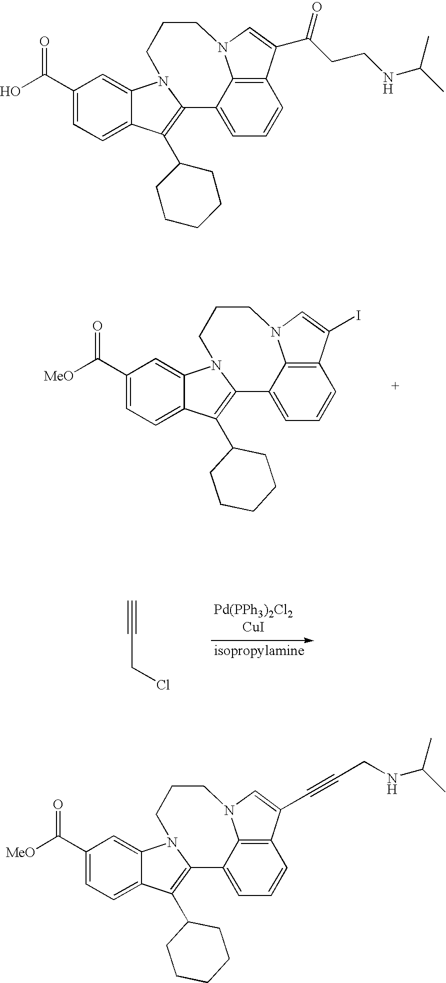

A reaction vessel was charged with 108 mg the above iodo indole (0.2 mmol), 7 mg Pd(PPh₃)₄ (0.01 mmol, 0.05 eq) and 4 mg CuI (0.02 mmol, 0.1 eq). Isopropylamine (2 mL) was then added and the reaction vessel was sealed, degassed and back-filled with argon. Propargyl chloride (29 μL, 0.4 mmol, 2 eq) was added via syringe and the reaction vessel was allowed to stir at 50° C. until complete by HPLC analysis. Water was then added to the reaction mixture to precipitate the desired material. The solids were collected by centrifuge, washed with additional water, dried under vacuum and used without further purification. MS: 508.3 (M+H⁺).

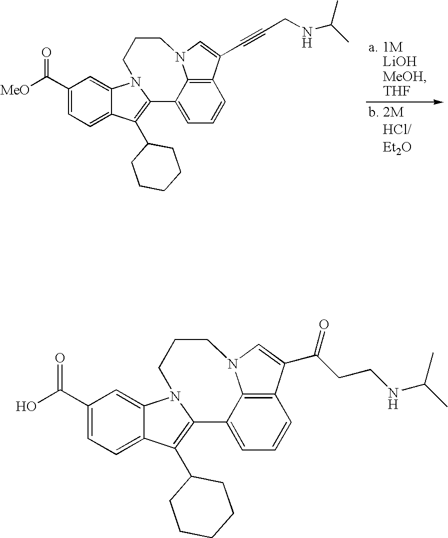

Approximately 102 mg of the above alkyne (0.2 mmol) was dissolved with 6 mL THF, 2 mL MeOH and 2 mL LiOH (1M, aqueous). The mixture was then allowed to stir at 35° C. overnight. The mixture was neutralized with 1 mL HCl (2M, aqueous) and concentrated. The resulting residue was dissolved with DMF and acidified with TFA. The solution was filtered and then purified by reverse-phase HPLC. The desired fractions were collected and concentrated using a warm water bath. The purified residue was dissolved with CH₃CN and acidified with 2M HCl/Et₂O. The acidic solution was concentrated, the resulting residue was suspended with water, frozen and lyophilized to afford 40 mg (39%) Compound 430 as a pale tan powder. MS: 512.3 (M+H⁺); ¹H NMR (DMSO-d6): δ 8.94 (br s, 2H), 8.55 (s, 1H), 8.41 (dd, J=8.0, 0.9, 1H), 8.20 (s, 1H), 7.96 (d, J=8.5, 1H), 7.72 (dd, J=8.5, 1.4, 1H), 7.46 (t, J=7.7, 1H), 7.28 (d, J=6.3, 1H), 4.71 (dd, J=14.5, 4.8, 1H), 4.32 (d, J=13.4, 1H), 3.71-3.61 (m, 1H), 3.45-3.26 (m, 7H), 2.83 (br s, 1H), 2.12-1.11 (m, 18H).

### Example 116

### Preparation of Compound 431

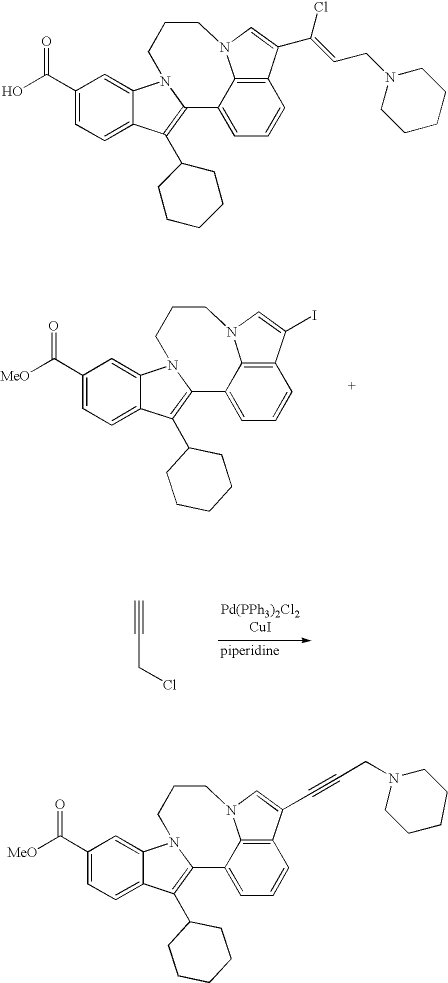

A reaction vessel was charged with 108 mg of the above iodo indole (0.2 mmol), 7 mg Pd(PPh₃)₄ (0.01 mmol, 0.05 eq) and 4 mg CuI (0.02 mmol, 0.1 eq). Piperidine (2 mL) was then added and the reaction vessel was sealed, degassed and back-filled with argon. Propargyl chloride (28 μL, 0.4 mmol, 2 eq) was added via syringe and the reaction vessel was allowed to stir at 50° C. until complete by HPLC analysis. Water was then added to the reaction mixture to precipitate the desired material. The solids were collected by centrifuge, washed with additional water, dried under vacuum and used without further purification. MS: 534.3 (M+H⁺).

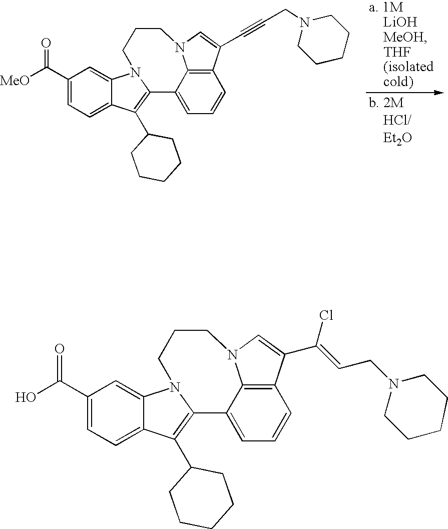

Approximately 107 mg of the above alkyne (0.2 mmol) was dissolved with 6 mL THF, 2 mL MeOH and 2 mL LiOH (1M, aqueous). The mixture was then allowed to stir at 50° C. until complete by HPLC. The mixture was neutralized with 1 mL HCl (2M, aqueous) and concentrated. The resulting residue was dissolved with DMF and acidified with TFA. The solution was filtered and then purified by reverse-phase HPLC. The desired fractions were collected and concentrated using a cold water bath. The purified residue was dissolved with CH₃CN and acidified with 2M HCl/Et₂O. The acidic solution was concentrated, the resulting residue was suspended with water, frozen and lyophilized to afford 86 mg (77%) Compound 431 as a white powder. MS: 556.3 (M+H⁺), 558.3 (M+2+H⁺); ¹H NMR (DMSO-d6): δ 9.81 (br s, 1H), 8.19 (d, J=1.1, 1H), 8.07 (d, J=8.1, 1H), 7.96 (d, J=8.5, 1H), 7.93 (s, 1H), 7.72 (dd, J=8.5, 1.4, 1H), 7.42 (t, J=7.3, 1H), 7.27 (d, J=6.8, 1H), 6.43 (t, J=7.1, 1H), 4.70 (dd, J=14.4, 5.4, 1H), 4.28 (d, J=14.4, 1H), 4.16 (t, J=4.8, 1H), 3.64-3.03 (m, 7H), 2.84 (br s, 1H), 2.12-1.12 (m, 18H).

### Example 117

### Preparation of Compound 432

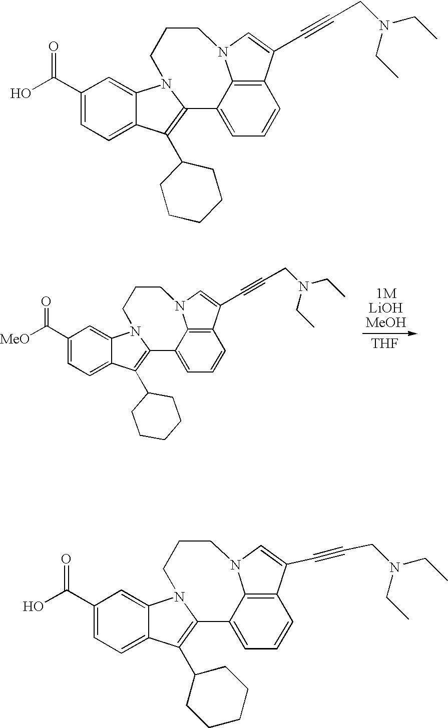

Approximately 102 mg of the above ester (0.2 mmol) was dissolved with 6 mL THF, 2 mL MeOH and 2 mL LiOH (1M, aqueous). The mixture was then allowed to stir at 35° C. overnight. The mixture was neutralized with 1 mL HCl (2M, aqueous) and concentrated. The resulting residue was dissolved with DMF and acidified with TFA. The solution was filtered and then purified by reverse-phase HPLC. The desired fractions were collected and concentrated using a warm water bath. The purified residue was dissolved with CH₃CN and acidified with 2M HCl/Et₂O. The acidic solution was concentrated, the resulting residue was suspended with water, frozen and lyophilized to afford 50 mg (49%) Compound 432 as a white powder. MS: 508.3 (M+H⁺); ¹H NMR (DMSO-d6): δ 10.19 (br s, 1H), 8.18 (d, J=1.1, 1H), 7.95 (d, J=8.3, 1H), 7.92 (s, 1H), 7.77 (dd, J=7.7, 0.9, 1H), 7.71 (dd, J=8.3, 1.4, 1H), 7.39 (t, J=7.2, 1H), 7.25 (dd, J=7.2, 0.9, 1H), 4.68 (dd, J=14.1, 4.6, 1H), 4.50 (s, 2H), 4.23 (d, J=14.7, 1H), 3.77-3.23 (m, 6H), 2.85-2.84 (m, 1H), 2.12-1.12 (m, 18H); ¹³C NMR (DMSO-d6): δ 168.02, 136.84, 134.90, 134.54, 134.47, 129.38, 126.29, 123.52, 120.69, 120.09, 119.95, 119.67, 118.78, 118.64, 114.63, 111.73, 93.93, 82.69, 80.18, 47.00, 43.94, 41.83, 36.44, 32.88, 32.57, 28.68, 26.70, 25.58, 9.14.

### Example 118

### Preparation of Compound 436

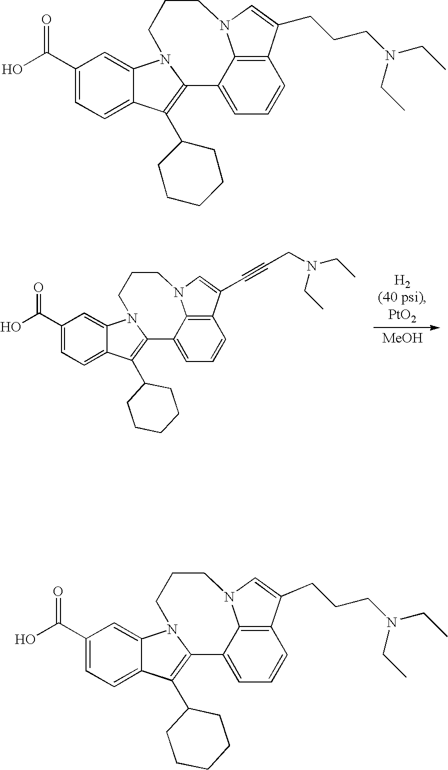

A Parr hydrogenation vessel was charged with 102 mg of Compound 432 (0.2 mmol), a catalytic quantity of PtO₂ and 30 mL MeOH. The vessel was sealed, degassed and back-filled with H₂ (3×). The vessel was then charged with 40 psi H₂ and allowed to shake until HPLC analysis indicated complete conversion. The reaction mixture was then filtered and concentrated. The resulting residue was dissolved with DMF and acidified with TFA. The solution was filtered and then purified by reverse-phase HPLC. The desired fractions were collected and concentrated. The purified residue was dissolved with CH₃CN and acidified with 2M HCl/Et₂O. The acidic solution was concentrated, the resulting residue was suspended with water, frozen and lyophilized to afford 77 mg (75%) Compound 436 as an off-white powder. MS: 512.3 (M+H⁺); ¹H NMR (DMSO-d6): δ 10.23 (br s, 1H), 8.16 (d, J=0.8, 1H), 7.94 (d, J=8.5, 1H), 7.77-7.68 (m, 2H), 7.30 (s, 1H), 7.23 (t, J=7.3, 1H), 7.13 (d, J=6.5, 1H), 4.63 (dd, J=15.0, 4.8, 1H), 4.11 (d, J=14.4, 1H), 3.65-3.56 (m, 1H), 3.27-3.10 (m, 7H), 2.87-2.82 (m, 3H), 2.12-1.23 (m, 20H).

### Example 119

### Preparation of Compound 433

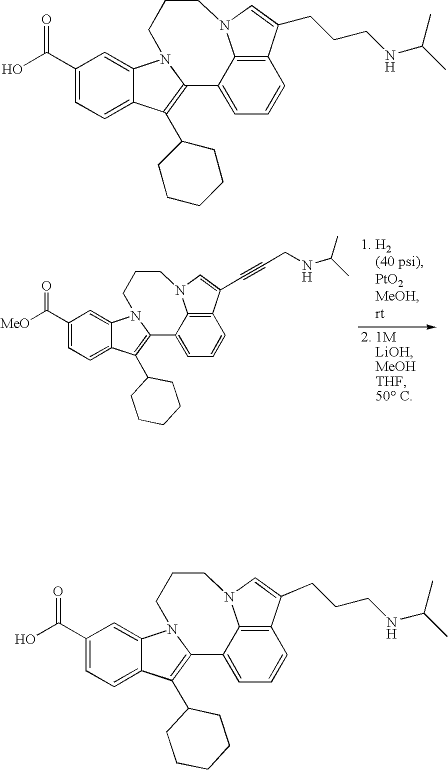

A Parr hydrogenation vessel was charged with 102 mg of the above alkyne (0.2 mmol), a catalytic quantity of PtO₂ and 30 mL MeOH. The vessel was sealed, degassed and back-filled with H₂ (3×). The vessel was then charged with 40 psi H₂ and allowed to shake until HPLC analysis indicated complete conversion. The reaction mixture was then filtered and concentrated. The resulting residue was dissolved with 6 mL THF, 2 mL MeOH and 2 mL LiOH (1M, aqueous). The mixture was then allowed to stir at 50° C. until complete by HPLC. The mixture was neutralized with 1 mL HCl (2M, aqueous) and concentrated. The resulting residue was dissolved with DMF and acidified with TFA. The solution was filtered and then purified by reverse-phase HPLC. The desired fractions were collected and concentrated. The purified residue was dissolved with CH₃CN and acidified with 2M HCl/Et₂O. The acidic solution was concentrated, the resulting residue was suspended with water, frozen and lyophilized to afford 46 mg (46%) Compound 433 as an off-white powder. MS: 498.3 (M+H⁺); ¹H NMR (DMSO-d6): δ 8.79 (br s, 2H), 8.16 (s, 1H), 7.94 (d, J=8.6, 1H), 7.74 (d, J=8.0, 1H), 7.70 (d, J=8.6, 1H), 7.28-7.07 (m, 3H), 4.64 (d, J=10.6, 1H), 4.12 (d, J=14.6, 1H), 3.65-2.77 (m, 8H), 2.06-1.13 (m, 20H).

### Example 120

### Preparation of Compound 434

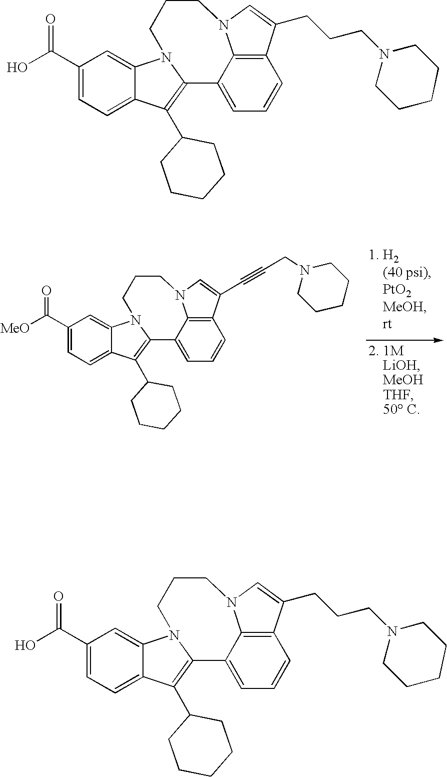

A Parr hydrogenation vessel was charged with 107 mg of the above alkyne (0.2 mmol), a catalytic quantity of PtO₂ and 30 mL MeOH. The vessel was sealed, degassed and back-filled with H₂ (3×). The vessel was then charged with 40 psi H₂ and allowed to shake until HPLC analysis indicated complete conversion. The reaction mixture was then filtered and concentrated. The resulting residue was dissolved with 6 mL THF, 2 mL MeOH and 2 mL LiOH (1M, aqueous). The mixture was then allowed to stir at 50° C. until complete by HPLC. The mixture was neutralized with 1 mL HCl (2M, aqueous) and concentrated. The resulting residue was dissolved with DMF and acidified with TFA. The solution was filtered and then purified by reverse-phase HPLC. The desired fractions were collected and concentrated. The purified residue was dissolved with CH₃CN and acidified with 2M HCl/Et₂O. The acidic solution was concentrated, the resulting residue was suspended with water, frozen and lyophilized to afford 52 mg (50%) Compound 434 as an off-white powder. MS: 524.3 (M+H⁺); ¹H NMR (DMSO-d6): δ 9.95 (br s, 1H), 8.16 (d, J=0.9, 1H), 7.94 (d, J=8.4, 1H), 7.75-7.69 (m, 2H), 7.29 (s, 1H), 7.23 (t, J=7.2, 1H), 7.14 (d, J=6.6, 1H), 4.64 (dd, J=14.7, 4.9, 1H), 4.10 (d, J=14.1, 1H), 3.67-3.58 (m, 1H), 3.47 (d, J=11.2, 2H), 3.28-3.11 (m, 3H), 2.93-2.77 (m, 5H), 2.19-1.12 (m, 20H).

### Example 121

### Preparation of Compound 435

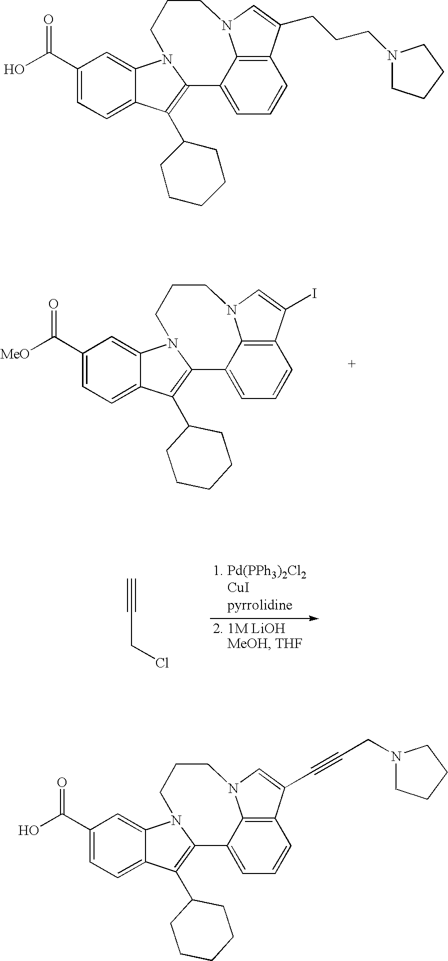

A reaction vessel was charged with 108 mg of the above ester (0.2 mmol), 7 mg Pd(PPh₃)₄ (0.01 mmol, 0.05 eq) and 4 mg CuI (0.02 mmol, 0.1 eq). Pyrrolidine (2 mL) was then added and the reaction vessel was sealed, degassed and back-filled with argon. Propargyl chloride (28 μL, 0.4 mmol, 2 eq) was added via syringe and the reaction vessel was allowed to stir at 50° C. until complete by HPLC analysis. Water was then added to the reaction mixture to precipitate the desired material. The solids were collected by centrifuge and washed with additional water. The resulting residue was then dissolved with 6 mL THF, 2 mL MeOH and 2 mL LiOH (1M, aqueous). The mixture was then allowed to stir at 35° C. overnight. The mixture was neutralized with 1 mL HCl (2M, aqueous), concentrated and used without further purification. MS: 506.3 (M+H⁺).

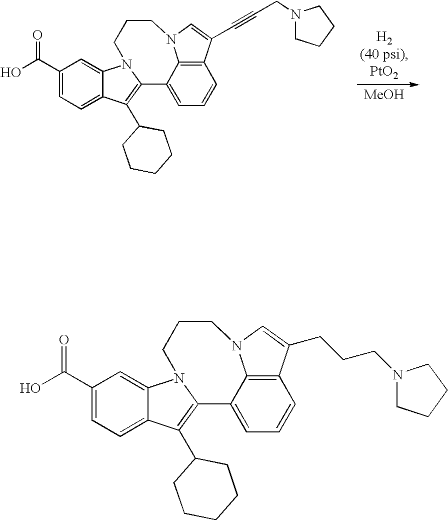

A Parr hydrogenation vessel was charged with 101 mg of the above alkyne (0.2 mmol), a catalytic quantity of PtO₂ and 30 mL MeOH. The vessel was sealed, degassed and back-filled with H₂ (3×). The vessel was then charged with 40 psi H₂ and allowed to shake until HPLC analysis indicated complete conversion. The reaction mixture was then filtered and concentrated. The resulting residue was dissolved with DMF and acidified with TFA. The solution was filtered and then purified by reverse-phase HPLC. The desired fractions were collected and concentrated. The purified residue was dissolved with CH₃CN and acidified with 2M HCl/Et₂O. The acidic solution was concentrated, the resulting residue was suspended with water, frozen and lyophilized to afford 66 mg (65%) Compound 435 as an off-white powder. MS: 510.3 (M+H⁺); ¹H NMR (DMSO-d6): δ 10.48 (br s, 1H), 8.16 (d, J=1.1, 1H), 7.94 (d, J=8.5, 1H), 7.75-7.68 (m, 2H), 7.29 (s, 1H), 7.23 (t, J=7.4, 1H), 7.13 (d, J=6.3, 1H), 4.62 (m, 1H), 4.11 (d, J=13.1, 1H), 3.62-3.54 (m, 3H), 3.28-3.20 (m, 3H), 3.05-3.00 (m, 2H), 2.92-2.83 (m, 3H), 2.14-1.39 (m, 20H).

### Example 122

### Preparation of Compound 437

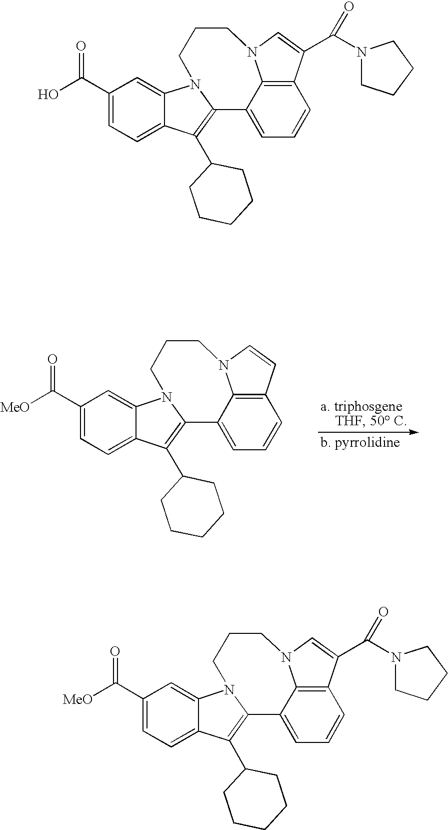

A reaction vessel was charged with 104 mg of the above ester (0.25 mmol), triphosgene (148 mg, 0.5 mmol, 2 eq) and 2.5 mL THF. The vessel was sealed and heated to 50° C. until HPLC analysis indicated complete conversion. Pyrrolidine (0.41 mL, 5 mmol, 20 eq) was then carefully added via pipet. A thick, white precipitate quickly formed. HPLC analysis indicated complete addition after 5 min. The reaction mixture was then concentrated and the desired material precipitated with water. The solids were collected by centrifuge, washed with additional water and used without further purification. MS: 510.2 (M+H⁺).

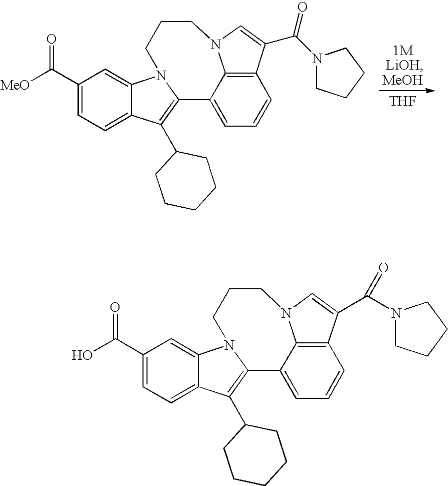

Approximately 127 mg of the above ester (0.25 mmol) was dissolved with 6 mL THF, 2 mL MeOH and 2 mL LiOH (1M, aqueous). The mixture was then allowed to stir at 50° C. until complete by HPLC. The mixture was neutralized with 1 mL HCl (2M, aqueous) and concentrated. The resulting residue was dissolved with DMF and acidified with TFA. The solution was filtered and then purified by reverse-phase HPLC. The desired fractions were collected and concentrated. The resulting residue was suspended with water, frozen and lyophilized to afford 79 mg (64%) Compound 437 as an off-white powder. MS: 496.3 (M+H⁺); ¹H NMR (DMSO-d6): δ 8.28 (d, J=8.0, 1H), 8.18 (s, 1H), 8.01 (s, 1H), 7.94 (d, J=8.3, 1H), 7.71 (d, J=8.6, 1H), 7.31 (t, J=7.1, 1H), 7.19 (d, J=6.9, 1H), 4.67 (dd, J=14.3, 3.7, 1H), 4.26 (d, J=13.7, 1H), 3.69-3.42 (m, 5H), 3.30-3.23 (m, 1H), 2.86 (br s, 1H), 2.11-1.10 (m, 16H).

### Example 123

### Preparation of Compound 438

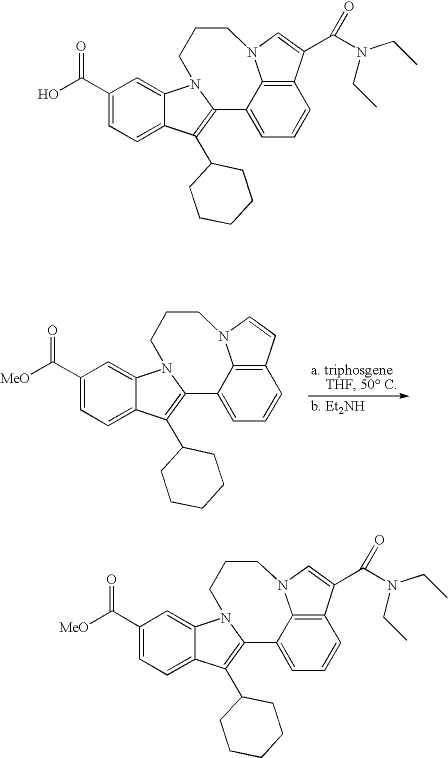

A reaction vessel was charged with 104 mg of the above ester (0.25 mmol), triphosgene (148 mg, 0.5 mmol, 2 eq) and 2.5 mL THF. The vessel was sealed and heated to 50° C. until HPLC analysis indicated complete conversion. Diethylamine (0.52 mL, 5 mmol, 20 eq) was then carefully added via pipet. A thick, white precipitate quickly formed. HPLC analysis indicated complete addition after 5 min. The reaction mixture was then concentrated and the desired material precipitated with water. The solids were collected by centrifuge, washed with additional water and used without further purification. MS: 512.3 (M+H⁺).

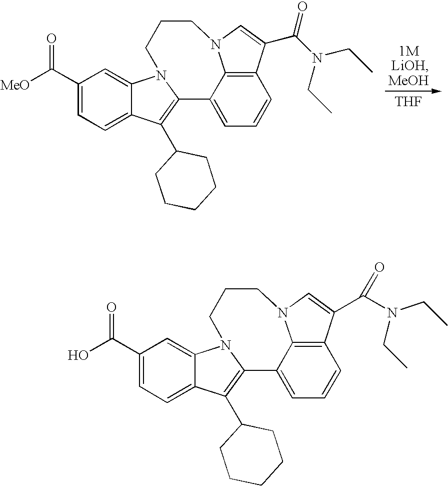

Approximately 128 mg of the above ester (0.25 mmol) was dissolved with 6 mL THF, 2 mL MeOH and 2 mL LiOH (1M, aqueous). The mixture was then allowed to stir at 50° C. until complete by HPLC. The mixture was neutralized with 1 mL HCl (2M, aqueous) and concentrated. The resulting residue was dissolved with DMF and acidified with TFA. The solution was filtered and then purified by reverse-phase HPLC. The desired fractions were collected and concentrated. The resulting residue was suspended with water, frozen and lyophilized to afford 85 mg (69%) Compound 438 as an off-white powder. MS: 498.2 (M+H⁺); ¹H NMR (DMSO-d6): δ 8.18 (s, 1H), 7.95 (d, J=8.5, 1H), 7.92 (d, J=7.3, 1H), 7.74 (s, 1H), 7.71 (d, J=8.5, 1H), 7.30 (t, J=7.6, 1H), 7.18 (d, J=7.1, 1H), 4.68 (d, J=16.1, 1H), 4.27 (d, J=13.8, 1H), 3.70-3.51 (m, 5H), 3.30-3.22 (m, 1H), 2.87 (br s, 1H), 2.08-1.13 (m, 18H).

### Example 124

### Preparation of Compound 439

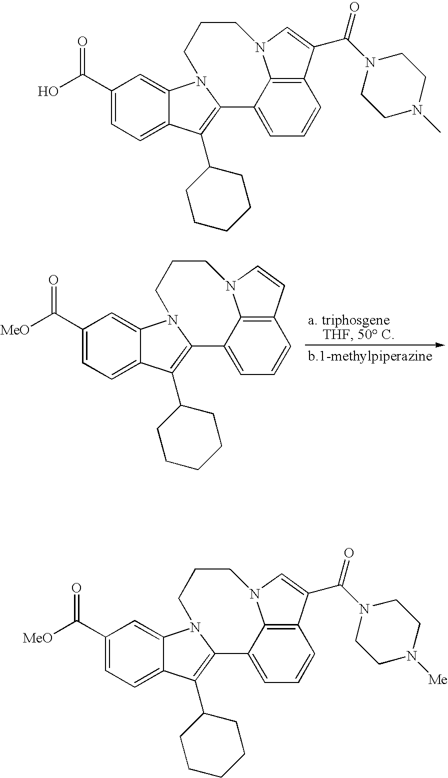

A reaction vessel was charged with 104 mg of the above ester (0.25 mmol), triphosgene (148 mg, 0.5 mmol, 2 eq) and 2.5 mL THF. The vessel was sealed and heated to 50° C. until HPLC analysis indicated complete conversion. 1-Methylpiperazine (0.56 mL, 5 mmol, 20 eq) was then carefully added via pipet. A thick, white precipitate quickly formed. HPLC analysis indicated complete addition after 5 min. The reaction mixture was then concentrated and the desired material precipitated with water. The solids were collected by centrifuge, washed with additional water and used without further purification. MS: 539.3 (M+H⁺).

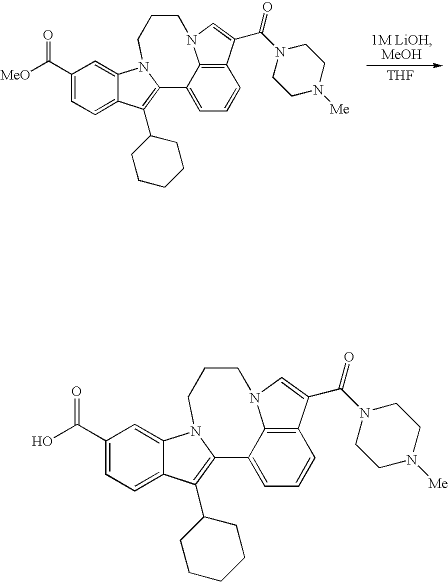

Approximately 135 mg of the above ester (0.25 mmol) was dissolved with 6 mL THF, 2 mL MeOH and 2 mL LiOH (1M, aqueous). The mixture was then allowed to stir at 50° C. until complete by HPLC. The mixture was neutralized with 1 mL HCl (2M, aqueous) and concentrated. The resulting residue was dissolved with DMF and acidified with TFA. The solution was filtered and then purified by reverse-phase HPLC. The desired fractions were collected and concentrated. The purified residue was dissolved with CH₃CN and acidified with 2M HCl/Et₂O. The acidic solution was concentrated, the resulting residue was suspended with water, frozen and lyophilized to afford 102 mg (78%) Compound 439 as an off-white powder. MS: 525.3 (M+H⁺); ¹H NMR (DMSO-d6): δ 10.70 (br s, 1H), 8.19 (d, J=0.9, 1H), 7.97-7.93 (m, 3H), 7.71 (dd, J=8.3, 1.1, 1H), 7.36 (t, J=7.5, 1H), 7.22 (d, J=6.6, 1H), 4.70 (dd, J=13.8, 4.3, 1H), 4.50 (d, J=13.5, 2H), 4.25 (d, J=14.4, 1H), 3.71-3.61 (m, 1H), 3.51-3.13 (m, 7H), 2.93-2.77 (m, 4H), 2.12-1.13 (m, 12H).

### Example 125

### Preparation of Compound 440

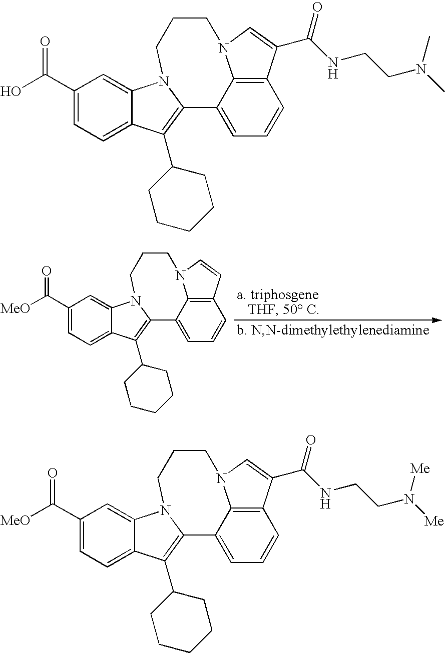

A reaction vessel was charged with 104 mg of the above ester (0.25 mmol), triphosgene (148 mg, 0.5 mmol, 2 eq) and 2.5 mL THF. The vessel was sealed and heated to 50° C. until HPLC analysis indicated complete conversion. 1-Methylpiperazine (0.56 mL, 5 mmol, 20 eq) was then carefully added via pipet. A thick, white precipitate quickly formed. HPLC analysis indicated complete addition after 5 min. The reaction mixture was then concentrated and the desired material precipitated with water. The solids were collected by centrifuge, washed with additional water and used without further purification. MS: 527.3 (M+H⁺).

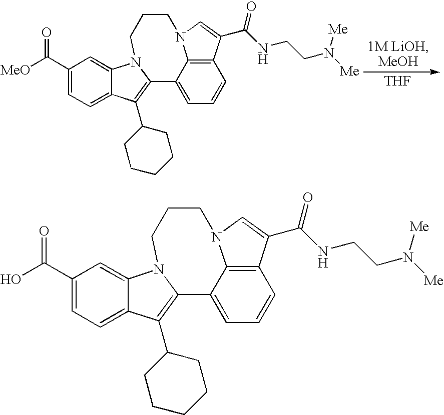

Approximately 132 mg of the above ester (0.25 mmol) was dissolved with 6 mL THF, 2 mL MeOH and 2 mL LiOH (1M, aqueous). The mixture was then allowed to stir at 50° C. until complete by HPLC. The mixture was neutralized with 1 mL HCl (2M, aqueous) and concentrated. The resulting residue was dissolved with DMF and acidified with TFA. The solution was filtered and then purified by reverse-phase HPLC. The desired fractions were collected and concentrated. The purified residue was dissolved with CH₃CN and acidified with 2M HCl/Et₂O. The acidic solution was concentrated, the resulting residue was suspended with water, frozen and lyophilized to afford 37 mg (29%) Compound 440 as a white powder. MS: 513.3 (M+H⁺); ¹H NMR (DMSO-d6): δ 10.42 (br s, 1H), 8.53 (t, J=5.3, 1H), 8.39 (d, J=8.1, 1H), 8.26 (s, 1H), 8.20 (s, 1H), 7.97 (d, J=8.4, 1H), 7.73 (dd, J=8.4, 1.0, 1H), 7.37 (t, J=7.4, 1H), 7.22 (d, J=7.1, 1H), 4.69 (dd, J=14.3, 4.7, 1H), 4.15 (d, J=12.1, 1H), 3.75-3.25 (m, 6H), 2.93-2.77 (m, 7H), 2.14-1.13 (m, 12H).

### Example 126

### Preparation of Compound 441

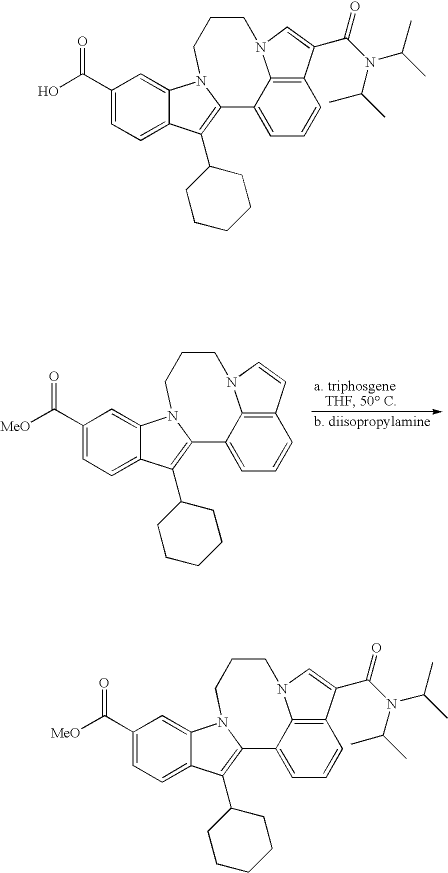

A reaction vessel was charged with 104 mg of the above ester (0.25 mmol), triphosgene (148 mg, 0.5 mmol, 2 eq) and 2.5 mL THF. The vessel was sealed and heated to 50° C. until HPLC analysis indicated complete conversion. 1-Methylpiperazine (0.56 mL, 5 mmol, 20 eq) was then carefully added via pipet. A thick, white precipitate quickly formed. HPLC analysis indicated complete addition after extended stirring. The reaction mixture was then concentrated and the desired material precipitated with water. The solids were collected by centrifuge, washed with additional water and used without further purification. MS: 540.3 (M+H⁺).

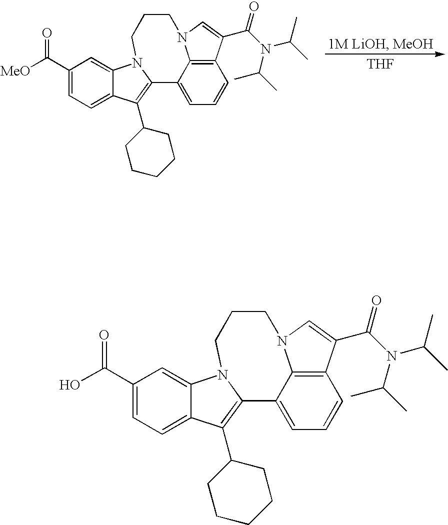

Approximately 135 mg of the above ester (0.25 mmol) was dissolved with 6 mL THF, 2 mL MeOH and 2 mL LiOH (1M, aqueous). The mixture was then allowed to stir at 50° C. until complete by HPLC. The mixture was neutralized with 1 mL HCl (2M, aqueous) and concentrated. The resulting residue was dissolved with DMF and acidified with TFA. The solution was filtered and then purified by reverse-phase HPLC. The desired fractions were collected and concentrated. The resulting residue was suspended with water, frozen and lyophilized to afford 100 mg (76%) Compound 441 as a white powder. MS: 526.3 (M+H⁺); ¹H NMR (DMSO-d6): δ 8.19 (d, J=1.0, 1H), 7.96 (d, J=8.7, 1H), 7.82 (dd, J=7.7, 1.0, 1H), 7.72 (dd, J=8.4, 1.3, 1H), 7.61 (s, 1H), 7.30 (t, J=7.4, 1H), 7.19 (d, J=6.4, 1H), 4.68 (d, J=12.8, 1H), 4.24 (d, J=14.4, 1H), 4.03 (br s, 2H), 3.72-3.61 (m, 1H), 3.32-3.22 (m, 1H), 2.93-2.85 (m, 1H), 2.11-1.18 (m, 24H).

### Example 127

### Preparation of Compound 442

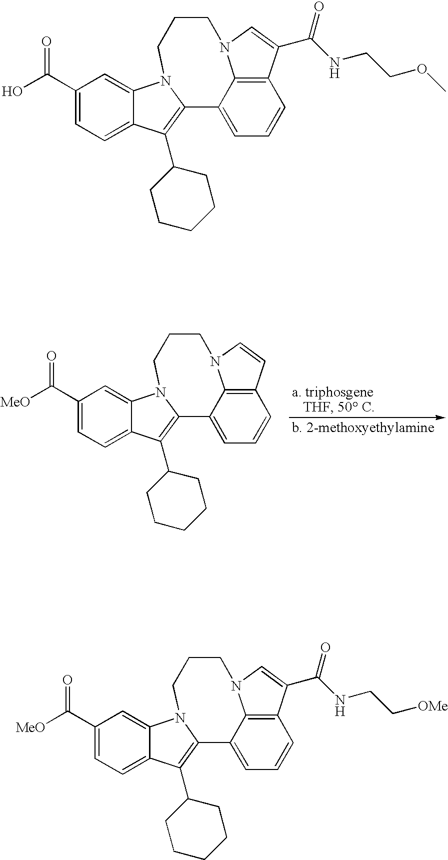

A reaction vessel was charged with 104 mg of the above ester (0.25 mmol), triphosgene (148 mg, 0.5 mmol, 2 eq) and 2.5 mL THF. The vessel was sealed and heated to 50° C. until HPLC analysis indicated complete conversion. 1-Methylpiperazine (0.56 mL, 5 mmol, 20 eq) was then carefully added via pipet. A thick, white precipitate quickly formed. HPLC analysis indicated complete addition after 5 min. The reaction mixture was then concentrated and the desired material precipitated with water. The solids were collected by centrifuge, washed with additional water and used without further purification. MS: 514.2 (M+H⁺).

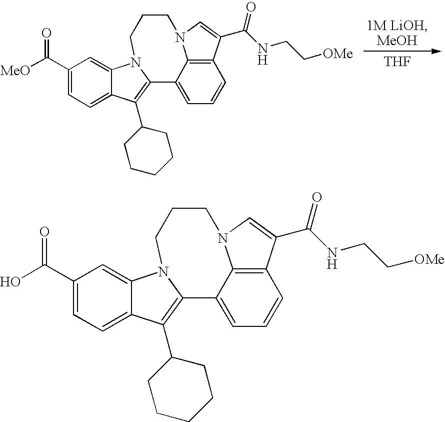

Approximately 128 mg of the above ester (0.25 mmol) was dissolved with 6 mL THF, 2 mL MeOH and 2 mL LiOH (1M, aqueous). The mixture was then allowed to stir at 50° C. until complete by HPLC. The mixture was neutralized with 1 mL HCl (2M, aqueous) and concentrated. The resulting residue was dissolved with DMF and acidified with TFA. The solution was filtered and then purified by reverse-phase HPLC. The desired fractions were collected and concentrated. The resulting residue was suspended with water, frozen and lyophilized to afford 83 mg (66%) Compound 442 as a white powder. MS: 500.3 (M+H⁺); ¹H NMR (DMSO-d6): δ 8.37 (dd, J=8.1, 1.0, 1H), 8.19 (d, J=1.0, 1H), 8.07 (s, 1.0, 1H), 8.04 (t, J=5.0, 1H), 7.96 (d, J=8.4, 1H), 7.72 (dd, J=8.4, 1.3, 1H), 7.35 (t, J=7.4, 1H), 7.21 (d, J=7.1, 1H), 4.68 (dd, J=14.4, 4.7, 1H), 4.14 (d, J=14.4, 1H), 3.71-3.60 (m, 1H), 3.56-3.41 (m, 4H), 3.33 (s, 3H), 3.33-3.23 (m, 1H), 2.87-2.83 (m, 1H), 2.07-1.13 (m, 12H).

### Example 128

### Preparation of Compound 443

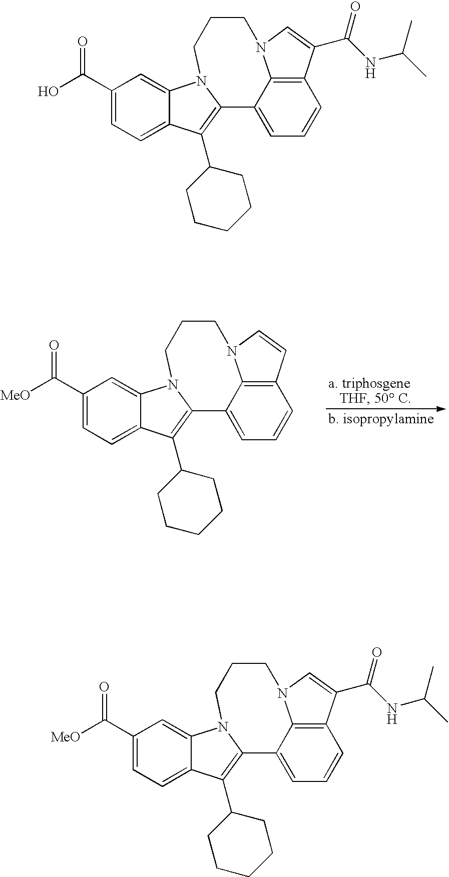

A reaction vessel was charged with 104 mg of the above ester (0.25 mmol), triphosgene (148 mg, 0.5 mmol, 2 eq) and 2.5 mL THF. The vessel was sealed and heated to 50° C. until HPLC analysis indicated complete conversion. 1-Methylpiperazine (0.56 mL, 5 mmol, 20 eq) was then carefully added via pipet. A thick, white precipitate quickly formed. HPLC analysis indicated complete addition after 5 min. The reaction mixture was then concentrated and the desired material precipitated with water. The solids were collected by centrifuge, washed with additional water and used without further purification. MS: 498.3 (M+H⁺).

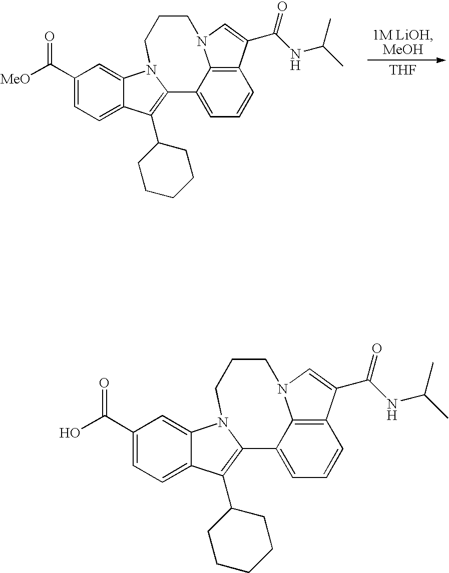

Approximately 124 mg of the above ester (0.25 mmol) was dissolved with 6 mL THF, 2 mL MeOH and 2 mL LiOH (1M, aqueous). The mixture was then allowed to stir at 50° C. until complete by HPLC. The mixture was neutralized with 1 mL HCl (2M, aqueous) and concentrated. The resulting residue was dissolved with DMF and acidified with TFA. The solution was filtered and then purified by reverse-phase HPLC. The desired fractions were collected and concentrated. The resulting residue was suspended with water, frozen and lyophilized to afford 100 mg (76%) Compound 443 as a white powder. MS: 484.2 (M+H⁺); ¹H NMR (DMSO-d6): δ 8.36 (dd, J=8.2, 1.2, 1H), 8.19 (d, J=1.2, 1H), 8.08 (s, 1H), 7.96 (d, J=8.5, 1H), 7.77-7.71 (m, 2H), 7.34 (t, J=7.3, 1H), 7.20 (dd, J=7.3, 0.9, 1H), 4.68 (dd, J=14.4, 4.1, 1H), 4.21-4.10 (m, 2H), 3.71-3.60 (m, 1H), 3.33-3.23 (m, 1H), 2.86-2.83 (m, 1H), 2.93-2.85 (m, 1H), 2.07-1.12 (m, 18H).

### Example 129

### Preparation of Compound 444

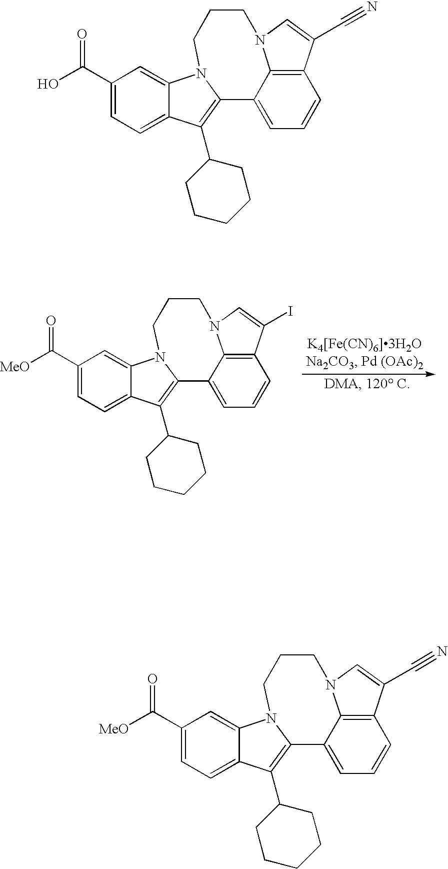

A reaction vessel was charged with 108 mg of the above ester (0.2 mmol), 4 mg Pd(OAc)₂ (0.02 mmol, 0.1 eq) 22 mg Na₂CO₃ (0.2 mmol, 1 eq) and 18 mg K₄[Fe(CN)₆].3H₂O (0.044 mmol, 0.22 eq). Dimethylacetamide (0.6 mL) was then added and the reaction vessel was sealed, degassed and back-filled with argon. The reaction mixture was allowed to stir at 120° C. until complete by HPLC analysis. Water was then added to the reaction mixture to precipitate the desired material. The solids were collected by centrifuge, washed with additional water, dried under vacuum and used without further purification. MS: 438.3 (M+H⁺).

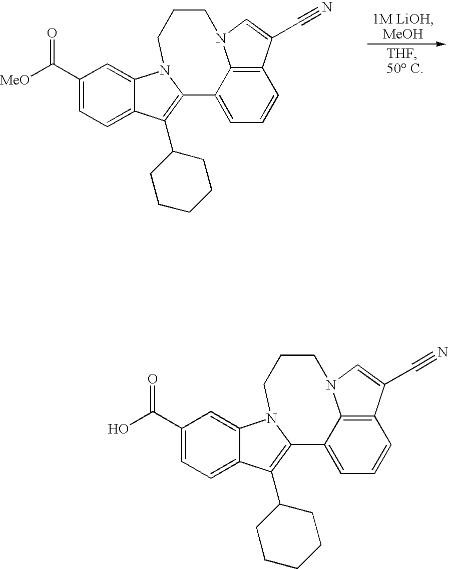

Approximately 88 mg of the above ester (0.2 mmol) was dissolved with 6 mL THF, 2 mL MeOH and 2 mL LiOH (1M, aqueous). The mixture was then allowed to stir at 50° C. until complete by HPLC. The mixture was neutralized with 1 mL HCl (2M, aqueous) and concentrated. The resulting residue was dissolved with DMF and acidified with TFA. The solution was filtered and then purified by reverse-phase HPLC. The desired fractions were collected and concentrated. The resulting residue was suspended with water, frozen and lyophilized to afford 39 mg (46%) Compound 444 as a white powder. MS: 424.2 (M+H⁺); ¹H NMR (DMSO-d6): δ 8.38 (s, 1H), 8.19 (d, J=1.2, 1H), 7.96 (d, J=8.7, 1H), 7.82 (dd, J=7.8, 0.9, 1H), 7.71 (dd, J=8.4, 1.2, 1H), 7.48 (t, J=7.5, 1H), 7.32 (dd, J=7.2, 0.9, 1H), 4.69 (dd, J=14.7, 4.9, 1H), 4.27 (d, J=14.2, 1H), 3.70-3.59 (m, 1H), 3.34-3.24 (m, 1H), 2.93-2.77 (m, 1H), 2.14-1.10 (m, 12H).

### Example 130

### Preparation of Compound 445

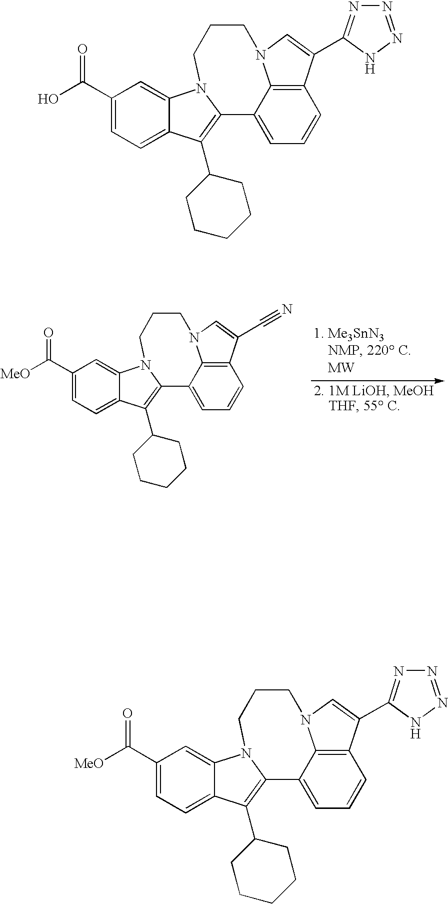

A microwave reaction vessel was charged with 88 mg of the methyl ester (0.2 mmol) and 208 mg Me₃SnN₃ (1 mmol, 5 eq). 1-Methylpyrollidinone (2 mL) was then added and the reaction vessel was sealed and heated to 220° C. for 20 min. by microwave. HPLC analysis indicated complete conversion. Water was then added to the reaction mixture to precipitate the desired material. The solids were collected by centrifuge and washed with additional water. The ester was then dissolved with 6 mL THF, 2 mL MeOH and 2 mL LiOH (1M, aqueous). The mixture was then allowed to stir at 55° C. until complete by HPLC. The mixture was neutralized with 1 mL HCl (2M, aqueous) and concentrated. The resulting residue was dissolved with DMF and acidified with TFA. The solution was filtered and then purified by reverse-phase HPLC. The desired fractions were collected and concentrated. The purified residue was dissolved with CH₃CN and acidified with 2M HCl/Et₂O. The acidic solution was concentrated, the resulting residue was suspended with water, frozen and lyophilized to afford 20 mg (21%) Compound 445 as a light brown powder. MS: 467.2 (M+H⁺); ¹H NMR (DMSO-d6): δ 8.43 (d, J=8.0, 1H), 8.20 (s, 1H), 8.18 (s, 1H), 7.97 (d, J=8.3, 1H), 7.72 (d, J=8.5, 1H), 7.46 (t, J=7.4, 1H), 7.30 (d, J=7.1, 1H), 4.71 (d, J=13.4, 1H), 4.33 (d, J=14.0, 1H), 3.76-3.65 (m, 1H), 3.38-3.20 (m, 1H), 2.93-2.73 (m, 1H), 2.12-1.13 (m, 12H).

### Example 131

### Preparation of Compound 446

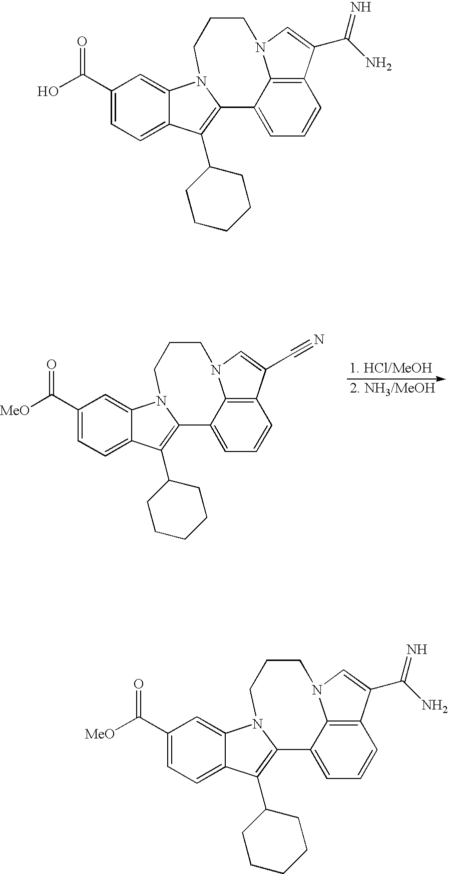

A reaction flask was charged with 200 mg of the above ester (0.46 mmol) and suspended with 10 mL MeOH. The vessel was sealed, cooled to 0° C. and carefully saturated with HCl. The mixture was allowed to sit at 4° C. for 1 day. The solution was then transferred to a larger flask and carefully concentrated. The resulting residue was taken up with toluene and again concentrated and dried on high vacuum to remove any remaining HCl. A concentrated solution of NH₃ in MeOH was then added and the mixture was allowed to stir at 40° C. overnight. HPLC and LC-MS analysis confirmed complete conversion to the desired product. The reaction mixture was then concentrated and used without further purification. MS: 455.2 (M+H⁺).

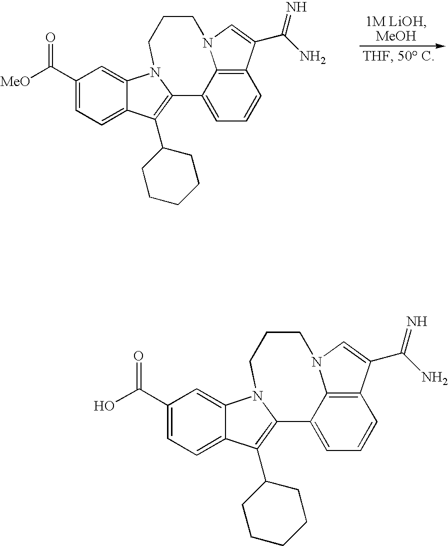

Approximately 88 mg of the above ester (0.46 mmol) was dissolved with 12 mL THF, 4 mL MeOH and 4 mL LiOH (1M, aqueous). The mixture was then allowed to stir at 50° C. until complete by HPLC. The mixture was neutralized with 1 mL HCl (2M, aqueous) and concentrated. The resulting residue was dissolved with DMF and acidified with TFA. The solution was filtered and then purified by reverse-phase HPLC. The desired fractions were collected and concentrated. The purified residue was dissolved with CH₃CN and acidified with 2M HCl/Et₂O. The acidic solution was concentrated, the resulting residue was suspended with water, frozen and lyophilized to afford 50 mg (25%) Compound 446 as a yellow-orange powder. MS: 441.2 (M+H⁺); ¹H NMR (DMSO-d6): δ 8.98 (br s, 2H), 8.91 (br s, 2H), 8.33 (s, 1H), 8.21 (s, 1H), 7.99 (d, J=8.0, 1H), 7.97 (d, J=8.6, 1H), 7.72 (d, J=8.3, 1H), 7.49 (t, J=7.7, 1H), 7.32 (d, J=7.1, 1H), 4.73 (d, J=10.0, 1H), 4.30 (d, J=13.1, 1H), 3.67-3.60 (m, 1H), 3.30 (t, J=12.3, 1H), 2.92-2.76 (m, 1H), 2.17-1.09 (m, 12H).

### Example 132

### Preparation of Compound 395

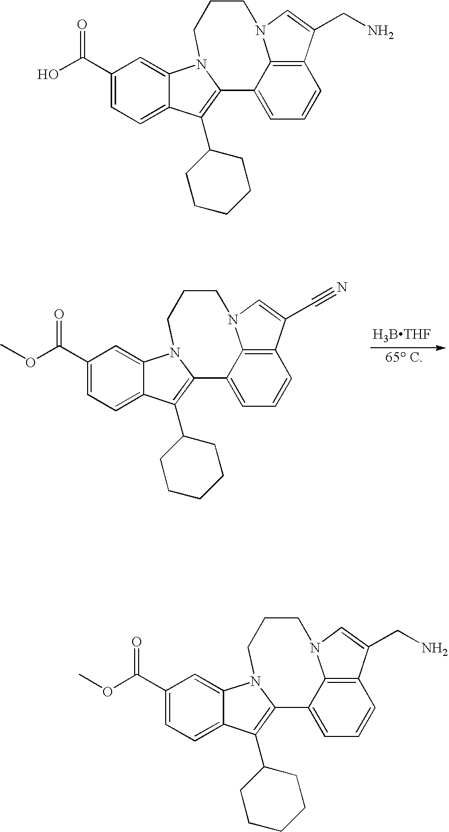

A solution of the above nitrile (567 mg, 1.08 mmol, 1 equiv) in THF (10 mL) under Ar was cooled to 0° C., then H₃B*THF (1M in THF, 11 mL, 11 mmol, 10 equiv) was added dropwise. The reaction was heated to reflux for 1 h. After the reaction had cooled to RT, it was SLOWLY quenched with aqueous 2N HCl (27 mL, 54 mmol, 50 equiv) and heated to 65° C. for 15 min. After the reaction had cooled to RT, it was basified with aqueous 1N NaOH (60 mL, 60 mmol, 60 equiv). The basic solution was taken in EtOAc, then the layers were separated. The organic layer was washed with brine 1×. The organic layer was dried with Na₂SO₄, filtered, concentrated, and dried in vacuo to give crude amine (413 mg, 87%) as a yellow brown solid that was used in the next step without further purification. MS: 425.2 (M+H⁺); ¹H-NMR (DMSO-d₆): δ (ppm) 8.14 (bs, 1H), 7.93 (d, J=8.4 Hz), 7.76-765 (m, 2H), 7.42 (s, 1H), 7.22-7.10 (m, 2H), 4.65-4.53 (m, 1H), 3.89-3.80 (m, 6H), 3.65-3.49 (m, 1H), 3.22-3.10 (m, 1H), 2.89-2.72 (m, 1H), 2.07-1.05 (m, 12H).

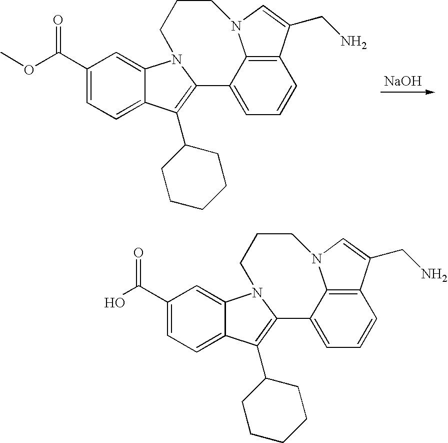

The above amine (413 mg, 0.935 mmol, 1 equiv) in THF/MeOH (2:1, 15 mL) was treated with aqueous NaOH (4 N, 2.34 mL, 9.35 mmol, 10 equiv) and heated to 80° C. for 0.5 h. After cooling to RT, the reaction mixture was concentrated. The crude product was taken in H₂O and acidified to pH=7 with 1 N aqueous HCl giving a precipitate. The precipitate was collected by centrifuging and purified by RP-HPLC. After concentrating to dryness, the pure solid was dissolved in ACN (2 mL) and charged with 2N HCl/EE (4 mL) to provide the HCl salt which was dried by lyophilizing to yield Compound 395 (31 mg). MS: 411.2 (M+H⁺); ¹H-NMR (DMSO-d₆): δ (ppm) 12.6 (bs, 1H), 8.09 (s, 1H), 8.11-8.00 (m, 4H), 7.84 (d, 2H, J=8.4 Hz), 7.61 (dd, 1H, J=8.4 Hz, J=1.2 Hz), 7.47 (s, 1H), 7.22 (t, 1H, J=7.2 Hz), 7.12-7.10 (m, 1H), 4.61-4.53 (m, 1H), 4.17-4.08 (m, 3H), 3.55-3.49 (m, 1H), 3.17-3.10 (m, 1H), 2.79 (bs, 1H), 2.10-1.01 (m, 12H).

### Example 133

### Preparation of Compound 396

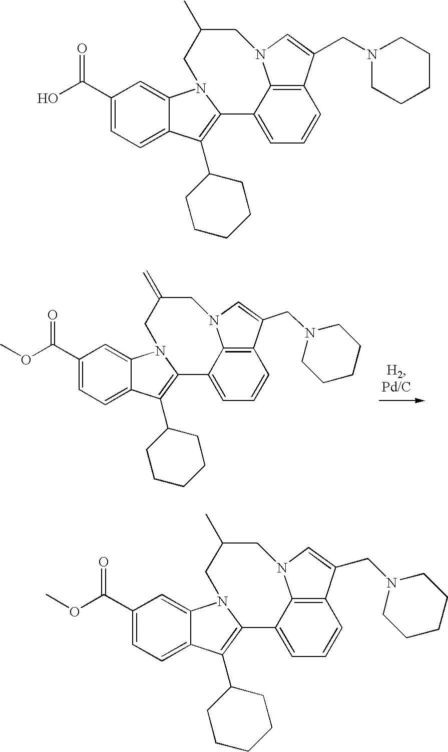

The above alkene (30 mg, 0.0575 mmol, 1 equiv) in MeOH (2 mL) was charged with 10% Pd/C (6 mg, 10 mol %) and hydrogenated at 50 psi for 1 h. The catalyst was filtered off using a celite pad and rinsed with MeOH. The filtrate was concentrated and dried in vacuo give the reduced product (30 mg, 99%) as a light-brown solid that was used in the next step without further purification. MS: 524.3 (M+H⁺); ¹H-NMR (DMSO-d₆): δ (ppm) 8.20 (s, 1H), 7.96 (d, 1H, J=4.2 Hz), 7.93 (d, 1H, J=4.8 Hz), 7.66 (d, 1H, J=1 Hz), 7.63 (s, 1H), 7.30 (t, 1H, J=7.5 Hz), 7.11 (d, 1H, J=6.6 Hz), 4.60 (d, 1H, J=15 Hz), 4.48 (d, 2H, J=4.8 Hz), 4.14 (m, 1H), 3.87 (s, 3H), 3.83-3.77 (m, 1H), 3.51-3.33 (m, 3H), 3.00-2.80 (m, 3H), 2.40 (bs, 1H), 2.10-1.09 (m, 16H), 1.02 (d, 3H, J=7 Hz).

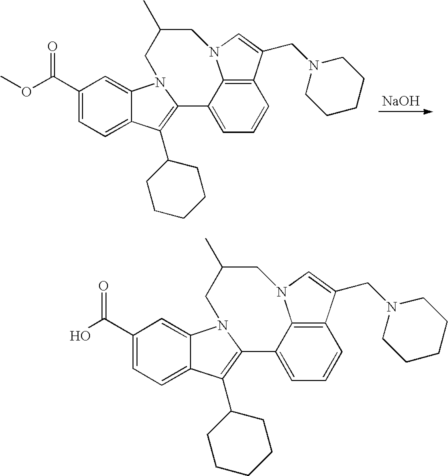

The above ester (100 mg, 0.191 mmol, 1 equiv) in THF/MeOH (2:1, 3 mL) was treated with aqueous NaOH (4 N, 480 uL, 1.91 mmol, 10 equiv) and heated to 50° C. for 2 h. After cooling to RT, the reaction mixture was concentrated. The crude product was taken in H₂O and acidified to pH=7 with 1 N aqueous HCl giving a precipitate. The precipitate was collected by centrifuging and purified by RP-HPLC. After concentrating to dryness, the pure solid was dissolved in ACN (2 mL) and charged with 2N HCl/EE (4 mL) to provide the HCl salt which was dried by lyophilizing to yield Compound 396 (30 mg). MS: 510.3 (M+H⁺); ¹H-NMR (DMSO-d₆): δ (ppm) 10.2 (bs, 1H), 8.09 (s, 1H), 7.91 (d, 1H, J=8.1 Hz), 7.84 (d, 1H, J=8.7 Hz), 7.61 (s, 1H), 7.57 (d, 1H, J=8.7 Hz), 7.22 (t, 1H, J=7.5 Hz), 7.01 (d, 1H, J=7.2 Hz), 4.49-4.33 (m, 3H), 4.04-3.98 (m, 1H), 3.75-3.69 (m, 1H), 3.47-3.25 (m, 3H), 2.83-2.70 (m, 3H), 2.29 (bs, 1H), 2.10-1.01 (m, 16H), 0.947 (d, 3H, J=5.4 Hz).

### Example 134

### Preparation of Compound 397

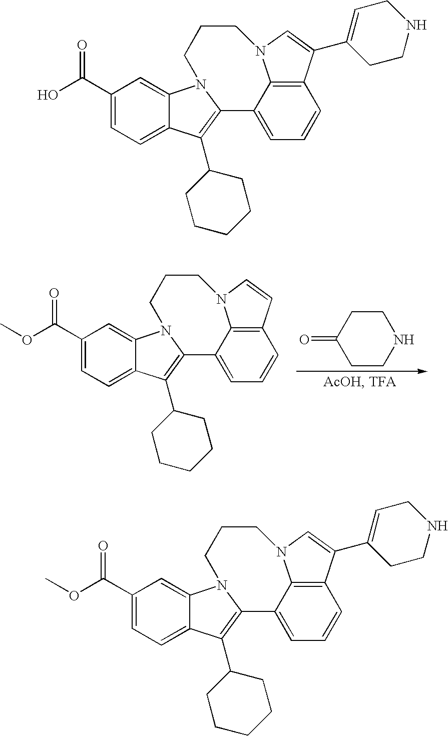

4-Piperidone.HCl.H₂O (603 mg, 3.9 mmol, 3.6 equiv) was charged with HOAc (1.36 mL) and TFA (1.82 mL), then the solution was heated to 110° C. The above ester (450 mg, 1.09 mmol, 1 equiv) in HOAc (5.46 mL) was added dropwise, and the mixture was heated at 110° C. for 1 h. Then the reaction mixture was cooled in an ice bath and quenched with ice-water. After neutralizing to pH=7 with solid NaOH, the neutral solution was taken in EtOAc. The layers were separated. The organic layer was washed with water 1× and brine 1×. The organic layer was dried with Na₂SO₄, filtered, concentrated, and dried in vacuo to give the above amine (511 mg, 95%) as a yellow brown solid that was used in the next step without further purification. MS: 494.3 (M+H⁺); ¹H-NMR (DMSO-d₆): δ (ppm) 9.17 (bs, 1H), 8.07 (d, 1H, J=1.2 Hz), 7.92 (d, 1H, J=8.4 Hz), 7.86 (d, 1H, J=8.4 Hz), 7.61 (dd, 1H, J=8.4 Hz, J=1.5 Hz), 7.52 (s, 1H), 7.21 (t, 1H, J=7.2 Hz), 7.07 (d, 1H, J=6.9 Hz), 6.12 (s, 1H), 4.59-4.52 (m, 1H), 4.04-3.96 (m, 1H), 3.79 (s, 3H), 3.71 (bs, 2H), 3.53-3.44 (m, 1H), 3.25-3.06 (m, 3H), 2.80-2.60 (m, 3H), 1.94-0.672 (m, 12H).

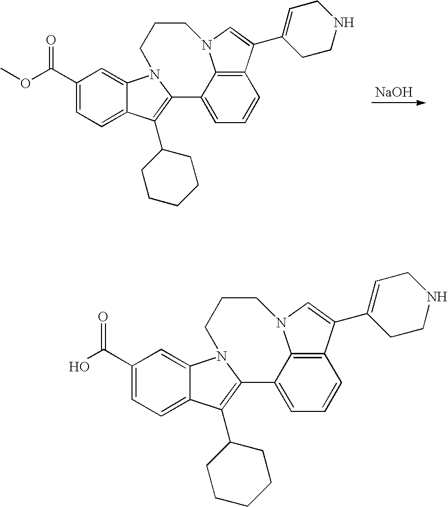

The above amine (125 mg, 0.253 mmol, 1 equiv) in THF/MeOH (2:1, 4.2 mL) was treated with aqueous NaOH (4 N, 633 uL, 2.53 mmol, 10 equiv) and heated to 80° C. for 1 h. After cooling to RT, the reaction mixture was concentrated. The crude product was taken in H₂O and acidified to pH=7 with 1 N aqueous HCl giving a precipitate. The precipitate was collected by centrifuging and purified by RP-HPLC. After concentrating to dryness, the pure solid was dissolved in ACN (2 mL) and charged with 2N HCl/EE (4 mL) to provide the HCl salt which was dried by lyophilizing to yield Compound 397 (32 mg). ¹H-NMR (DMSO-d₆): δ (ppm) 12.54 (s, 1H), 9.26-9.17 (m, 2H), 8.07 (d, 1H, J=1.2 Hz), 7.93 (d, 1H, J=8.4 Hz), 7.85 (d, 1H, J=8.4 Hz), 7.61 (dd, 1H, J=8.4 Hz, J=1.5 Hz), 7.53 (s, 1H), 7.22 (t, 1H, J=7.2 Hz), 7.08 (d, 1H, J=6.9 Hz), 6.14 (s, 1H), 4.58-4.52 (m, 1H), 4.06-4.02 (m, 1H), 3.72 (bs, 2H), 3.54-3.47 (m, 1H), 3.28-3.08 (m, 4H), 2.80-2.60 (m, 2H), 2.00-0.956 (m, 12H).

### Example 135

### Preparation of Compound 398

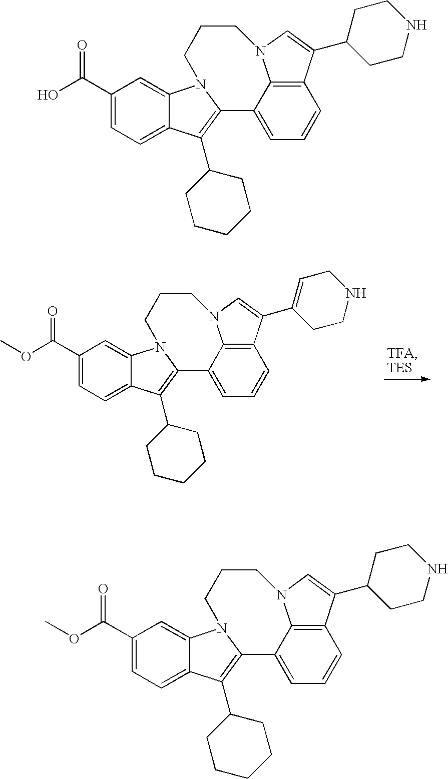

The above ester (125 mg, 0.253 mmol, 1 equiv) in TFA (2.53 mL) was treated with TES (60 uL, 0.380 mmol, 1.5 equiv) and stirred at RT for 1 h. The reaction mixture was concentrated and dried in vacuo to give the piperidine product (143 mg, 95%) as a dark brown solid that was used in the next step without further purification. MS: 496.3 (M+H⁺); ¹H-NMR (DMSO-d₆): δ (ppm) 8.38 (bs, 1H), 8.11 (d, 1H, J=1.2 Hz), 7.90 (d, 1H, J=8.4 Hz), 7.76 (d, 1H, J=8.4 Hz), 7.65 (dd, 1H, J=8.4 Hz, J=1.5 Hz), 7.19 (s, 1H), 7.18 (t, 1H, J=7.2 Hz), 7.07 (d, 1H, J=6.9 Hz), 4.59-4.55 (m, 1H), 4.08-4.04 (m, 1H), 3.84 (s, 3H), 3.75-3.79 (m, 1H), 3.35 (bs, 2H), 3.21-3.06 (m, 4H), 2.49 (bs, 1H), 2.04-1.05 (m, 16H).

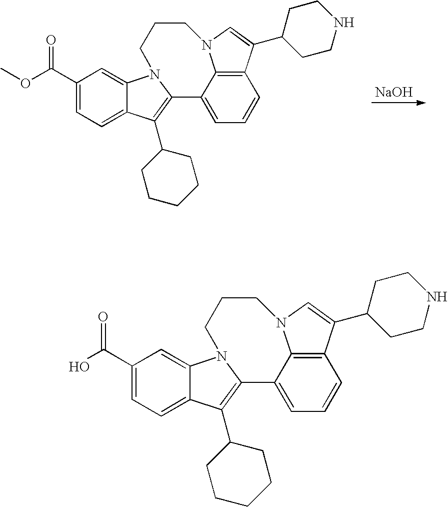

The above ester (143 mg, 0.288 mmol, 1 equiv) in THF/MeOH (2:1, 5 mL) was treated with aqueous NaOH (4 N, 721 uL, 2.88 mmol, 10 equiv) and heated to 80° C. for 1 h. After cooling to RT, the reaction mixture was concentrated. The crude product was taken in H₂O and acidified to pH=7 with 1 N aqueous HCl giving a precipitate. The precipitate was collected by centrifuging and purified by RP-HPLC. After concentrating to dryness, the pure solid was dissolved in ACN (2 mL) and charged with 2N HCl/EE (4 mL) to provide the HCl salt which was dried by lyophilizing to yield Compound 398 (35 mg). MS: 482.3 (M+H⁺); ¹H-NMR (DMSO-d₆): δ (ppm) 12.5 (bs, 1H), 9.04-8.95 (m, 2H), 8.04 (s, 1H), 7.83 (d, 1H, J=8.7 Hz), 7.76 (d, 1H, J=8.7 Hz), 7.61 (dd, 1H, J=8.4 Hz, J=0.90 Hz), 7.13 (s, 1H), 7.11 (d, 1H, J=7.8 Hz), 7.02 (d, 1H, J=6.9 Hz), 4.55-4.45 (m, 1H), 4.04-3.98 (m, 1H), 3.55-3.40 (m, 1H), 3.30 (bs, 4H), 3.15-2.86 (m, 2H), 2.81-2.68 (m, 1H), 2.14-0.955 (m, 16H).

### Example 136

### Preparation of Compound 399

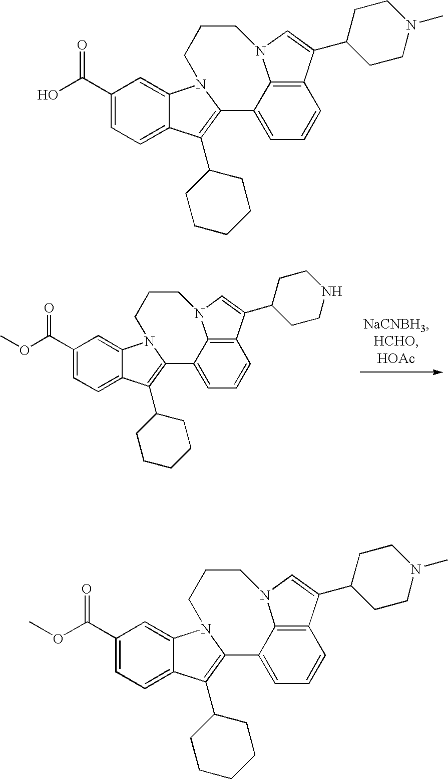

To a solution of the above ester (215 mg, 0.434 mmol, 1 equiv) in MeOH (7 mL) was added formaldehyde (37% in water, 42 uL, 0.564 mmol, 1.3 equiv), HOAc (150 ul, 2.60 mmol, 6 equiv), and NaCNBH₃ (82 mg, 1.30 mmol, 3 equiv), and the reaction was stirred at RT for 4 h. Then the reaction mixture was quenched with ice-water and SLOWLY added dropwise sat-bicarb. The reaction mixture was diluted with EtOAc. The layers were separated. The organic layer was washed with sat-bicarb 1×. The organic layer was dried with Na₂SO₄, filtered, concentrated, and dried in vacuo to give the N-methyl piperidine (245 mg, 90%) as a lemon yellow foam that was used in the next step without further purification. MS: 510.3 (M+H⁺); ¹H-NMR (DMSO-d₆): δ (ppm) 8.11 (d, 1H, J=1.2 Hz), 7.91 (d, 1H, J=8.4 Hz), 7.39-7.63 (m, 2H), 7.15-7.04 (m, 3H), 4.62-4.55 (m, 1H), 4.05-3.98 (m, 1H), 3.84 (s, 3H), 3.61-3.49 (m, 1H), 3.30-3.09 (m, 2H), 2.85-2.69 (m, 4H), 2.19 (s, 3H), 2.04-1.05 (m, 17H).

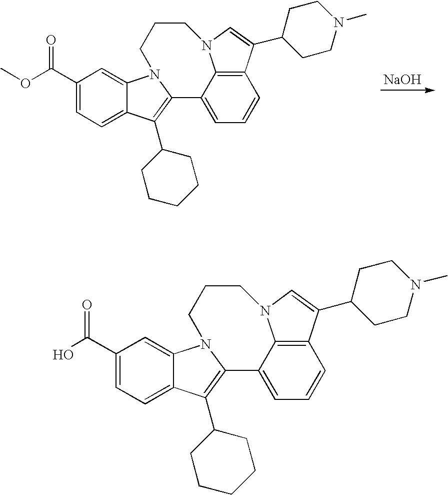

The above ester (245 mg, 0.481 mmol, 1 equiv) in THF/MeOH (2:1, 8 mL) was treated with aqueous NaOH (4 N, 1.20 mL, 4.8 mmol, 10 equiv) and heated to 80° C. for 1 h. After cooling to RT, the reaction mixture was concentrated. The crude product was taken in H₂O and acidified to pH=7 with 1 N aqueous HCl giving a precipitate. The precipitate was collected by centrifuging and purified by RP-HPLC. After concentrating to dryness, the pure solid was dissolved in ACN (2 mL) and charged with 2N HCl/EE (4 mL) to provide the HCl salt which was dried by lyophilizing to yield Compound 399 (100 mg). MS: 496.3 (M+H⁺); ¹H-NMR (DMSO-d₆): δ (ppm) 12.5 (bs, 1H), 9.28 (bs, 1H), 8.07 (s, 1H), 7.86 (d, 1H, J=8.7 Hz), 7.76 (d, 1H, J=8.7 Hz), 7.63-7.59 (m, 1H), 7.18 (s, 1H), 7.16 (t, 1H, J=7.2 Hz), 7.05 (d, 1H, J=7.2 Hz), 4.62-4.50 (m, 1H), 4.07-3.99 (m, 1H), 3.55-2.98 (m, 6H), 2.82-2.69 (m, 4H), 2.16-1.02 (m, 17H).

### Example 137

### Preparation of Compound 400

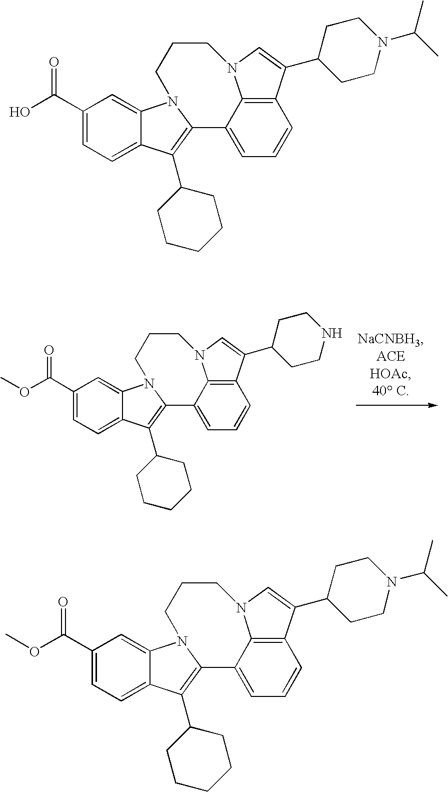

To a solution of the above amine (162 mg, 0.327 mmol, 1 equiv) in MeOH (5 mL) was added acetone (72 uL, 0.981 mmol, 3 equiv), HOAc (113 ul, 1.96 mmol, 6 equiv), and NaCNBH₃ (62 mg, 0.981 mmol, 3 equiv), and the reaction was heated at 40° C. for 3 d. HPLC showed a mixture of Reactant:Product=34:66. Then the reaction mixture was quenched with ice-water and SLOWLY added dropwise sat-bicarb. The reaction mixture was diluted with EtOAc. The layers were separated. The organic layer was washed with sat-bicarb 1×. The organic layer was dried with Na₂SO₄, filtered, concentrated, and dried in vacuo to give crude isopropyl amine (191 mg mixture of Reactant:Product=34:66) as a yellow brown solid that was used in the next step without further purification. MS: 538.3 (M+H⁺).

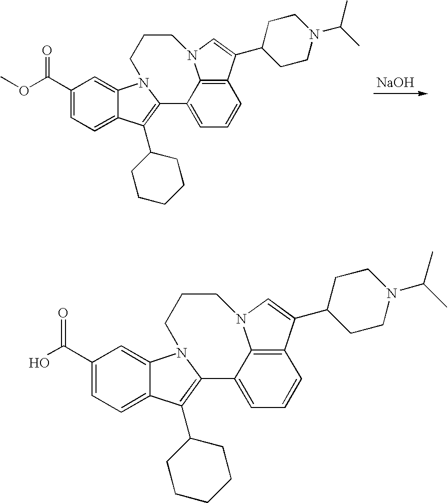

The above ester (191 mg, 0.355 mmol, 1 equiv) in THF/MeOH (2:1, 6 mL) was treated with aqueous NaOH (4 N, 887 uL, 3.55 mmol, 10 equiv) and heated to 80° C. for 1 h. After cooling to RT, the reaction mixture was concentrated. The crude product was taken in H₂O and acidified to pH=7 with 1 N aqueous HCl giving a precipitate. The precipitate was collected by centrifuging and purified by RP-HPLC. After concentrating to dryness, the pure solid was dissolved in ACN (2 mL) and charged with 2N HCl/EE (4 mL) to provide the HCl salt which was dried by lyophilizing to yield Compound 400 (40 mg). MS: 524.3 (M+H⁺); ¹H-NMR (DMSO-d₆): δ (ppm) 10.7 (bs, 1H), 8.12 (d, 1H, J=1.2 Hz), 7.91 (d, 1H, J=8.7 Hz), 7.84 (d, 1H, J=8.7 Hz), 7.67 (dd, 1H, J=8.7 Hz, J=1.2 Hz), 7.21-7.05 (m, 3H), 4.62-4.54 (m, 1H), 4.11-4.02 (m, 1H), 3.60-3.38 (m, 4H), 3.32-3.05 (m, 3H), 2.82 (bs, 1H), 2.26-1.09 (m, 23H).

### Example 138

### Preparation of Compound 401

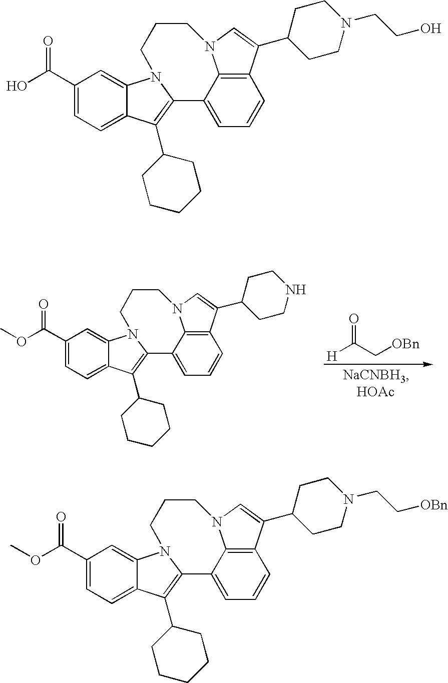

To a solution of the above ester (215 mg, 0.434 mmol, 1 equiv) in MeOH (7 mL) was added benzyloxyacetaldehyde (183 uL, 1.30 mmol, 3 equiv), HOAc (150 ul, 2.60 mmol, 6 equiv), and NaCNBH₃ (82 mg, 1.30 mmol, 3 equiv), and the reaction was stirred at RT for 1 h. Then the reaction mixture was quenched with ice-water and slowly added dropwise sat-bicarb. The reaction mixture was diluted with EtOAc. The layers were separated. The organic layer was washed with sat-bicarb 1×. The organic layer was dried with Na₂SO₄, filtered, concentrated, and dried in vacuo to give the benzylether (303 mg, 90%) as a yellow syrup that was used in the next step without further purification. MS: 630.4 (M+H⁺), 8.15 (d, 1H, J=0.9 Hz), 7.95 (d, 1H, J=8.7 Hz), 7.72-7.67 (m, 2H), 7.38-7.24 (m, 5H), 7.18-7.07 (m, 3H), 4.60-4.45 (m, 3H), 3.88 (s, 3H), 3.60-3.49 (m, 7H), 3.21-2.56 (m, 4H), 2.22-2.14 (m, 1H), 2.04-1.05 (m, 17H).

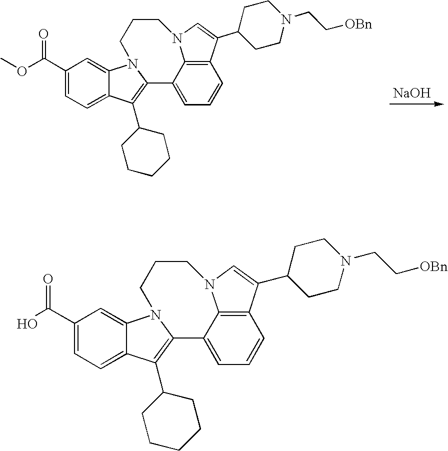

The above ester (303 mg, 0.481 mmol, 1 equiv) in THF/MeOH (2:1, 8 mL) was treated with aqueous NaOH (4 N, 1.20 uL, 4.81 mmol, 10 equiv) and heated to 80° C. for 1 h. After cooling to RT, the reaction mixture was concentrated. The crude product was taken in H₂O and acidified to pH=7 with 1 N aqueous HCl giving a precipitate. The precipitate was collected by centrifuging and dried by lyophilizing to yield the corresponding acid (287 mg, 97%) as a pale brown solid that was used in the next step without further purification. MS: 616.3 (M+H⁺); ¹H-NMR (DMSO-d₆): δ (ppm) 8.11 (s, 1H), 7.91 (d, 1H, J=8.4 Hz), 7.71-7.63 (m, 2H), 7.35-7.04 (m, 8H), 4.60-4.45 (m, 3H), 4.07-3.98 (m, 1H), 3.61-3.41 (m, 4H), 3.21-3.09 (m, 4H), 2.85-2.60 (m, 1H), 2.69-2.60 (m, 1H), 2.32-2.20 (m, 1H), 2.04-1.05 (m, 17H).

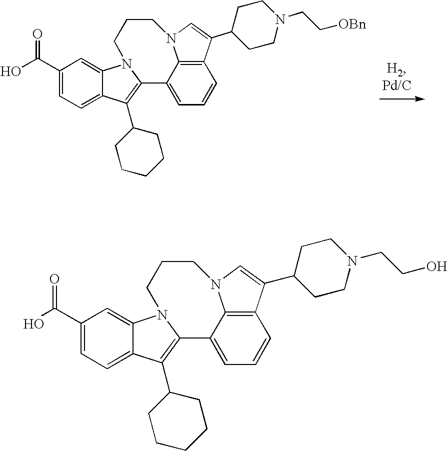

The above benzylether (185 mg, 0.300 mmol, 1 equiv) in MeOH (10 mL) and HOAc (5 mL) was charged with 10% Pd/C (65 mg, 20 mol %) and hydrogenated at 50 psi for ON. HPLC showed a mixture of Reactant:Product=11:89. The catalyst was filtered off using a celite pad and rinsed with MeOH. The crude product was purified by RP-HPLC. After concentrating to dryness, the pure solid was dissolved in ACN (2 mL) and charged with 2N HCl/EE (4 mL) to provide the HCl salt which was dried by lyophilizing to yield Compound 401 (50 mg). MS: 526.3 (M+H⁺); ¹H-NMR (DMSO-d₆): δ (ppm) 9.81 (bs, 1H), 8.07 (d, 1H, J=1.2 Hz), 7.86 (d, 1H, J=8.7 Hz), 7.80 (d, 1H, J=6.9 Hz), 7.86 (dd, 1H, J=8.1 Hz, J=1.2 Hz), 7.15-7.04 (m, 3H), 4.57-4.50 (m, 1H), 4.07-3.98 (m, 1H), 3.77-3.41 (m, 5H), 3.18-3.01 (m, 6H), 2.85-2.70 (m, 1H), 2.14-1.01 (m, 17H).

### Example 139

### Preparation of Compound 402

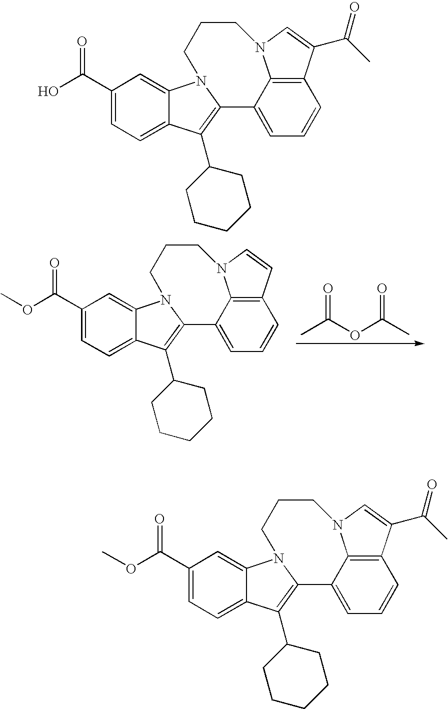

A solution of the above ester (300 mg, 0.727 mmol, 1 equiv) in HOAc (6 mL) and Ac₂O (6 mL) was treated with H₃PO₄ (85%, 150 uL, 2.18 mmol, 3 equiv) and heated to 80° C. for 1 h. After cooling to RT, the reaction mixture was concentrated and dried in vacuo to give the above ketone (328 mg, 99%) as a dark purple solid that was used in the next step without further purification. MS: 455.2 (M+H⁺); ¹H-NMR (DMSO-d₆): δ (ppm) 8.44 (s, 1H), 8.59 (dd, 1H, J=8.1 Hz, J=1.2 Hz), 8.20 (d, 1H, J=1.2 Hz), 7.97 (d, 1H, J=8.4 Hz), 7.71 (dd, 1H, J=8.1 Hz, J=1.2 Hz), 7.41 (t, 1H, J=7.2 Hz), 7.24-7.21 (m, 1H), 4.71-4.64 (m, 1H), 4.24-4.20 (m, 1H), 3.88 (s, 3H), 3.68-3.60 (m, 1H), 3.27-3.20 (m, 1H), 2.88-2.72 (m, 2H), 2.13-1.13 (m, 15H).

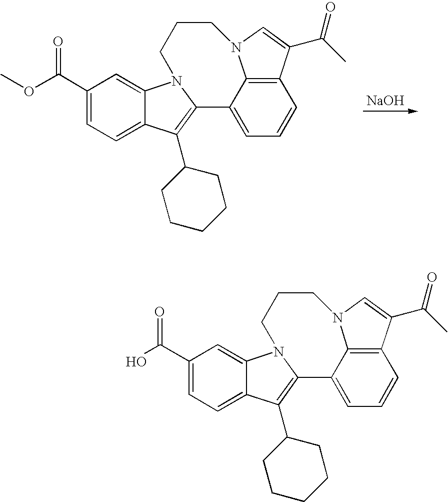

The above ester (65 mg, 0.143 mmol, 1 equiv) in THF/MeOH (2:1, 3 mL) was treated with aqueous NaOH (4 N, 357 uL, 1.43 mmol, 10 equiv) and heated to 80° C. for 1.5 h. After cooling to RT, the reaction mixture was concentrated. The crude product was taken in H₂O and acidified to pH=7 with 1 N aqueous HCl giving a precipitate. The precipitate was collected by centrifuging, purified by RP-HPLC, and dried by lyophilizing to yield Compound 402 (21 mg). MS: 441.2 (M+H⁺); ¹H-NMR (DMSO-d₆): δ (ppm) 8.37 (s, 1H), 8.32 (dd, 1H, J=8.1 Hz, J=1.2 Hz), 8.10 (d, 1H, J=1.2 Hz), 7.87 (d, 1H, J=8.4 Hz), 7.64 (dd, 1H, J=8.1 Hz, J=1.2 Hz), 7.35 (t, 1H, J=7.2 Hz), 7.17 (dd, 1H, J=7.2 Hz, J=0.9 Hz), 4.62-4.56 (m, 1H), 4.20-4.02 (m, 1H), 3.60-3.50 (m, 1H), 3.27-3.10 (m, 1H), 2.78-2.70 (m, 1H), 2.40 (s, 3H), 2.13-0.987 (m, 12H).

### Example 140

### Preparation of Compound 403

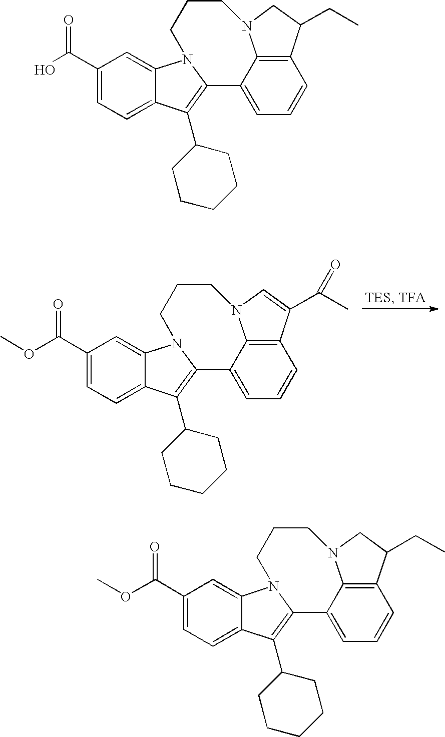

The above ketone (120 mg, 0.264 mmol, 1 equiv) in TFA (5 mL) was charged with TES (253 uL, 1.58 mmol, 6 equiv) and stirred at RT for 3 d. The reaction mixture was concentrated and dried in vacuo to give crude product (125 mg, 108%) as a dark purple semisolid that was used in the next step without further purification. MS: 443.3 (M+H⁺); ¹H-NMR (DMSO-d₆): δ (ppm) 8.44 (s, 1H), 8.59 (dd, 1H, J=8.1 Hz, J=1.2 Hz), 8.05 (s, 1H), 7.82 (d, 1H, J=8.4 Hz), 7.61 (d, 1H, J=8.1 Hz), 7.01 (d, 1H, J=6 Hz), 6.82 (d, 1H, J=7.8 Hz), 6.60 (t, 1H, J=7.8 Hz), 4.55-4.44 (m, 1H), 3.83-3.50 (m, 4H), 3.53-3.30 (m, 2H), 3.28-2.70 (m, 4H), 1.97-0.989 (m, 17H).

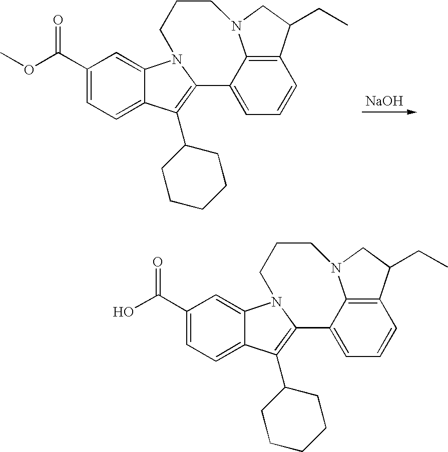

The above ester (120 mg, 0.271 mmol, 1 equiv) in THF/MeOH (2:1, 4.5 mL) was treated with aqueous NaOH (4 N, 678 uL, 2.71 mmol, 10 equiv) and heated to 80° C. for 3 h. After cooling to RT, the reaction mixture was concentrated. The crude product was taken in H₂O and acidified to pH=7 with 1 N aqueous HCl giving a precipitate. The precipitate was collected by centrifuging, purified by RP-HPLC, and dried by lyophilizing to yield Compound 403 (26 mg). MS: 429.2 (M+H⁺); ¹H-NMR (DMSO-d₆): δ (ppm)) 8.03 (s, 1H), 7.81 (d, 1H, J=8.1 Hz), 7.61 (d, 1H, J=8.1 Hz), 7.06 (d, 1H, J=7 Hz), 6.81 (d, 1H, J=7.5 Hz), 6.60 (t, 1H, J=7.5 Hz), 4.55-4.44 (m, 1H), 3.85-3.48 (m, 2H), 3.33-3.10 (m, 2H), 2.90-2.70 (m, 3H), 2.10-1.20 (m, 14H), 0.955 (t, 3H, J=7.5 Hz).

The following compounds were similarly prepared according to the Examples described herein.

### Example 141

### Preparation of Compound 378

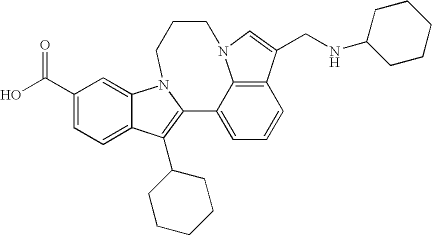

MS: 411.2 (M-C₆H₁₃N+H⁺); ¹H-NMR (DMSO-d₆): δ (ppm) 12.6 (s, 1H), 8.09 (bs, 2H), 8.06 (d, 1H, J=0.9 Hz), 7.84 (d, 2H, J=8.4 Hz), 7.81 (d, 1H, J=8.4 Hz), 7.62 (dd, 1H, J=8.7 Hz, J=1.5 Hz), 7.52 (s, 1H), 7.25 (t, 1H, J=7.2 Hz), 7.11 (d, 1H, J=6.9 Hz), 4.61-4.53 (m, 1H), 4.27-4.08 (m, 3H), 3.55-3.46 (m, 1H), 3.37-3.04 (m, 2H), 2.79 (bs, 1H), 2.13-0.996 (m, 22H).

### Example 142

### Preparation of Compound 379

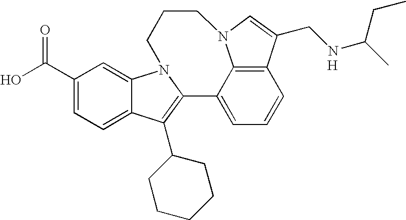

MS: 411.2 (M-C₄H₁₁N+H⁺); ¹H-NMR (DMSO-d₆): δ (ppm) 12.6 (s, 1H), 8.79-8.60 (m, 2H), 8.06 (bs, 1H), 7.87 (d, 2H, J=8.4 Hz), 7.84 (d, 1H, J=8.4 Hz), 7.62 (dd, 1H, J=8.7 Hz, J=1.5 Hz), 7.55 (s, 1H), 7.25 (t, 1H, J=7.2 Hz), 7.11 (d, 1H, J=6.9 Hz), 4.61-4.54 (m, 1H), 4.27-4.09 (m, 3H), 3.54-3.46 (m, 1H), 3.19-3.11 (m, 2H), 2.75 (bs, 1H), 2.03-0.840 (m, 20H).

### Example 143

### Preparation of Compound 380

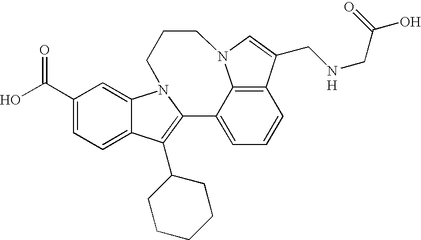

MS: 411.2 (M-C₂H₅NO₂+H⁺); ¹H-NMR (DMSO-d₆): δ (ppm) 12.6 (s, 1H), 8.06 (bs, 1H), 7.85-7.83 (m, 2H), 7.61 (dd, 1H, J=8.4 Hz, J=1.2 Hz), 7.50 (s, 1H), 7.24 (t, 1H, J=7.8 Hz), 7.10 (d, 1H, J=7.2 Hz), 4.59-4.54 (m, 1H), 4.30-4.09 (m, 3H), 3.75 (s, 2H), 3.55-3.11 (m, 3H), 2.76 (bs, 1H), 2.03-0.960 (m, 12H).

### Example 144

### Preparation of Compound 381

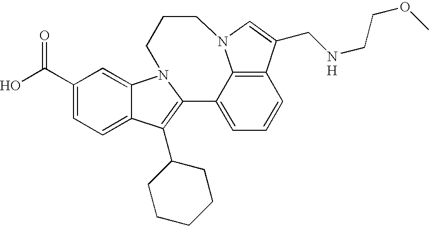

MS: 411.2 (M-C₃H₉NO+H⁺); ¹H-NMR (DMSO-d₆): δ (ppm) 12.6 (s, 1H), 8.87 (bs, 2H), 8.06 (d, 2H, J=1.2 Hz), 7.87 (d, 1H, J=5.4 Hz), 7.84 (d, 1H, J=5.7 Hz), 7.62 (dd, 1H, J=8.1 Hz, J=1.2 Hz), 7.53 (s, 1H), 7.25 (t, 1H, J=7.8 Hz), 7.11 (d, 1H, J=6.6 Hz), 4.60-4.54 (m, 1H), 4.27 (bs, 2H), 4.13-4.08 (m, 1H), 3.54-3.46 (m, 4H), 3.24 (s, 3H), 3.13-3.07 (m, 2H), 2.75 (bs, 1H), 2.03-0.956 (m, 12H).

### Example 145

### Preparation of Compound 382

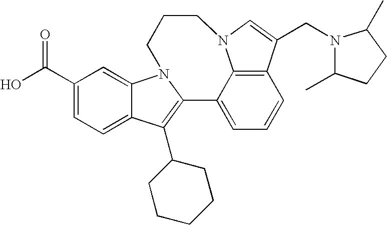

MS: 510.3 (M+H⁺); ¹H-NMR (DMSO-d₆): δ (ppm) 9.70-9.60 (m, 1H), 8.06 (bs, 1H), 7.87-7.79 (m, 2H), 7.65 (s, 1H), 7.62 (dd, 1H, J=8.7 Hz, J=1.5 Hz), 7.27-7.09 (m, 1H), 7.11 (d, 1H, J=6.9 Hz), 4.61-4.10 (m, 4H), 3.82-3.16 (m, 4H), 2.77 (bs, 1H), 2.23-0.947 (m, 22H).

### Example 146

### Preparation of Compound 383

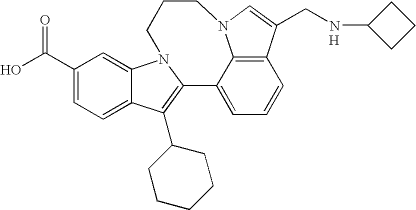

MS: 411.2 (M-C₄H₉N+H⁺); ¹H-NMR (DMSO-d₆): δ (ppm) 12.6 (s, 1H), 9.10 (bs, 2H), 8.07 (d, 1H, J=1.2 Hz), 7.85 (d, 1H, J=8.4 Hz), 7.62 (dd, 1H, J=8.7 Hz, J=1.5 Hz), 7.53 (s, 1H), 7.25 (t, 1H, J=7.8 Hz), 7.11 (d, 1H, J=6.6 Hz), 4.60-4.54 (m, 1H), 4.14-4.09 (m, 2H), 3.74-3.46 (m, 2H), 3.28 (s, 1H), 3.17-3.07 (m, 1H), 2.75 (bs, 1H), 2.13-0.956 (m, 18H).

### Example 147

### Preparation of Compound 384

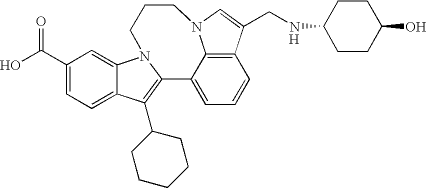

MS: 411.2 (M-C₆H₁₃NO+H⁺); ¹H-NMR (DMSO-d₆): δ (ppm) 8.71 (bs, 2H), 8.06 (d, 1H, J=0.9 Hz), 7.85 (d, 2H, J=8.7 Hz), 7.61 (dd, 1H, J=8.7 Hz, J=1.5 Hz), 7.53 (s, 1H), 7.25 (t, 1H, J=7.5 Hz), 7.11 (d, 1H, J=6.9 Hz), 4.60-4.54 (m, 1H), 4.29-4.07 (m, 3H), 3.54-3.26 (m, 2H), 3.19-2.97 (m, 2H), 2.75 (bs, 1H), 2.13-0.956 (m, 20H).

### Example 148

### Preparation of Compound 385

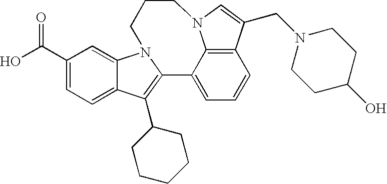

MS: 512.3 (M+H⁺); ¹H-NMR (DMSO-d₆): δ (ppm) 10.2 (bs, 1H), 8.07 (s, 1H), 7.95-7.82 (m, 2H), 7.61 (d, 2H, J=8.7 Hz), 7.25 (t, 1H, J=7.5 Hz), 7.10 (d, 1H, J=6.9 Hz), 4.60-4.54 (m, 1H), 4.41-4.35 (m, 2H), 4.12-4.07 (m, 1H), 3.85 (bs, 1H), 3.57-3.00 (m, 6H), 3.99-2.72 (m, 2H), 2.10-1.03 (m, 16H).

### Example 149

### Preparation of Compound 386

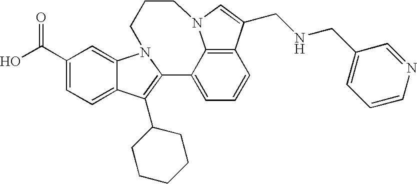

MS: 519.3 (M+H⁺); ¹H-NMR (DMSO-d₆): δ (ppm) 9.52 (bs, 2H), 8.80 (s, 1H), 8.65 (d, 1H, J=4.8 Hz), 8.21 (bs, 1H), 8.07 (s, 1H), 7.88 (t, 1H, J=8.1 Hz), 7.61-7.56 (m, 3H), 7.25 (t, 1H, J=7.2 Hz), 7.10 (d, 1H, J=6.6 Hz), 4.60-4.54 (m, 1H), 4.36-4.25 (m, 4H), 4.15-4.02 (m, 1H), 3.55-3.4 (m, 1H), 3.20-3.11 (m, 1H), 2.84-2.72 (m, 1H), 2.10-1.03 (m, 12H).

### Example 150

### Preparation of Compound 387

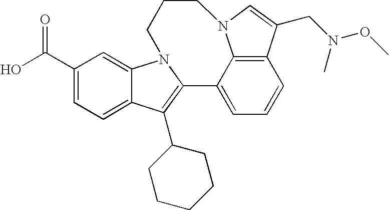

MS: 411.2 (M-C₂H₇NO+H⁺); ¹H-NMR (DMSO-d₆): δ (ppm) 8.12 (s, 1H), 7.91 (d, 2H, J=8.4 Hz), 7.83 (d, 2H, J=7.8 Hz), 7.68 (dd, 1H, J=8.4 Hz, J=1.5 Hz), 7.43 (bs, 1H), 7.24 (t, 1H, J=7.2 Hz), 7.12 (d, 1H, J=7.2 Hz), 4.66-4.55 (m, 1H), 4.29-4.07 (m, 2H), 3.58 (bs, 5H), 3.27-3.16 (m, 1H), 2.89-2.60 (m, 4H), 2.10-1.09 (m, 12H).

### Example 151

### Preparation of Compound 388

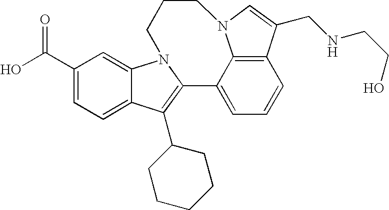

MS: 411.2 (M-C₂H₇NO+H⁺); ¹H-NMR (DMSO-d₆): δ (ppm) 12.6 (s, 1H), 8.74 (bs, 2H), 8.06 (d, 2H, J=1.2 Hz), 7.87 (d, 1H, J=5.4 Hz), 7.84 (d, 1H, J=5.7 Hz), 7.61 (dd, 1H, J=8.1 Hz, J=1.2 Hz), 7.52 (s, 1H), 7.25 (t, 1H, J=7.8 Hz), 7.10 (d, 1H, J=6.6 Hz), 5.17 (bs, 1H), 4.61-4.52 (m, 1H), 4.30 (bs, 2H), 4.15-4.08 (m, 1H), 3.64-3.46 (m, 3H), 3.31-3.08 (m, 2H), 3.00-2.91 (m, 2H), 2.75 (bs, 1H), 2.03-0.956 (m, 12H).

### Example 152

### Preparation of Compound 389

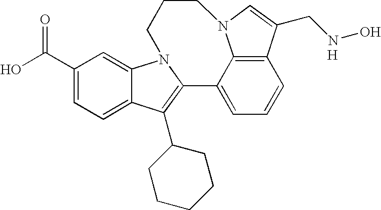

MS: 411.2 (M-CH₅NO+H⁺); ¹H-NMR (DMSO-d₆): δ (ppm) 11.2 (bs, 1H), 10.8 (bs, 1H), 8.06 (s, 1H), 7.85 (t, 2H, J=7.8 Hz), 7.61 (dd, 1H, J=8.4 Hz, J=1.2 Hz), 7.51 (bs, 1H), 7.24 (t, 1H, J=7.2 Hz), 7.12 (d, 1H, J=7.2 Hz), 4.60-4.45 (m, 2H), 4.19-4.07 (m, 1H), 3.50-3.48 (m, 3H), 3.17-3.11 (m, 1H), 2.75 (bs, 1H), 2.05-1.04 (m, 12H).

### Example 153

### Preparation of Compound 390

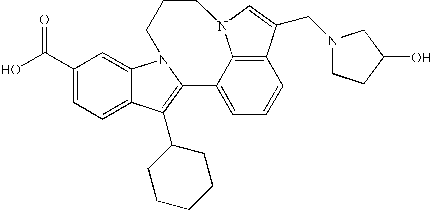

MS: 498.3 (M+H⁺); ¹H-NMR (DMSO-d₆): δ (ppm) 8.14 (s, 1H), 7.98 (d, 1H, J=7.8 Hz), 7.93 (d, 1H, J=8.4 Hz), 7.68-7.65 (m, 2H), 7.35 (t, 1H, J=9 Hz), 7.18 (d, 1H, J=8.1 Hz), 4.70-4.54 (m, 3H), 4.44-4.37 (m, 1H), 4.22-4.13 (m, 1H), 3.59-3.11 (m, 7H), 2.82 (bs, 1H), 2.13-0.956 (m, 14H).

### Example 154

### Preparation of Compound 391

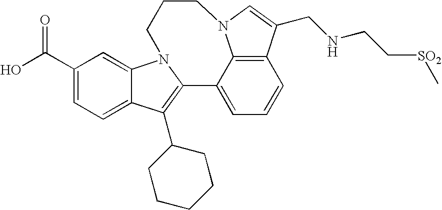

MS: 411.2 (M-C₃H₉NO₂S+H⁺); ¹H-NMR (DMSO-d₆): δ (ppm) 12.6 (bs, 1H), 9.08 (bs, 2H), 8.06 (d, 1H, J=1.2 Hz), 7.91 (dd, 1H, J=7.2 Hz, J=0.9 Hz), 7.85 (d, 1H, J=8.4 Hz), 7.62 (dd, 1H, J=8.1 Hz, J=1.2 Hz), 7.55 (s, 1H), 7.26 (t, 1H, J=7.2 Hz), 7.11 (d, 1H, J=6.9 Hz), 4.60-4.53 (m, 1H), 4.37 (bs, 2H), 4.17-4.08 (m, 1H), 3.55-3.26 (m, 6H), 3.08 (s, 3H), 2.75 (bs, 1H), 2.03-0.961 (m, 12H).

### Example 155

### Preparation of Compound 392

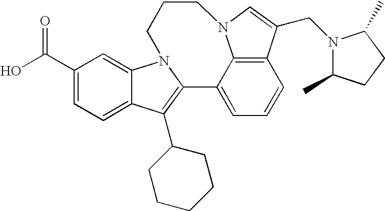

MS: 510.3 (M+H⁺); ¹H-NMR (DMSO-d₆): δ (ppm) 9.84-9.67 (m, 1H), 8.06 (bs, 1H), 7.87-7.82 (m, 2H), 7.74 (d, 1H, J=9.9 Hz), 7.61 (dd, 1H, J=8.7 Hz, J=1.2 Hz), 7.27 (t, 1H, J=7.8 Hz), 7.11 (d, 1H, J=7.2 Hz), 4.61-4.28 (m, 3H), 4.12-4.02 (m, 1H), 3.92-3.40 (m, 3H), 3.25-3.10 (m, 1H), 2.77 (bs, 1H), 2.31-2.22 (m, 1H), 2.03-0.947 (m, 22H).

### Example 156

### Preparation of Compound 393

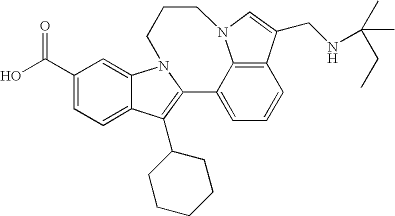

MS: 411.2 (M-C₅H₁₃N+H⁺); ¹H-NMR (DMSO-d₆): δ (ppm), 8.66 (bs, 2H), 8.13 (s, 1H), 7.92 (d, 1H, J=8.4 Hz), 7.87 (d, 1H, J=7.8 Hz), 7.69 (dd, 1H, J=8.4 Hz, J=1.2 Hz), 7.62 (s, 1H), 7.33 (t, 1H, J=7.2 Hz), 7.18 (d, 1H, J=7.2 Hz), 4.70-4.61 (m, 1H), 4.31-4.13 (m, 3H), 3.65-3.52 (m, 1H), 3.28-3.13 (m, 1H), 2.81 (bs, 1H), 2.09-0.935 (m, 23H).

### Example 157

### Preparation of Compound 394

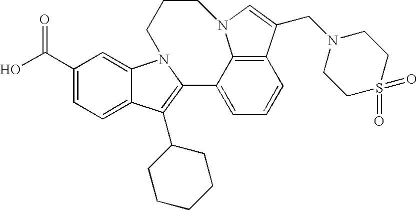

MS: 411.2 (M-C₄H₉NO₂S+H⁺); ¹H-NMR (DMSO-d₆): δ (ppm), 8.07 (bs, 1H), 7.97 (d, 1H, J=7.8 Hz), 7.86 (d, 1H, J=8.4 Hz), 7.61 (dd, 1H, J=8.4 Hz, J=1.2 Hz), 7.27 (t, 1H, J=7.5 Hz), 7.18 (d, 1H, J=7.5 Hz), 4.60-4.53 (m, 3H), 4.15-4.03 (m, 1H), 3.75-3.42 (m, 9H), 3.28-3.13 (m, 1H), 2.75 (bs, 1H), 2.09-0.937 (m, 12H).

### Example 160

### Preparation of Compound 329

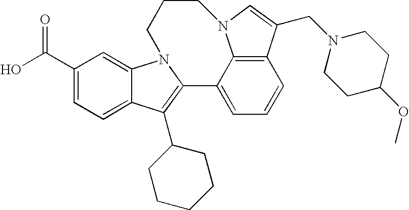

MS (M+H⁺): 526.3; H¹-NMR (DMSO d₆): δ (ppm) 9.87 (br s, 1H), 8.13 (s, 1H), 8.0 (d, 1H, J=7.2 Hz), 7.90 (d, 1H, J=8.8 Hz), 7.65 (d, 1H, J=12.4 Hz), 7.64 (s, 1H), 7.30 (t, 1H, J=7.4 Hz), 7.15 (d, 1H, J=7.2 Hz), 4.63 (d, 1H, J=10.2 Hz), 4.48 (s, 3H), 4.18 (d, 1H, J=14.3 Hz), 3.60-3.45 (m, 2H), 3.45-3.30 (m, 2H), 3.23 (d, 2H, J=4.7 Hz), 3.12-3.0 (m, 2H), 2.81 (br s, 2H), 2.20-1.10 (m, 16H).

### Example 161

### Preparation of Compound 330

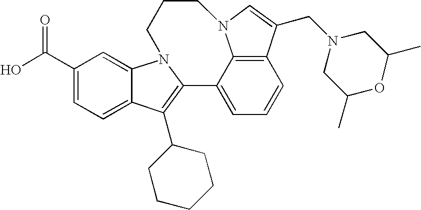

MS (M+H⁺): 526.3; H¹-NMR (DMSO d₆): δ (ppm) 10.50 (br s, 1H), 10.23 (br s, 1H), 8.14 (s, 1H), 7.97 (d, 1H, J=7.4 Hz), 7.90 (d, 1H, J=8.3 Hz), 7.67-7.64 (m, 2H), 7.30 (t, 1H, J=7.7 Hz), 7.16 (d, 1H, J=7.2 Hz), 4.64-4.61 (m, 3H), 4.25-4.16 (m, 1H), 3.92-3.83 (m, 1H), 3.65-3.51 (m, 1H), 3.50-3.40 (m, 1H), 3.28-3.20 (m, 2H), 2.88-2.65 (m, 3H), 2.14-1.86 (m, 6H), 1.75-1.64 (m, 2H), 1.58-1.50 (m, 1H), 1.42-1.30 (m, 3H), 1.15 (d, 6H, J=6.3 Hz).

### Example 162

### Preparation of Compound 331

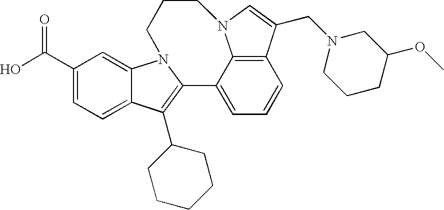

MS (M+H⁺): 526.3; H¹-NMR (DMSO d₆): δ (ppm) 9.88 (br s, 1H), 9.32 (br s, 1H), 8.13 (s, 1H), 7.96 (d, 1H, J=7.9 Hz), 7.91 (d, 1H, J=8.3 Hz), 7.68-7.60 (m, 2H), 7.30 (t, 1H, J=6.3 Hz), 7.16 (d, 1H, J=7.2 Hz), 4.68-4.64 (m, 1H), 4.54-4.42 (m, 2H), 4.24-4.18 (m, 1H), 3.72-3.58 (m, 2H), 3.54-3.40 (m, 2H), 3.32-3.26 (m, 2H), 3.12-2.65 (m, 4H), 2.14-1.08 (m, 15H) 0.95-0.8 (m, 1H).

### Example 163

### Preparation of Compound 332

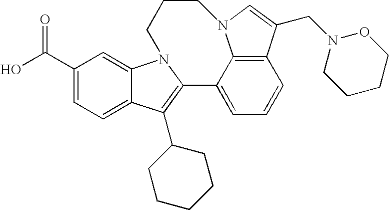

MS (M-C₄H₉NO+H⁺): 411.2; H¹-NMR (DMSO d₆): δ (ppm) 8.12 (s, 1H), 7.90 (d, 1H, J=8.3 Hz), 7.82 (d, 1H, J=8.0 Hz), 7.67-7.64 (m, 1H), 7.47 (s, 1H), 7.23 (t, 1H, J=7.7 Hz), 7.15-7.10 (m, 1H), 4.64-4.58 (m, 2H), 4.50-4.22 (m, 2H), 4.21-4.16 (m, 3H), 3.30-3.10 (m, 2H), 2.94-2.70 (m, 3H), 2.14-1.06 (m, 16H).

### Example 164

### Preparation of Compound 333

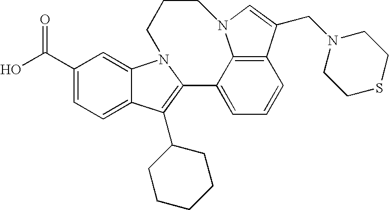

MS (M-C₄H₉SN+H⁺): 411.2; H¹-NMR (DMSO d₆): δ (ppm) 10.11 (br s, 1H), 8.14 (s, 1H), 7.98 (d, 1H, J=8.0 Hz), 7.91 (d, 1H, J=8.5 Hz), 7.68-7.64 (m, 2H), 7.31 (t, 1H, J=7.5 Hz), 7.18-7.13 (m, 1H), 4.68-4.58 (m, 1H), 4.56-4.50 (m, 2H), 4.24-4.14 (m, 1H), 3.82-3.70 (m, 2H), 3.66-3.52 (m, 1H), 3.28-3.00 (m, 3H), 2.88-2.76 (m, 3H), 2.36-0.80 (m, 14H).

### Example 165

### Preparation of Compound 334

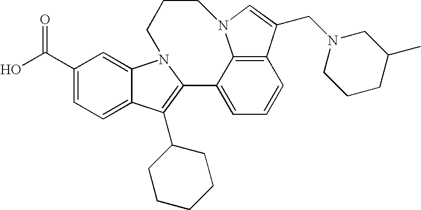

MS (M+H⁺): 510.3; H¹-NMR (DMSO d₆): δ (ppm) 9.94 (br s, 2H), 8.14 (s, 1H), 7.96 (d, 1H, J=8.0 Hz), 7.91 (d, 1H, J=8.5 Hz), 7.68-7.64 (m, 2H), 7.35-7.27 (m, 1H), 7.18-7.14 (m, 1H), 4.68-4.58 (m, 1H), 4.54-4.36 (m, 2H), 4.24-4.14 (m, 1H), 3.32-3.18 (m, 2H), 2.92-2.70 (m, 2H), 2.68-2.52 (m, 1H), 2.14-0.94 (m, 18H), 0.89 (d, 3H, J=4.7 Hz).

### Example 166

### Preparation of Compound 335

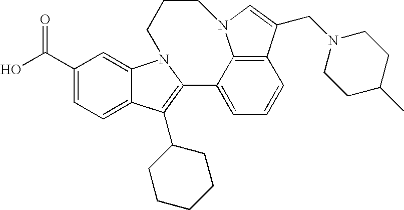

MS (M+H⁺): 510.3; H¹-NMR (DMSO d₆): δ (ppm) 9.58 (br s, 2H), 8.14 (s, 1H), 7.95 (d, 1H, J=8.0 Hz), 7.91 (d, 1H, J=8.5 Hz), 7.68-7.63 (m, 2H), 7.30 (t, 1H, J=7.7 Hz), 7.16 (d, 1H, J=7.2 Hz), 4.68-4.58 (m, 1H), 4.48-4.42 (m, 2H), 4.24-4.14 (m, 1H), 3.32-3.18 (m, 2H), 3.06-2.76 (m, 3H), 2.14-1.09 (m, 18H), 0.90 (d, 3H, J=6.3 Hz).

### Example 167

### Preparation of Compound 336

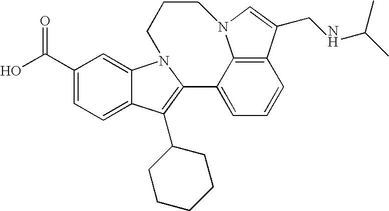

MS (M-C₃H₉N+H⁺): 411.2; H¹-NMR (DMSO d₆): δ (ppm) 8.65 (br s, 2H), 8.13 (s, 1H), 7.90 (d, 2H, J=9.35 Hz), 7.66 (d, 1H, J=8.5 Hz), 7.60 (s, 1H), 7.30 (t, 1H, J=7.7 Hz), 7.17 (d, 1H, J=7.2 Hz), 4.68-4.58 (m, 1H), 4.38-4.30 (m, 2H), 4.24-4.14 (m, 1H), 3.64-3.50 (m, 1H), 3.28-3.16 (m, 1H), 2.88-2.70 (m, 2H), 2.30-1.40 (m, 9H), 1.33 (d, 6H, J=6.3 Hz) 1.20-0.80 (m, 3H).

### Example 168

### Preparation of Compound 337

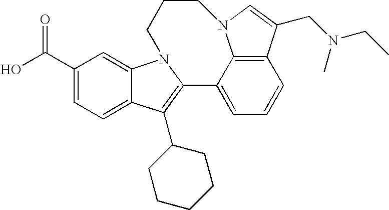

MS (M+H⁺): 470.3; H¹-NMR (DMSO d₆): δ (ppm) 9.90 (br s, 1H), 8.13 (s, 1H), 7.95 (d, 1H, J=8.3 Hz), 7.90 (d, 1H, J=8.5 Hz), 7.68-7.65 (m, 2H), 7.30 (t, 1H, J=8.0 Hz), 7.16 (d, 1H, J=6.6 Hz), 4.68-4.38 (m, 3H), 4.24-4.14 (m, 1H), 3.66-3.52 (m, 1H), 3.32-3.18 (m, 2H), 3.10-2.98 (m, 1H), 2.90-2.76 (m, 1H), 2.71 (d, 3H, J=4.1 Hz) 2.18-1.34 (m, 9H), 1.29 (t, 3H, J=7.2 Hz), 1.26-0.80 (m, 3H).

### Example 169

### Preparation of Compound 338

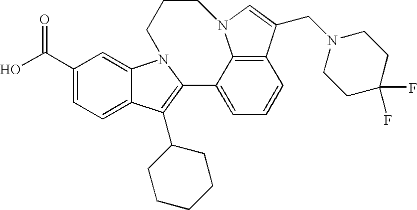

MS (M-C₅H₉F₂N+H⁺): 411.2; H¹-NMR (DMSO d₆): δ (ppm) 10.50 (br s, 1H), 8.14 (s, 1H), 8.00 (d, 1H, J=7.4 Hz), 7.91 (d, 1H, J=8.5 Hz), 7.68-7.65 (m, 2H), 7.31 (t, 1H, J=7.7 Hz), 7.16 (d, 1H, J=6.9 Hz), 4.68-4.54 (m, 3H), 4.24-4.14 (m, 1H), 3.78-3.48 (m, 3H), 3.32-3.16 (m, 3H), 2.88-2.72 (m, 1H), 2.44-1.04 (m, 16H).

### Example 170

### Preparation of Compound 233

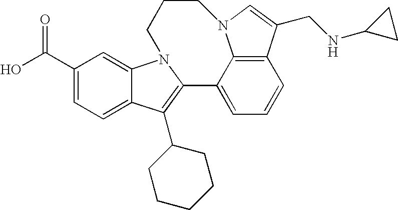

MS (M-C₃H₇N+H⁺): 411.2; H¹-NMR (DMSO d₆): δ (ppm) 9.19 (br s, 2H), 8.13 (s, 1H), 7.93 (d, 1H, J=7.9 Hz), 7.90 (d, 1H, J=8.5 Hz), 7.68-7.60 (m, 2H), 7.28 (t, 1H, J=7.7 Hz), 7.15 (d, 1H, J=6.6 Hz), 4.68-4.58 (m, 1H), 4.44-4.36 (m, 2H), 4.22-4.12 (m, 1H), 3.64-3.50 (m, 1H), 3.28-3.14 (m, 1H), 2.88-2.66 (m, 2H), 2.14-0.70 (m, 16H).

### Example 172

### Preparation of Compound 341

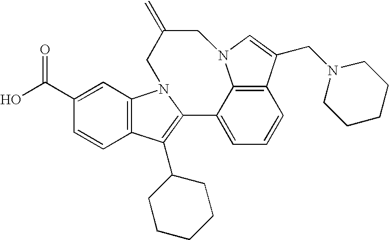

MS (M+H⁺): 508.3; H¹-NMR (DMSO d₆): δ (ppm) 9.75 (s, br, 1H), 8.02 (d, 1H, J=0.9 Hz), 7.90 (d, 1H, J=8.7 Hz), 7.83 (d, 1H, J=8.4 Hz), 7.60 (dd, 1H), J=8.7 and 1.2 Hz), 7.56 (s, 1H), 7.25 (m, 1H), 7.13 (d, 1H), 5.36 (s, 1H), 5.20 (s, 1H), 5.12 (d, 1H, J=15.9 Hz), 4.60 (d, 1H, J=15.3 Hz), 4.38 (m, 2H), 4.18 (d, 1H, J=15.9 Hz), 3.90 (d, 1H, J=15.3 Hz), 2.81 (m, 3H), 2.1-1.0 (m, 17H).

### Example 173

### Preparation of Compound 343

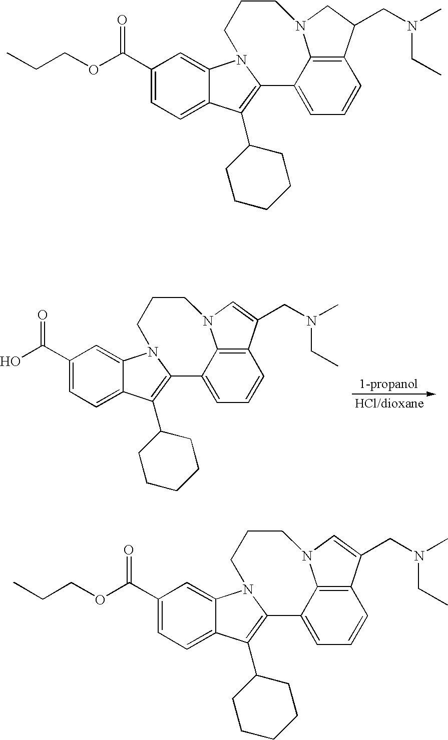

47 mg of Compound 337 (0.1 mmol) was dissolved in a mixture of 2 mL n-propanol and 0.5 mL 4M HCl/dioxane. The mixture was heated in a sealed tube at 100 deg C. for 3 hrs when it was evaporated, co-evaporated with n-propanol (1×) acetonitrile (1×) then dissolved in water and lyophilized to give Compound 337 in quantitative yield. MS (M+H⁺): 512.3; H¹-NMR (DMSO d₆): δ (ppm) 10.61 (s, br, 1H), 8.08 (d, 1H, J=1.2 Hz), 7.88 (m, 2H), 7.62 (m, 2H), 7.23 (m, 1H), 7.10 (dd, 1H), 4.56 (m, 1H), 4.38 (m, 2H), 4.18 (m, 3H) 3.52 (m, 1H), 3.17 (m, 2H), 2.96 (m, 1H), 2.75 (m, 1H), 2.63 (d, 2H), 2.0-1.0 (m, 12H, 0.93 (t, 3H, J=7.2 Hz).

### Example 174

### Preparation of Compound 344

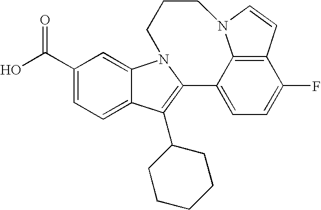

MS (M+H⁺): 417.2; H¹-NMR (DMSO d₆): δ (ppm) 12.60 (s, 1H), 8.11 (d, 1H, J=1.2 Hz), 7.89 (d, 1H, J=8.7 Hz), 7.65 (dd, 1H, J=8.4 and 1.2 Hz), 7.45 (d, 1H, J=3 Hz), 7.02 (m, 2H), 6.61 (d, 1H, J=3 Hz), 4.60 (m, 1H), 4.15 (m, 1H), 3.58 (m, 1H), 3.20 (m, 1H), 2.80 (m, 1H), 2.1-1.0 (m, 12H).

### Example 175

### Preparation of Compound 345

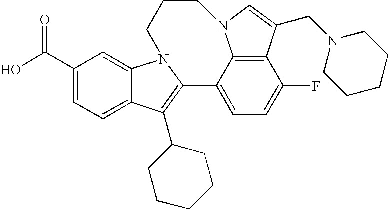

MS (M+H⁺): 514.3; H¹-NMR (DMSO d₆): δ (ppm) 9.57 (s, br, 1H), 8.14 (d, 1H, J=1.2 Hz), 7.90 (d, 1H, J=8.4 Hz), 7.65 (m, 2H), 7.11 (m, 2H), 4.63 (m, 1H), 4.45 (d, 2H, J=3.9 Hz), 4.20 (m, 1H), 3.58 (m, 1H), 3.44 (m, 2H), 3.22 (m, 1H), 2.93 (m, 1H), 2.77 (m, 1H), 2.1-1.0 (m, 16H).

### Example 176

### Preparation of Compound 346

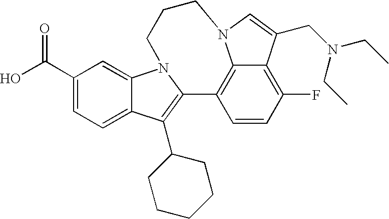

MS (M+H⁺): 502.3; H¹-NMR (DMSO d₆): δ (ppm) 9.24 (s, br, 1H), 8.08 (d, 1H, J=1.2 Hz), 7.85 (d, 1H, J=8.4 Hz), 7.66 (s, 1H), 7.61 (dd, 1H, J=8.7 and 0.9 Hz), 7.07 (m, 2H), 4.57 (m, 1H), 4.42 (m, 2H), 4.10 (m, 1H), 3.11 (m, 5H), 2.70 (m, 1H), 2.1-1.0 (m, 19H).

### Example 177

### Preparation of Compound 347

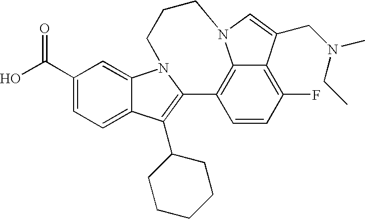

MS (M+H⁺): 488.3; H¹-NMR (DMSO d₆): δ (ppm) 9.2 (s, 1H), 8.08 (d, 1H), 7.85 (d, (1H), 7.61 (m, 2H), 7.07 (m, 2H), 4.57 (m, 2H), 4.30 (m, 1H), 4.15 (m, 1H), 3.50 (m, 1H), 3.15 (m, 1H), 3.09 (m, 1H), 2.68 (m, 4H), 2.1-1.0 (m, 14H).

### Example 178

### Preparation of Compound 348

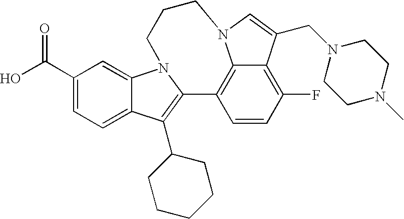

MS (M+H⁺): 529.3; H¹-NMR (DMSO d₆): δ (ppm) 8.08 (d, 1H), 7.84 (d, 1H), 7.60 (dd, 2H), 7.04 (m, 2H), 4.58 (m, 1H), 4.10 (m, 1H) 4.0-3.0 (m, br, 17H), 2.1-1.0 (m, 9H).

### Example 179

### Preparation of Compound 349

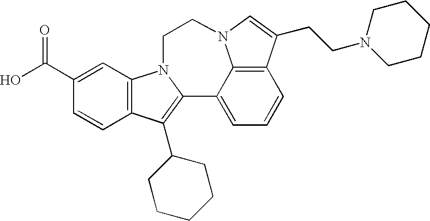

MS (M+H⁺): 496.3; H¹-NMR (DMSO d₆): δ (ppm) 8.21 (d, 1H), 7.92 (d, 1H, J=8.4 Hz), 7.76 (d, 1H) 7.63 (dd, 1H, J=8.4 and 1.2 Hz), 7.38 (s, 1H), 7.35-7.24 (m, 2H), 4.40 (br, 2H), 3.57-2.89 (m, 10H), 2.20-1.20 (m, 17H).

### Example 180

### Preparation of Compound 350

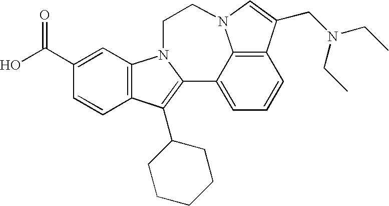

MS (M+H⁺): 470.3; H¹-NMR (DMSO d₆): δ (ppm) 8.18 (d, 1H), 7.86 (m, 2H), 7.75 (s, 1H), 7.59 (dd, 1H, J=8.1 and 1.2 Hz), 7.31 (m, 2H), 4.43 (d, 2H), 3.29 (underwater, 4H), 3.05 (m, 5H), 2.2-1.10 (m, 16H).

### Example 181

### Preparation of Compound 351

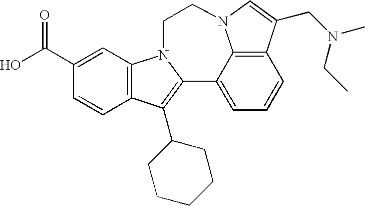

MS (M+H⁺): 456.3; H¹-NMR (DMSO d₆): δ (ppm) 8.18 (d, 1H, J=1.2 Hz), 7.88 (m, 1H), 7.70 (s, 1H), 7.59 (dd, 1H, J=8.7 and 1.5 Hz), 7.31 (m, 2H), 4.40 (m, 2H), 3.4 (br under water, 4H), 3.3-2.9 (m, 3H), 2.91 (d, 3H, J=4.8 Hz), 2.2-1.2 (m, 13H).

### Example 182

### Preparation of Compound 352

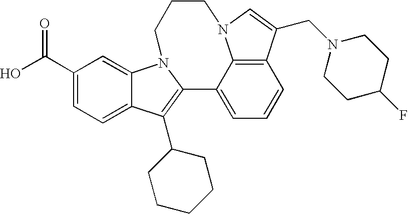

MS (M-C₅H₁₀NF+H⁺): 411.2; H¹-NMR (DMSO d₆): δ (ppm) 10.16 (br s, 1H), 8.13 (s, 1H), 8.01-7.94 (m, 1H), 7.90 (d, 1H, J=8.2 Hz), 7.67-7.64 (m, 2H), 7.29 (t, 1H, J=7.6 Hz), 7.15 (d, 1H, J=6.6 Hz), 4.98 (d, 1H, J=47.0 Hz), 4.68-4.46 (m, 3H), 4.22-4.12 (m, 2H), 3.64-3.50 (m, 2H), 3.30-3.0 (m, 3H), 2.81 (br s, 1H), 2.28-1.02 (m, 16H).

### Example 183

### Preparation of Compound 353

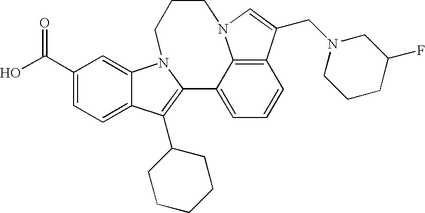

MS (M+H⁺): 514.3; H¹-NMR (DMSO d₆): δ (ppm) 10.35 (br s, 1H), 9.81 (br s, 1H), 8.13 (s, 1H), 7.95 (d, 1H, J=8.2 Hz), 7.90 (d, 1H, J=8.5 Hz), 7.67-7.62 (m, 2H), 7.30 (t, 1H, J=7.4 Hz), 7.16 (d, 1H, J=7.1 Hz), 5.10 (d, 1H, J=44.8 Hz), 4.68-4.48 (m, 3H), 4.26-4.16 (m, 2H), 3.75-3.60 (m, 2H), 3.40-2.95 (m, 3H), 2.81 (br s, 1H), 2.15-1.02 (m, 15H).

### Example 184

### Preparation of Compound 354

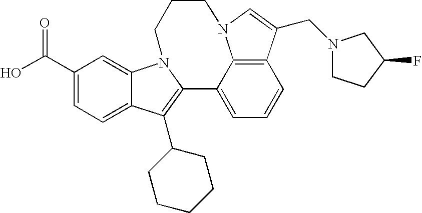

MS (M-C₄H₈NF+H⁺): 411.2; H¹-NMR (DMSO d₆): δ (ppm) 10.73 (br s, 1H), 8.13 (s, 1H), 7.97 (d, 1H, J=7.9 Hz), 7.90 (d, 1H, J=8.5 Hz), 7.71-7.64 (m, 2H), 7.31 (t, 1H, J=7.4 Hz), 7.16 (d, 1H, J=6.6 Hz), 5.43 (d, 1H, J=53.6 Hz), 4.68-4.54 (m, 2H), 4.22-4.12 (m, 1H), 3.90-3.70 (m, 2H), 3.65-3.48 (m, 2H), 3.30-3.18 (m, 2H), 2.81 (m, 2H), 2.20-1.00 (m, 14H).

### Example 186

### Preparation of Compound 356

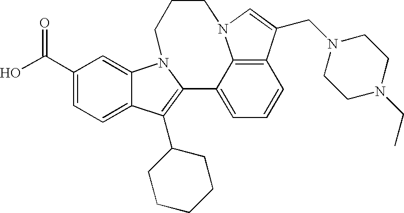

MS (M+H⁺): 525.3; H¹-NMR (DMSO d₆): δ (ppm) 8.09 (s, 1H), 7.96-7.90 (m, 1H), 7.68-7.63 (d, 1H J=7.5 Hz), 7.63-7.60 (m, 2H), 7.24 (t, 1H, J=6.4 Hz), 7.12-7.07 (m, 1H), 4.58-4.54 (m, 3H), 4.13-4.09 (m, 2H), 3.62-3.46 (m, 3H), 3.40-3.26 (m, 3H), 3.24-3.0 (m, 3H), 2.82-2.70 (m, 2H), 3.12-3.02 (m, 1H), 2.0-1.04 (m, 15H).

### Example 187

### Preparation of Compound 357

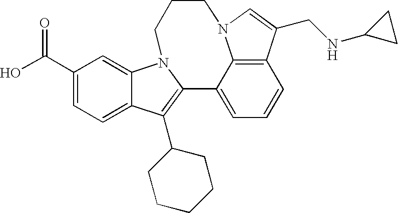

MS (M-C₃H₆N+H⁺): 411.2; H¹-NMR (DMSO d₆): δ (ppm) 9.05 (br s, 2H), 8.12 (s, 1H), 7.93-7.88 (m, 2H), 7.66 (d, 1H J=8.5 Hz), 7.59 (s, 1H), 7.28 (t, 1H, J=7.4 Hz), 7.15 (d, 1H, J=6.6 Hz), 4.68-4.58 (m, 1H), 4.46-4.38 (m, 2H), 4.22-4.12 (m, 1H), 3.64-3.50 (m, 1H), 3.28-3.14 (m, 1H), 2.88-2.70 (m, 2H), 2.12-1.04 (m, 12H), 0.94-0.74 (m, 4H).

### Example 188

### Preparation of Compound 358

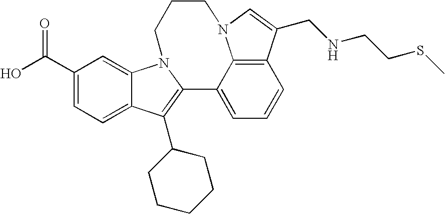

MS (M-C₃H₉NS+H⁺): 411.2; H¹-NMR (DMSO d₆): δ (ppm) 8.80 (br s, 2H), 8.13 (s, 1H), 7.95-7.89 (m, 2H), 7.66 (d, 1H J=8.5 Hz), 7.59 (s, 1H), 7.30 (t, 1H, J=7.4 Hz), 7.16 (d, 1H, J=6.3 Hz), 4.68-4.60 (m, 1H), 4.42-4.36 (m, 2H), 4.26-4.16 (m, 1H), 3.64-3.50 (m, 1H), 3.28-3.16 (m, 3H), 2.88-2.72 (m, 3H), 2.14-1.04 (m, 15H).

### Example 189

### Preparation of Compound 359

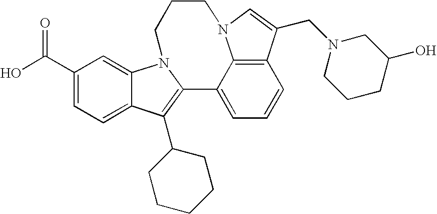

MS (M+H⁺): 512.3; H¹-NMR (DMSO d₆): δ (ppm) 10.37 (br s, 1H), 9.33 (br s, 1H), 8.11 (s, 1H), 7.95 (d, 1H, J=8.2 Hz), 7.88 (d, 1H J=8.5 Hz), 7.65-7.61 (m, 2H), 7.27 (t, 1H, J=7.4 Hz), 7.13 (d, 1H, J=7.1 Hz), 4.66-4.56 (m, 1H), 4.50-4.38 (m, 2H), 4.24-4.12 (m, 1H), 4.04-3.78 (br s, 1H), 3.84-3.72 (m, 1H), 3.64-3.50 (m, 1H), 3.44-3.16 (m, 2H), 3.14-2.92 (m, 1H), 2.88-2.74 (br s, 1H), 2.66-2.52 (m, 1H), 2.12-1.02 (m, 16H).

### Example 190

### Preparation of Compound 360

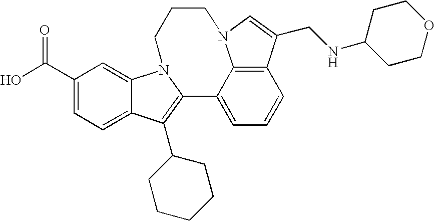

MS (M-C₅H₁₁NO+H⁺): 411.2; H¹-NMR (DMSO d₆): δ (ppm) 9.03 (br s, 2H), 8.11 (s, 1H), 7.91-7.86 (m, 2H), 7.64 (d, 1H J=8.2 Hz), 7.61 (s, 1H), 7.27 (t, 1H, J=7.4 Hz), 7.14 (d, 1H, J=6.8 Hz), 4.66-4.58 (m, 1H), 4.34 (br s, 2H), 4.20-4.12 (m, 1H), 3.96-3.88 (m, 2H), 3.62-3.48 (m, 1H), 3.40-3.30 (m, 2H), 3.32-3.14 (m, 2H), 2.79 (br s, 1H), 2.14-1.0 (m, 16H).

### Example 191

### Preparation of Compound 361

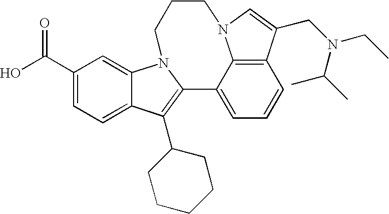

MS (M+H⁺): 498.3; H¹-NMR (DMSO d₆): δ (ppm) 8.96 (br s, 1H), 8.14 (s, 1H), 7.91 (d, 1H, J=8.5 Hz), 7.85 (d, 1H, J=7.9 Hz), 7.70-7.65 (m, 2H), 7.32 (t, 1H, J=7.4 Hz), 7.17 (d, 1H, J=7.1 Hz), 4.70-4.52 (m, 2H), 4.42-4.32 (m, 1H), 4.24-4.14 (m, 1H), 3.72-3.50 (m, 2H), 3.32-3.12 (m, 3H), 2.81 (br s, 1H), 2.12-1.50 (m, 10H), 1.45-1.42 (m, 3H), 1.33 (d, 6H, J=6.32), 1.30-1.24 (m, 2H).

### Example 192

### Preparation of Compound 362

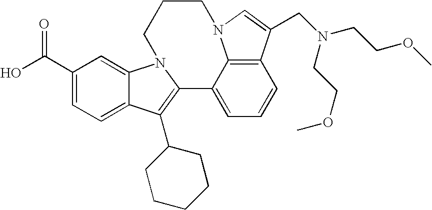

MS (M+H⁺): 544.3; H¹-NMR (DMSO d₆): δ (ppm) 9.55 (br s, 1H), 8.13 (s, 1H), 7.91-7.88 (m, 2H), 7.67-7.64 (m, 2H), 7.31 (t, 1H, J=7.4 Hz), 7.16 (d, 1H, J=6.8 Hz), 4.68-4.56 (m, 3H), 4.24-4.16 (m, 1H), 3.78-3.72 (m, 3H), 3.62-3.50 (m, 2H), 3.46-3.36 (m, 3H), 3.32 (d, 6H, J=2.2 Hz), 3.32-3.18 (m, 2H), 2.80 (br s, 1H), 2.07-1.06 (m, 12H).

### Example 193

### Preparation of Compound 363

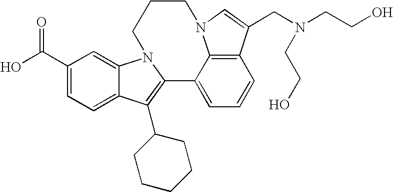

MS (M+H⁺): 516.3; H¹-NMR (DMSO d₆): δ (ppm) 9.60 (br s, 1H), 8.13 (s, 1H), 7.94 (d, 1H, J=7.7 Hz), 7.90 (d, 1H, J=8.8 Hz), 7.73-7.64 (m, 2H), 7.30 (t, 1H, J=7.4 Hz), 7.16 (d, 1H, J=7.1 Hz), 4.68-4.58 (m, 3H), 4.24-4.14 (m, 1H), 3.88-3.82 (m, 4H), 3.66-3.38 (m, 4H), 3.32-3.20 (m, 4H), 2.82 (br s, 1H), 2.12-1.06 (m, 12H).

### Example 194

### Preparation of Compound 364

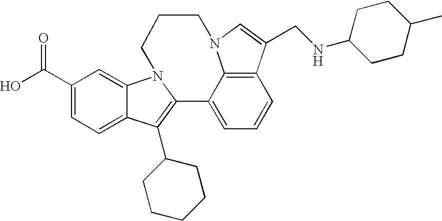

MS (M-C₇H₁₅N+H⁺): 411.2; H¹-NMR (DMSO d₆): δ (ppm) 8.70 (br s, 2H), 8.13 (s, 1H), 7.91-7.88 (m, 2H), 7.68-7.58 (m, 2H), 7.29 (t, 1H, J=7.4 Hz), 7.16 (d, 1H, J=6.8 Hz), 4.67-4.60 (m, 1H), 4.35 (br s, 2H), 4.20-4.16 (m, 1H), 3.62-3.50 (m, 2H), 3.28-3.16 (m, 2H), 2.81 (br s, 1H), 2.22-0.98 (m, 18H), 0.94 (d, 3H, J=6.8 Hz), 0.92-0.82 (m, 2H).

### Example 195

### Preparation of Compound 365

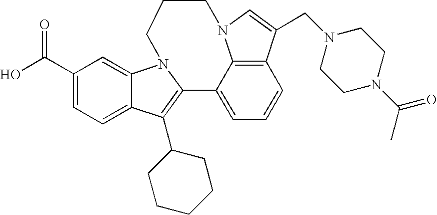

MS (M-C₆H₁₂N₂O+H⁺): 411.2; H¹-NMR (DMSO d₆): δ (ppm) 10.82 (br s, 1H), 8.13 (s, 1H), 7.98 (d, 1H, J=7.9 Hz), 7.90 (d, 1H, J=8.5 Hz), 7.67-7.64 (m, 2H), 7.29 (t, 1H, J=7.4 Hz), 7.15 (d, 1H, J=6.8 Hz), 4.68-4.58 (m, 1H), 4.54-4.42 (m, 3H), 4.26-3.94 (m, 2H), 3.64-3.36 (m, 4H), 3.32-3.18 (m, 1H), 3.14-2.92 (m, 3H), 2.82 (br s, 1H), 2.06 (s, 3H), 1.94-1.02 (m, 12H).

### Example 196

### Preparation of Compound 366

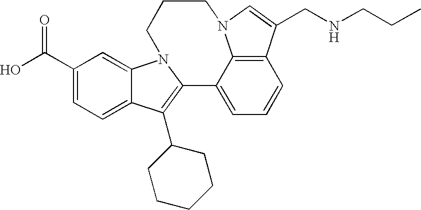

MS (M-C₃H₉N+H⁺): 411.2; H¹-NMR (DMSO d₆): δ (ppm) 8.80 (br s, 1H), 8.13 (s, 1H), 7.93-7.88 (m, 2H), 7.66 (d, 1H, J=8.2 Hz), 7.59 (s, 1H), 7.28 (t, 1H, J=7.4 Hz), 7.15 (d, 1H, J=6.6 Hz), 4.66-4.60 (m, 1H), 4.38-4.28 (m, 2H), 4.22-4.14 (m, 1H), 3.64-3.44 (m, 2H), 3.28-3.16 (m, 1H), 2.98-2.72 (m, 3H), 2.10-1.04 (m, 14H), 0.92 (t, 3H, J=7.4 Hz).

### Example 197

### Preparation of Compound 367

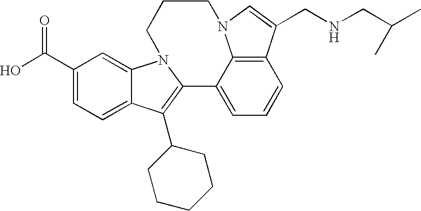

MS (M-C₄H₁₁N+H⁺): 411.2; H¹-NMR (DMSO d₆): δ (ppm) 8.65 (br s, 2H), 8.11 (s, 1H), 7.90 (d, 1H, J=6.0 Hz), 7.88 (d, 1H, J=6.6 Hz), 7.65-7.62 (m, 1H), 7.58 (s, 1H), 7.27 (t, 1H, J=7.4 Hz), 7.13 (d, 1H, J=6.8 Hz), 4.66-4.56 (m, 1H), 4.31 (br s, 2H), 4.22-4.12 (m, 1H), 3.62-3.48 (m, 1H), 3.26-3.14 (m, 2H), 2.88-2.68 (m, 3H), 2.08-1.02 (m, 12H), 0.93 (d, 6H, J=6.8 Hz).

### Example 198

### Preparation of Compound 368

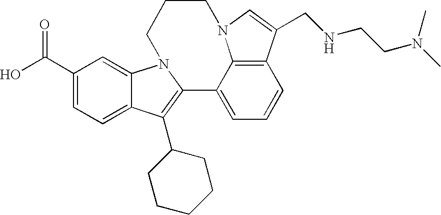

MS (M+H⁺): 499.3; H¹-NMR (DMSO d₆): δ (ppm) 10.95 (br s, 1H), 9.49 (br s, 2H), 8.13 (s, 1H), 8.00 (d, 1H, J=7.1 Hz), 7.90 (d, 1H, J=8.5 Hz), 7.67-7.65 (m, 2H), 7.40 (s, 1H), 7.31-7.24 (m, 2H), 7.16 (d, 1H, J=6.6 Hz), 7.07 (s, 1H), 4.68-4.58 (m, 1H), 4.41 (br s, 2H), 4.22-4.12 (m, 1H), 3.64-3.42 (m, 2H), 3.28-3.14 (m, 1H), 2.83 (s, 6H), 2.14-1.02 (m, 12H).

### Example 199

### Preparation of Compound 369

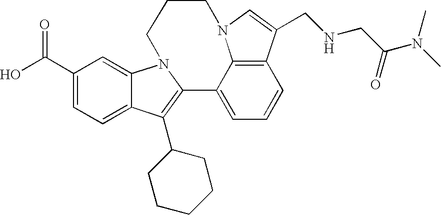

MS (M-C₄H₁₀N₂O+H⁺): 411.2; H¹-NMR (DMSO d₆): δ (ppm) 8.99 (br s, 2H), 8.13 (s, 1H), 7.92-7.89 (m, 2H), 7.66 (d, 1H, J=8.2 Hz), 7.57 (s, 1H), 7.29 (t, 1H, J=7.4 Hz), 7.16 (d, 1H, J=6.8 Hz), 4.68-4.58 (m, 1H), 4.40-4.30 (m, 2H), 4.24-4.14 (m, 1H), 4.08-4.02 (m, 2H), 3.64-3.52 (m, 1H), 3.28-3.16 (m, 1H), 2.90 (d, 6H, J=5.2 Hz), 2.86-2.76 (m, 1H), 2.12-1.04 (m, 12H).

### Example 200

### Preparation of Compound 370

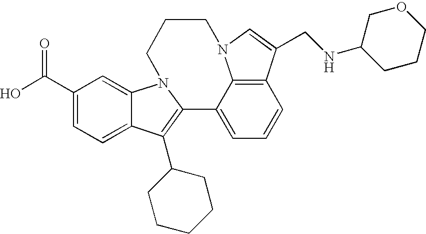

MS (M-C₅H₁₁NO+H⁺): 411.2; H¹-NMR (DMSO d₆): δ (ppm) 9.18 (br s, 2H), 8.15 (s, 1H), 7.96 (d, 1H, J=7.9 Hz), 7.92 (d, 1H, J=8.5 Hz), 7.70-7.66 (m, 2H), 7.30 (t, 1H, J=7.3 Hz), 7.17 (d, 1H, J=7.3 Hz), 4.70-4.60 (m, 1H), 4.38 (br s, 2H), 4.22-4.14 (m, 1H), 4.06-3.98 (m, 1H), 3.74-3.68 (m, 1H), 3.64-3.56 (m, 2H), 3.48-3.38 (m, 1H), 3.28-3.18 (m, 2H), 2.82 (br s, 1H), 2.24-1.04 (m, 16H).

### Example 201

### Preparation of Compound 371

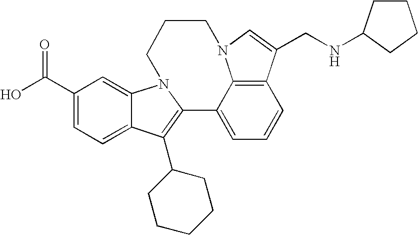

MS (M-C₅H₁₁N+H⁺): 411.2; H¹-NMR (DMSO d₆): δ (ppm) 8.82 (br s, 2H), 8.13 (s, 1H), 7.90 (d, 2H, J=8.2 Hz), 7.68-7.64 (m, 1H), 7.60 (s, 1H), 7.29 (t, 1H, J=7.4 Hz), 7.16 (d, 1H, J=6.8 Hz), 4.68-4.60 (m, 1H), 4.33 (br s, 2H), 4.24-4.14 (m, 1H), 3.64-3.52 (m, 2H), 3.28-3.16 (m, 1H), 2.81 (br s, 1H), 2.12-1.04 (m, 20H).

### Example 202

### Preparation of Compound 372

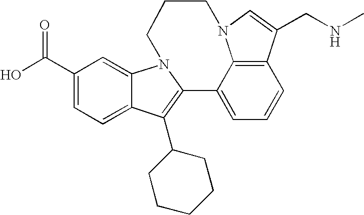

MS (M-CH₃N+H⁺): 411.2; H¹-NMR (DMSO d₆): δ (ppm) 8.69 (br s, 2H), 8.12 (s, 1H), 7.94-7.86 (m, 2H), 7.68-7.64 (m, 1H), 7.55 (s, 1H), 7.27 (t, 1H, J=7.4 Hz), 7.15 (d, 1H, J=6.6 Hz), 4.68-4.58 (m, 1H), 4.31 (br s, 2H), 4.22-4.12 (m, 1H), 3.62-3.50 (m, 1H), 3.28-3.14 (m, 1H), 2.80 (br s, 1H), 2.58 (s, 3H), 2.12-1.02 (m, 12H).

### Example 203

### Preparation of Compound 373

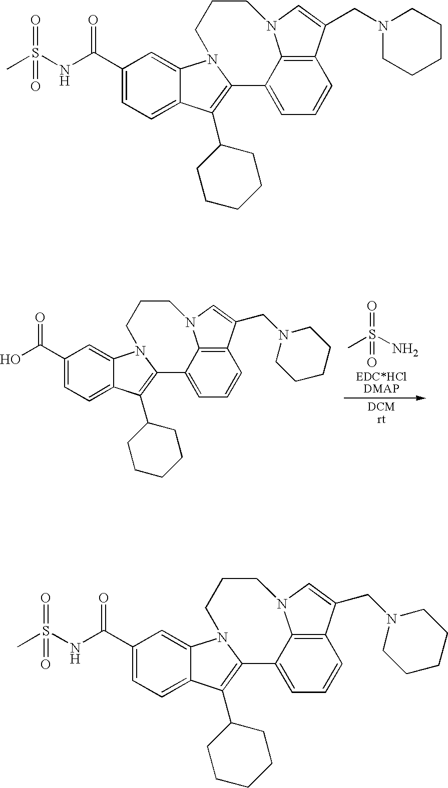

To a solution of Compound 105 (75 mg, 0.150 mmol) in 3 mL DCM, 4-(Dimethylamino)pyridine (27.5 mg, 0.225 mmole) and EDC hydrochloride (43.3 mg, 0.225 mmole) were added. The reaction was stirred at room temperature for 10 minutes. After 10 minutes, Methanesulfonamide (42.9 mg, 0.451 mmole) was added and the reaction was stirred at room temperature over night. The crude product was concentrated and re-dissolved in DMF (8 mL). Purification by HPLC gave 39.3 mg of the title compound. MS (M+H⁺): 573.3; H¹-NMR (DMSO d₆): δ (ppm) 11.96 (s, 1H), 9.99 (br s, 1H), 8.29 (s, 1H), 7.97 (d, 1H, J=7.9 Hz), 7.93 (d, 1H, J=8.5 Hz), 7.68-7.62 (m, 2H), 7.30 (t, 1H, J=7.4 Hz), 7.16 (d, 1H, J=6.8 Hz), 4.64-4.52 (m, 1H), 4.46 (br s, 2H), 4.26-3.84 (m, 4H), 3.70-3.58 (m, 1H), 3.52-3.38 (m, 1H), 3.30-3.18 (m, 1H), 2.98-2.78 (m, 3H), 2.26-1.04 (m, 18H).

### Example 204

### Preparation of Compound 374

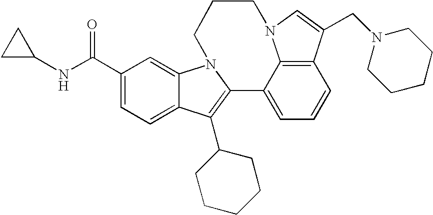

This compound was prepared as described in Example 203 on a 0.100 mmole scale, using Cyclopropylamine. Yield: 41.8 mg; MS (M+H⁺): 535.3; H¹-NMR (DMSO d₆): δ (ppm) 10.20 (br s, 1H), 8.40 (s, 1H), 8.07 (s, 1H), 7.95 (d, 1H, J=7.4 Hz), 7.83 (d, 1H, J=8.5 Hz), 7.66 (s, 1H), 7.57 (d, 1H, J=8.5 Hz), 7.28 (t, 1H, J=7.7 Hz), 7.14 (d, 1H, J=6.8 Hz), 4.58-4.50 (m, 1H), 4.46 (br s, 2H), 4.22-4.12 (m, 1H), 3.68-3.54 (m, 1H), 3.48-3.36 (m, 2H), 3.32-3.18 (m, 1H), 2.98-2.78 (m, 3H), 2.18-1.04 (m, 18H), 0.76-0.58 (m, 4H).

### Example 205

### Preparation of Compound 375

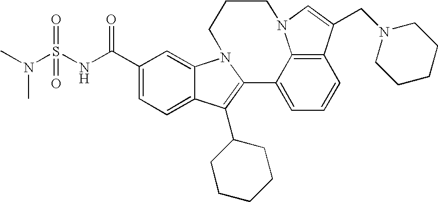

This compound was prepared as described in Example 203 0.150 mmole scale, using N,N-Dimethylsulfamide. Yield: 42.9 mg; MS (M+H⁺): 602.3; H¹-NMR (DMSO d₆): δ (ppm) 11.66 (s, 1H), 9.88 (br s, 1H), 8.29 (s, 1H), 7.97 (d, 1H, J=7.9 Hz), 7.92 (d, 1H, J=8.8 Hz), 7.68-7.62 (m, 2H), 7.30 (t, 1H, J=7.4 Hz), 7.16 (d, 1H, J=6.6 Hz), 4.62-4.52 (m, 1H), 4.48-4.40 (m, 2H), 4.26-4.16 (m, 1H), 4.00-3.74 (m, 1H), 3.68-3.56 (m, 1H), 3.52-3.38 (m, 2H), 3.30-3.18 (m, 1H), 3.02-3.76 (m, 7H), 2.28-1.04 (m, 18H).

### Example 206

### Preparation of Compound 376

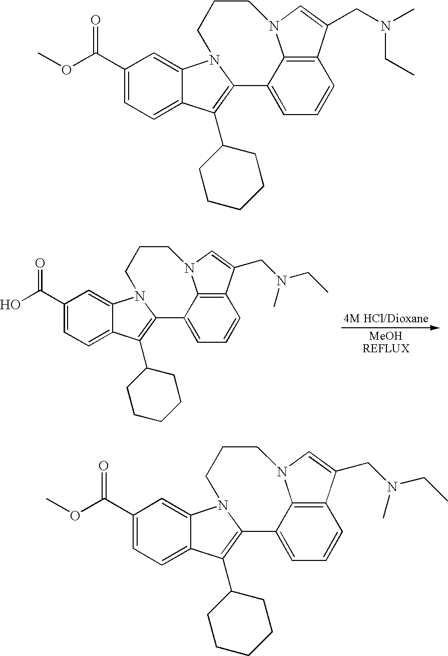

To a solution of Compound 337 (50 mg, 0.106 mmol) in 350 μL Methanol, 4N HCl in Dioxane (37.2 L, 0.148 mmole). The reaction was refluxed overnight at 70° C. The crude product was concentrated and re-dissolved in DMF (8 mL). Purification by HPLC gave 30 mg of the title compound. MS (M+H⁺): 484.3; H¹-NMR (DMSO d₆): δ (ppm) 9.44 (br s, 1H), 8.16 (s, 1H), 7.94 (d, 2H, J=8.2 Hz), 7.69-7.65 (m, 2H), 7.31 (t, 1H, J=7.6 Hz), 7.17 (d, 1H, J=6.7 Hz), 4.70-4.62 (m, 1H), 4.60-4.50 (m, 1H), 4.48-4.38 (m, 1H), 4.24-4.14 (m, 1H), 3.86 (s, 3H), 3.32-3.18 (m, 2H), 3.10-3.00 (m, 1H), 2.88-2.76 (m, 1H), 2.72 (d, 3H, J=4.9 Hz), 2.12-1.32 (m, 10H), 1.27 (t, 3H, J=7.3 Hz), 1.18-1.02 (m, 2H).

### Example 207

### Preparation of Compound 377

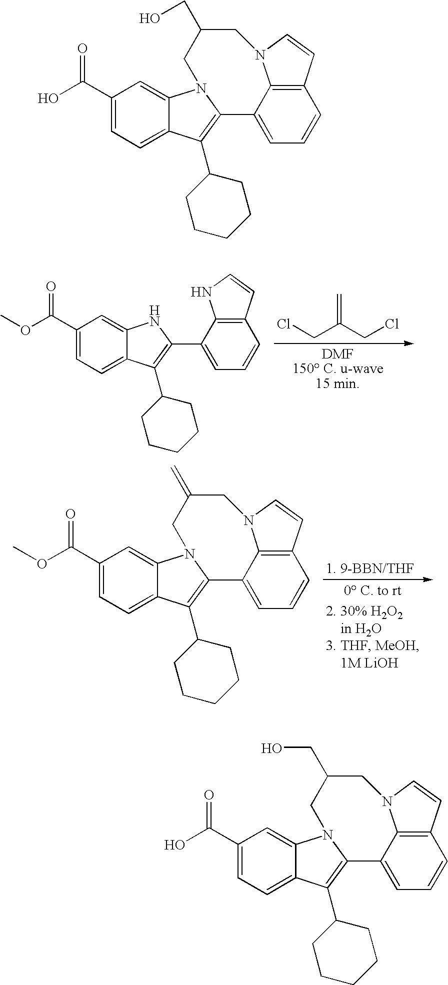

The above ester (100 mg, 0.268 mmol), 3-chloro-2-chloromethyl-1-propene (34 μL, 0.322 mmol, 1.2 eq), and Potassium Carbonate (111 mg, 0.805 mmol, 3 eq) were dissolved in DMF (2.7 mL). The reaction was run in a 5 mL vial in a microwave synthesis unit at 150° C. for 15 minutes. The resulting crude was concentrated and precipitated with H₂O to receive 110 mg of the desired alkene as a yellow solid. MS (M+H⁺): 425.2; H¹-NMR (DMSO d₆): δ (ppm) 8.09 (s, 1H), 7.92 (d, 1H, J=8.4 Hz), 7.71-7.65 (m, 2H), 7.36 (d, 1H, J=3.2 Hz), 7.19 (t, 1H, J=7.3 Hz), 7.13-7.10 (m, 1H), 6.55 (d, 1H, J=2.9 Hz), 5.39 (s, 1H), 5.21-5.14 (m, 1H), 4.64-4.56 (m, 1H), 4.26-4.18 (m, 1H), 3.85 (s, 3H), 3.29 (s, 2H), 2.82 (br s, 1H), 2.04-1.06 (m, 10H).

The above alkene (100 mg, 0.235 mmol) was dissolved in THF (1.25 mL) in a 20 mL screw cap vial with a stir bar, the temperature of the reaction was then brought down to 0° C. 9-BBN in THF (0.5M solution, 1.41 mL, 0.706 mmol, 3 eq) was added at 0° C. and the reaction was warmed slowly to room temperature and stirred overnight. The reaction was monitored by LCMS and quenched by 30% H₂O₂ in H₂O (1.06 mL, 2.35 mmole, 10 eq) at 0° C. The reaction was then warmed to room temperature and let to stir for 2 hours. The crude product was concentrated and re-dissolved in a mixture of THF (3 mL), Methanol (1 mL), and 1M LiOH (1 mL) then heated to 50° C. After 2 hours the reaction was complete by LCMS/HPLC. The crude product was concentrated and re-dissolved in DMF (8 mL). Purification by HPLC gave 26 mg of the title compound Compound 377 as a mixture of diastereomers. MS (M+H⁺): 429.2; H¹-NMR (DMSO d₆): δ (ppm) 8.24-8.13 (m, 1H), 7.88-7.83 (m, 1H), 7.69-7.62 (m, 2H), 7.41-7.34 (m, 1H), 7.20-7.13 (m, 1H), 7.09-7.00 (m, 1H), 6.55-6.53 (m, 1H), 5.20 (br s, 1H), 4.63-4.51 (m, 1H), 4.25-4.12 (m, 1H), 3.72-3.65 (m, 1H), 3.55-3.40 (m, 2H), 3.35-3.22 (m, 2H), 2.70-2.78 (m, 2H), 2.32-1.04 (m, 10H).

### Example 208

### Preparation of Compound 447

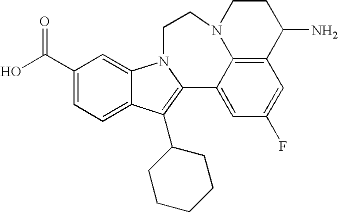

A mixture of 2-bromo-4-fluoroaniline (7.6 g, 40.0 mmol, 1.0 equiv), acrylic acid (4.3 g, 60.0 mmol, 1.5 equiv) and water (10.0 mL) was heated at 70° C. for 3 hours. The precipitate was collected by filtration to give 8.6 g. MS: 264 [M+H⁺].

To a 100 ml round bottom flask containing N-(2-bromo-4-fluorophenyl)-β-alanine (6.5 g, 24.8 mmol, 1.0 equiv) was added a solution of phosphorus pentoxide (3.87 g, 27.3 mmol, 1.1 equiv) in methanesulfonic acid (65 ml). The mixture was heated to 65° C. with stirring under nitrogen for 5 hours after which it was poured to 50 g of ice-water. The mixture then basified to pH=10 by addition of 50% NaOH aq. solution. EtOAc was added to the mixture and the phases were separated. The aqueous layer was extracted with EtOAc. The organic layers were combined, washed with brine, dried over MgSO₄ and concentrated to give product 5.2 g. The crude material was used in the next step with no further purification. MS: 246 [M+H⁺].

To a solution of 8-bromo-6-fluoro-2,3-dihydroquinolin-4(1H)-one (5.1 g, 21.0 mmol, 1.0 equiv) in MeOH (80.0 mL) was added hydroxylamine HCl salt (1.5 g, 22.1 mmol, 1.05 equiv) and pyridine (1.8 mL. 22.1 mmol, 1.05 equiv). The mixture was stirred at 65° C. for 3 hours, then at room temperature for 16 hours. The solvent was then removed under vacuum and the residue was added EtOAc. The solution was washed with sat. aq. NaHCO₃ solution, brine, dried over MgSO₄ and concentrated to give product 5.3 g. The crude material was used in the next step with no further purification. MS: 261 [M+H⁺].

To a slurry of NaBH₄ (1.5 g, 40 mmol, 4.0 equiv) in dimethoxyethane (50.0 mL) at 0° C. was slowly added TiCl₄ (2.2 mL, 20.0 mmol, 2.0 equiv) and the resultant mixture was stirred at room temperature for 1 hour. The mixture was cooled to 0° C. and added to a solution of 8-bromo-6-fluoro-N-hydroxy-2,3-dihydroquinolin-4(1H)-imine (2.6 g, 10.0 mmol, 1.0 equiv) in dimethoxyethane (20.0 mL). After stirring at room temperature for 24 hours, the solution was cooled to 0° C. and 50% NaOH aq. solution was added until pH=10. The mixture was then added to EtOAc and the layers were separated. The organic layer was washed with brine, dried over MgSO₄ and concentrated. The residue was dissolved in CH₂Cl₂ (100.0 mL), cooled to 0° C. and (Boc)₂O (4.1 g, 18.9 mmol, 2.0 equiv) was added. The solution was stirred at room temperature for 2 hours, after which the solvent was removed under vacuum. The residue was dissolved in EtOAc and the resultant solution was washed with sat. aq. NaHCO₃ solution, brine, dried over MgSO₄ and concentrated. The residue was purified by silica gel column chromatography (heptane/EtOAc, 3/1) to give product 2.1 g.

A mixture of tert-butyl (8-bromo-6-fluoro-1,2,3,4-tetrahydroquinolin-4-yl)carbamate (0.5 g, 1.5 mmol, 1.0 equiv), 4,4,4′,4′,5,5,5′,5′-octamethyl-2,2′-bi-1,3,2-dioxaborolane (1.1, 4.3 mmol, 3.0 equiv), 1,1′-bis(diphenylphosphino)ferrocenedichloro palladium(II) dichloromethane complex (32 mg, 0.043 mmol, 0.03 equiv) and potassium acetate (0.43 g, 4.4 mmol, 3.0 equiv) in dimethoxyethane (12 mL) was purged with nitrogen gas for 10 minutes in a glass tube. The tube was sealed and the mixture was stirred at 125° C. with microwave irradiation for 35 minutes and at 150° C. for 35 minutes. The mixture was then filtered through Celite and washed with heptane. The filtrate was concentrated and the residue was purified by silica gel column chromatography (heptane/EtOAc, 3/1) to give product 650 mg. MS: 393 [M+H⁺].

To a solution of tert-butyl [6-fluoro-8-(4,4,5,5-tetramethyl-1,3,2-dioxaborolan-2-yl)-1,2,3,4-tetrahydroquinolin-4-yl]carbamate (0.65 g, 1.6 mmol, 1.0 equiv) in dioxane (3.0 mL), EtOH (1.0 mL) and water (1.0 mL) was added methyl 2-bromo-1-(2-{[tert-butyl(dimethyl)silyl]oxy}ethyl)-3-cyclohexyl-1H-indole-6-carboxylate (0.63 g, 1.3 mmol, 0.78 equiv), Pd(PPh₃)₄ (0.19 g, 0.17 mmol, 0.1 equiv) and K₂CO₃ (0.69 g, 5.0 mmol, 3.0 equiv). The mixture was degassed and stirred at 105° C. for 4 hours. The mixture was filtered through Celite and washed with EtOAc. The filtrate was washed with brine, dried over MgSO₄ and concentrated. The residue was purified by silica gel column chromatography (EtOAc/heptane, 15%) to give product 370 mg. MS: 508 [M+H⁺].

To a solution of methyl 2-{4-[(tert-butoxycarbonyl)amino]-6-fluoro-1,2,3,4-tetrahydroquinolin-8-yl}-1-(2-{[tert-butyl(dimethyl)silyl]oxy}ethyl)-3-cyclohexyl-1H-indole-6-carboxylate (370 mg, 0.55 mmol, 1.0 equiv) in THF (5.0 mL) was added TBAF (1.0 M in THF, 2.7 mmol, 5.0 equiv) at 0° C. The resultant solution was stirred at room temperature for 30 minutes, after which the reaction was quenched by addition of sat. aq. NaHCO₃ solution (5.0 mL). The mixture was diluted with EtOAc and the solution was washed with sat. aq. NaHCO₃ solution, brine, dried over MgSO₄ and concentrated to give product 342 mg. MS: 566 [M+H⁺].

To a solution of Methyl 2-{4-[(tert-butoxycarbonyl)amino]-6-fluoro-1,2,3,4-tetrahydroquinolin-8-yl}-3-cyclohexyl-1-(2-hydroxyethyl)-1H-indole-6-carboxylate (340 mg, 0.6 mmol, 1.0 equiv) in CH₂Cl₂ (20 mL) at 0° C. was added triethylamine (0.25 mL, 1.8 mmol, 3.0 equiv) and methanesulfonyl chloride (0.13 mL, 1.68 mmol, 2.8 equiv). The resultant solution was stirred at 0° C. for 30 minutes, after which sat. aq. Na₂CO₃ solution was added. The phases were separated and the aqueous layer was extracted with CH₂Cl₂. The organic layers were combined, washed with brine, dried over MgSO₄ and concentrated. The residue was dissolved in CH₃CN (30 mL) and Cs₂CO₃ (0.59 g, 1.8 mmol, 3.0 equiv) was added to the solution. The resultant solution was stirred at 75° C. for 4 hours and at room temperature for 16 hours. The mixture was then filtered and the collected solid was washed with water to give product 280 mg.

To a solution of Boc-amine (0.28 g, 0.51 mmol, 1.0 equiv) in CH₂Cl₂ (5.0 mL) was added TFA (0.788 mL, 10.2 mmol, 20.0 equiv) and the resultant mixture was stirred at room temperature for 2 hours. The mixture was then concentrated under vacuum to give 240 mg of product as a TFA salt. MS: 448 [M+H⁺].

To a solution of methyl ester (100 mg, 0.22 mmol) in THF (1.5 mL), MeOH (0.5 mL) and water (0.5 mL) was added a solution of LiOH (54 mg, 2.2 mmol, 10.0 equiv) in water (0.5 mL). After stirring at 45° C. for 4 hours, the reaction mixture was concentrated under vacuum. The aqueous residue was acidified by addition of 1.0 N HCl aqueous solution until pH=5. The precipitate was collected by filtration and dried under vacuum. The crude material was recrystallized from MeOH/CH₃CN to give product 46 mg. MS: 432 [M-H⁺]. ¹H NMR (DMSO-d6): 8.17 (m, 1H), 7.86 (m, 1H), 7.61 (m, 1H), 7.45 (m, 1H), 6.90 (m, 1H), 4.70 (br, 2H), 3.94 (m, 2H), 3.00 (m, 2H), 2.76 (m, 2H), 2.18-0.95 (m, 14H).

### Example 209

### Preparation of Compound 448

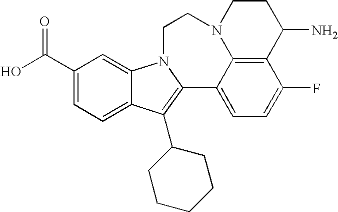

Compound 448 was prepared as described for Compound 447 using 2-bromo-5-fluoroaniline. MS: 432 [M-H⁺]. ¹H NMR (DMSO-d6): 8.15 (m, 1H), 7.83 (m, 1H), 7.61 (m, 1H), 7.15 (m, 1H), 6.92 (m, 1H), 4.68 (br, 2H), 4.15 (m, 1H), 2.93 (m, 2H), 2.72 (m, 2H), 2.13-1.06 (m, 14H).

### Example 210

### Preparation of Compound 449

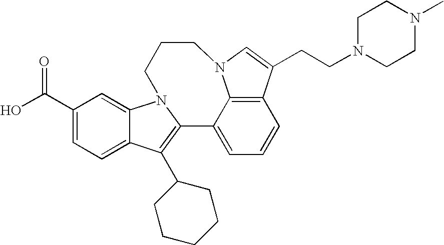

Compound 123 (75 mg, 0.136 mmol) was dissolved in THF (6 mL) and to the solution was added sodium borohydride (102.7 mg, 2.72 mmol) at room temperature. Then trifluoroacetic acid (0.2 mL) was added dropwise. The mixture was heated to reflux for 24 hours. The crude product was cooled to room temperature, concentrated in vacuo, and then diluted with EtOAc and water. Extraction and purification by HPLC gave 26 mg (36%) of Compound 449. ¹H NMR (DMSO-d₆, 300 MHz): 8.047 (s, 1H), 7.825 (d, 1H, J=8.4 Hz), 7.718 (m, 1H), 7.59 (d, 1H, J=8.1 Hz) 7.21 (s, 1H), 7.146 (t, 1H, J=7.8 Hz), 7.04 (d, 1H, J=6.9 Hz), 4.54 (m, 1H), 4.00 (m, 1H), 3.58-3.05 (m, 14H), 2.749 (m, 4H), 2.00-1.75 (m, 6H), 1.70-1.55 (m, 2H), 1.50-0.95 (m, 4H); MS (M+1): 525.3.

### Example 211

### Preparation of Compound 214

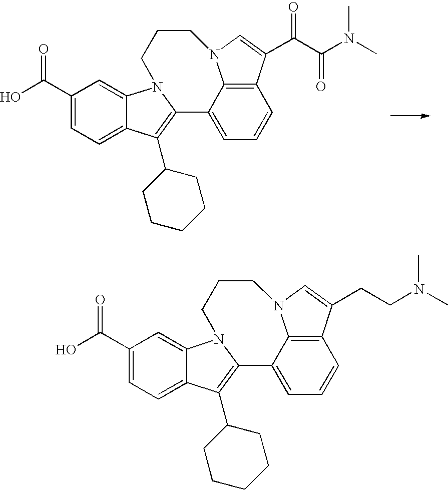

The diketoamide above (67 mg, 0.134 mmol) was dissolved in THF (6 mL) and to the solution was added sodium borohydride (101.38 mg, 2.7 mmol) at room temperature. Then trifluoroacetic acid (0.21 mL) was added dropwise. The mixture was heated to reflux for 24 hours. The crude product was cooled to room temperature, concentrated in vacuo, and then diluted with EtOAc and water. Extraction and purification by HPLC gave 26 mg (40%) of Compound 450. ¹H NMR (DMSO-d₆, 300 MHz): 9.937 (s, 1H), 8.047 (s, 1H), 7.835 (d, 1H, J=8.4 Hz), 7.712 (d, 1H, J=7.8 Hz), 7.59 (d, 1H, J=8.4 Hz) 7.23 (s, 1H), 7.154 (t, 1H, J=7.8 Hz), 7.04 (d, 1H, J=6.9 Hz), 4.53 (m, 1H), 4.02 (m, 1H), 3.58-3.05 (m, 6H), 2.749 (m, 7H), 2.00-1.75 (m, 6H), 1.70-1.6 (m, 2H), 1.50-0.95 (m, 4H); MS (M+1): 470.3.

The following compounds were similarly prepared according to the Examples described herein.

### Example 212

### Preparation of Compound 451

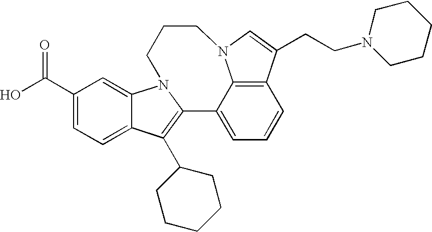

¹H NMR (DMSO-d₆, 300 MHz): 9.901 (s, 1H), 8.048 (s, 1H), 7.826 (d, 1H, J=8.4 Hz), 7.712 (d, 1H, J=7.8 Hz), 7.59 (d, 1H, J=8.4 Hz) 7.22 (s, 1H), 7.152 (t, 1H, J=7.8 Hz), 7.04 (d, 1H, J=6.9 Hz), 4.53 (m, 1H), 4.02 (m, 1H), 3.58-3.05 (m, 8H), 3.0-2.70 (m, 3H), 2.00-0.95 (m, 18H); MS (M+1): 510.3.

### Example 213

### Preparation of Compound 452

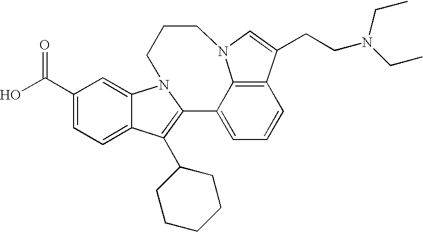

¹H NMR (DMSO-d₆, 300 MHz): 9.913 (s, 1H), 8.043 (s, 1H), 7.826 (d, 1H, J=8.4 Hz), 7.70 (d, 1H, J=7.8 Hz), 7.59 (d, 1H, J=8.4 Hz) 7.26 (s, 1H), 7.156 (t, 1H, J=7.8 Hz), 7.04 (d, 1H, J=6.9 Hz), 4.53 (m, 1H), 4.02 (m, 1H), 3.506 (m, 1H), 3.168 (m, 7H), 2.76 (m, 1H), 2.00-1.75 (m, 6H), 1.70-1.6 (m, 2H), 1.50-0.95 (m, 10H); MS (M+1): 498.3.

### Example 214

### Preparation of Compound 453

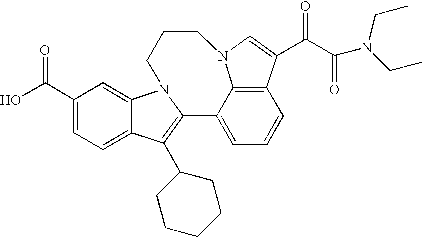

¹H NMR (DMSO-d₆, 300 MHz): 8.215 (d, 1H, J=7.8 Hz), 8.143 (s, 1H), 8.082 (s, 1H), 7.850 (d, 1H, J=8.1 Hz), 7.608 (d, 1H, J=8.7 Hz), 7.385 (t, 1H, J=7.8 Hz), 7.20 (d, 1H, J=6.9 Hz), 4.59 (m, 1H), 4.30 (m, 1H), 3.58 (m, 1H), 3.40-3.05 (m, 5H), 2.76 (m, 1H), 2.05-1.70 (m, 6H), 1.70-1.58 (m, 2H), 1.55-1.00 (m, 10H); MS (M+1): 526.3.

### Example 215

### Preparation of Compound 454

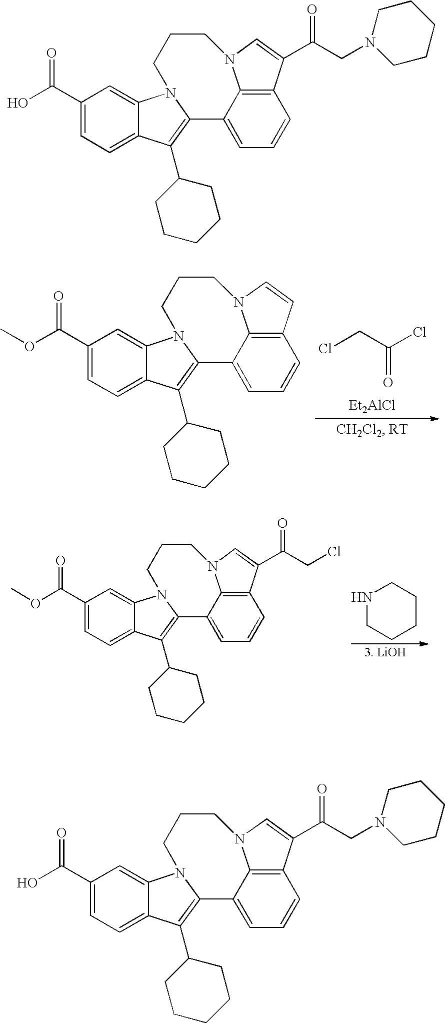

To a solution of the above indole starting material (100 mg, 0.24 mmol) in dichloromethane (10 mL) was added chloroacetyl chloride (191 μL, 2.4 mmol) and diethylaluminum chloride (1M, 1.44 mL, 1.44 mmol). The reaction was stirred at room temperature overnight. The reaction was quenched with water and extracted with CH₂Cl₂. The organic layers were concentrated to dryness and purified by HPLC to give 57 mg (48%) of the chloromethyl intermediate. MS (M+H⁺): 489.2.

To the intermediate (57 mg, 0.121 mmol) in dichloromethane (5 mL) was added piperidine

(115.4 μL, 1.2 mmol) and the reaction was stirred at room temperature for 2 hours. Then the mixture was concentrated to dryness and re-dissolved in 10 mL of mixture of methanol, THF, and water in the ratio of 1:2:1. Saponification by LiOH at 50° C. for 2 hours provided the target molecule. Purification by HPLC gave 31 mg (51%) of Compound 454. ¹H NMR (DMSO-d₆, 300 MHz): 9.75 (s, 1H), 8.494 (s, 1H), 8.28 (d, 1H, J=8.4 Hz), 8.095 (s, 1H), 7.855 (d, 1H, J=8.7 Hz), 7.624 (d, 1H, J=8.7 Hz), 7.415 (t, 1H, J=7.8 Hz), 7.22 (d, 1H, J=6.9 Hz), 4.633 (m, 3H), 4.18 (m, 1H), 3.55 (m, 1H), 3.50-2.90 (m, 5H), 2.706 (m, 1H), 2.19-0.95 (m, 18H); MS (M+1): 524.3.

### Example 216

### Preparation of Compound 455

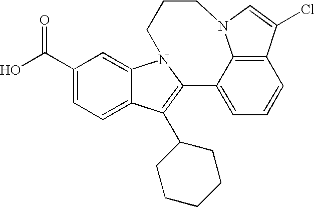

¹H NMR (DMSO-d₆, 300 MHz): δ 8.06 (d, 1H, J=1.2 Hz), 7.845 (d, 1H, J=8.4 Hz), 7.560 (m, 3H), 7.239 (t, 1H, J=7.8 Hz) 7.11 (d, 1H, J=6.9 Hz), 4.53 (m, 1H), 4.035 (m, 1H), 3.524 (m, 1H), 3.38-3.08 (m, 1H), 2.738 (m, 1H), 2.05-1.75 (m, 6H), 1.70-1.55 (m, 2H), 1.54-1.25 (m, 3H), 1.05 (m, 1H); MS (M+1): 433.2.

### Example 217

### Preparation of Compound 456

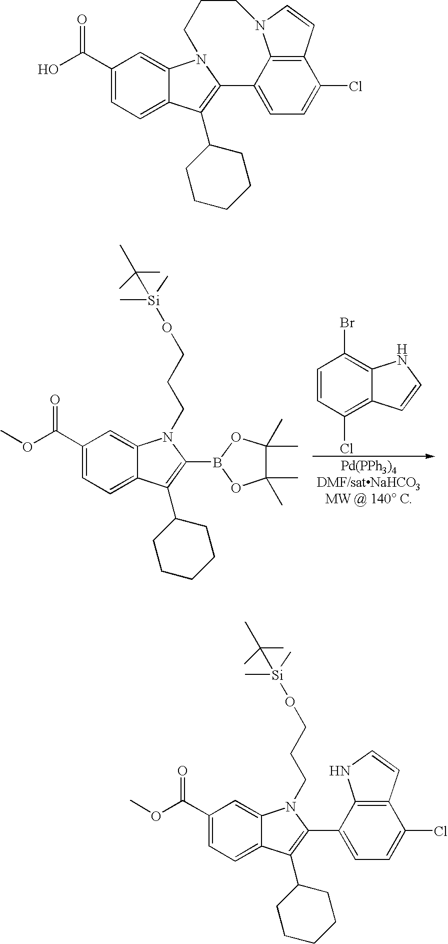

7-Bromo-4-chloro-1H-indole was prepared from 1-bromo-4-chloro-2-nitrobenzene and vinylmagnesium bromide using Bartoli indole synthesis condition (Tetrahedron Letters, 1989, Vol. 30, 2129-2132). The above borolane (1.24 g, 2.2 mmole), 7-bromo-4-chloro-1H-indole (752 mg, 3.26 mmole), Pd(PPh₃)₄ (131 mg, 0.11 mmole), and aqueous saturated sodium bicarbonate (2.2 mL) were added to 8.7 mL DMF. The mixture was degassed and reacted in microwave at 140° C. for 15 minutes. The crude product was then concentrated and purified via silica gel chromatography. Yield 700 mg (55%) MS (M+H⁺): 579.3. ¹H NMR (CDCl₃, 300 MHz): 8.337 (s, 1H), 8.24 (s, 1H), 7.933 (m, 2H), 7.359 (m, 2H), 7.16 (d, 1H, J=7.5 Hz), 6.844 (m, 1H), 4.173 (m, 1H), 4.06 (s, 3H), 4.002 (m, 1H), 3.535 (t, 1H, J=5.4 Hz), 2.60 (m, 1H), 2.09-0.95 (m, 12H), 0.888 (s, 9H), 0.02 (s, 3H), 0.00 (s, 3H).

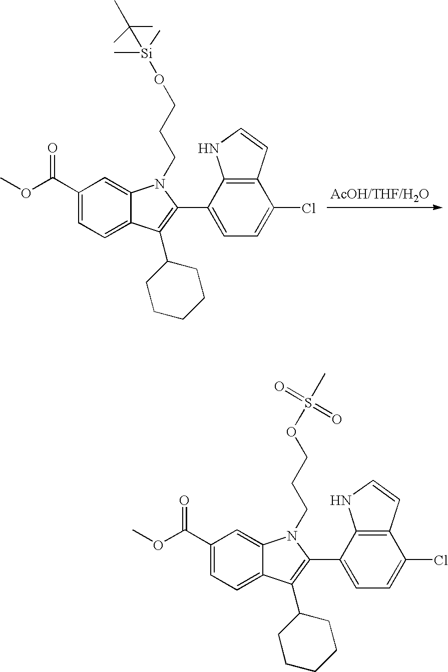

The above silane (700 mg, 1.2 mmole) was dissolved in a solution of 3:1:1 Acetic acid:water:THF (50 mL) and heated to 60° C. for 60 minutes. The completed reaction was then concentrated to an oil, co-evaporated 3 times with DMF and used directly in the next step. Yield:

338 mg (60%). MS (M+H⁺): 465.2. The alcohol (338 mg, 0.73 mmole) and triethylamine (0.3 mL, 2.18 mmole) were dissolved in anhydrous THF (15 mL) and methanesulfonyl chloride (0.17 mL, 2.18 mmole) was added drop wise at room temperature. The reaction was complete instantaneously. The reaction was then diluted with EtOAc, washed with water and brine, dried over magnesium sulfate, and concentrated to give crude product: 396 mg (100%). MS (M+H⁺): 543.2.

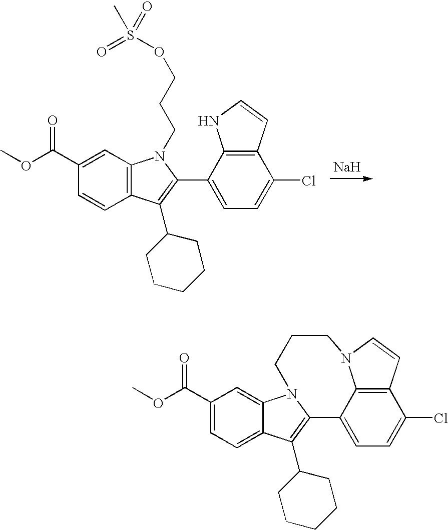

The above mesylate (396 mg, 0.73 mmole) was dissolved in 5 mL DMF and a 60% suspension of NaH in mineral oil (58.4 mg, 1.46 mmole) was added. The reaction was complete in 30 minutes, at which point 5 mL cold saturated sodium bicarbonate solution was added to quench. The reaction was diluted with 7 mL water, and extracted with 40 mL ethyl acetate. The organic layer was then washed with water, brine, dried over magnesium sulfate, and concentrated. Yield of crude product: 293 mg (90%). MS (M+H⁺): 447.2.

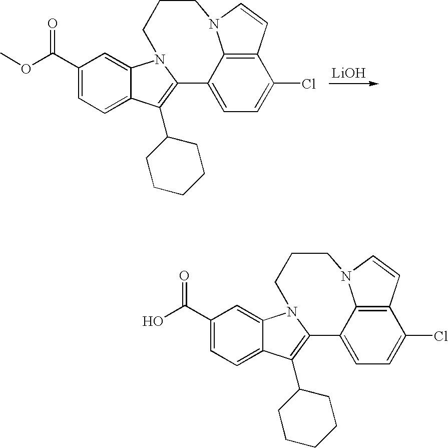

The above ester was saponified with LiOH (120 mg, 5 mmole) in 10 mL of a 2:1:1 THF:H2O:MeOH solution at 50° C. for 2 hours. The completed reaction was then purified via RP HPLC to give 275 mg (97%) of Compound 456. ¹H NMR (DMSO-d₆, 300 MHz): 12.597 (s, 1H), 8.133 (d, 1H, J=1.2 Hz), 7.901 (d, 1H, J=8.4 Hz), 7.665 (d, 1H, J=8.7 Hz), 7.521 (d, 1H, J=3.0 Hz), 7.272 (d, 1H, J=7.5 Hz), 7.07 (d, 1H, J=7.8 Hz), 6.589 (d, 1H, J=3.3 Hz), 4.610 (m, 1H), 4.165 (m, 1H), 3.574 (m, 1H), 3.50-2.90 (m, 1H), 2.79 (m, 1H), 2.19-0.95 (m, 12H); MS (M+1): 433.3.

### Example 218

### Preparation of Compound 457

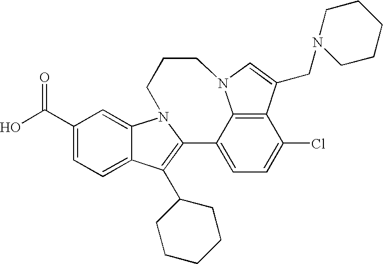

¹H NMR (DMSO-d₆, 300 MHz): 9.082 (s, 1H), 8.088 (s, 1H), 7.85 (d, 1H, J=8.4 Hz), 7.713 (s, 1H), 7.606 (d, 1H, J=8.7 Hz), 7.292 (d, 1H, J=7.8 Hz), 7.07 (d, 1H, J=7.8 Hz), 4.58 (m, 3H), 4.12 (m, 1H), 3.574-2.90 (m, 6H), 2.674 (m, 1H), 2.09-0.95 (m, 18H); MS (M+1): 530.2.

### Example 219

### Preparation of Compound 458

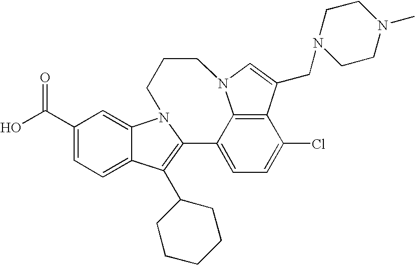

¹H NMR (DMSO-d₆, 300 MHz): 8.088 (s, 1H), 7.845 (d, 1H, J=8.4 Hz), 7.609 (m, 2H), 7.260 (d, 1H, J=7.8 Hz), 7.04 (d, 1H, J=7.8 Hz), 4.58 (m, 1H), 4.08 (m, 1H), 3.574-3.0 (m, 12H), 2.724 (m, 4H), 2.09-0.95 (m, 12H); MS (M+1): 545.3.

### Example 220

### Preparation of Compound 459

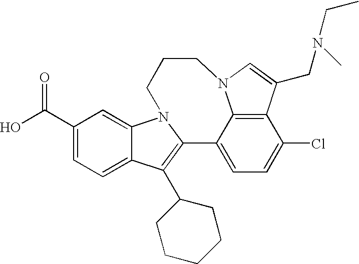

¹H NMR (DMSO-d₆, 300 MHz): 9.115 (s, 1H), 8.092 (s, 1H), 7.85 (d, 1H, J=8.4 Hz), 7.725 (s, 1H), 7.606 (d, 1H, J=8.7 Hz), 7.292 (d, 1H, J=7.8 Hz), 7.077 (d, 1H, J=7.8 Hz), 4.8-4.38 (m, 3H), 4.12 (m, 1H), 3.574-2.90 (m, 4H), 2.8-2.6 (m, 4H), 2.09-0.95 (m, 15H); MS (M+1): 504.2.

### Example 221

### Preparation of Compound 460

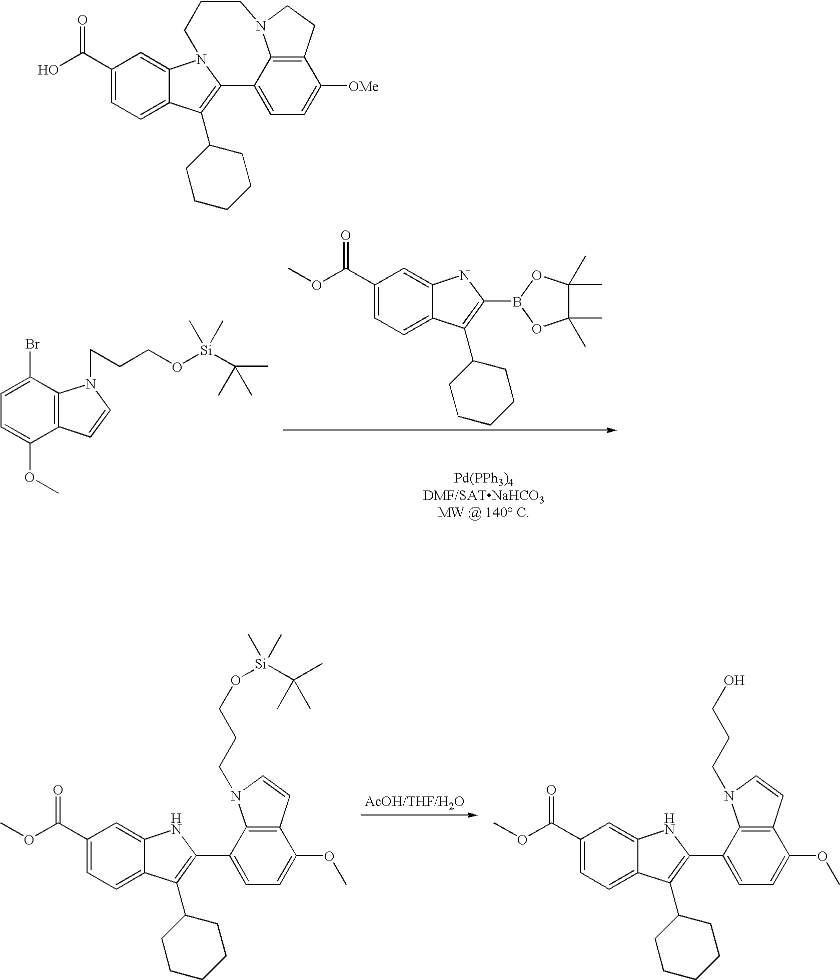

The above silyloxane (865 mg, 2.14 mmole), borolane (821 mg, 2.14 mmole), Pd(PPh₃)₄ (125.3 mg, 0.107 mmole), and aqueous saturated sodium bicarbonate (2 mL) were added to 8.6 mL DMF. The mixture was degassed and reacted under microwave condition at 140° C. for 15 minutes. The completed reaction was then filtrated concentrated and used directly in the next step. Yield of crude product: 1.23 g (100%). MS (M+H⁺): 575.3. The product (1.23 g, 2.14 mmole) was dissolved in a solution of 3:1:1 Acetic acid:water:THF (50 mL) and heated to 60° C. for 90 minutes. The completed reaction was then concentrated to an oil, co-evaporated 2 times with DMF and used directly in the next step. Yield of crude product: 0.98 g (100%). MS (M+H⁺): 461.2. The product (1.15 g, 2.14 mmole) and triethylamine (0.9 mL, 6.42 mmole) were dissolved in anhydrous THF (15 mL) and methanesulfonyl chloride (0.5 mL, 26.42 mmole) was added drop wise at room temperature. The reaction was complete instantaneously. The reaction was then diluted with EtOAc, washed with water and brine, dried over magnesium sulfate, and concentrated to give crude sulfone: 1.15 g (100%). MS (M+H⁺): 539.2.

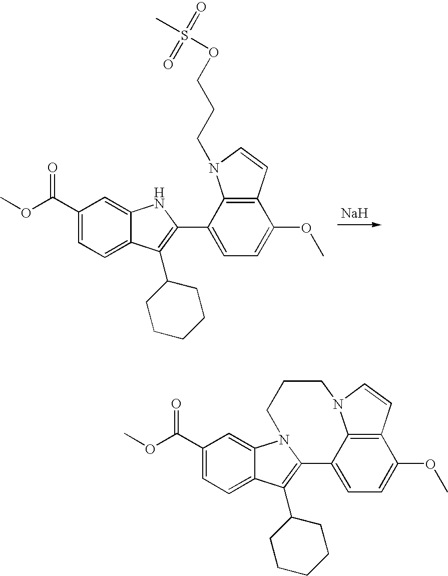

The above sulfone (750 mg, 1.17 mmole) was dissolved in 6 mL DMF and a 60% suspension of NaH in mineral oil (171 mg, 4.28 mmole) was added. The reaction was complete in 180 minutes, at which point 5 mL cold saturated sodium bicarbonate solution was added to quench. The reaction was diluted with 7 mL water, and extracted with 30 mL ethyl acetate. The organic layer was then washed with water, brine, dried over magnesium sulfate, and concentrated. Yield of crude product: 541 mg (57% in 4 steps). MS (M+H⁺): 443.3.

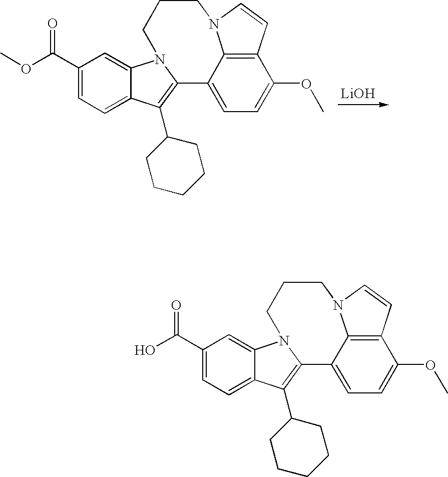

The above ester was saponified with LiOH (86 mg, 3.66 mmole) in 10 mL of a 2:1:1 THF:H2O:MeOH solution at 50° C. for 3 hours. The completed reaction was then purified via RP HPLC to give 281 mg (54%) of the corresponding acid. ¹H NMR (DMSO-d₆, 300 MHz): 12.488 (s, 1H), 8.02 (d, 1H, J=1.2 Hz), 7.803 (d, 1H, J=8.4 Hz), 7.572 (d, 1H, J=8.7 Hz), 7.20 (d, 1H, J=3.0 Hz), 6.949 (d, 1H, J=7.8 Hz), 6.648 (d, 1H, J=8.1 Hz), 6.456 (d, 1H, J=3.3 Hz), 4.510 (m, 1H), 4.03 (m, 1H), 3.878 (s, 3H), 3.513 (m, 1H), 3.128 (m, 1H), 2.79 (m, 1H), 2.09-0.95 (m, 12H); MS (M+1): 429.2.

### Example 222

### Preparation of Compound 461

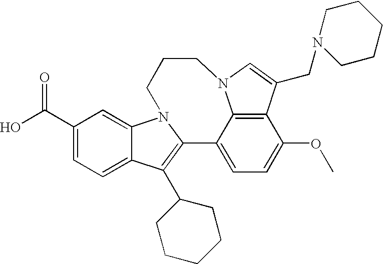

¹H NMR (DMSO-d₆, 300 MHz): 9.152 (s, 1H), 8.048 (s, 1H), 7.815 (d, 1H, J=8.4 Hz), 7.585 (d, 1H, J=8.7 Hz), 7.45 (s, 1H), 7.018 (d, 1H, J=7.8 Hz), 6.76 (d, 1H, J=7.8 Hz), 4.58-4.32 (m, 3H), 4.063 (m, 1H), 3.931 (s, 3H), 3.504 (m, 1H), 3.4-3.04 (m, 3H), 2.873 (m, 2H), 2.74 (m, 1H), 2.09-0.95 (m, 18H); MS (M+1): 526.3.

### Example 223

### Preparation of Compound 462

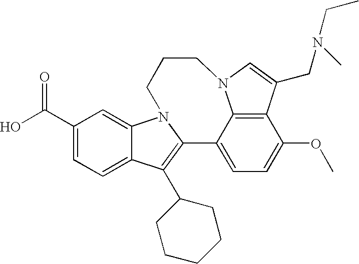

¹H NMR (DMSO-d₆, 300 MHz): 8.828 (s, 1H), 8.048 (s, 1H), 7.815 (d, 1H, J=8.7 Hz), 7.585 (d, 1H, J=8.4 Hz), 7.455 (s, 1H), 7.029 (d, 1H, J=7.8 Hz), 6.768 (d, 1H, J=7.8 Hz), 4.64 (m, 2H), 4.25 (m, 1H), 4.066 (m, 1H), 3.917 (s, 3H), 3.504 (m, 1H), 3.4-3.04 (m, 3H), 2.72 (m, 1H), 2.63 (m, 3H), 2.09-0.95 (m, 15H); MS (M+1): 500.3.

### Example 224

### Preparation of Compound 463

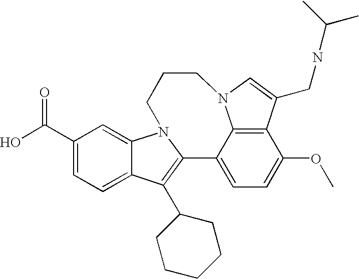

¹H NMR (DMSO-d₆, 300 MHz): 8.53 (s, 1H), 8.053 (s, 1H), 7.825 (d, 1H, J=8.7 Hz), 7.600 (d, 1H, J=8.4 Hz), 7.407 (s, 1H), 7.029 (d, 1H, J=7.8 Hz), 6.768 (d, 1H, J=7.8 Hz), 4.54 (m, 1H), 4.298 (m, 2H), 4.086 (m, 1H), 3.909 (s, 3H), 3.504 (m, 1H), 3.4-3.04 (m, 3H), 2.72 (m, 1H), 2.09-0.95 (m, 18H); MS (M+1): 500.3.

### Example 225

### Preparation of Compound 464

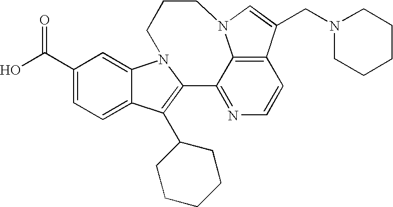

¹H NMR (DMSO-d₆, 300 MHz): 8.625 (d, 1H, J=6.6 Hz), 8.6-8.34 (m, 3H), 8.12 (d, 1H, J=8.4 Hz), 7.8 (d, 1H, J=8.4 Hz), 4.86 (m, 1H), 4.61 (m, 2H), 4.48 (m, 1H), 3.902 (m, 1H), 3.309 (m, 1H), 2.99-2.86 (m, 4H), 2.42 (m, 1H), 2.19-0.95 (m, 18H); MS (M+1): 497.3.

### Example 226

### Preparation of Compound 465

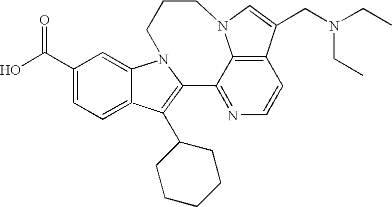

¹H NMR (DMSO-d₆, 300 MHz): 8.605 (d, 1H, J=6.6 Hz), 8.6-8.34 (m, 3H), 8.105 (d, 1H, J=8.4 Hz), 7.8 (d, 1H, J=8.4 Hz), 4.852 (m, 1H), 4.658 (m, 2H), 4.48 (m, 1H), 3.902 (m, 1H), 3.6-3.1 (m, 4H), 2.92 (m, 1H), 2.42 (m, 1H), 2.19-0.95 (m, 18H); MS (M+1): 485.3.

### Example 227

### Preparation of Compound 466

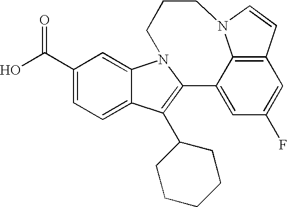

¹H NMR (DMSO-d₆, 300 MHz): 12.579 (s, 1H), 8.09 (d, 1H, J=1.2 Hz), 7.87 (d, 1H, J=8.4 Hz), 7.62 (d, 1H, J=8.7 Hz), 7.43 (m, 2H), 6.849 (m, 1H), 6.498 (d, 1H, J=2.7 Hz), 4.560 (m, 1H), 4.085 (m, 1H), 3.549 (m, 1H), 3.4-3.08 (m, 1H), 2.763 (m, 1H), 2.09-1.0 (m, 12H); MS (M+1): 417.2.

### Example 228

### Preparation of Compound 467

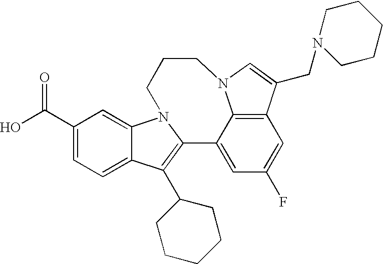

¹H NMR (DMSO-d₆, 300 MHz): 9.926 (s, 1H), 8.113 (s, 1H), 7.867 (m, 2H), 7.643 (m, 2H), 6.905 (d, 1H, J=9.3 Hz), 4.60 (m, 1H), 4.351 (m, 2H), 4.10 (m, 1H), 3.558 (m, 1H), 3.4-3.04 (m, 3H), 2.823 (m, 3H), 2.09-1.0 (m, 18H); MS (M+1): 514.3.

### Example 229

### Preparation of Compound 468

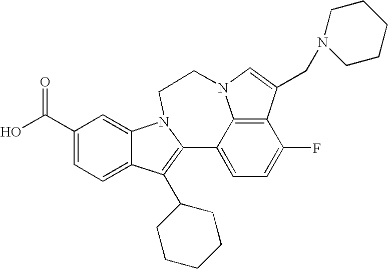

¹H NMR (DMSO-d₆, 300 MHz): 10.04 (s, 1H), 8.236 (s, 1H), 7.930 (d, 1H, J=8.4 Hz), 7.795 (s, 1H), 7.650 (d, 1H, J=8.4 Hz), 7.319 (m, 1H), 7.170 (m, 1H), 4.43 (m, 3H), 3.50-3.25 (m, 5H), 3.043 (m, 1H), 2.823 (m, 2H), 2.20-1.0 (m, 16H); MS (M+1): 500.3.

### Example 230

### Preparation of Compound 469

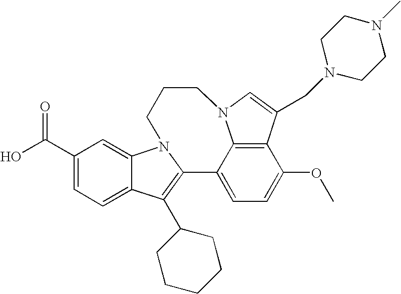

¹H NMR (DMSO-d₆, 300 MHz): 8.05 (s, 1H), 7.815 (d, 1H, J=8.7 Hz), 7.585 (d, 1H, J=8.4 Hz), 7.481 (s, 1H), 7.025 (d, 1H, J=7.8 Hz), 6.774 (d, 1H, J=7.8 Hz), 4.564 (m, 3H), 4.068 (m, 1H), 3.948 (s, 3H), 3.752-3.04 (m, 10H), 2.72 (m, 4H), 2.09-0.95 (m, 12H); MS (M+1): 541.3.

### Example 231

### Preparation of Compound 470

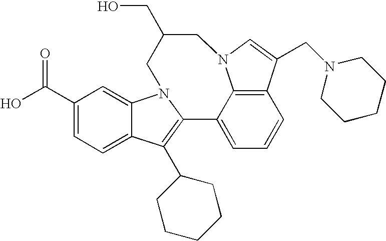

Prepared as mixture of diastereomers. Yield: 40 mg; MS (M+H⁺): 526.3; H¹-NMR (DMSO d₆): δ (ppm) 9.53 (br s, 1H), 8.22-8.11 (m, 1H), 7.92-7.82 (m, 2H), 7.64-7.49 (m, 2H), 7.28-7.22 (m, 1H), 7.13-7.04 (m, 1H), 4.66-4.52 (m, 1H), 4.44-4.38 (m, 2H), 4.22-4.08 (m, 1H), 3.74-3.52 (m, 3H), 3.48-3.12 (m, 4H), 2.92-2.72 (m, 4H), 2.30-1.02 (m, 16H).

### Example 232

### Preparation of Compound 496

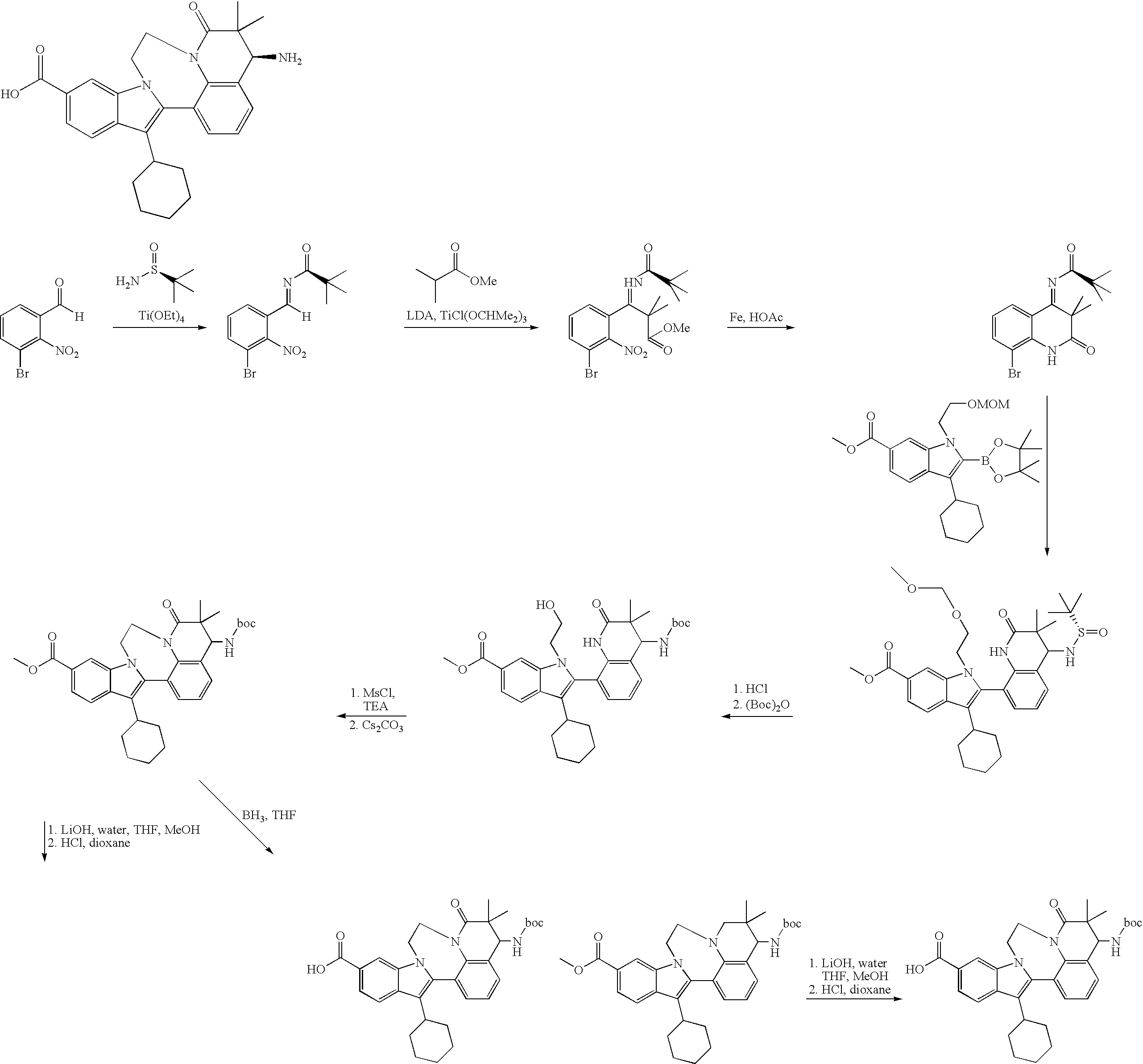

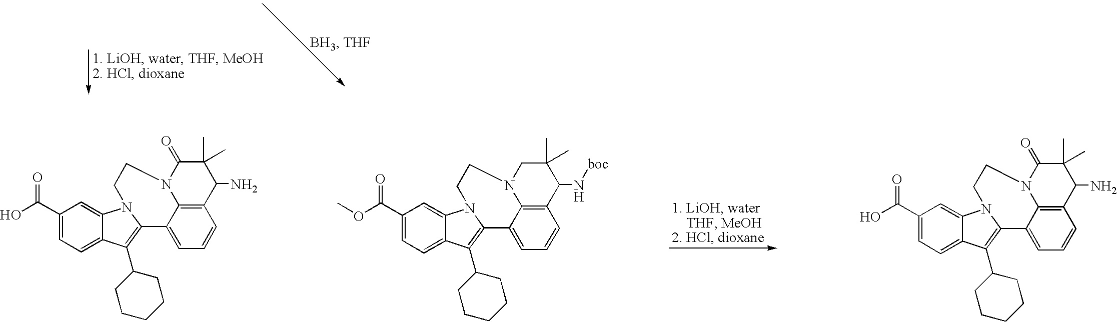

### (S)—N-[(1E)-(3-bromo-2-nitrophenyl)methylene]-2-methylpropane-2-sulfinamide

To a solution of 3-bromo-2-nitrobenzaldehyde (Tetrahedron 2008, 64, 856-865) (1.5 g, 6.5 mmol, 1.0 equiv) and (S)-2-methylpropane-2-sulfinamide (1.2 g, 9.8 mmol, 1.5 equiv) in THF (5.0 mL) was added Ti(OEt)₄ (4.1 mL, 19.6 mmol, 3.0 equiv) and the resultant mixture was heated to 70° C. for 1 hour. After cooled at room temperature, the mixture was and poured into a solution of brine with rapid stirring. The resulting suspension was filtered and washed with EtOAc. The phases were separated and the aqueous layer was extracted with EtOAc. The combined organic phases were washed with brine, dried (over Na₂SO₄) and concentrated. The residue was purified by silica gel column chromatography (heptane/EtOAc, 1/1) to give product 1.6 g. MS: 335 [M+H⁺].

### Methyl (3R)-3-(3-bromo-2-nitrophenyl)-3-{[(S)-tert-butylsulfinyl]amino}-2,2-dimethylpropanoate and Methyl (3S)-3-(3-bromo-2-nitrophenyl)-3-{[(S)-tert-butylsulfinyl]amino}-2,2-dimethylpropanoate

To a solution of DIPA (0.44 mL, 3.1 mmol, 3.5 equiv) in THF (3.0 mL) at 0° C. was added n-BuLi (1.1 mL, 2.5 M, 2.9 mmol, 3.2 equiv) and the resultant solution was stirred at 0° C. for 10 minutes. The solution was then placed in an dry ice/acetone bath and methyl isobutyrate (0.31 mL, 2.7 mmol, 3.0 equiv) was added. After stirring at this temperature for 15 minutes, to the solution was added TiCl(OCHMe₂)₃ (5.7 mL, 1.0 M, 5.7 mmol, 6.4 equiv) and the resultant solution was stirred at −78° C. for 30 minutes. To the solution was then added (S)—N-[(1E)-(3-bromo-2-nitrophenyl)methylene]-2-methylpropane-2-sulfinamide (300 mg, 0.90 mmol, 1.0 equiv) and the solution was stirred at −78° C. for 1 hour. The reaction was quenched by addition of sat. aq. NH₄Cl solution at −78° C. After warming to room temperature, the mixture was diluted with EtOH and filtered through a pad of celite. The solvent was then removed under vacuum and the residue was purified by silica gel column chromatography (heptane/acetone, 2/1) to give product 320 mg. MS: 437 [M+H⁺].

### (S)—N-[(4S)-8-bromo-3,3-dimethyl-2-oxo-1,2,3,4-tetrahydroquinolin-4-yl]-2-methylpropane-2-sulfinamide and (S)—N-[(4R)-8-bromo-3,3-dimethyl-2-oxo-1,2,3,4-tetrahydroquinolin-4-yl]-2-methylpropane-2-sulfinamide

To a solution of methyl 3-(3-bromo-2-nitrophenyl)-3-{[(S)-tert-butylsulfinyl]amino}-2,2-dimethylpropanoate (1.4 g, 3.2 mmol, 1.0 equiv) in AcOH (20.0 mL) at room temperature was added iron powder (1.1 g, 19.3 mmol, 6.0 equiv) and the mixture was heated at 100° C. for 1 hour. The mixture was then diluted with EtOAc and filtered. The filtrate was concentrated under vacuum and the residue was purified by silica gel column chromatography (heptane/acetone 1/1) to give two diasteromeric mixtures. First fraction 610 mg, second fraction 530 mg. MS: 375 [M+H⁺].

### Methyl 2-[(4S)-4-{[(S)-tert-butylsulfinyl]amino}-3,3-dimethyl-2-oxo-1,2,3,4-tetrahydroquinolin-8-yl]-3-cyclohexyl-1-[2-(methoxymethoxy)ethyl]-1H-indole-6-carboxylate

To a solution of first fraction product from previous step (0.53 g, 1.42 mmol, 1.0 equiv) in dioxane (5.0 mL) and EtOH (5.0 mL) was added methyl 3-cyclohexyl-1-[2-(methoxymethoxy)ethyl]-2-(4,4,5,5-tetramethyl-1,3,2-dioxaborolan-2-yl)-1H-indole-6-carboxylate (0.80 g, 1.70 mmol, 1.2 equiv), Pd(PPh₃)₄ (0.16 g, 0.14 mmol, 0.1 equiv) and K₂CO₃ (2.0 M solution in water, 2.1 mL, 4.2 mmol, 3.0 equiv). The mixture was degassed and stirred at 95° C. for 3 hours. The solvent was then removed under vacuum and the residue was diluted with EtOAc. The solution was washed with water, brine, dried over Na₂SO₄ and concentrated. The crude material was purified by silica gel column chromatography (heptane/acetone, 1/1) to give product 0.71 g. MS: 638 [M+H⁺].

### Methyl 2-{(4S)-4-[(tert-butoxycarbonyl)amino]-3,3-dimethyl-2-oxo-1,2,3,4-tetrahydroquinolin-8-yl}-3-cyclohexyl-1-(2-hydroxyethyl)-1H-indole-6-carboxylate

To a solution of methyl 2-[(4S)-4-{[(S)-tert-butylsulfinyl]amino}-3,3-dimethyl-2-oxo-1,2,3,4-tetrahydroquinolin-8-yl]-3-cyclohexyl-1-[2-(methoxymethoxy)ethyl]-1H-indole-6-carboxylate (0.71 g, 1.1 mmol, 1.0 equiv) in MeOH (4.0 mL) was added 4.0 N HCl in dioxane (4.17 mL). The solution was stirred at room temperature for 1 hour, after which the solvent was removed under vacuum. The crude material was redissolved in CH₂Cl₂ and heptane. After removing the solvent under vacuum, the residue was dissolved in CH₂Cl₂ (4.0 mL) and to the solution was added DIPEA (0.39 mL, 2.2 mmol, 2.0 equiv) and (Boc)₂O (0.32 g, 1.4 mmol, 1.3 equiv). The mixture was then stirred at 0° C. for 40 hours, after which the solvent was removed under vacuum and the residue was purified by silica gel column chromatography (heptane/acetone, 1/1) to give product 0.60 g. MS: 590 [M+H⁺].

### Methyl (4S)-4-[(tert-butoxycarbonyl)amino]-15-cyclohexyl-5,5-dimethyl-6-oxo-5,6,8,9-tetrahydro-4H-indolo[1′,2′:4,5][1,4]diazepino[6,7,1-ij]quinoline-12-carboxylate

To a solution of methyl 2-{(4S)-4-[(tert-butoxycarbonyl)amino]-3,3-dimethyl-2-oxo-1,2,3,4-tetrahydroquinolin-8-yl}-3-cyclohexyl-1-(2-hydroxyethyl)-1H-indole-6-carboxylate (0.60 g, 1.0 mmol, 1.0 equiv) in CH₂Cl₂ (5.0 mL) was added Et₃N (0.29 mL, 2.0 mmol, 2.0 equiv) and MsCl (0.10 mL, 1.3 mmol, 1.3 equiv). After stirring at 0° C. for 20 minutes, the reaction was quenched by addition of ice/water mixtures. The mixture was diluted with CH₂Cl₂ and the phases were separated. The organic layer was washed with brine, dried (Na₂SO₄) and concentrated. The crude material was used in the next step with no further purification.

The product from previous step was dissolved in DMF (5.0 mL) and Cs₂CO₃ (994 mg, 3.0 mmol, 3.0 equiv) was added. The mixture was stirred at room temperature for 18 hours, after which the mixture was filtered. After the solvent was removed under vacuum, the residue was purified by silica gel column chromatography (heptane/EtOAc, 1/1) to give product 550 mg. MS: 572 [M+H⁺].

### (4S)-4-amino-15-cyclohexyl-5,5-dimethyl-6-oxo-5,6,8,9-tetrahydro-4H-indolo[1′,2′:4,5][1,4]diazepino[6,7,1-ij]quinoline-12-carboxylic Acid

To a solution of methyl ester (100 mg, 0.18 mmol, 1.0 equiv) in THF (2.0 mL) was added MeOH (1.0 mL), water (1.0 mL) and LiOH.H₂O (110 mg, 2.6 mmol, 15.0 equiv). After stirring at 58° C. for 2 hours, the mixture was concentrated under vacuum and to the residue was added 1.0 N HCl aq. solution until pH=5. To the mixture was added EtOAc and the phases were separated. The organic layer was washed with brine, dried (Na₂SO₄) and concentrated. The material was used in the next step with no further purification.

The product from previous step was dissolved in dioxane (1.5 mL) and to the solution was added 4.0 N HCl in dioxane (4.0 mL). After stirring at room temperature for 2 hours, the mixture was concentrated under vacuum. To the residue was added CH₂Cl₂/heptane and the solvent was again removed under vacuum. The residue was dissolved in CH₃CN/water and the solvent was removed with freeze drying method to give product 80 mg. MS: 458 [M+H⁺]. ¹H NMR (DMSO-d₆): 12.6 (s, 1H), 8.55 (br, 2H), 8.24 (s, 1H), 7.89 (d, 1H), 7.68 (d, 1H), 7.58 (d, 1H), 7.43 (m, 2H), 5.00 (br, 1H), 4.40 (br, 1H), 4.19 (br, 2H), 3.70 (br, 1H), 2.75 (m, 1H), 1.60-2.06 (m, 6H), 1.44-1.60 (m, 1H), 1.20 (s, 6H), 0.74-1.02 (m, 3H).

### Example 233

### Preparation of Compound 497

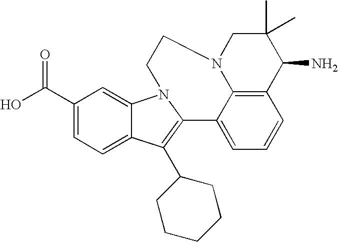

### Methyl (4R)-4-[(tert-butoxycarbonyl)amino]-15-cyclohexyl-5,5-dimethyl-5,6,8,9-tetrahydro-4H-indolo[1′,2′:4,5][1,4]diazepino[6,7,1-ij]quinoline-12-carboxylate

To a solution of methyl (4S)-4-[(tert-butoxycarbonyl)amino]-15-cyclohexyl-5,5-dimethyl-6-oxo-5,6,8,9-tetrahydro-4H-indolo[1′,2′:4,5][1,4]diazepino[6,7,1-ij]quinoline-12-carboxylate (100 mg, 0.17 mmol, 1.0 equiv) in THF (5 mL) was added BH₃.THF (10.5 mL, 1.0 M, 10.5 mmol, 60.0 equiv) and Me₃SiCl (0.1 mL). The mixture was stirred at 45° C. for 5 hours, after which the solvent was removed under vacuum. The residue was purified by silica gel column chromatography (heptane/EtOAc, 1/1) to give product 15 mg. MS: 558, [M+H⁺].

### (4R)-4-amino-15-cyclohexyl-5,5-dimethyl-5,6,8,9-tetrahydro-4H-indolo[1′,2′:4,5][1,4]diazepino[6,7,1-ij]quinoline-12-carboxylic Acid

To a solution of methyl (4R)-4-[(tert-butoxycarbonyl)amino]-15-cyclohexyl-5,5-dimethyl-5,6,8,9-tetrahydro-4H-indolo[1′,2′:4,5][1,4]diazepino[6,7,1-ij]quinoline-12-carboxylate (15 mg, 0.03 mmol, 1.0 equiv) in THF (0.6 mL) was added MeOH (0.3 mL), water (0.3 mL) and LiOH.H₂O (45.2 mg, 1.0 mmol, 40.0 equiv). After stirring at 57° C. for 2 hours, the mixture was filtered and purified by HPLC.

The product from previous step was dissolved in dioxane (1.0 mL) and to the solution was added 4.0 N HCl in dioxane (1.5 mL). After stirring at room temperature for 2.5 hours, the mixture was concentrated under vacuum. To the residue was added CH₂Cl₂/heptane and the solvent was again removed under vacuum. The resultant solid was dissolved in CH₃CN/water and the solvent was removed by freeze dry method to give product 9 mg. MS: 444 [M+H⁺]. ¹H NMR (DMSO-d₆): 8.15 (br, 4H), 7.86 (d, 1H), 7.62 (d, 1H), 7.37 (d, 1H), 7.24 (d, 1H), 6.95 (t, 1H), 4.65 (br, 1H), 4.09 (br, 1H), 3.41-3.75 (m, 5H), 2.96-3.10 (m, 1H), 2.82-2.96 (m, 1H), 1.90-2.09 (m, 2H), 1.52-1.86 (m, 3H), 1.10-1.51 (m, 2H), 1.05 (s, 6H), 0.73-1.00 (m, 2H).

### Example 234

### Preparation of Compound 498

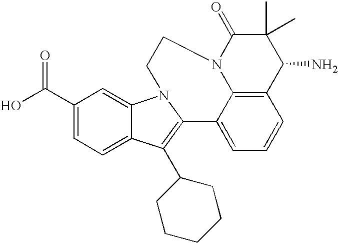

This compound was prepared as described for compound 496 using (S)—N-[(4R)-8-bromo-3,3-dimethyl-2-oxo-1,2,3,4-tetrahydroquinolin-4-yl]-2-methylpropane-2-sulfinamide. MS: 458 [M+H⁺]. ¹H NMR (DMSO-d₆): 12.7 (s, 1H), 8.55 (br, 2H), 8.24 (s, 1H), 7.89 (d, 1H), 7.68 (d, 1H), 7.58 (d, 1H), 7.43 (m, 2H), 5.00 (br, 1H), 4.40 (br, 1H), 4.19 (br, 2H), 3.70 (br, 1H), 2.75 (m, 1H), 1.60-2.06 (m, 6H), 1.44-1.60 (m, 1H), 1.20 (s, 6H), 0.74-1.05 (m, 3H).

### Example 235

### Preparation of Compound 499

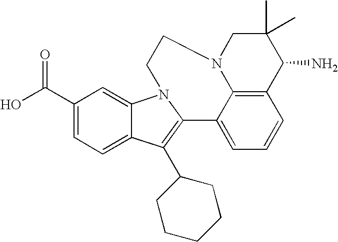

This compound was prepared as described for compound 467 using (S)—N-[(4R)-8-bromo-3,3-dimethyl-2-oxo-1,2,3,4-tetrahydroquinolin-4-yl]-2-methylpropane-2-sulfinamide. MS: 444 [M+H⁺]. ¹H NMR (DMSO-d₆): 12.6 (br, 1H), 8.15 (br, 3H), 7.86 (d, 1H), 7.62 (d, 1H), 7.37 (d, 1H), 7.24 (d, 1H), 6.95 (t, 1H), 4.65 (br, 1H), 4.09 (br, 1H), 3.41-3.75 (m, 5H), 2.96-3.10 (m, 1H), 2.82-2.96 (m, 1H), 1.90-2.09 (m, 2H), 1.52-1.86 (m, 3H), 1.10-1.51 (m, 2H), 1.05 (s, 6H), 0.73-1.00 (m, 2H).

### BIOLOGICAL EXAMPLES

### Biological Example 1

### Anti-Hepatitis C Activity

Compounds can exhibit anti-hepatitis C activity by inhibiting HCV polymerase, by inhibiting other enzymes needed in the replication cycle, or by other pathways. A number of assays have been published to assess these activities. A general method that assesses the gross increase of HCV virus in culture was disclosed in U.S. Pat. No. 5,738,985 to Miles et al In vitro assays have been reported in Ferrari et al. Jnl. of Vir., 73:1649-1654, 1999; Ishii et al., Hepatology, 29:1227-1235, 1999; Lohmann et al., Jnl of Bio. Chem., 274:10807-10815, 1999; and Yamashita et al., Jnl. of Bio. Chem., 273:15479-15486, 1998.

WO 97/12033, filed on Sep. 27, 1996, by Emory University, listing C. Hagedorn and A. Reinoldus as inventors, which claims priority to U.S. Provisional Patent Application Ser. No. 60/004,383, filed on September 1995, described an HCV polymerase assay that can be used to evaluate the activity of the of the compounds described herein. Another HCV polymerase assay has been reported by Bartholomeusz, et al., Hepatitis C Virus (HCV) RNA polymerase assay using cloned HCV non-structural proteins; Antiviral Therapy 1996:1 (Supp 4) 18-24.

Screens that measure reductions in kinase activity from HCV drugs were disclosed in U.S. Pat. No. 6,030,785, to Katze et al., U.S. Pat. No. 6,228,576, Delvecchio, and U.S. Pat. No. 5,759,795 to Jubin et al. Screens that measure the protease inhibiting activity of proposed HCV drugs were disclosed in U.S. Pat. No. 5,861,267 to Su et al., U.S. Pat. No. 5,739,002 to De Francesco et al., and U.S. Pat. No. 5,597,691 to Houghton et al.

### Biological Example 2

### Replicon Assay

A cell line, ET (Huh-lucubineo-ET) was used for screening of compounds for inhibiting HCV RNA dependent RNA polymerase. The ET cell line was stably transfected with RNA transcripts harboring a I₃₈₉luc-ubi-neo/NS3-3′/ET; replicon with firefly luciferase-ubiquitin-neomycin phosphotransferase fusion protein and EMCV-IRES driven NS3-5B polyprotein containing the cell culture adaptive mutations (E1202G; T1280I; K1846T) (Krieger at al, 2001 and unpublished). The ET cells were grown in DMEM (Dulbeco's Modified Eagle's Medium), supplemented with 10% fetal calf serum, 2 mM Glutamine, Penicillin (100 IU/mL)/Streptomycin (100 μg/mL), 1× nonessential amino acids, and 250 μg/mL G418 (“Geneticin”). Reagents are all available through Life Technologies (Bethesda, Md.). The cells were plated at 0.5-1.0×10⁴ cells/well in the 96 well plates and incubated for 24 hrs before adding test compound. The compounds were added to the cells to achieve a final concentration of 0.1 nM to 50 μM and a final DMSO (dimethylsulfoxide) concentration of 0.5%. Luciferase activity was measured 48-72 hours later by adding a lysis buffer and the substrate (Catalog number Glo-lysis buffer E2661 and Bright-Glo luciferase system E2620 Promega, Madison, Wis.). Cells should not be too confluent during the assay. Percent inhibition of replication data is plotted relative to no compound control. Under the same condition, cytotoxicity of the compounds were determined using cell proliferation reagent, WST-1 (Roche, Germany). The compounds showing antiviral activities, but no significant cytotoxicities were chosen to determine EC₅₀ and TC₅₀ the effective concentration and toxic concentration at which 50% of the maximum inhibition is observed. For these determinations, a 10 point, 2-fold serial dilution for each compound was used, which spans a concentration range of 1000 fold. EC₅₀ and similarly TC₅₀ values were calculated by fitting % inhibition at each concentration to the following equation:

% inhibition=100%/[(EC₅₀/[I])_{b}+1]

where b is Hill's coefficient.

In some aspects, certain compounds of Formula (I), exhibited EC₅₀ of equal to or less than 50 μM when tested according to the assay of Example 2. In other aspects the EC₅₀ was equal to or less than 10 μM. In still other aspects the EC₅₀ was equal to or less than 1 μM.

### Biological Example 3

### Cloning and Expression of Recombinant HCV-NS5b

The coding sequence of NS5b protein was cloned by PCR from pFKI₃₈₉luc/NS3-3′/ET as described by Lohmann, V., et al. (1999) Science 285, 110-113 using the primers shown on page 266 of WO 2005/012288

The cloned fragment is missing the C terminus 21 amino acid residues. The cloned fragment was inserted into an IPTG-inducible (isopropyl-β-D-thiogalactopyranoside) expression plasmid that provides an epitope tag (His)6 at the carboxy terminus of the protein.

The recombinant enzyme was expressed in XL-1 cells and after induction of expression, the protein was purified using affinity chromatography on a nickel-NTA (nitrilotriacetic acid) column. Storage condition was 10 mM Tris-HCl pH 7.5, 50 mM NaCl, 0.1 mM EDTA (ethylenediaminetetraacetic acid), 1 mM DTT (dithiothreotol), 20% glycerol at −20° C.

### Biological Example 4

### HCV-NS5b Enzyme Assay Using Heteropolymer Substrate

The polymerase activity was assayed by measuring incorporation of radiolabeled UTP into a RNA product using a biotinylated, heteropolymeric template, which included a portion of the HCV genome. Typically, the assay mixture (50 L) contained 10 mM Tris-HCl (pH 7.5), 5 mM MgCl₂, 0.2 mM EDTA, 10 mM KCl, 1 unit/μL RNAsin, 1 mM DTT, 10 μM each of NTP (nucleoside triphosphate), including [³H]-UTP (uridine triphosphate), and 10 ng/μL heteropolymeric template. Test compounds were initially dissolved in 100% DMSO and further diluted in aqueous buffer containing 5% DMSO. Typically, compounds were tested at concentrations between 1 nM and 100 μM. Reactions were started with addition of enzyme and allowed to continue at 37° C. for 2 hours. Reactions were quenched with 8 μL of 100 mM EDTA and reaction mixtures (30 μL) were transferred to streptavidin-coated scintillation proximity microtiter plates (FlashPlates) and incubated at room temperature overnight. Incorporation of radioactivity was determined by scintillation counting.

### Biological Example 5

### HCV-NS5b Enzyme Assay Using Homopolymer Substrate

The polymerase activity was assayed by measuring incorporation of radiolabeled UTP into a RNA product using a biotinylated, homopolymeric template. The template was formed by annealing adenosine homopolymer to uridine 20-mer capped with a 5′-biotin group (biotin-U²⁰) in the ratio of 1:4. Typically, the assay mixture (50 μL) contained 25 mM Tris-HCl (pH 7.5), 40 mM KCl, 0.3 mM MgCl₂, 0.05 mM EDTA, 0.2 unit/μL Superase RNAse Inhibitor, 5 mM DTT, 30 μM UTP (Uridine triphosphate), including [³H]-UTP (uridine triphosphate) at 0.4 μCi/μL with final concentration of 1 μM, and 50 nM of homopolymeric template. Test compounds were initially dissolved in 100% DMSO and further diluted in aqueous buffer containing 5% DMSO. Typically, compounds were tested at concentrations between 2 nM and 50 μM. Reactions were started with addition of enzyme and allowed to continue at 30° C. for 90 minutes. Reactions were quenched with 8 μL of 100 mM EDTA and reaction mixtures (30 μL) were transferred to streptavidin-coated scintillation proximity microtiter plates (FlashPlates) and incubated at room temperature overnight. Incorporation of radioactivity was determined by scintillation counting.

Inhibitor IC₅₀ values were determined by adding test compound as ten point, two-fold serial dilutions in 100% DMSO with a final reaction concentration of 5%. IC₅₀ was calculated by plotting the % inhibition against compound concentration and fitting the data to a constrained four parameter sigmoidal curve, equivalent to the “four parameter logistic equation”:

$$Y = Bottom + \frac{( Top - Bottom )}{( 1 + ( {10}^{( ( logEC50 - X ) * Hillslope )} ) )}$$

Where Bottom is the minimum Y value, Top is the maximum Y value, and Hillslope is the slope of the linear portion of the semi-log curve. Top and Bottom were constrained to values of 0% and 100%, respectively. These analyses were performed using Graphpad Prism v.4.0 (Graphpad Software, Inc.) in conjunction with DS Accord for EXCEL 6.0 (Accelrys, Microsoft Corp.).

The table below lists the IC₅₀ values of compounds determined using the homopolymer substrates.

| Compound No. | IC₅₀ (μM) |
|---|---|
| 105 | 0.131 |
| 106 | 0.128 |
| 107 | 0.019 |
| 108 | 0.083 |
| 109 | 0.113 |
| 110 | 0.096 |
| 111 | 0.106 |
| 112 | 0.109 |
| 113 | 0.084 |
| 114 | 0.089 |
| 115 | 0.087 |
| 116 | 0.138 |
| 117 | 0.108 |
| 118 | 0.048 |
| 119 | 0.114 |
| 120 | 0.085 |
| 121 | 0.049 |
| 122 | 0.051 |
| 123 | 0.1554 |
| 124 | 0.114 |
| 125 | 0.068 |
| 126 | 0.096 |
| 127 | 0.2905 |
| 128 | 0.101 |
| 129 | 0.096 |
| 130 | 0.111 |
| 131 | 0.208 |
| 132 | 0.223 |
| 204 | 0.118 |
| 214 | 0.278 |
| 276 | 0.065 |
| 277 | 0.056 |
| 281 | 0.061 |
| 282 | 0.059 |
| 285 | 0.079 |
| 329 | 0.087 |
| 330 | 0.113 |
| 337 | 1.292 |
| 341 | 0.135 |
| 344 | 0.133 |
| 345 | 0.127 |
| 346 | 0.119 |
| 347 | 0.122 |
| 348 | 0.143 |
| 349 | 0.098 |
| 350 | 0.065 |
| 351 | 0.056 |
| 352 | 0.089 |
| 353 | 0.109 |
| 354 | 0.11 |
| 356 | 0.12 |
| 357 | 0.208 |
| 358 | 0.153 |
| 359 | 0.155 |
| 360 | 0.149 |
| 361 | 0.176 |
| 362 | 0.337 |
| 363 | 0.124 |
| 364 | 0.277 |
| 365 | 0.088 |
| 366 | 0.121 |
| 367 | 0.158 |
| 368 | 0.303 |
| 369 | 0.171 |
| 370 | 0.094 |
| 371 | 0.109 |
| 372 | 0.106 |
| 373 | 0.153 |
| 374 | 0.365 |
| 375 | 0.177 |
| 376 | 0.641 |
| 377 | 0.28 |
| 378 | 0.199 |
| 379 | 0.117 |
| 380 | 0.093 |
| 381 | 0.117 |
| 382 | 0.141 |
| 383 | 0.085 |
| 384 | 0.065 |
| 385 | 0.133 |
| 386 | 0.196 |
| 387 | 0.18 |
| 388 | 0.159 |
| 389 | 0.197 |
| 390 | 0.237 |
| 391 | 0.211 |
| 392 | 0.105 |
| 393 | 0.138 |
| 394 | 0.159 |
| 395 | 0.14 |
| 396 | 0.126 |
| 397 | 0.218 |
| 398 | 0.204 |
| 399 | 0.22 |
| 400 | 0.25 |
| 401 | 0.14 |
| 402 | 0.168 |
| 403 | 0.207 |
| 404 | 0.257 |
| 405 | 0.139 |
| 406 | 0.136 |
| 407 | 0.253 |
| 408 | 0.142 |
| 409 | 0.114 |
| 410 | 0.384 |
| 411 | 0.277 |
| 412 | 0.179 |
| 413 | 0.105 |
| 414 | 0.098 |
| 415 | 0.17 |
| 416 | 0.146 |
| 417 | 0.099 |
| 418 | 0.144 |
| 419 | 0.18 |
| 421 | 0.472 |
| 427 | 0.121 |
| 428 | 0.166 |
| 429 | 0.099 |
| 430 | 0.073 |
| 431 | 0.201 |
| 432 | 0.324 |
| 433 | 0.181 |
| 434 | 0.172 |
| 435 | 0.171 |
| 436 | 0.333 |
| 437 | 0.151 |
| 438 | 0.069 |
| 439 | 0.182 |
| 440 | 0.107 |
| 441 | 0.271 |
| 442 | 0.123 |
| 443 | 0.121 |
| 444 | 0.215 |
| 445 | 0.103 |
| 446 | 0.169 |
| 447 | 0.059 |
| 449 | 0.253 |
| 452 | 0.325 |
| 453 | 0.212 |
| 454 | 0.348 |
| 455 | 0.16 |
| 456 | 0.125 |
| 457 | 0.195 |
| 458 | 0.196 |
| 459 | 0.16 |
| 460 | 0.211 |
| 461 | 0.368 |
| 462 | 0.414 |
| 463 | 2.061 |
| 464 | 0.96 |
| 465 | 1.72 |
| 466 | 0.085 |
| 467 | 0.095 |
| 468 | 0.084 |
| 469 | 0.662 |

### Biological Example 6

The polymerase activity was also assayed by measuring incorporation of radiolabeled GTP into an RNA product using a biotinylated oligoG13 primer with a polycytidylic acid RNA template. Typically, the assay mixture (40 μL) contains 50 mM HEPES (pH 7.3), 2.5 mM magnesium acetate, 2 mM sodium chloride, 37.5 mM potassium acetate, 5 mM DTT, 0.4 U/mL RNasin, 2.5% glycerol, 3 nM NS5B, 20 nM polyC RNA template, 20 nM biotin-oligoG13 primer, and 0.2 μM tritiated guanosine triphosphate. Test compounds were initially dissolved and diluted in 100% DMSO and further diluted into aqueous buffer, producing a final concentration of 5% DMSO. Typically, compounds were tested at concentrations between 0.2 nM and 10 μM. Reactions were started with addition of tritiated guanosine triphosphate and allowed to continue at 30° C. for 2 hours. Reactions were quenched with 100 μL stop buffer containing 10 mM EDTA and 1 μg/mL streptavidin-coated scintillation proximity beads. Reaction plates were incubated at 4° C. for 10 hours and then incorporation of radioactivity was determined by scintillation counting. The table below lists the IC₅₀ values of compounds determined using this procedure.

| Compound # | IC₅₀ (μM) |
|---|---|
| 261 | 0.003 |
| 265 | 0.009 |
| 266 | 0.011 |
| 267 | 0.010 |
| 269 | 0.021 |
| 270 | 0.026 |
| 271 | 0.015 |
| 272 | 0.030 |
| 273 | 0.0043922 |
| 274 | 0.0024472 |
| 275 | 0.0101305 |
| 278 | 0.002574 |
| 279 | 0.004021 |
| 280 | 0.002183 |
| 283 | 0.0043015 |
| 284 | 0.006223 |
| 286 | 0.004331 |
| 287 | 0.002708 |
| 288 | 0.001798 |
| 289 | 0.002031 |
| 290 | 0.001623 |
| 291 | 0.001941 |
| 292 | 0.00202 |
| 293 | 0.001712 |
| 294 | 0.0015535 |
| 295 | 0.008471 |
| 296 | 0.001008 |
| 297 | 0.001672 |
| 298 | 0.0071685 |
| 299 | 0.0010015 |
| 300 | 0.000769 |
| 301 | 0.003354 |
| 302 | 0.002684 |
| 303 | 0.006815 |
| 304 | 0.009 |
| 306 | 0.008 |
| 307 | 0.004 |
| 308 | 0.007 |
| 309 | 0.006 |
| 310 | 0.006 |
| 311 | 0.005 |
| 312 | 0.004 |
| 313 | 0.002 |
| 314 | 0.011 |
| 315 | 0.002 |
| 316 | 0.005 |
| 317 | 0.005 |
| 318 | 0.009 |
| 319 | 0.011 |
| 320 | 0.003 |
| 321 | 0.008 |
| 322 | 0.009 |
| 323 | 0.009 |
| 324 | 0.006 |
| 325 | 0.007 |
| 326 | 0.006 |
| 448 | 0.005053 |
| 496 | 0.200 |
| 497 | 0.023 |
| 498 | 0.020 |
| 499 | 0.004 |

### FORMULATION EXAMPLES

The following are representative pharmaceutical formulations containing a compound of Formula (I).

### Formulation Example 1

### Tablet Formulation

The following ingredients are mixed intimately and pressed into single scored tablets.

| | Quantity per |
|---|---|
| Ingredient | tablet, mg |
| compound | 400 |
| cornstarch | 50 |
| croscarmellose sodium | 25 |
| lactose | 120 |
| magnesium stearate | 5 |

### Formulation Example 2

### Capsule Formulation

The following ingredients are mixed intimately and loaded into a hard-shell gelatin capsule.

| | Quantity per |
|---|---|
| Ingredient | capsule, mg |
| compound | 200 |
| lactose, spray-dried | 148 |
| magnesium stearate | 2 |

### Formulation Example 3

### Suspension Formulation

The following ingredients are mixed to form a suspension for oral administration.

| Ingredient | Amount | |
|---|---|---|
| compound | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |

| | |
|---|---|
| distilled water | q.s. (quantity |
| | sufficient) to 100 mL |

### Formulation Example 4

### Injectable Formulation

The following ingredients are mixed to form an injectable formulation.

| Ingredient | Amount |
|---|---|
| compound | 0.2 mg-20 mg |
| sodium acetate buffer solution, 0.4 M | 2.0 mL |
| HCl (1N) or NaOH (1N) | q.s. to suitable pH |
| water (distilled, sterile) | q.s. to 20 mL |

### Formulation Example 5

### Suppository Formulation

A suppository of total weight 2.5 g is prepared by mixing the compound with Witepsol® H-15 (triglycerides of saturated vegetable fatty acid; Riches-Nelson, Inc., New York), and has the following composition:

| Ingredient | Amount |
|---|---|
| compound | 500 mg |
| Witepsol ® H-15 | balance |

1. A compound of Formula (I) or a pharmaceutically acceptable salt thereof 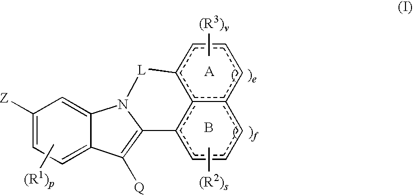
wherein:
ring A and B together contain 1 to 4 ring heteroatoms independently selected from O, N, NR^{b}, S, S(O), and S(O)₂; [symbol]
represents a single or double bond;
e is 0 or 1;
f is 0 or 1;
L is C₂ to C₆ alkylene optionally substituted with (R^{a})ₙ, wherein one —CH₂— group is optionally replaced with —NR^{b}—, >(C═O), —S—, —S(O)—, —S(O)₂—, or —O— and optionally two —CH₂— groups together form a double bond;
R^{a} is selected from the group consisting of halo, amino, substituted amino, acyl, acylamino, aminocarbonyl, alkyl, substituted alkyl, alkenyl, substituted alkenyl, carboxy ester, hydroxyl, alkoxy, substituted alkoxy, oxo, heterocyclyl, and substituted heterocyclyl or two R^{a} attached to a common carbon atom together from a spiro cycloalkyl, substituted cycloalkyl, heterocyclic, or substituted heterocyclic ring;
n is 0, 1, or 2;
R^{b} is independently selected from the group consisting of hydrogen, acyl, aminocarbonyl, alkyl, substituted alkyl, and carboxy ester;
R¹ is selected from the group consisting of alkyl, substituted alkyl, haloalkyl, acyl, acylamino, aminocarbonyl, alkoxy, substituted alkoxy, amino, substituted amino, cyano, halo, and hydroxy;
R² and R³ are independently selected from the group consisting of alkyl, substituted alkyl, alkenyl, substituted alkenyl, alkynyl, substituted alkynyl, amidino, haloalkyl, acyl, acyl-C(O)—, acylamino, aminocarbonyl, alkoxy, substituted alkoxy, amino, substituted amino, aminocarbonylamino, (carboxyl ester)amino, carboxyl, carboxyl ester, cyano, halo, hydroxy, heterocyclyl, substituted heterocyclyl, aryl, substituted aryl, heteroaryl, substituted heteroaryl, and oxo, or two of R² or two of R³ together form a fused or spiro cycloalkyl, substituted cycloalkyl, heterocyclic, or substituted heterocyclic ring or a fused aryl, substituted aryl, heteroaryl, or substituted heteroaryl ring;
p is 0, 1, 2, or 3;
v and s are independently 0, 1, 2, 3, 4, or 5, provided that when ring A is aromatic, at least one of R² or R³ is selected from the group consisting of substituted alkyl, acyl, acyl-C(O)—, aminocarbonyl, acylamino, alkoxy, substituted alkoxy, amino, substituted amino, halo, hydroxy, heterocyclyl, substituted heterocyclyl, aryl, substituted aryl, heteroaryl, and substituted heteroaryl;
Q is selected from the group consisting of cycloalkyl, substituted cycloalkyl, cycloalkenyl, substituted cycloalkenyl, heterocyclic, and substituted heterocyclic;
Z is selected from the group consisting of
(a) carboxy and carboxy ester;
(b) —C(X⁴)NR¹⁸R¹⁹, wherein X⁴ is ═O, ═NH, or ═N-alkyl, R¹⁸ and R¹⁹ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic or, alternatively, R¹⁸ and R¹⁹ together with the nitrogen atom pendent thereto, form a heterocyclic, a substituted heterocyclic, a heteroaryl or a substituted heteroaryl ring group;
(c) —C(X³)NR²¹S(O)₂R⁴ or —C(X³)NR²¹S(O)R⁴, wherein X³ is selected from ═O, ═NR²⁴, and ═S, wherein R²⁴ is hydrogen, alkyl, or substituted alkyl; R⁴ is selected from alkyl, substituted alkyl, aryl, substituted aryl, heteroaryl, substituted heteroaryl, heterocyclic, substituted heterocyclic, and NR²²R²³ wherein R²¹, R²², and R²³ are independently hydrogen, alkyl, substituted alkyl, cycloalkyl, or substituted cycloalkyl; or alternatively, R²¹ and R²² or R²² and R²³ together with the atoms bound thereto join together to form an optionally substituted heterocyclic group;
(d) —C(X²)—N(R³¹)CR³²R³³C(═O)R³⁴, wherein X² is selected from ═O, ═S, and ═NR¹¹, where R¹¹ is hydrogen or alkyl, R³⁴ is selected from —OR¹⁷ and —NR¹⁸R¹⁹ where R¹⁷ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R¹⁸ and R¹⁹ are as defined above;
R³² and R³³ are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic;
or, alternatively, R³² and R³³ as defined are taken together with the carbon atom pendent thereto to form a cycloalkyl, substituted cycloalkyl, heterocyclic or substituted heterocyclic group,
or, still further alternatively, one of R³² or R³³ is hydrogen, alkyl or substituted alkyl, and the other is joined, together with the carbon atom pendent thereto, with either the R¹⁷ and the oxygen atom pendent thereto or R¹⁸ and the nitrogen atom pendent thereto to form a heterocyclic or substituted heterocyclic group;
R³¹ is selected from hydrogen and alkyl or, when R³² and R³³ are not taken together to form a ring and when R³² or R³³ and R¹⁷ or R¹⁸ are not joined to form a heterocyclic or substituted heterocyclic group, then R³¹, together with the nitrogen atom pendent thereto, may be taken together with one of R³² and R³³ to form a heterocyclic or substituted heterocyclic ring group;
(e) —C(X²)—N(R³¹)CR²⁵R²⁶R²⁷, wherein X² and R³¹ are defined above, and R²⁵, R²⁶ and R²⁷ are independently selected from the group consisting of alkyl, substituted alkyl, aryl, substituted aryl, heterocyclic, substituted heterocyclic, heteroaryl and substituted heteroaryl, or R²⁵ and R²⁶ together with the carbon atom pendent thereto form a cycloalkyl, substituted cycloalkyl, heterocyclic or substituted heterocyclic group; and
(f) a carboxylic acid isostere wherein said isostere is not as defined in (a)-(e).

2. A compound of claim 1 of Formula (II) or a pharmaceutically acceptable salt thereof 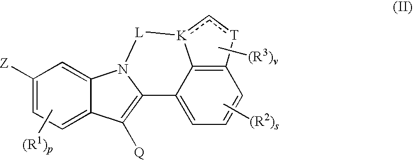
wherein:
Z, Q, L, R^{b}, R¹, R², R³, p, v, s, and [symbol] are previously defined; K is N or C, and
T is selected from the group consisting of N, NR^{b}, CH, CH₂, CHR³, CR³, O, S, S(O), and S(O)₂, wherein at least one of K or T is N or NR^{b}, and when one of [symbol] is a double bond, at least one of R² or R³ is selected from the group consisting of substituted alkyl, acyl, acyl-C(O)—, aminocarbonyl, acylamino, alkoxy, substituted alkoxy, amino, substituted amino, halo, hydroxy, heterocyclyl, substituted heterocyclyl, aryl, substituted aryl, heteroaryl, and substituted heteroaryl or two of R² or two of R³ together form a fused cycloalkyl, substituted cycloalkyl, heterocyclic, substituted heterocyclic, aryl, substituted aryl, heteroaryl, or substituted heteroaryl ring.

3. A compound of claim 2 of Formula (IIa) or a pharmaceutically acceptable salt thereof 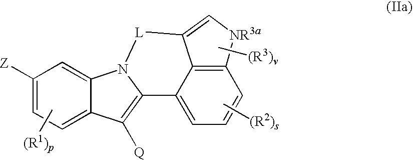
wherein:
Z, Q, L, R¹, R², R³, p, v, and s are previously defined; R^{3a} is H or R³; and at least one of R², R³, or R^{3a} is selected from the group consisting of substituted alkyl, acyl, acyl-C(O)—, aminocarbonyl, acylamino, alkoxy, substituted alkoxy, amino, substituted amino, halo, hydroxy, heterocyclyl, substituted heterocyclyl, aryl, substituted aryl, heteroaryl, and substituted heteroaryl.

4. A compound of claim 2 of Formula (IIb), (IIe), (IId), (IIe), or (IIf) or a pharmaceutically acceptable salt thereof 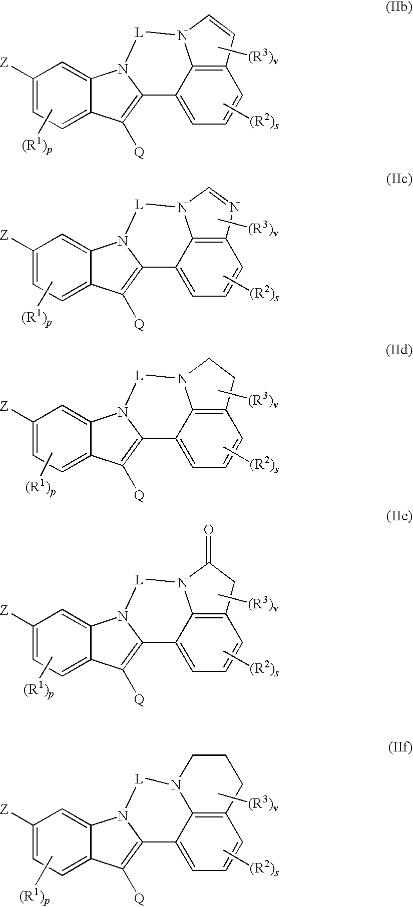
wherein:
Z, Q, L, R¹, R², R³, p, v, and s are previously defined; R^{3a} is H or R³; and for (IIb) and (IIc) at least one of R², R³, or R^{3a} is selected from the group consisting of substituted alkyl, acyl, aminocarbonyl, acylamino, alkoxy, substituted alkoxy, amino, substituted amino, halo, hydroxy, heterocyclyl, substituted heterocyclyl, aryl, substituted aryl, heteroaryl, substituted heteroaryl, and oxo.

5. A compound of claim 1 of Formula (IIIa), (IIIb) or (IIIc) or a pharmaceutically acceptable salt thereof 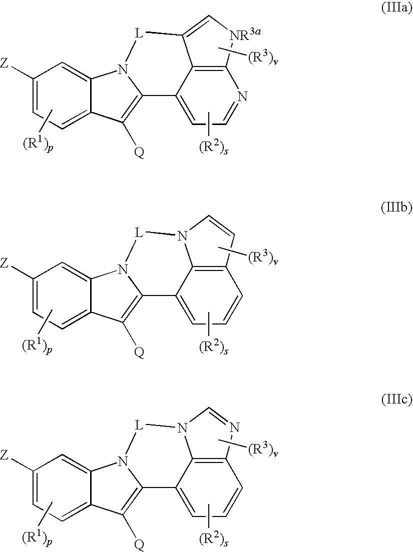
wherein:
Z, Q, L, R¹, R², R³, p, v, and s are previously defined; R^{3a} is H or R³; and at least one of R², R³, or R^{3a} is selected from the group consisting of substituted alkyl, acyl, substituted acyl, alkoxy, substituted alkoxy, amino, substituted amino, halo, hydroxy, heterocyclyl, substituted heterocyclyl, aryl, substituted aryl, heteroaryl, and substituted heteroaryl.

6. A compound of claim 1 wherein L is —CH₂(CH₂)ₙCH₂— where n is 0, 1 or 2.

7. A compound of claim 1 wherein L is C₂ to C₄ alkylene optionally substituted with R^{a}, wherein one —CH₂— group is —NR^{b}—.

8. A compound of claim 7 wherein R^{b} is selected from the group consisting of

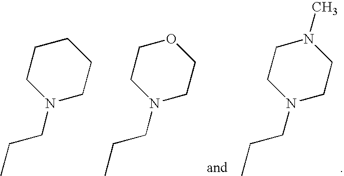

9. A compound of claim 1 wherein L is substituted with R^{a}, and R^{a} is selected from the group consisting of substituted alkyl, amino, substituted amino, aminocarbonyl, heterocyclyl, hydroxy, and substituted alkoxy.

10. A compound of claim 9 wherein R^{a} is selected from the group consisting of: 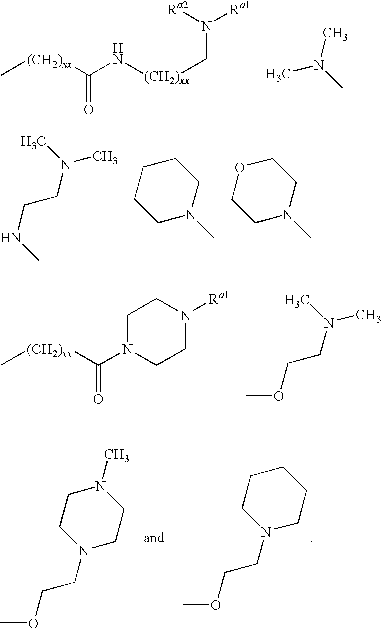
where each xx is independently 0, 1, 2, 3, or 4; and
R^{a1} and R^{a2} are independently selected from the group consisting of hydrogen, alkyl, substituted alkyl, acyl, sulfonyl and substituted sulfonyl.

11. A compound of claim 1 wherein R³ is selected from the group consisting of substituted alkyl, amino, substituted amino, acyl, acyl-C(O)—, heterocyclyl, hydroxy, and substituted alkoxy, or two R³ together form a spiro cycloalkyl, substituted cycloalkyl, heterocyclic, or substituted heterocyclic ring.

12. A compound of claim 11 wherein R³ is selected from the group consisting of:

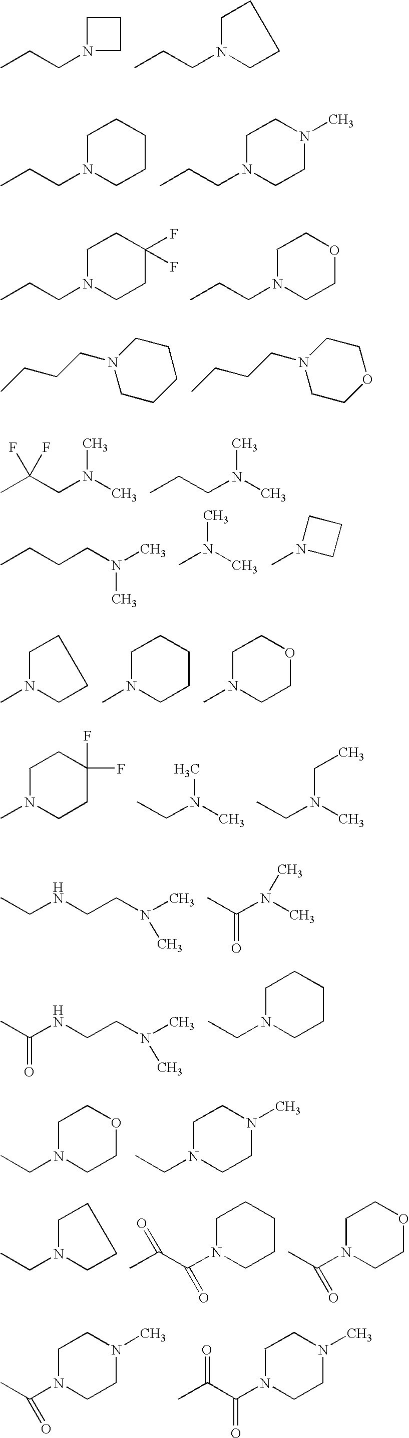

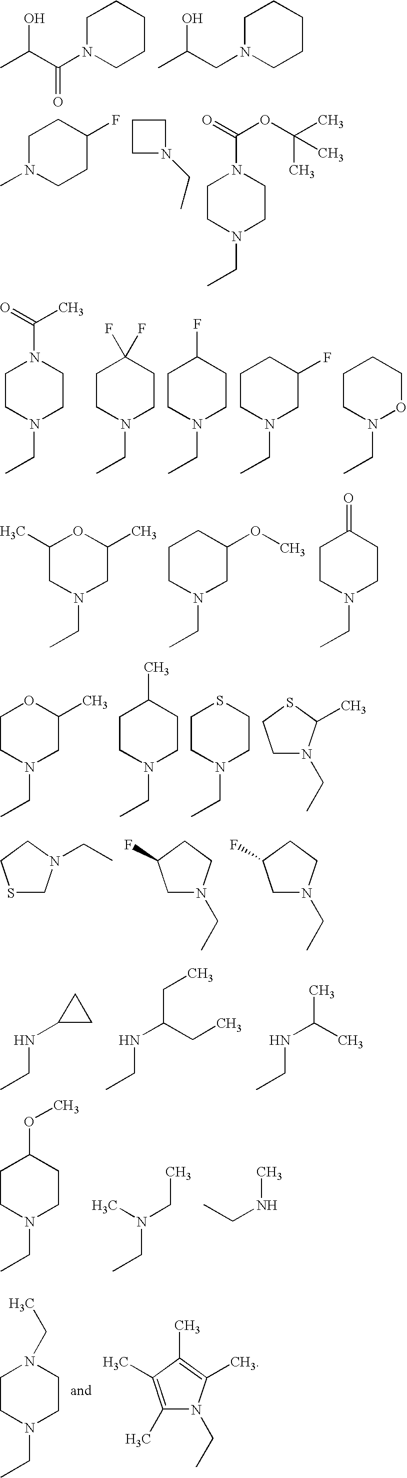

13. A compound of claim 1 wherein R² is selected from the group consisting of substituted alkoxy and heteroaryl.

14. A compound of claim 13 wherein R² is

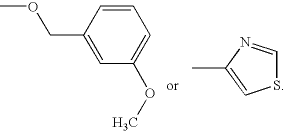

15. A compound of claim 1 wherein Z is carboxy, carboxy ester, carboxylic acid isostere, —C(O)NR¹⁸R¹⁹, or —C(O)NHS(O)₂R⁴, wherein R¹⁸ and R¹⁹ are as defined in claim 1 and R⁴ is alkyl or aryl.

16. A compound of claim 15 wherein Z is carboxy, methyl carboxylate, ethyl carboxylate, 6-(β-D-glucuronic acid) ester, 1H-tetrazol-5-yl, 5-oxo-4,5-dihydro-1,2,4-oxadiazol-3-yl, N-2-cyano-ethylamide, N-2-(1H-tetrazol-5-yl)ethylamide, methylsulfonylaminocarbonyl, trifluoromethylsulfonylaminocarbonyl, cyclopropylsulfonylamino, or phenylsulfonylaminocarbonyl.

17. A compound of claim 16 wherein Z is carboxy.

18. A compound of claim 1 wherein Q is cycloalkyl or substituted cycloalkyl.

19. A compound of claim 18 wherein Q is cyclohexyl or fluoro substituted cyclohexyl.

20. A compound of claim 1 wherein p is 0.

21. A compound of claim 1, wherein the compound is: 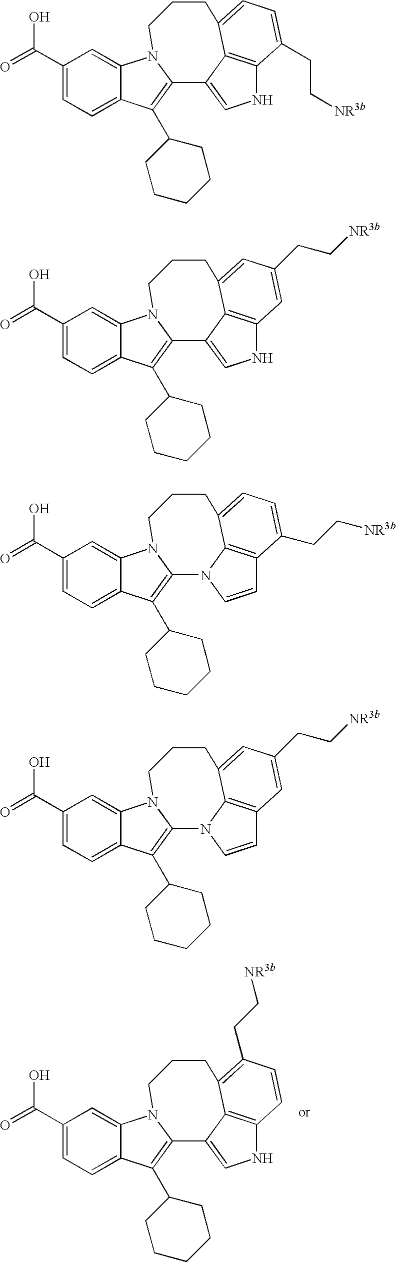 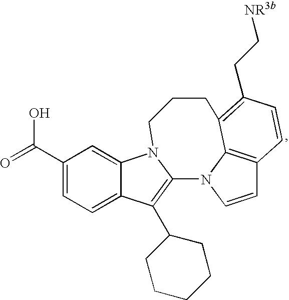
wherein R^{3b} is selected from the group consisting of hydrogen, alkyl, substituted alkyl, acyl, sulfonyl, substituted sulfonyl, and aminocarbonyl.

22. A compound or a pharmaceutically acceptable salt thereof, which compound is selected from Table 1.

23. A compound or a pharmaceutically acceptable salt thereof, which compound is selected from Table 2.

24. A pharmaceutical composition comprising a pharmaceutically acceptable carrier and a therapeutically effective amount of a compound of any one of claims 1, 22, or 23.

25. A method for treating a viral infection in a patient mediated at least in part by a virus in the Flaviviridae family of viruses which method comprises administering to the patient a compound of claim 1.

26. The method of claim 25 wherein said viral infection is a hepatitis C mediated viral infection.

27. The method of claim 25 in combination with the administration of a therapeutically effective amount of one or more agents active against hepatitis C virus.

28. The method of claim 27 wherein said agent active against hepatitis C virus is an inhibitor of HCV proteases, HCV polymerase, HCV helicase, HCV NS4B protein, HCV entry, HCV assembly, HCV egress, HCV NS5A protein, or inosine 5′-monophosphate dehydrogenase.

29. The method of claim 27 wherein said agent active against hepatitis C virus is interferon.
